(19) United States
(12) Patent Application Publication
(10) Pub. No.: US 2025/0263695 A1
(43) Pub. Date: Aug. 21, 2025

(54) NOVEL SITES FOR SAFE GENOMIC INTEGRATION AND METHODS OF USE THEREOF

(71) Applicant: BlueRock Therapeutics LP, Cambridge, MA (US)

(72) Inventors: Chew-Li SOH, Cambridge, MA (US); Mark J. TOMISHIMA, Cambridge, MA (US); Conor B. MCAULIFFE, Cambridge, MA (US); Dan Charles WILKINSON, Jr., Cambridge, MA (US); Benjamin BURNETT, Cambridge, MA (US)

(21) Appl. No.: 18/857,388

(22) PCT Filed: Apr. 28, 2023
(86) PCT No.: PCT/US2023/066396

§ 371 Date: Oct. 16, 2024

Related U.S. Application Data
(60) Provisional application No. 63/336,248, filed on Apr. 28, 2022.

(51) Int. Cl. C12N 15/11; C12N 5/074; C12N 5/078; C12N 9/22; C12N 15/86; C12N 15/90

(52) U.S. Cl. CPC C12N 15/111; C12N 5/0634; C12N 5/0696; C12N 9/224; C12N 9/226; C12N 15/86; C12N 15/907; C12N 2310/20; C12N 2510/00; C12N 2740/10043; C12N 2740/15043; C12N 2750/14143

(57) ABSTRACT

The present disclosure is directed to genetically modified cells that express one or more transgenes at a sustained expression level from a site for safe genomic integration and stable expression. Also provided are methods of making the cells and nucleic acid vectors that can be used to make the cells.

### CROSS-REFERENCE TO RELATED APPLICATION

This application claims priority from U.S. Provisional Application No. 63/336,248, filed Apr. 28, 2022, the content of which is incorporated herein by reference in its entirety.

### SEQUENCE LISTING

The instant application contains a Sequence Listing submitted electronically IN XML format and is hereby incorporated by reference in its entirety. The electronic copy of the Sequence Listing, created on Apr. 27, 2023, is named 025450_WO017_SL.xml and is 379,876 bytes in size.

### BACKGROUND

Many efforts to safely integrate transgenes into a genome have been made at so-called “genomic safe harbor” sites. Safe harbor sites in the genome are those where a nucleic acid (e.g., an exogenous gene) can be introduced without disrupting the expression or regulation of adjacent genes, and therefore the normal functioning of the cell. Three genomic sites—AAVS1, CCR5, and ROSA26—are traditionally considered safe harbor sites and have been used in most targeted transgene integrations. AAVS1 is a region for the rare genomic integration of AAV genome and has been found to allow robust expression without disrupting cell function. CCR5 was serendipitously identified because a naturally-occurring CCR5-delta-32 mutation results in an HIV-resistant phenotype; the disposability of the gene makes it an ideal integration site. The ROSA26 locus was originally identified in mouse embryonic stem cells through a lentiviral gene trap approach.

While these genomic safe harbor sites allow robust transgene expression under a given cell context, they may not support faithful transgene expression in other cell lineages or after a change in cell state. This is because reciprocal interactions between a transgene and the host cell's genomic context can affect the expression of the transgene, leading to attenuation or complete silencing of transgene expression (e.g., through DNA methylation). More critically, these sites of genomic integration may also affect the expression of endogenous genes in the vicinity of the insertion site, thus affecting normal host cell function.

### SUMMARY OF THE DISCLOSURE

The present disclosure is based, at least in part, on the identification of intergenic sites in the genome that remain transcriptionally active in different cell types and under different cell states, including maturation phases, such that an exogenous nucleotide sequence of interest (e.g., a transgene encoding a protein or an RNA) integrated therein remains expressed and functional as the cell undergoes proliferation and cell state changes.

Accordingly, in one aspect, the present disclosure provides a genetically modified cell, e.g., a mammalian (e.g., human) cell, comprising an exogenous nucleotide sequence integrated in a sustained transcriptionally active payload region (STAPLR) in the genome of the cell, wherein the STAPLR is selected from the group consisting of the intergenic region between the RPL34 gene and the OSTC gene; the intergenic region between the ACTB gene and the FSCN1 gene; the intergenic region between the AKIRIN1 gene and the NDUFS5 gene; the intergenic region between the PRDX1 gene and the AKR1A1 gene; the intergenic region between the PTGES3 gene and the NACA gene; the intergenic region between the MLF2 gene and the PTMS gene; the intergenic region between the RAB13 gene and the RPS27 gene; the intergenic region between the JTB gene and the RAB13 gene; the intergenic region between the AKR1A1 gene and the NASP gene; the intergenic region between the NDUFS5 gene and the MA CF1 gene; the intergenic region between the SRSF9 gene and the DYNLL1 gene; the intergenic region between theMYL6B gene and theMYL6 gene; the intergenic region between the GPX1 gene and the RHOA gene; the intergenic region between the HNRNPA2B1 gene and the CBX3 gene; the intergenic region between the ROMO gene and the RBM39 gene; the intergenic region between the PA2G4 gene and the RPL41 gene; and the intergenic region between the NDUFB10 gene and the RPS2 gene.

In some embodiments, the intergenic region between the RPL34 gene and the OSTC gene comprises a nucleotide sequence at least 95% (e.g., at least 96%, at least 97%, at least 98%, at least 99%, or 100%) identical to SEQ ID NO: 1, or a nucleotide sequence sufficiently similar to SEQ ID NO: 1 as necessary for the function of the sequence to remain intact (e.g., without adverse effects on the cell).

In some embodiments, the intergenic region between the ACTB gene and the FSCN1 gene comprises a nucleotide sequence at least 95% (e.g., at least 96%, at least 97%, at least 98%, at least 99%, or 100%) identical to SEQ ID NO: 2, or a nucleotide sequence sufficiently similar to SEQ ID NO: 2 as necessary for the function of the sequence to remain intact (e.g., without adverse effects on the cell).

In some embodiments, the intergenic region between the AKIRIN1 gene and the NDUFS5 gene comprises a nucleotide sequence at least 95% (e.g., at least 96%, at least 97%, at least 98%, at least 99%, or 100%) identical to SEQ ID NO: 3, or a nucleotide sequence sufficiently similar to SEQ ID NO: 3 as necessary for the function of the sequence to remain intact (e.g., without adverse effects on the cell).

In some embodiments, the intergenic region between the PRDX1 gene and the AKR1A1 gene comprises a nucleotide sequence at least 95% (e.g., at least 96%, at least 97%, at least 98%, at least 99%, or 100%) identical to SEQ ID NO: 4, or a nucleotide sequence sufficiently similar to SEQ ID NO: 4 as necessary for the function of the sequence to remain intact (e.g., without adverse effects on the cell).

In some embodiments, the intergenic region between the PTGES3 gene and the NACA gene comprises a nucleotide sequence at least 95% (e.g., at least 96%, at least 97%, at least 98%, at least 99%, or 100%) identical to SEQ ID NO: 5, or a nucleotide sequence sufficiently similar to SEQ ID NO: 5 as necessary for the function of the sequence to remain intact (e.g., without adverse effects on the cell).

In some embodiments, the intergenic region between the MLF2 gene and the PTMS gene comprises a nucleotide sequence at least 95% (e.g., at least 96%, at least 97%, at least 98%, at least 99%, or 100%) identical to SEQ ID NO: 6, or a nucleotide sequence sufficiently similar to SEQ ID NO: 6 as necessary for the function of the sequence to remain intact (e.g., without adverse effects on the cell).

In some embodiments, the intergenic region between the RAB13 gene and the RPS27 gene comprises a nucleotide sequence at least 95% (e.g., at least 96%, at least 97%, at least 98%, at least 99%, or 100%) identical to SEQ ID NO: 7, or a nucleotide sequence sufficiently similar to SEQ ID NO: 7 as necessary for the function of the sequence to remain intact (e.g., without adverse effects on the cell).

In some embodiments, the intergenic region between the JTB gene and the RAB13 gene comprises a nucleotide sequence at least 95% (e.g., at least 96%, at least 97%, at least 98%, at least 99%, or 100%) identical to SEQ ID NO: 8, or a nucleotide sequence sufficiently similar to SEQ ID NO: 8 as necessary for the function of the sequence to remain intact (e.g., without adverse effects on the cell).

In some embodiments, the intergenic region between the AKR1A1 gene and the NASP gene comprises a nucleotide sequence at least 95% (e.g., at least 96%, at least 97%, at least 98%, at least 99%, or 100%) identical to SEQ ID NO: 9, or a nucleotide sequence sufficiently similar to SEQ ID NO: 9 as necessary for the function of the sequence to remain intact (e.g., without adverse effects on the cell).

In some embodiments, the intergenic region between the NDUFS5 gene and the MACF1 gene comprises a nucleotide sequence at least 95% (e.g., at least 96%, at least 97%, at least 98%, at least 99%, or 100%) identical to SEQ ID NO: 10, or a nucleotide sequence sufficiently similar to SEQ ID NO: 10 as necessary for the function of the sequence to remain intact (e.g., without adverse effects on the cell).

In some embodiments, the intergenic region between the SRSF9 gene and the DYNLL1 gene comprises a nucleotide sequence at least 95% (e.g., at least 96%, at least 97%, at least 98%, at least 99%, or 100%) identical to SEQ ID NO: 11, or a nucleotide sequence sufficiently similar to SEQ ID NO: 11 as necessary for the function of the sequence to remain intact (e.g., without adverse effects on the cell).

In some embodiments, the intergenic region between the MYL6B gene and the MYL6 gene comprises a nucleotide sequence at least 95% (e.g., at least 96%, at least 97%, at least 98%, at least 99%, or 100%) identical to SEQ ID NO: 12, or a nucleotide sequence sufficiently similar to SEQ ID NO: 12 as necessary for the function of the sequence to remain intact (e.g., without adverse effects on the cell).

In some embodiments, the intergenic region between the GPX1 gene and the RHOA gene comprises a nucleotide sequence at least 95% (e.g., at least 96%, at least 97%, at least 98%, at least 99%, or 100%) identical to SEQ ID NO: 13, or a nucleotide sequence sufficiently similar to SEQ ID NO: 13 as necessary for the function of the sequence to remain intact (e.g., without adverse effects on the cell).

In some embodiments, the intergenic region between the HNRNPA2B1 gene and the CBX3 gene comprises a nucleotide sequence at least 95% (e.g., at least 96%, at least 97%, at least 98%, at least 99%, or 100%) identical to SEQ ID NO: 14, or a nucleotide sequence sufficiently similar to SEQ ID NO: 14 as necessary for the function of the sequence to remain intact (e.g., without adverse effects on the cell).

In some embodiments, the intergenic region between the ROMO gene and the RBM39 gene comprises a nucleotide sequence at least 95% (e.g., at least 96%, at least 97%, at least 98%, at least 99%, or 100%) identical to SEQ ID NO: 15, or a nucleotide sequence sufficiently similar to SEQ ID NO: 15 as necessary for the function of the sequence to remain intact (e.g., without adverse effects on the cell).

In some embodiments, the intergenic region between the PA2G4 gene and the RPL41 gene comprises a nucleotide sequence at least 95% (e.g., at least 96%, at least 97%, at least 98%, at least 99%, or 100%) identical to SEQ ID NO: 16, or a nucleotide sequence sufficiently similar to SEQ ID NO: 16 as necessary for the function of the sequence to remain intact (e.g., without adverse effects on the cell).

In some embodiments, the intergenic region between the NDUFB10 and the RPS2 gene comprises a nucleotide sequence at least 95% (e.g., at least 96%, at least 97%, at least 98%, at least 99%, or 100%) identical to SEQ ID NO: 16, or a nucleotide sequence sufficiently similar to SEQ ID NO: 97 as necessary for the function of the sequence to remain intact (e.g., without adverse effects on the cell).

Also provided herein are methods of generating these genetically modified mammalian cells, as well as DNA constructs for introducing nucleotide sequences of interest into the novel genomic integration sites herein. Accordingly, in one aspect, the present disclosure provides a method for modifying a mammalian cell, comprising integrating a nucleotide sequence of interest (i.e., an exogenous nucleotide sequence) into a STAPLR described herein. In some embodiments, the integrating step is performed by using a CRISPR/Cas system; a Cre/Lox system; a FLP-FRT system; a TALEN system; a ZFN system; homing endonucleases; random integration; homologous recombination; a transposase; or a non-nuclease-dependent viral vector, optionally selected from a retroviral vector, an adeno-associated viral (AAV) vector, and a lentiviral vector. In further embodiments, the CRISPR/Cas system comprising a guide RNA, and wherein the STAPLR is the intergenic region between (i) the RPL34 gene and the OSTC gene and the gRNA is selected from SEQ ID NOs: 25-32, (ii) the ACTB gene and the FSCN1 gene and the gRNA is selected from SEQ ID NOs: 33-54, (iii) the AKIRIN1 gene and the NDUFS5 gene and the gRNA is selected from SEQ ID NOs: 55-70, or (iv) the PRDX1 gene and the AKR1A1 gene and the gRNA is selected from SEQ ID NOs: 71-92.

In some embodiments, the CRISPR/Cas system comprises a gRNA-dependent nuclease of type I, type II, type III, type IV, or type V, or a variant thereof. In further embodiments, the CRISPR/Cas system comprises a gRNA-dependent nuclease selected from the group consisting of Cas9, Cpf1, Cas1, Cas1B, Cas2, Cas3, Cas4, Cas5, Cas6, Cas7, Cas8, Cas12, Cas13, Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, CasX, CasY, Csx3, Csx1, Csx15, Csf1, Csf2, Csf3, CasPhi, MAD7, and Csf4.

In another aspect, the present disclosure provides a DNA molecule comprising a nucleotide sequence of interest flanked by a 5′ homologous region (HR) and a 3′ HR, wherein the 5′ and 3′ HRs are at least 85% (e.g., at least 90, 95, 96, 97, 98, or 99%) homologous, or 100% identical, to a first genomic region (GR) and a second GR, respectively, in a STAPLR described herein. In some embodiments, each of the 5′ and 3′ HRs is independently about at least 50, at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1000, at least 1100, at least 1200, at least 1300, at least 1400, at least 1500, at least 1600, at least 1700, at least 1800, at least 1900, or at least 2000 base pairs long. In some embodiments, the HRs are each 200 to 2000 (e.g., 300 to 2500, 400 to 2000, or 500 to 1500) base pairs long. In further embodiments, the 5′ and 3′ HRs are at least 90% (e.g., at least 95%) homologous to SEQ ID NOs: 17 and 18, SEQ ID NOs: 19 and 20, SEQ ID NOs: 21 and 22, SEQ ID NOs: 23 and 24, SEQ ID NOs: 93 and 94, or SEQ ID NOs: 95 and 96, respectively.

In some embodiments, the exogenous nucleotide sequence or the nucleotide sequence of interest comprises a transgene. In further embodiments, the transgene comprises a coding sequence (e.g., for a protein or an RNA) and one or more regulator elements. In some embodiments, the one or more regulator elements include a constitutive or inducible promoter directing the transcription of the coding sequence. In some embodiments, the transgene encodes a therapeutic protein (e.g., a protein the deficiency or defectiveness of which leads to a disease such as a genetic disease; a cytokine; or a recombinant antigen receptor); a cellular marker; or a protein that regulates the differentiation state or activity of the cell (e.g., a reprogramming factor). In some embodiments, the transgene encodes SOX10, IL-10, IL-12, CD19t, or ThPOK.

In some embodiments of the present disclosure, the mammalian cell is a human cell. In some embodiments, the mammalian cell (e.g., human cell) is a pluripotent stem cell (PSC; e.g., an induced PSC (iPSC) or an embryonic stem cell (ESC)). In some embodiments, the mammalian cell (e.g., human cell) is a) a cell in the immune system (e.g., a T cell, a natural killer cell, a dendritic cell, a macrophages/monocyte, or a hematopoietic progenitor or precursor cell thereof); b) a cell in the cardiovascular system (e.g., a ventricular cardiomyocyte, a nodal cell, or a cardiac progenitor or precursor cell thereof); c) a cell in the metabolic system (e.g., a hepatocyte or a pancreatic beta-cell, or a progenitor or precursor cell thereof); d) a cell in the central nervous system (e.g., a sensory neuron, a motor neuron, an interneuron, a microglial cell, an oligodendrocyte, or a progenitor or precursor cell thereof); e) a muscle cell (e.g., a skeletal muscle cell or a smooth muscle cell, or a progenitor or precursor cell thereof); f) an adipose cell or a progenitor or precursor cell thereof); or g) a cell in the ocular system (e.g., a retinal pigment epithelium cell, a photoreceptor cell, or a progenitor or precursor cell thereof). Additional cell types of the present disclosure include those described below.

Also provided herein are pharmaceutical compositions comprising the genetically engineered cells herein and a pharmaceutically acceptable carrier, and gene editing systems comprising the DNA molecule as disclosed herein and the requisite gene editing system for incorporating the nucleotide sequence of interest on the DNA molecule (e.g., a nuclease and gRNA) into the STAPLR.

In another aspect, the present disclosure provides a method for identifying a sustained transcriptionally active payload region (STAPLR) in the genome of a mammalian cell, the method comprising: (i) performing single cell RNA sequencing analysis on a set of two or more mammalian cell types, wherein the sequencing analysis assigns a unique transcriptome to each cell type; (ii) assigning a Prevalence Score to a constituent gene in the transcriptome, wherein the Prevalence Score represents the fraction of the mammalian cell types containing at least one transcript of the gene in the set of mammalian cell types; (iii) identifying the constituent gene's neighboring gene(s) in the mammalian cell's genome, wherein the neighboring gene(s) do not overlap with the constituent gene; (iv) determining a Neighbor Score for pairs of non-overlapping genes or for regions comprising three or more genes identified in step (iii), wherein the Neighbor Score is the product of the Prevalence Scores of the individual genes in a pair or in a region; (v) ranking the Neighbor Scores; and (vi) selecting a pair of non-overlapping genes or a region comprising three or more non-overlapping genes based on a high ranking, thereby identifying the intergenic region between genes of the selected pair or region as a STAPLR. In some embodiments, the method further comprises (vii) selecting a targetable intergenic subregion in the STAPLR; and (viii) inserting a transgene at the selected subregion, wherein transcription of the transgene or gene circuit is sustained. In some embodiments, the targetable subregion comprises: no known promoter or enhancer regions, a minimal number of conserved regions, repetitive regions, epigenetic marks, and/or enzymatic hypersensitivity regions, and/or the nuclease is a CRISPR nuclease. In some embodiments, wherein the intergenic region is at least 30 (e.g., at least 40, at least 50, at least 75, or at least 100) base pairs in length, and/or does not comprise or comprises a minimal number of promoter regions, a CpG Island, an H3K4MeT epigenetic mark, an H3K4Me3 epigenetic mark, an H3K27Ac epigenetic mark, a DNase I hypersensitivity region, a conserved region, or a repetitive region.

Other features, objectives, and advantages of the invention are apparent in the detailed description that follows. It should be understood, however, that the detailed description, while indicating embodiments and aspects of the invention, is given by way of illustration only, not limitation. Various changes and modification within the scope of the invention will become apparent to those skilled in the art from the detailed description.

### BRIEF DESCRIPTION OF THE DRAWINGS

FIG. 1 is a dot plot showing the indel editing percentage obtained after Sanger sequencing examination using Synthego's ICE Analysis Tool. For each different STAPLR site, three different gRNAs were tested and the gRNA with the highest indel editing percentage is encircled. The solid horizontal line indicates the mean indel editing percentage of three different gRNAs per STAPLR site.

FIG. 2 is a diagram illustrating integration of a sequence coding for a 2A peptide and a sequence coding for the Tet-On 3G version of rtTA at the GAPDH locus. Left and right homology arms were designed to enable in-frame integration of the transgene immediately 5′ to the STOP codon of GAPDH. This permits expression of rtTA under endogenous GAPDH promoter control. iPSCs that have been edited with the targeting construct constitutively express the rtTA protein.

FIG. 3 is a diagram illustrating integration of each of the four STAPLR targeting constructs comprising the pTRE3G-eGFP-Sv40 transgene flanked by left and right homology arms at each STAPLR site in iPSCs constitutively expressing the rtTA protein. The addition of doxycycline allows binding of the rtTA protein and activation of GFP expression from the TRE3G promoter.

FIG. 4 is a panel of fluorescent microscope images depicting the expression of GFP in a pooled population of cells that had received doxycycline for 24 hours. The doxycycline was added to media 24 hours after Nucleofection of iPSCs with the STAPLR targeting construct and corresponding RNP. No GFP was observed in cells that did not receive doxycycline. Control iPSCs constitutively expressing rtTA that were treated with doxycycline but were not nucleofected with the STAPLR targeting construct and RNP also did not express GFP.

FIG. 5 is a panel of fluorescent microscope images depicting the expression of GFP in a pooled population of cells that had received doxycycline for 6 days. The doxycycline was added to media 24 hours after Nucleofection of iPSCs with the STAPLR targeting construct and corresponding RNP. No GFP was observed in cells that did not receive doxycycline. Control iPSCs constitutively expressing rtTA that were treated with doxycycline but were not nucleofected with the STAPLR targeting construct and RNP also did not express GFP.

FIG. 6 is a panel of flow cytometric histograms depicting induction of GFP expression in four different clonally-derived STAPLR iPSC lines over time under different concentrations of doxycycline. Cells were collected for analysis after 0, 3, 8, 24, 48 and 68 hours of doxycycline administration.

FIG. 7 is a panel of flow cytometric histograms depicting induction of GFP expression after treatment with 2 μg/ml doxycycline in four different clonally-derived STAPLR iPSC lines over time. The left panel shows the PRDX1-AKR1A1, ACTB-FSCN1, and RPL34-OSTC STAPLR lines and a wildtype unedited iPSC control line either without doxycycline treatment or with doxycycline treatment for 72 hours. The right panel shows the AKIRIN1-NDUFS5 STAPLR line either without doxycycline treatment or with doxycycline treatment for 6 days.

FIG. 8 is a panel of flow cytometric histograms depicting induction of GFP expression after treatment with 2 μg/ml doxycycline in four different clonally-derived STAPLR iPSC lines differentiated into myeloid progenitor cells. Doxycycline was added to the culture medium at day 12 of differentiation. The left panel shows the PRDX1-AKR1A1, ACTB-FSCN1, and RPL34-OSTC STAPLR lines and a wildtype unedited iPSC control line after 15 days of myeloid differentiation either without doxycycline treatment or with doxycycline treatment for 72 hours. The right panel shows theAKIRIN1-NDUFS5 STAPLR line after 18 days of myeloid differentiation either without doxycycline treatment or with doxycycline treatment for 6 days.

FIG. 9 is a panel of flow cytometric dot plots showing expression of the myeloid progenitor markers CD45, CD14 and CX3CR1 in the non-adherent myeloid population of STAPLR-targeted iPSC lines that had been differentiated past 30 days. The CD14 and CX3CR1 panel of cells was gated on CD45-positive cells.

FIG. 10 is a panel of flow cytometric histograms depicting induction of GFP expression in non-adherent myeloid progenitor cells after treatment with 2 μg/ml doxycycline in four differentiated clonally-derived STAPLR iPSC lines and a wildtype unedited iPSC control line. Doxycycline was added to the culture medium after day 30 of differentiation for six days.

FIG. 11 is a diagram illustrating integration of a targeting construct comprising the pTRE3G-CD19t-IL12 transgene flanked by left and right homology arms to allow integration at the PRDX1-AKR1A1 STAPLR site. This construct was transfected in iPSCs constitutively expressing the rtTA protein from the GAPDH endogenous promoter.

FIG. 12 is a panel of photographs showing live cell imaging of CD19t (truncated to prevent intracellular signal transduction) staining after 48 h of treatment with 2 μg/mL doxycycline either in a pooled sample of cells post-targeting with the PRDX1-AKR1A1 pTRE3G-CD19t-IL12 donor template, or in a clonal population of cells after single cell clonal density seeding compared to untreated cells. Panel A shows cells after targeting with a Cpf1-based RNP. Panel B shows cells after targeting with a Cas9-based RNP.

FIG. 13 is a panel of fluorescent microscope images depicting the expression of GFP in a pooled population of cells that had received doxycycline for 24 hours. The doxycycline was added to media 48 hours after Nucleofection of iPSCs with the PRDX1-AKR1A1 Site 2 targeting construct and three different RNPs which comprise three different gRNAs targeting Site 2. No GFP was observed in cells that did not receive doxycycline.

FIG. 14 is a panel of fluorescent microscope images depicting the expression of GFP in a pooled population of cells that had received doxycycline for 24 hours. The doxycycline was added to media 24 hours after Nucleofection of iPSCs with the PRDX1-AKR1A1 Site 3 targeting construct and three different RNPs which comprise three different gRNAs targeting Site 3. No GFP was observed in cells that did not receive doxycycline.

FIG. 15 is a panel of flow cytometric histograms depicting induction of GFP expression in a pooled population of cells after treatment with 2 μg/ml doxycycline. The doxycycline was added to media 48 hours after Nucleofection of iPSCs with the PRDX1-AKR1A1 Site 2 targeting construct and three different RNPs which comprise three different gRNAs targeting Site 2. Flow cytometric analysis was performed 5 days after doxycycline treatment. No GFP was observed in cells that did not receive doxycycline and in parental GAPDH::rtTA iPSCs that did not receive the targeting construct and RNP.

FIG. 16 is a panel of flow cytometric histograms depicting induction of GFP expression in a pooled population of cells after treatment with 2 μg/ml doxycycline. The doxycycline was added to media 24 hours after Nucleofection of iPSCs with the PRDX1-AKR1A1 Site 3 targeting construct and three different RNPs which comprise three different gRNAs targeting Site 3. Flow cytometric analysis was performed 6 days after doxycycline treatment. No GFP was observed in cells that did not receive doxycycline and in parental GAPDH::rtTA iPSCs that did not receive the targeting construct and RNP.

### DETAILED DESCRIPTION

Genetically engineered cells are important tools for cell therapy. But artificial gene circuitry in engineered cells is often subverted by transgene silencing over time, as the cells undergo proliferation, or changes in cell states or in vivo environment. Thus, there is a need for identifying genomic regions that are safe for transgene integration and also provide a chromatin landscape that remains open for transcription across cell types, cell states, and in vivo milieus. Integration of a transgene into such a site would allow the transgene to remain transcriptionally active during the life time of a cell therapy product.

Provided herein are compositions (e.g., of nucleic acid molecules and cells) and methods for genomically (genetically) engineering cells to achieve expression of a transgene across various cell or differentiation states, without affecting endogenous gene expression that may be detrimental to the cell or the therapeutic purpose of the cell in a cell therapy. The provided compositions and methods are based, at least in part, on the identification of chromatin landscapes comprising sustained transcriptionally active payload regions (STAPLRs) that remain transcriptionally active across cell types and differentiation cell states.

### I. STAPLRs

The present inventors have discovered that certain intergenic regions in the mammalian genome allow consistent levels of expression of transgenes integrated therein, regardless of cell type and/or even as the cell undergoes changes in its state (e.g., differentiation state, maturation, or activity state). This discovery greatly expands the repertoire of genomic sites where transgenes can be stably integrated and their expression can be maintained over changing cell states. The discovery thus solves a long-standing problem in transgene expression, for example, in the context of cell therapy. These intergenic regions are termed “sustained transcriptionally active payload region” (STAPLR) herein, where “payload” or “genomic payload” refers to one or more exogenous or heterologous nucleotide sequences introduced to the region. A STAPLR comprise an open chromatin landscape for landing genomic payloads. The chromosomal DNA in the STAPLR is in a conformation that is accessible to components of gene editing machinery and that allows integration of genetic material. In some instances, a STAPLR is in the vicinity of transcriptionally active genes.

One application of this discovery is the efficient generation of cells (e.g., therapeutic cells) that are first genetically modified and then made to change cell states, e.g., by differentiating or dedifferentiating. For example, the present genetic engineering method can be applied to iPSCs that are then differentiated into various cell types. In the past, when iPSCs are engineered to incorporate a transgene into their genome and then differentiated into the desired cell types, the transgene can become inactive upon iPSC differentiation. However, transgenes integrated into the STAPLRs as disclosed herein do not become inactive upon iPSC differentiation. Thus, the STAPLRs provide universal “landing pads” for transgene expression.

This stability in transgene expression is also advantageous after the therapeutic cells in a cell therapy are administered to a subject in need thereof (e.g., a human patient), where they may encounter different and varying milieus that would have shut down transgenes integrated elsewhere.

Furthermore, integrating transgenes within intergenic regions, rather than within genes, will cause minimal disruption to the expression or regulation of adjacent genes and therefore allow the normal functioning of the genetically engineered cell. Transgene integration at the STAPLRs also reduces the risk of causing unwanted effects in the cells (e.g., activating an oncogene or disrupting an essential gene such as a tumor suppressor gene). Furthermore, the STAPLRs, with their constantly transcriptionally active status, will allow for the testing and use of a wider range of regulatory elements (e.g., promoters and enhancers).

As used herein, an “intergenic region” is a stretch of nucleotide sequence located between two neighboring genes. An intergenic region can be of various sizes. For example, the intergenic region can be at least 30, 40, 50, 75, or 100 base pairs in length. In some embodiments, the intergenic region can be at least 150, 200, 300, 400, 500, 750, or 1000 base pairs length. In some embodiments, the intergenic region can be at least 1500, 2000, 2500, 3000, 3500, 5000, or 10000 base pairs in length. In some embodiments, the intergenic region can be at least 15000, 20000, 30000, 40000, 50000, 75000, or 100000 base pairs in length. In some embodiments, the intergenic region is 30 base pairs to 100000 base pairs in length. In some embodiments, the intergenic region is 50 base pairs to 75000 base pairs in length. In some embodiments, the intergenic region is 75 base pairs to 70000 in length.

STAPLRs of the present disclosure include, without limitation (with the NCBI Gene IDs for the human genes shown in parentheses): the intergenic region between the RPL34 gene (Gene ID: 6164) and the OSTC gene (Gene ID: 58505), the intergenic region between the ACTB gene (Gene ID: 60) and the FSCN1 gene (Gene ID: 6624), the intergenic region between the AKIRIN1 gene (Gene ID: 79647) and the NDUFS5 gene (Gene ID: 4725), the intergenic region between the PRDX1 gene (Gene ID: 5052) and the AKR1A1 gene (Gene ID: 10327), the intergenic region between the PTGES3 gene (Gene ID: 10728) and the NACA gene (Gene ID: 4666), the intergenic region between the MLF2 gene (Gene ID: 8079) and the PTMS gene (Gene ID: 5763), the intergenic region between the RAB13 gene (Gene ID: 5872) and the RPS27 gene (Gene ID: 4840565), the intergenic region between the JTB gene (Gene ID: 10899) and the RAB13 gene (Gene ID: 5872), the intergenic region between the AKR1A1 gene (Gene ID: 10327) and the NASP gene (Gene ID: 4678), the intergenic region between the NDUFS5 gene (Gene ID: 4725) and the MACF1 gene (Gene ID: 23499), the intergenic region between the SRSF9 gene (Gene ID: 8683) and the DYNLL1 gene (Gene ID: 8655), the intergenic region between the MYL6B gene (Gene ID: 140465) and the MYL6 gene (Gene ID: 4637), the intergenic region between the GPX1 gene (Gene ID: 2876) and the RHOA gene (Gene ID: 387), the intergenic region between the HNRNPA2B1 gene (Gene ID: 3181) and the CBX3 gene (Gene ID: 11335), the intergenic region between the ROMO gene (Gene ID: 140823) and the RBM39 gene (Gene ID: 9584), the intergenic region between the PA2G4 gene (Gene ID: 5036) and the RPL41 gene (Gene ID: 6171), and the intergenic region between the NDUFB10 (Gene ID: 4716) and the RPS2 gene (Gene ID: 6187). In some embodiments, the genes herein refer to human genes and the mammalian cells are human cells.

The start and end genomic coordinates and the sizes of the aforementioned STAPLR intergenic regions in the human genome are listed in Table 1 below. The coordinates are as defined by information available at NCBI's RefSeq database.

TABLE 1
Intergenic Regions Between Select Genes

| SEQ | | Start | End | |
|---|---|---|---|---|
| ID NO | STAPLR | coordinate | coordinate | size |
| 1 | Intergenic region | chr4: | chr4: | 20100 |
| | between the RPL34 gene | 108,630,485 | 108,650,584 | |
| | and the OSTC gene | | | |
| 2 | Intergenic region | chr7: | chr7: | 62214 |
| | between the ACTB gene | 5,530,602 | 5,592,815 | |
| | and the FSCN1 gene | | | |
| 3 | Intergenic region | chr1: | chr1: | 20229 |
| | between the AKIRIN1 | 39,006,066 | 39,026,294 | |
| | gene and the NDUFS5 | | | |
| | gene | | | |
| 4 | Intergenic region | chr1: | chr1: | 27888 |
| | between the PRDX1 | 45,522,891 | 45,550,778 | |
| | gene and the AKR1A1 | | | |
| | gene | | | |
| 5 | Intergenic region | chr12: | chr12: | 24142 |
| | between the PTGES3 and | 56,688,285 | 56,712,426 | |
| | the NACA gene | | | |
| 6 | Intergenic region | chr12: | chr12: | 13221 |
| | between the MLF2 and | 6,753,142 | 6,766,362 | |
| | the PTMS gene | | | |
| 7 | Intergenic region | chr1: | chr1: | 4422 |
| | between the RAB13 and | 153,986,340 | 153,990,761 | |
| | the RPS27 gene | | | |
| 8 | Intergenic region | chr1: | chr1: | 3975 |
| | between the JTB and the | 153,977,675 | 153,981,649 | |
| | RAB13 gene | | | |
| 9 | Intergenic region | chr1: | chr1: | 13991 |
| | between the AKR1A1 | 45,570,050 | 45,584,040 | |
| | and the NASP gene | | | |
| 10 | Intergenic region | chr1: | chr1: | 49551 |
| | between the NDUFS5 | 39,034,616 | 39,084,166 | |
| | and the MACF1 gene | | | |
| 11 | Intergenic region | chr12: | chr12: | 93 |
| | between the SRSF9 and | 120,469,749 | 120,469,841 | |
| | the DYNLL1 gene | | | |
| 12 | Intergenic region | chr12: | chr12: | 376 |
| | between the MYL6B and | 56,157,983 | 56,158,358 | |
| | the MYL6 gene | | | |
| 13 | Intergenic region | chr3: | chr3: | 791 |
| | between the GPX1 and | 49,358,354 | 49,359,144 | |
| | the RHOA gene | | | |
| 14 | Intergenic region | chr7: | chr7: | 696 |
| | between the | 26,200,747 | 26,201,442 | |
| | HNRNPA2B1 and the | | | |
| | CBX3 gene | | | |
| 15 | Intergenic region | chr20: | chr20: | 362 |
| | between the ROMO and | 35,700,985 | 35,701,346 | |
| | the RBM39 gene | | | |
| 16 | Intergenic region | chr12: | chr12: | 2722 |
| | between the PA2G4 and | 56,113,911 | 56,116,632 | |
| | the RPL41 gene | | | |
| 97 | Intergenic region | chr16: | chr16: | 81 |
| | between the NDUFB10 | 1,961,976 | 1,962,057 | |
| | and the RPS2 gene | | | |

Due to variations between humans and variations between mammalian species, the intergenic regions between the aforementioned gene pairs may differ to some degree from the corresponding SEQ ID NOs shown in Table 1.

In some embodiments, the intergenic region between the RPL34 gene and the OSTC gene comprises a nucleotide sequence at least 80%, at least 85%, at least 90%, at least 91%, at least 92%, at least 93%, at least 94%, at least 95%, at least 96%, at least 97%, at least 98%, or at least 99% identical to SEQ ID NO: 1 or is sufficiently similar to SEQ ID NO: 1 so that the intergenic region retains the functionality of SEQ ID NO: 1, i.e., the functions (e.g., transcription regulation) of the intergenic region between the RPL34 gene and the OSTC gene remain intact (e.g., without adverse effects on the cell).

In some embodiments, the intergenic region between the ACTB gene and the FSCN1 gene comprises a nucleotide sequence at least 80%, at least 85%, at least 90%, at least 91%, at least 92%, at least 93%, at least 94%, at least 95%, at least 96%, at least 97%, at least 98%, or at least 99% identical to SEQ ID NO: 2 or is sufficiently similar to SEQ ID NO: 2 so that the intergenic region retains the functionality of SEQ ID NO: 2, i.e., the functions (e.g. transcription regulation) of the intergenic region between the ACTB gene and the FSCN1 gene remain intact (e.g., without adverse effects on the cell).

In some embodiments, the intergenic region between the AKIRIN1 gene and the NDUFS5 gene comprises a nucleotide sequence at least 80%, at least 85%, at least 90%, at least 91%, at least 92%, at least 93%, at least 94%, at least 95%, at least 96%, at least 97%, at least 98%, or at least 99% identical to SEQ ID NO: 3 or is sufficiently similar to SEQ ID NO: 3 so that the intergenic region retains the functionality of SEQ ID NO: 3, i.e., the functions (e.g., transcription regulation) of the intergenic region between the AKIRIN1 gene and the NDUFS5 gene remain intact (e.g., without adverse effects on the cell).

In some embodiments, the intergenic region between the PRDX1 gene and the AKR1A1 gene comprises a nucleotide sequence at least 80%, at least 85%, at least 90%, at least 91%, at least 92%, at least 93%, at least 94%, at least 95%, at least 96%, at least 97%, at least 98%, or at least 99% identical to SEQ ID NO: 4 or is sufficiently similar to SEQ ID NO: 4 so that the intergenic region retains the functionality of SEQ ID NO: 4, i.e., the functions (e.g., transcription regulation) of the intergenic region between the PRDX1 gene and the AKR1A1 gene remain intact (e.g., without adverse effects on the cell).

In some embodiments, the intergenic region between the PTGES3 gene and the NACA gene comprises a nucleotide sequence at least 80%, at least 85%, at least 90%, at least 91%, at least 92%, at least 93%, at least 94%, at least 95%, at least 96%, at least 97%, at least 98%, or at least 99% identical to SEQ ID NO: 5 or is sufficiently similar to SEQ ID NO: 5 so that the intergenic region retains the functionality of SEQ ID NO: 5, i.e., the functions (e.g., transcription regulation) of the intergenic region between the PTGES3 gene and the NACA gene remain intact (e.g., without adverse effects on the cell).

In some embodiments, the intergenic region between the MLF2 gene and the PTMS gene comprises a nucleotide sequence at least 80%, at least 85%, at least 90%, at least 91%, at least 92%, at least 93%, at least 94%, at least 95%, at least 96%, at least 97%, at least 98%, or at least 99% identical to SEQ ID NO: 6 or is sufficiently similar to SEQ ID NO: 6 so that the intergenic region retains the functionality of SEQ ID NO: 6, i.e., the functions (e.g., transcription regulation) of the intergenic region between the MLF2 gene and the PTMS gene remain intact (e.g., without adverse effects on the cell).

In some embodiments, the intergenic region between the RAB13 gene and the RPS27 gene comprises a nucleotide sequence at least 80%, at least 85%, at least 90%, at least 91%, at least 92%, at least 93%, at least 94%, at least 95%, at least 96%, at least 97%, at least 98%, or at least 99% identical to SEQ ID NO: 7 or is sufficiently similar to SEQ ID NO: 7 so that the intergenic region retains the functionality of SEQ ID NO: 7, i.e., the functions (e.g., transcription regulation) of the intergenic region between the RAB13 gene and the RPS27 gene remain intact (e.g., without adverse effects on the cell).

In some embodiments, the intergenic region between the JTB gene and the RAB13 gene comprises a nucleotide sequence at least 80%, at least 85%, at least 90%, at least 91%, at least 92%, at least 93%, at least 94%, at least 95%, at least 96%, at least 97%, at least 98%, or at least 99% identical to SEQ ID NO: 8 or is sufficiently similar to SEQ ID NO: 8 so that the intergenic region retains the functionality of SEQ ID NO: 8, i.e., the functions (e.g., transcription regulation) of the intergenic region between the JTB gene and the RAB13 gene remain intact (e.g., without adverse effects on the cell).

In some embodiments, the intergenic region between the AKR1A1 gene and the NASP gene comprises a nucleotide sequence at least 80%, at least 85%, at least 90%, at least 91%, at least 92%, at least 93%, at least 94%, at least 95%, at least 96%, at least 97%, at least 98%, or at least 99% identical to SEQ ID NO: 9 or is sufficiently similar to SEQ ID NO: 9 so that the intergenic region retains the functionality of SEQ ID NO: 9, i.e., the functions (e.g., transcription regulation) of the intergenic region between the AKR1A1 gene and the NASP gene remain intact (e.g., without adverse effects on the cell).

In some embodiments, the intergenic region between the NDUFS5 gene and MACF1 gene comprises a nucleotide sequence at least 80%, at least 85%, at least 90%, at least 91%, at least 92%, at least 93%, at least 94%, at least 95%, at least 96%, at least 97%, at least 98%, or at least 99% identical to SEQ ID NO: 10 or is sufficiently similar to SEQ ID NO: 10 so that the intergenic region retains the functionality of SEQ ID NO: 10, i.e., the functions (e.g., transcription regulation) of the intergenic region between the NDUFS5 gene and the MACF1 gene remain intact (e.g., without adverse effects on the cell).

In some embodiments, the intergenic region between the SRSF9 gene and DYNLL1 gene comprises a nucleotide sequence at least 80%, at least 85%, at least 90%, at least 91%, at least 92%, at least 93%, at least 94%, at least 95%, at least 96%, at least 97%, at least 98%, or at least 99% identical to SEQ ID NO: 11 or is sufficiently similar to SEQ ID NO: 11 so that the intergenic region retains the functionality of SEQ ID NO: 11, i.e., the functions (e.g., transcription regulation) of the intergenic region between the SRSF9 gene and the DYNLL1 gene remain intact (e.g., without adverse effects on the cell).

In some embodiments, the intergenic region between theMYL6B gene andMYL6 gene comprises a nucleotide sequence at least 80%, at least 85%, at least 90%, at least 91%, at least 92%, at least 93%, at least 94%, at least 95%, at least 96%, at least 97%, at least 98%, or at least 99% identical to SEQ ID NO: 12 or is sufficiently similar to SEQ ID NO: 12 so that the intergenic region retains the functionality of SEQ ID NO: 12, i.e., the functions (e.g., transcription regulation) of the intergenic region between the MYL6B gene and the MYL6 gene remain intact (e.g., without adverse effects on the cell).

In some embodiments, the intergenic region between the GPX1 gene and RHOA gene comprises a nucleotide sequence at least 80%, at least 85%, at least 90%, at least 91%, at least 92%, at least 93%, at least 94%, at least 95%, at least 96%, at least 97%, at least 98%, or at least 99% identical to SEQ ID NO: 13 or is sufficiently similar to SEQ ID NO: 13 so that the intergenic region retains the functionality of SEQ ID NO: 13, i.e., the functions (e.g., transcription regulation) of the intergenic region between the GPX1 gene and the RHOA gene remain intact (e.g., without adverse effects on the cell).

In some embodiments, the intergenic region between the HNRNPA2B1 gene and CBX3 gene comprises a nucleotide sequence at least 95%, at least 96%, at least 97%, at least 98%, or at least 99% identical to SEQ ID NO: 14 or is sufficiently similar to SEQ ID NO: 14 so that the intergenic region retains the functionality of SEQ ID NO: 14, i.e., the functions (e.g., transcription regulation) of the intergenic region between the HNRNPA2B1 gene and the CBX3 gene remain intact (e.g., without adverse effects on the cell).

In some embodiments, the intergenic region between the ROMO gene and RBM39 gene comprises a nucleotide sequence at least 80%, at least 85%, at least 90%, at least 91%, at least 92%, at least 93%, at least 94%, at least 95%, at least 96%, at least 97%, at least 98%, or at least 99% identical to SEQ ID NO: 15 or is sufficiently similar to SEQ ID NO: 15 so that the intergenic region retains the functionality of SEQ ID NO: 15, i.e., the functions (e.g., transcription regulation) of the intergenic region between the ROMO gene and the RBM39 gene remain intact (e.g., without adverse effects on the cell).

In some embodiments, the intergenic region between the PA2G4 gene and RPL41 gene comprises a nucleotide sequence at least 80%, at least 85%, at least 90%, at least 91%, at least 92%, at least 93%, at least 94%, at least 95%, at least 96%, at least 97%, at least 98%, or at least 99% identical to SEQ ID NO: 16 or is sufficiently similar to SEQ ID NO: 16 so that the intergenic region retains the functionality of SEQ ID NO: 16, i.e., the functions (e.g., transcription regulation) of the intergenic region between the PA2G4 gene and the RPL41 gene remain intact (e.g., without adverse effects on the cell).

In some embodiments, the intergenic region between the NDUFB10 and the RPS2 gene comprises a nucleotide sequence at least 80%, at least 85%, at least 90%, at least 91%, at least 92%, at least 93%, at least 94%, at least 95%, at least 96%, at least 97%, at least 98%, or at least 99% identical to SEQ ID NO: 16 or is sufficiently similar to SEQ ID NO: 16 so that the intergenic region retains the functionality of SEQ ID NO: 97, i.e., the functions (e.g., transcription regulation) of the intergenic region between the NDUFB10 and the RPS2 gene remain intact (e.g., without adverse effects on the cell).

The percent identity of two nucleotide sequences can be determined by, e.g., BLAST® using default parameters (available at the U.S. National Library of Medicine's National Center for Biotechnology Information website). In some embodiments, the length of a reference sequence aligned for comparison purposes is at least 30%, (e.g., at least 40, 50, 60, 70, 80, or 90% of the reference sequence.

II. Integration of Exogenous Sequences into STAPLRs

### A. Integration Sites

An exogenous nucleotide sequence of interest may be integrated at any site within a STAPLR. For example, the integration site, or the junction between the exogenous sequence and the adjacent endogenous sequence, may be located in the first half or the second half of the STAPLR; in the 5′, middle, or 3′ third of the STAPLR; or in the first, second, third, or fourth quarter of the STAPLR. In some embodiments, the integration site of the exogenous sequence, or the junction between the exogenous sequence and the adjacent endogenous sequence, is located within the STAPLR and at least 10, 20, 30, 40, 50, 80, 90, 100, 200, 300, 400, 500, 1000, 1500, 2000, 2500, 3000, 3500, 4000, 5000, 10000, 15000, or 20000 base pairs away from the nearest gene, i.e., from the 5′ or 3′ boundary of the STAPLR (e.g., from the start or end coordinate shown in Table 1).

In a single genome, one or more exogenous nucleotide sequences may be integrated into one or more STAPLRs. In some embodiments, one or more (e.g., two, three, or four) exogenous nucleotide sequences may be integrated into one or more sites within a single given STAPLR. In some embodiments, more than one STAPLR in a single genome is targeted for integration of exogenous nucleotide sequences.

In some embodiments, exogenous sequences are introduced into at least one STAPLR and at least one sustained transgene expression locus (STEL) as described in WO 2021/072329. A STEL site is the locus of an endogenous gene that is robustly and consistently expressed in the pluripotent state as well as during differentiation (e.g., as examined by single-cell RNA sequencing (scRNAseq) analysis). While a STAPLR can be associated with a STEL site, it does not need to be associated with a STEL site. STEL sites may be identified from single cell RNA sequence data. A defining characteristic of a desirable STEL site is the ubiquity of expression. STEL sites may be identified by analyzing a candidate gene locus's expression across diverse cell types and cell maturity states such as PSCs and PSC-derived dopamine neurons (and select progenitor states), microglia (and select progenitor states), and cardiomyocytes (and select cardiomyocyte progenitor states). Adding publicly available single cell RNA sequencing data of adult human tissue allows for the refining of such a STEL analysis. STEL include, without limitation, certain housekeeping genes that are active in multiple cell types such as those involved in gene expression (e.g., transcription factors and histones), cellular metabolism (e.g., GAPDH and NADH dehydrogenase), or cellular structures (e.g., actin), or those that encode ribosomal proteins (e.g., large or small ribosomal subunits, such as RPL13A, RPLP0 and RPL7). Examples of STEL are genes encoding ribosomal proteins such as RPL genes (e.g., RPL13A, RPLP0, RPL10, RPL13, RPS18, RPL3, RPLP1, RPL15, RPL41, RPL11, RPL32, RPL18A, RPL19, RPL28, RPL29, RPL9, RPL8, RPL6, RPL18, RPL7, RPL7A, RPL21, RPL37A, RPL12, RPL5, RPL34, RPL35A, RPL30, RPL24, RPL39, RPL37, RPL14, RPL27A, RPLP2, RPL23A, RPL26, RPL36, RPL35, RPL23, RPL4, and RPL22) and RPS genes (e.g., RPS2, RPS19, RPS14, RPS3A, RPS12, RPS3, RPS6, RPS23, RPS27A, RPS8, RPS4X, RPS7, RPS24, RPS27, RPS15A, RPS9, RPS28, RPS13, RPSA, RPS5, RPS16, RPS25, RPS15, RPS20, and RPS11); genes encoding mitochondria proteins (e.g., MT-CO1, MT-C02, MT-ND4, MT-ND1, and MT-ND2); genes encoding actin proteins (ACTG1 and ACTB); genes encoding eukaryotic translation factors (e.g., EEF1A1, EEF2, and EIF1); and genes encoding histones (e.g., H3F3A and H3F3B). Additional STELs are those that encode proteins involved in focal adhesion, cell-substrate adherens junction, cell-substrate junction, cell anchoring, extracellular exosome, extracellular vesicle, intracellular organelle, or anchoring junction. Additional examples of STELs are FTL, FTH1, TPT1, TMSB10, GAPDH, PTMA, GNB2L1, NACA, YBX1, NPM1, FAU, UBA52, HSP90AB1, MYL6, SERF2, and SRP14.

In some embodiments, in a single mammalian (e.g., human) genome, exogenous sequences are introduced into a STAPLR such as the RPL34-OSTC or PRDX1-AKRIA1 STAPLR and a STEL such as the GAPDH locus. In some embodiments, exogenous sequences are introduced in multiple STAPLRs in a single genome, such as the RPL34-OSTC and PRDX1-AKRIA1 STAPLRs.

The integration site of an exogenous nucleotide sequence may be within the STAPLR or in gene sequences adjacent to the STAPLR (e.g., in exon, intron, or UTRs of a gene). In some embodiments, an endonuclease generates DNA breaks within a STAPLR. In other embodiments, an endonuclease generates DNA breaks in a gene adjacent to a STAPLR such that after integration, the exogenous nucleotide sequence is still integrated within the STAPLR. In some embodiments, screening of improper integration events may be performed in accordance with methods described in WO 2021/226151, wherein a DNA break is introduced in an exon of a gene that is adjacent to a STAPLR and is necessary for cell survival, and those cells in which integration is not properly achieved do not survive.

### B. Methods of Integration

Any method of genomic integration can be used to take advantage of the STAPLRs described herein. In some embodiments, integration of the exogenous nucleotide sequence in the STAPLR is achieved by using a genomic editing system selected from the group consisting of a CRISPR/Cas system, a Cre/Lox system, a FLP-FRT system, a Transcription Activator-Like Effector Nuclease (TALEN) system, a zinc finger nuclease (ZFN) system, a homing endonuclease, a sequence-specific endonuclease, random integration (e.g., through transposons), a meganuclease, homologous recombination, transposases, and non-nuclease dependent viral vectors (e.g., retroviral, AAV, or lentiviral vectors). In some embodiments, the integration causes no deletion of the endogenous sequence in the region, and/or no addition of nucleotide sequences other than the exogenous donor sequence to be integrated. In some embodiments, the integration causes insertions (of non-donor sequence) and/or deletions (indels) at the integration site.

In some embodiments, the exogenous sequence may be incorporated into a STAPLR site via homologous recombination at DNA breaks generated by a suitable endonuclease such as a CRISPR-associated endonuclease, which may be, for example, a Cas endonuclease selected from, without limitation, a type I (e.g., subtype I-A, I-B, I-C, I-C variant, I-D, I-E, I-F, I-F variant 1, or I-F variant 2), type II (e.g., subtype II-A, II-B, II-B, or II-C), type III (e.g., subtype III-A, III-B, or III-B variant), type IV, or type V Cas protein, or a variant thereof. In some embodiments, the nuclease is selected from the group consisting of Cas1, Cas1B, Cas2, Cas3, Cas4, Cas5, Cas6, Cas7, Cas8, Cas9, Cas12 (e.g., Cas12a or Cpf1, or Cas12b), Cas13, Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, CasX, CasY, Csx3, Csx1, Csx15, Csf1, Csf2, Csf3, CasPhi, MAD7, Csf4, and homologs thereof, or modified versions thereof (e.g., truncated versions or variants of a wildtype Cas protein with a nuclease activity).

In some embodiments, the Cas endonuclease is a Cpf1 (Cas12a) endonuclease, or a variant, derivative, or fragment thereof, such as, for example, Cpf1 derived from Francisella novicida U112 (FnCpf1), Acidaminococcus sp. BV3L6 (AsCpf1, including improved variants such as enAsCpf1), Lachnospiraceae bacterium ND2006 (LbCpf1), Lachnospiraceae bacterium MA2020 (Lb2Cpf1), Lachnospiraceae bacterium MC2017 (Lb3Cpf1), Moraxella bovoculi 237 (MbCpf1), or Prevotella disiens (PdCpf1).

In some embodiments, the Cas endonuclease is a Cas9 protein or a variant, derivative, or fragment thereof. In some embodiments, the Cas9 protein is SaCas9, SpCas9, SpCas9n, Cas9-HF, Cas9-H840A, FokI-dCas9, or DGTA nickase.

In some embodiments, the Cas endonuclease is a Type V RNA programmable nuclease, as disclosed in WO 2022/258753.

In some embodiments, the Cas endonuclease is a MAD nuclease, such as MAD7 nuclease, as disclosed in U.S. Pat. No. 10,337,028.

Non-limiting examples of suitable endonucleases are set forth in Table A below.

TABLE A
Exemplary Endonucleases

| Enzyme | Sequence |
|---|---|
| Cas12a | MTQFEGFTNLYQVSKTLRFELIPQGKTLKHIQEQGFIEEDKARNDHYKELKPI |
| | IDRIYKTYADQCLQLVQLDWENLSAAIDSYRKEKTEETRNALIEEQATYRNAI |
| | HDYFIGRTDNLTDAINKRHAEIYKGLFKAELFNGKVLKQLGTVTTTEHENALL |
| | RSFDKFTTYFSGFYENRKNVFSAEDISTAIPHRIVQDNFPKFKENCHIFTRLI |
| | TAVPSLREHFENVKKAIGIFVSTSIEEVFSFPFYNQLLTQTQIDLYNQLLGGI |
| | SREAGTEKIKGLNEVLNLAIQKNDETAHIIASLPHRFIPLFKQILSDRNTLSF |
| | ILEEFKSDEEVIQSFCKYKTLLRNENVLETAEALFNELNSIDLTHIFISHKKL |
| | ETISSALCDHWDTLRNALYERRISELTGKITKSAKEKVQRSLKHEDINLQEII |
| | SAAGKELSEAFKQKTSEILSHAHAALDQPLPTTLKKQEEKEILKSQLDSLLGL |
| | YHLLDWFAVDESNEVDPEFSARLTGIKLEMEPSLSFYNKARNYATKKPYSVEK |
| | FKLNFQMPTLASGWDVNKEKNNGAILFVKNGLYYLGIMPKQKGRYKALSFEPT |
| | EKTSEGFDKMYYDYFPDAAKMIPKCSTQLKAVTAHFQTHTTPILLSNNFIEPL |
| | EITKEIYDLNNPEKEPKKFQTAYAKKTGDQKGYREALCKWIDFTRDFLSKYTK |
| | TTSIDLSSLRPSSQYKDLGEYYAELNPLLYHISFQRIAEKEIMDAVETGKLYL |
| | FQIYNKDFAKGHHGKPNLHTLYWTGLFSPENLAKTSIKLNGQAELFYRPKSRM |
| | KRMAHRLGEKMLNKKLKDQKTPIPDTLYQELYDYVNHRLSHDLSDEARALLPN |
| | VITKEVSHEIIKDRRFTSDKFFFHVPITLNYQAANSPSKFNQRVNAYLKEHPE |
| | TPIIGIDRGERNLIYITVIDSTGKILEQRSLNTIQQFDYQKKLDNREKERVAA |
| | RQAWSVVGTIKDLKQGYLSQVIHEIVDLMIHYQAVVVLENLNFGFKSKRTGIA |
| | EKAVYQQFEKMLIDKLNCLVLKDYPAEKVGGVLNPYQLTDQFTSFAKMGTQSG |
| | FLFYVPAPYTSKIDPLTGFVDPFVWKTIKNHESRKHFLEGFDFLHYDVKTGDF |
| | ILHFKMNRNLSFQRGLPGFMPAWDIVFEKNETQFDAKGTPFIAGKRIVPVIEN |
| | HRFTGRYRDLYPANELIALLEEKGIVFRDGSNILPKLLENDDSHAIDTMVALI |
| | RSVLQMRNSNAATGEDYINSPVRDLNGVCFDSRFQNPEWPMDADANGAYHIAL |
| | KGQLLLNHLKESKDLKLONGISNODWLAYIQELRN (SEQ ID NO: 98) |
| Cas12a variant | MTQFEGFTNLYQVSKTLRFELIPQGKTLKHIQEQGFIEEDKARNDHYKELKPI |
| 1 | IDRIYKTYADQCLQLVQLDWENLSAAIDSYRKEKTEETRNALIEEQATYRNAI |
| | HDYFIGRTDNLTDAINKRHAEIYKGLFKAELFNGKVLKQLGTVTTTEHENALL |
| | RSFDKFTTYFSGFYENRKNVFSAEDISTAIPHRIVQDNFPKFKENCHIFTRLI |
| | TAVPSLREHFENVKKAIGIFVSTSIEEVFSFPFYNQLLTQTQIDLYNQLLGGI |
| | SREAGTEKIKGLNEVLNLAIQKNDETAHIIASLPHRFIPLFKQILSDRNTLSF |
| | ILEEFKSDEEVIQSFCKYKTLLRNENVLETAEALFNELNSIDLTHIFISHKKL |
| | ETISSALCDHWDTLRNALYERRISELTGKITKSAKEKVQRSLKHEDINLQEII |
| | SAAGKELSEAFKQKTSEILSHAHAALDQPLPTTLKKQEEKEILKSQLDSLLGL |
| | YHLLDWFAVDESNEVDPEFSARLTGIKLEMEPSLSFYNKARNYATKKPYSVEK |
| | FKLNFQRPTLASGWDVNKEKNNGAILFVKNGLYYLGIMPKQKGRYKALSFEPT |
| | EKTSEGFDKMYYDYFPDAAKMIPKCSTQLKAVTAHFQTHTTPILLSNNFIEPL |
| | EITKEIYDLNNPEKEPKKFQTAYAKKTGDQKGYREALCKWIDFTRDFLSKYTK |
| | TTSIDLSSLRPSSQYKDLGEYYAELNPLLYHISFQRIAEKEIMDAVETGKLYL |
| | FQIYNKDFAKGHHGKPNLHTLYWTGLFSPENLAKTSIKLNGQAELFYRPKSRM |
| | KRMAARLGEKMLNKKLKDQKTPIPDTLYQELYDYVNHRLSHDLSDEARALLPN |
| | VITKEVSHEIIKDRRFTSDKFLFHVPITLNYQAANSPSKFNQRVNAYLKEHPE |
| | TPIIGIDRGERNLIYITVIDSTGKILEQRSLNTIQQFDYQKKLDNREKERVAA |
| | RQAWSVVGTIKDLKQGYLSQVIHEIVDLMIHYQAVVVLENLNFGFKSKRTGIA |
| | EKAVYQQFEKMLIDKLNCLVLKDYPAEKVGGVLNPYQLTDQFTSFAKMGTQSG |
| | FLFYVPAPYTSKIDPLTGFVDPFVWKTIKNHESRKHFLEGFDFLHYDVKTGDF |
| | ILHFKMNRNLSFQRGLPGFMPAWDIVFEKNETQFDAKGTPFIAGKRIVPVIEN |
| | HRFTGRYRDLYPANELIALLEEKGIVFRDGSNILPKLLENDDSHAIDTMVALI |
| | RSVLQMRNSNAATGEDYINSPVRDLNGVCFDSRFQNPEWPMDADANGAYHIAL |
| | KGQLLLNHLKESKDLKLQNGISNQDWLAYIQELRNGRSSDDEATADSQHAAPP |
| | KKKRKV (SEQ ID NO: 99) |
| Type V RNA | MTIRSMKLKLKIYSGRSAPQLRQGLWRLHRLLNEGTAYYMDWLVHMRQEALPG |
| programmable | KSKEEIRAELERRVRQQQEKNGVQNDQVPMDEVLSALRQLYELLVPSAVNNSG |
| nuclease | DAQTLSRKFLSPLVDPNSEGGKGTSNAGAKPGWRKKQEAGDPSWEKDYERWLK |
| example 1 | RKQADPTAEILGKLETAGLKPLFPLYTNEVKDIRWMPLTSKQYVRNWDRDMFQ |
| | QAIEHLLSWETWNRKVNEERAKLKETVRRFEEQHLANGKDWLSPLQAYEANRE |
| | QALRDMAISPSDRFRITRRQIKGWSELYERWNKLAPTASVEAYMQEVRHVQKK |
| | LGGTFGDADLYRFLAKPENVHIWRDHQERLHYYAAYNDLHKRLMSAKEQAAFT |
| | LPDPVAHPLWVRFDARDGNLFTYILQADSSKQRSRRYVNFSRFLWPVEDGYFE |
| | ETENVKVELALSKQFYRQVIVHDNPTGKQKITFQDYSSKEILEGHLGGAKLQL |
| | DRNFLRKSGRDFETGDFGPAFLNVVLDLKPKQEVKNGRLQSPLGQALLVKSRP |
| | NDIPKVYGYKPDALAAWLEQASGEESLGSESLRQGFRVMSIDLGVRSAAAISV |
| | FSVKGEKTREGDKVCYPVGETGLFAVHDRSFLLRLPGESSEKRVNVERDKRKT |
| | ERMQIRYHIRTLARVLRLANKATPMDRIKAVQDVLNDIESTRFMNDHDHHVYN |
| | HALETLRTYAPDHQGIWEEQVIAAHRQLEHHVGVIVGEWRKNWGKDRRGTVGL |
| | SMDNIEELDEMRRLLISWSRRARYPREAKPFQVNESNPVHLLRHLQNLKEDRL |
| | KQLANLIVMTALGYVYDSKEKKWKAAYPACQLILFEDLQRYRFHLDRSARENS |
| | QLMKWAHRSIPKYVWMQGEPYGLQIGDVWAGFTSRYHAKTGAPGIRCKALTEK |
| | DFQQGRLLESLVAEGMFTLQEVGTLKPGDIVPAEGGELFVTLADDSGDRIVIT |
| | HADINAAQNVQKRFWLANSERFRVACRSVQIASQECFIPSSESVAKKMGKGVF |
| | VRDFSFHKDMEVYHWNNQVKLTAKNVPTDHSDDLQDLQDYQAILEEARESSSS |
| | YKTLFRDPSGFFFPDDVWVPQNIYWREVKKTITALLRKRIMST (SEQ ID |
| | NO: 100) |
| Type V RNA | MPIRSFKLKLVTHNGDSTYMDKLRRGLWKTHVIINRGIAYYMNTLALMRQEPY |
| programmable | GSKSREEVRLDLLSTLREQQRRNNWSEQTGTDDELLSLSRRVYELLVPSAIGE |
| nuclease | KGDAQMLSRKFLSPLVDPNSEGGRGTAKSGRKPRWKKMMEEGHPDWEKEKEKD |
| example 1 | AAKKAEDPTASILADLEAVGLLPLFPLFSDEQKEIRWLPKKKRQFVRTWDRDM |
| | FQQALERMLSWESWNRRVAEEYLKLQAQRDEVYAKYLEDAGSWLNDLQTFEKQ |
| | REEELAEVSFEPNSEYLITRRQIRGWKEVYEKWSKTSENASQEQLWRMVADVQ |
| | TAMAGAFGDPKVYQFLSQPKHHHIWREHPNRLFYYSKYNEVREKLNRAKKQAA |
| | FTLPDPVEHPLWTRFDARGGNIHDYEISKVGKQYHVTFSSLILPEAQSWVEIE |
| | NVTVGIGNSLQLKRQIRLDGYADKKQKVKYYDYSSRFELTGVLGGAKIQFDRK |
| | HLKKAAHRLAEGETGPIFLNVVVDVEPFLEVKNGRLRTPLGQVLQVNTRDWPK |
| | VVDYKAKELSVLMENTQIGNENGVSTIEAGMRIMSIDLGQRTAAAVSIFEVIS |
| | KKPDEKETKLFYPIADTDLYAVHRRSLLLRLPGEEISSKKMIEKRKERARIRS |
| | LVRYQIRLLSEVLRLHTQGTAEQRRFKLDELLVSIQKKLELDQSEWISELEKL |
| | FDYIDESAEKWKEALVVAHRTLEPIVVEAVRNWKKSLSKENKDRRRIAGISIW |
| | SIEELEETRKLLIAWSKHSREPGIPKRLEKEETFAPEHLQHIQNVKDDRLKQM |
| | ANLFVMTALGYKYDEGNKRWVEAYPACQVILFEDLSRYRFALDRPRRENNRLM |
| | KWAHRSIPRLTYMQAELFGIQVGDVYSAYTSRFHAKTGAPGIRCHALTEADLQ |
| | SNSYVVNQLIKDKFIQDNQTEILKAGQIVPWQGGELFVTFADRSGASLAVIHA |
| | DINAAQNLQKRFWQHNSEVFRVPCKVVKGGLVPVYEKMRKLFGKGLFVNIDDP |
| | ESKEVYRWEHSTKMKSKTTPVDLESEDIDHEELSDEWEDMQEGYKTLLRDPSG |
| | FFWSSDSWIPQKDFWIRVKSRIGKSLREQIR (SEQ ID NO: 101) |
| Type V RNA | MPIRSFKLKLVTHNGDSTYMDKLRRGLWKTHVIINRGIAYYMNTLALMRQEPY |
| programmable | GSKSREEVRLDLLSTLREQQRRNNWSEQTGTDDELLSLSRRVYELLVPSAIGE |
| nuclease | KGDAQMLSRKFLSPLVDPNSEGGRGTAKSGRKPRWKKMMEEGHPDWEKEKEKD |
| example 3 | AAKKAEDPTASILADLEAVGLLPLFPLFSDEQKEIRWLPKKKRQFVRTWDRDM |
| | FQQALERMLSWESWNRRVAEEYQKLQAQRDEVYAKYLEDAGSWLNDLQTFEKQ |
| | REEELAEVSFEPNSEYLITRRQIRGWKEVYEKWSKTSENASQEQLWRMVADVQ |
| | TAMAGAFGDPKVYQFLSQPKHHHIWREHPNRLFYYSKYNEVREKLNRAKKQAA |
| | FTLPDPVEHPLWTRFDARGGNIHDYEISKVGKQYHVTFSSLILPEAQSWVEIE |
| | NVTVGIGNSLQLKRQIRLDGYADKKQKVKYYDYSSRFELTGVLGGAKIQFDRK |
| | HLKKAAHRLAEGETGPIFLNVVVDVEPFLEVKNGRLRTPLGQVLQVNTRDWPK |
| | VVDYKAKELSVLMENTQIGNENGVSTIEAGMRIMSIDLGQRTAAAVSIFEVIS |
| | KKPDEKETKLFYPIADTDLYAVHRRSLLLRLPGEEISSKKMIEKRKERARIRS |
| | LVRYQIRLLSEVLRLHTQGTAEQRRFKLDELLVSIQRKLELDQSEWISELEKL |
| | FDYIDESAEKWKEALVVAHRTLEPIVVEAVRNWKKSLSKENKDRRRIAGISIW |
| | SIEELEETRKLLIAWSKHSREPGIPKRLEKEETFAPEHLQHIQNVKDDRLKQM |
| | ANLFVMTALGYKYDEGNKRWVEAYPACQVILFEDLSRYRFALDRPRRENNRLM |
| | KWAHRSIPRLTYMQAELFGIQVGDVYSAYTSRFHAKTGAPGIRCHALTEADLQ |
| | SNSYVVNQLIKDKFIQDNQTEILKAGQIVPWQGGELFVTFADRSGASLAVIHA |
| | DINAAQNLQKRFWQHNSEVFRVPCKVVKGGLVPVYEKMRKLFGKGLFVNIDDP |
| | ESKEVYRWEHSTKMKSKTTPVDLESEDIEHEELSDEWEDMQEGYKTLLRDPSG |
| | FFWSSDSWIPQKDFWIRVKSRIGKSLREQIR (SEQ ID NQ: 102) |

In some embodiments, the CRISPR/Cas system comprises a gRNA-dependent nuclease (or a coding sequence thereof) targeting a selected intergenic region, a gRNA (or a coding sequence thereof), and a donor DNA comprising the exogenous nucleotide sequence.

In some embodiments, the STAPLR is the intergenic region between the RPL34 gene and the OSTC gene, and the gRNA is selected from SEQ ID NOs: 25-32.

In some embodiments the STAPLR is the intergenic region between the ACTB gene and the FSCN1 gene, and the gRNA is selected from SEQ ID NOs: 33-54.

In some embodiments, the STAPLR is the intergenic region between the AKIRIN1 gene and the NDUFS5 gene, and the gRNA is selected from SEQ ID NOs: 55-70.

In some embodiments, the STAPLR is the intergenic region between the PRDX1 gene and the AKR1A1 gene, and the gRNA is selected from SEQ ID NOs: 71-92.

### C. Exogenous Nucleotide Sequences

In some embodiments, the exogenous nucleotide sequence of interest for integration may comprise a transgene encoding a protein (as used herein, including a peptide) or an RNA. The transgene may comprise a coding sequence for the gene product and optionally one or more transcription regulatory elements. In some embodiments, the transgene comprises one or more regulatory elements wherein the one or more regulatory elements may be optionally linked operably to the coding sequence.

Nonlimiting examples of regulatory elements are promoters, enhancers, silencers, chromatin insulators, intronic sequences, Kozak sequences, ubiquitous chromatin opening elements (UCOE), transcription activator binding elements, sequences that enhance gene expression or RNA stability (e.g., a WPRE element), polyadenylation signal sequences (e.g., SV40 polyA signal), and the like.

In some embodiments, the promoter directing the expression of the transgene is a constitutive promoter, including, without limitation, EFIa, EFS, UBC, PGK, CAGGS, CMV, SV40, B2M, and ROSA26 promoters. In some embodiments, the promoter is a cell type-specific, tissue-specific or lineage-specific promoter. For example, the promoters may be a tyrosine hydroxylase promoter for dopaminergic neurons; a Hb9 promoter for motor neurons; a SIRPA promoter for cardiomyocytes; a CD14, CD33, CD45, or CD11b promoter for cells of myeloid lineages; or a CD3, FOXP3, CD25, CD8, or CD4 promoter for T lymphocytes. In some embodiments, the expression of the transgene is under the control of an inducible promoter (e.g., lac operon, which can be triggered by Isopropyl β-D-1-thiogalactopyranoside (IPTG); TRE promoter, which can be triggered by tetracycline and its derivatives).

In some embodiments, the exogenous sequence comprises one or more regulatory elements that respond to factors expressed from another site (e.g., from an endogenous gene or a transgene integrated at a STEL or STAPLR). A non-limiting example of such a regulatory element is a transcription factor binding site. In some embodiments, such a regulatory element is integrated at a STAPLR site in the vicinity of other one or more regulatory elements and/or the coding sequence of a transgene. For example, a cell may be modified with a DNA molecule as disclosed herein comprising an exogenous nucleotide sequence comprising a transgene and a transcription factor binding site, where the transcription factor that can bind to the transcription factor binding site is expressed from an endogenous gene, or another transgene in any part of the genome (e.g., in a STAPLR, STEL, or another safe harbor site) or ectopically.

In some embodiments, the transgene encodes an RNA (e.g., a small interfering RNA or a micro-RNA) or a protein of interest. The protein of interest (as used herein, including a peptide) may be, for example, a globular protein (e.g., an albumin, a globulin, a glutelin, a prolamine, a histone, a globin, or a protamine), a fibrous protein (e.g., a scleroprotein such as a collagen, an elastin, a keratin, or a fibroin), or an intermediate protein. In some embodiments, the protein of interest is a complex protein such a metalloprotein, a chromoprotein, a glycoprotein, a mucoprotein, a phosphoprotein, a lipoprotein. In some embodiments, the protein of interest is a therapeutic protein (e.g., a protein that can improve or prevent symptoms of a disease or condition). Nonlimiting examples of therapeutic proteins include proteins that are deficient or defective in genetic diseases such as hemophilia and lysosome storage diseases, hormones, enzymes, cytokines that regulate immunity, recombinant antigen receptors (e.g., chimeric antigen receptors), antibodies, proteins that regulate differentiation or activity of the modified cells (e.g., transcription factors or proteins maintaining cells in M1 or M2 polarity), and the like. In some embodiments, the protein of interest is a cellular marker, a protein used for immune evasion, or a safety or kill switch used in cell therapy. Examples of proteins of interest are, without limitation, SOX10, IL-10, IL-12, CD19t, and ThPOK.

### D. Targeting Vectors

The present disclosure provides targeting vectors for integrating exogenous nucleotide sequences into the STAPLRs. As used herein, a “targeting vector” is a nucleic acid comprising an exogenous nucleotide sequence of interest and sequences homologous to endogenous chromosomal nucleotide sequences that flank the desired integration location in the genome. These flanking homology sequences are referred to as “homology arms.” Homology arms direct the targeting vector to a specific chromosomal location within the genome by virtue of the homology existing between the homology arms and the corresponding endogenous nucleotide sequences. In some embodiments, the targeting vector is a DNA molecule comprising a nucleotide sequence of interest, flanked by a 5′ nucleotide sequence (a left homology arm or homology region) and a 3′ nucleotide sequence (a right homology arm or homology region), wherein the 5′ nucleotide sequence and the 3′ nucleotide sequence are homologous to the nucleotide sequences flanking the integration site in the genome of the cell and mediate integration of the nucleic acid of interest through homology recombination into the integration site.

The 5′ and 3′ sequences are sufficiently similar to the endogenous nucleotide sequences being targeted for homology recombination such that the homology arms if integrated (either wholly or partially) do not cause adverse effects on the genetic environment of the integration (e.g., not impact the neighboring genes' functions). In some embodiments, the homology arms are at least 80%, at least 85%, at least 90%, at least 91%, at least 92%, at least 93%, at least 94%, at least 95%, at least 96%, at least 97%, at least 98%, or at least 99% identical to the nucleotide sequences in the targeted STAPLR.

For example, in some embodiments, the intergenic region between the RPL34 gene and the OSTC gene comprises a nucleotide sequence at least 80% identical to SEQ ID NO: 1 in that the functions of the intergenic region between the RPL34 gene and the OSTC gene remains intact after integration. In some embodiments, the intergenic region between the RPL34 gene and the OSTC gene comprises a nucleotide sequence at least 85%, at least 90%, at least 91%, at least 92%, at least 93%, at least 94%, at least 95%, at least 96%, at least 97%, at least 98%, or at least 99% identical to SEQ ID NO: 1 so that the functions of the intergenic region between the RPL34 gene and the OSTC gene remains intact after integration. The same may be said for the intergenic region between the ACTB gene and the FSCN1 gene and its identity to SEQ ID NO: 2, the intergenic region between the AKIRIN1 gene and the NDUFS5 gene and its identity to SEQ ID NO: 3, and the intergenic region between the PRDX1 gene and the AKR1A1 gene and its identity to SEQ ID NO: 4, the intergenic region between the PTGES3 gene and the NACA gene and its identity to SEQ ID NO: 5, the intergenic region between the MLF2 gene and the PTMS gene and its identity to SEQ ID NO: 6, the intergenic region between the RAB13 gene and the RPS27 gene and its identity to SEQ ID NO: 7, the intergenic region between the JTB gene and the RAB13 gene and its identity to SEQ ID NO: 8, the intergenic region between the AKR1A1 gene and the NASP gene and its identity to SEQ ID NO: 9, the intergenic region between the NDUFS5 gene and the MA CF1 gene and its identity to SEQ ID NO: 10, the intergenic region between the SRSF9 gene and the DYNLL1 gene and its identity to SEQ ID NO: 11, the intergenic region between the MYL6B gene and the MYL6 gene and its identity to SEQ ID NO: 12, the intergenic region between the GPX1 gene and the RHOA gene and its identity to SEQ ID NO: 13, the intergenic region between the HNRNPA2B1 gene and the CBX3 gene and its identity to SEQ ID NO: 14, the intergenic region between the ROMO gene and the RBM39 gene and its identity to SEQ ID NO: 15, the intergenic region between the PA2G4 gene and the RPL41 gene and its identity to SEQ ID NO: 16, and the intergenic region between the NDUFB10 and the RPS2 gene and its identity to SEQ ID NO: 97.

In the methods of the present disclosure, the homology arms vary in length. In some embodiments, each of the homology arms is independently about at least 50, at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1000, at least 1100, at least 1200, at least 1300, at least 1400, at least 1500, at least 1600, at least 1700, at least 1800, at least 1900, or at least 2000 base pairs long. In some embodiments, each of the homology arms is independently 50-2000, 50-1500, 100-1900, 150-1800, 200-1700, 250-1600, 300-1500, 350-1400, 400-1300, 450-1200, 500-1100, 550-1000, 600-950, 650-900, 700-850, or 750-800 base pairs in length.

In the methods of the disclosure, the homology arms (i.e., the 5′ and 3′ nucleotide sequences) can be designed to target anywhere within the disclosed intergenic region. The homology arms can be designed based on genomic sequences available in sequence databases (e.g., the NCBI database).

In some embodiments, the 5′ nucleotide sequence comprises a nucleotide sequence that is sufficiently similar to SEQ ID NO: 17 as necessary for the function of the sequence to remain intact and the 3′ nucleotide sequence comprises a nucleotide sequence that is sufficiently similar to SEQ ID NO: 18 as necessary for the function of the sequence to remain intact. In some embodiments, the 5′ nucleotide sequence comprises a nucleotide sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 17 for the function of SEQ ID NO: 17 to remain intact. In some embodiments, the 3′ nucleotide sequence comprises a nucleotide sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 18 for the function of SEQ ID NO: 18 to remain intact.

In some embodiments, the 5′ nucleotide sequence comprises a nucleotide sequence that is sufficiently similar to SEQ ID NO: 19 as necessary for the function of the sequence to remain intact and the 3′ nucleotide sequence comprises a nucleotide sequence that is sufficiently similar to SEQ ID NO: 20 as necessary for the function of the sequence to remain intact. In some embodiments, the 5′ nucleotide sequence comprises a nucleotide sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 19 for the function of SEQ ID NO: 19 to remain intact. Similarly, in some embodiments, the 3′ nucleotide sequence comprises a nucleotide sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 20 for the function of SEQ ID NO: 20 to remain intact.

In some embodiments, the 5′ nucleotide sequence comprises a nucleotide sequence that is sufficiently similar to SEQ ID NO: 21 as necessary for the function of the sequence to remain intact and the 3′ nucleotide sequence comprises a nucleotide sequence that is sufficiently similar to SEQ ID NO: 22 as necessary for the function of the sequence to remain intact. In some embodiments, the 5′ nucleotide sequence comprises a nucleotide sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 21 for the function of SEQ ID NO: 21 to remain intact. Similarly, in some embodiments, the 3′ nucleotide sequence comprises a nucleotide sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 22 for the function of SEQ ID NO: 22 to remain intact.

In some embodiments, the 5′ nucleotide sequence comprises a nucleotide sequence that is sufficiently similar to SEQ ID NO: 23 as necessary for the function of the sequence to remain intact and the 3′ nucleotide sequence comprises a nucleotide sequence that is sufficiently similar to SEQ ID NO: 24 as necessary for the function of the sequence to remain intact. In some embodiments, the 5′ nucleotide sequence comprises a nucleotide sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 23 for the function of SEQ ID NO: 23 to remain intact. Similarly, in some embodiments, the 3′ nucleotide sequence comprises a nucleotide sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 24 for the function of SEQ ID NO: 24 to remain intact.

In some embodiments, the 5′ nucleotide sequence comprises a nucleotide sequence that is sufficiently similar to SEQ ID NO: 93 as necessary for the function of the sequence to remain intact and the 3′ nucleotide sequence comprises a nucleotide sequence that is sufficiently similar to SEQ ID NO: 94 as necessary for the function of the sequence to remain intact. In some embodiments, the 5′ nucleotide sequence comprises a nucleotide sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 93 for the function of SEQ ID NO: 93 to remain intact. Similarly, in some embodiments, the 3′ nucleotide sequence comprises a nucleotide sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 94 for the function of SEQ ID NO: 94 to remain intact.

In some embodiments, the 5′ nucleotide sequence comprises a nucleotide sequence that is sufficiently similar to SEQ ID NO: 95 as necessary for the function of the sequence to remain intact and the 3′ nucleotide sequence comprises a nucleotide sequence that is sufficiently similar to SEQ ID NO: 96 as necessary for the function of the sequence to remain intact. In some embodiments, the 5′ nucleotide sequence comprises a nucleotide sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 95 for the function of SEQ ID NO: 95 to remain intact. Similarly, in some embodiments, the 3′ nucleotide sequence comprises a nucleotide sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 96 for the function of SEQ ID NO: 96 to remain intact.

In some embodiments, the homology arms completely fall within the targeted STAPLR. In other embodiments, the homology arms may overlap with a portion of a neighboring gene without disrupting its function after integration and the exogenous sequence still is integrated within the STAPLR.

In some embodiments, the targeting vector is a circular vector. In some embodiments, the targeting vector is linear vector. In some embodiments, a targeting vector as provided herein comprises one or more endonuclease targeting sequences, e.g., to linearize the vector when being used with an endonuclease-guide combination. In some embodiments, the target vector is a viral vector (e.g., an AAV vector, an adenoviral vector, a lentiviral vector, a herpes simplex viral vector), or a plasmid vector.

The present disclosure provides a STAPLR-targeting system that comprises the targeting vector herein and an appropriate gene editing system such as those described herein, for incorporating the nucleotide sequence of interest on the targeting vector into the STAPLR.

### III. Genetically Modified Mammalian Cells

Provided herein are genetically modified cells comprising modifications in one or more of the STAPLRs disclosed herein. The mammalian cells targeted for STAPLR integration may be of any cell type or in any cell state of interest. For example, the cells may be pluripotent cells (e.g., pluripotent stem cells) or differentiated cells. The cells, such as human cells, may be engineered in vitro, in vivo, or ex vivo by gene editing methods such as those described herein. The cells may also be non-human cells, such as cells from laboratory animals (e.g., non-human primates, mice, rats and rabbits), farm animals (e.g., cattle and horses), and pets (e.g., dogs and cats).

### A. Stem Cells

In some embodiments, the mammalian cells targeted for modification at their STAPLRs are stem cells, particularly pluripotent stem cells (PSCs) such as induced pluripotent stem cells (iPSCs; e.g., human iPSCs) or embryonic stem cells (ESCs; e.g., human ESCs). Engineered stem cells can be subsequently induced to differentiate into a desired cell type, referred to herein as PSC-derivatives, PSC-derivative cells, or PSC-derived cells. Stem cells can be the starting point for the potential generation of large numbers of cells of a specific cell type that are delivered for regenerative medicine in patients with different diseases.

As used herein, the term “pluripotent” or “pluripotency” refers to the capacity of a cell to self-renew and to differentiate into cells of any of the three germ layers: endoderm, mesoderm, or ectoderm. “Pluripotent stem cells” or “PSCs” include, for example, ESCs derived from the inner cell mass of a blastocyst or derived by somatic cell nuclear transfer, and iPSCs derived from non-pluripotent cells.

As used herein, the terms “embryonic stem,” “ES” cells, and “ESCs” refer to pluripotent stem cells obtained from early embryos. In some embodiments, the term excludes stem cells involving destruction of a human embryo; that is, the ESCs are obtained from a previously established ESC line.

The term “induced pluripotent stem cell” or “iPSC” refers to a type of pluripotent stem cell artificially prepared from a non-pluripotent cell, such as an adult somatic cell, partially differentiated cell or terminally differentiated cell, such as a fibroblast, a cell of hematopoietic lineage, a myocyte, a neuron, an epidermal cell, or the like, by introducing or contacting the cell with one or more reprogramming factors. Methods of producing iPSCs include, for example, inducing expression of one or more genes (e.g., POU5F1/OCT4 (Gene ID: 5460) in combination with, but not restricted to, SOX2 (Gene ID: 6657), KLF4 (Gene ID: 9314), c-MYC (Gene ID: 4609, NANOG (Gene ID: 79923), and/or LIN28/LIN28A (Gene ID: 79727)). Reprogramming factors may be delivered by various means (e.g., viral, non-viral, RNA, DNA, or protein delivery); alternatively, endogenous genes may be activated by using, e.g., CRISPR tools to reprogram non-pluripotent cells into PSCs. See, e.g., WO 2013/177133 and WO 2022/204567.

Methods for inducing differentiation of PSCs into cells of various lineages are known in the art. For example, methods for inducing differentiation of PSCs into dendritic cells are described in Slukvin et al., J Imm. (2006) 176:2924-32; and Su et al., Clin Cancer Res. (2008) 14(19):6207-17; and Tseng et al., Regen Med. (2009) 4(4):513-26. Methods for inducing PSCs into hematopoietic progenitor cells, cells of myeloid lineage, and T lymphocytes are described in, e.g., Kennedy et al., Cell Rep. (2012) 2:1722-35.

The recombinant PSCs can be differentiated into cells suitable for therapy, including the cells in the endoderm (e.g., lung, thyroid, or pancreatic cells, or progenitors thereof), ectoderm (e.g., skin, neuronal, or pigment cells, or progenitors thereof) and mesoderm (e.g., cardiac cells, skeletal muscle cells, red blood cells, smooth muscle cells, or progenitors or precursors thereof) lineages.

In some embodiments, the recombinant PSCs are differentiated into cells in the endoderm (e.g., lung, thyroid, or pancreatic cells, or progenitors or precursors thereof), ectoderm (e.g., skin, neuronal, or pigment cells, or progenitors or precursors thereof) or mesoderm (e.g., cardiac cells, skeletal muscle cells, red blood cells, smooth muscle cells, or progenitors or precursors thereof) lineages.

In some embodiments, a recombinant PSC of the disclosure is differentiated into a cardiac cell. In various embodiments, the cardiac cell is a cardiac progenitor cell or a mature or immature (atrial or ventricular) cardiomyocyte. In other embodiments, the cardiac cell is a cardiac endothelial cell or a nodal cell.

In some embodiments, a recombinant PSC of the disclosure is differentiated into a human immune cell, optionally selected from a T cell, a T cell expressing a chimeric antigen receptor (CAR) or recombinant TCR, a regulatory T cell, a myeloid cell, a dendritic cell, and/or a macrophage/monocyte (e.g., an immunosuppressive macrophage), or a progenitor or precursor thereof.

In some embodiments, a recombinant PSC of the disclosure is differentiated into an oligodendrocyte progenitor cell or precursor cell, or an oligodendrocyte. In some embodiments, a recombinant PSC of the disclosure is differentiated into a microglial progenitor cell or precursor cell, or a microglial cell.

In some embodiments, a recombinant PSC of the disclosure is differentiated into a neural lineage cell, for example a neural crest cells, an astrocyte, a dopaminergic neuron progenitor cell, a dopaminergic neuron, a midbrain dopaminergic neuron progenitor cell, a midbrain dopaminergic neuron, an authentic midbrain dopamine (DA) neuron, a dopaminergic neuron precursor cell, a floor plate midbrain progenitor cell, a floor plate midbrain DA neuron, or a progenitor or precursor thereof.

In some embodiments, a recombinant PSC of the disclosure is differentiated into a cell of the ocular system, such as a photoreceptor cell, a photoreceptor progenitor or precursor cell, a retinal pigmented epithelium (RPE) cell or a progenitor or precursor thereof, a neural retinal cell or a progenitor or precursor thereof. In other embodiments, an unedited PSC is differentiated into a cell of the ocular system, which is then engineered with a targeting construct of the disclosure.

In further embodiments, a recombinant PSC of the disclosure is differentiated into a microglial cell or a microglial progenitor or precursor cell.

In further embodiments, a recombinant PSC of the disclosure is differentiated into a cell in the human metabolic system, optionally selected from a hepatocyte, a cholangiocyte, and a pancreatic beta cell, or a progenitor or precursor thereof.

In further embodiments, a recombinant PSC of the disclosure is differentiated into an enteric progenitor or precursor cell or an enteric cell.

### B. Differentiated Cells

In still other embodiments, the cells to be engineered are differentiated cells (e.g., partially or terminally differentiated cells). Partially differentiated cells may be, for example, tissue-specific progenitor or stem cells, such as hematopoietic progenitor or stem cells, skeletal muscle progenitor or stem cells, cardiac progenitor or stem cells, neuronal progenitor or stem cells, and mesenchymal stem cells.

Exemplary differentiated cell types that can be engineered at one or more of their STAPLRs include the cells in the endoderm (e.g., lung, thyroid, or pancreatic cells, or progenitors thereof), ectoderm (e.g., skin, neuronal, or pigment cells, or progenitors or precursors thereof) and mesoderm (e.g., cardiac cells, skeletal muscle cells, red blood cells, smooth muscle cells, or progenitors or precursors thereof) lineages. Alternatively, PSCs can be differentiated into cells in these lineages and then engineered with a targeting construct of the disclosure.

In some embodiments, a cardiac cell is engineered. In some embodiments, the cardiac cell is a cardiac progenitor cell or a mature or immature (atrial or ventricular) cardiomyocyte. In other embodiments, the cardiac cell is a cardiac endothelial cell or a nodal cell.

In some embodiments, a human immune cell is engineered. The human immune cell is optionally selected from a T cell (e.g., a CD4+ T cell, a CD8+ T cell, or a Treg cell), a T cell expressing a chimeric antigen receptor (CAR) or recombinant TCR, a regulatory T cell, a myeloid cell, a dendritic cell, and/or a macrophage (e.g., an immunosuppressive macrophage), or a progenitor or precursor thereof such as a hematopoietic stem or progenitor cell.

In some embodiments, an oligodendrocyte progenitor cell or precursor cell or an oligodendrocyte is engineered.

In some embodiments, a neural lineage cell is engineered. In various embodiments, the neural lineage cell is a neural crest cell, an astrocyte, a dopaminergic neuron progenitor cell, a dopaminergic neuron cell, a midbrain dopaminergic neuron progenitor cell, a midbrain dopaminergic neuron, an authentic midbrain dopamine (DA) neuron, a dopaminergic neuron precursor cell, a floor plate midbrain progenitor cell, a floor plate midbrain DA neuron, or a progenitor or precursor thereof.

In some embodiments, a cell of the ocular system is engineered. In various embodiments, the cell of the ocular system is a photoreceptor cell, a photoreceptor progenitor or precursor cell, a retinal pigmented epithelium cell or a progenitor or precursor thereof, a neural retinal cell or a progenitor or precursor thereof.

In further embodiments, a microglial cell or a microglial progenitor or precursor cell is engineered.

In further embodiments, a cell in the human metabolic system is engineered. In various embodiments, the cell in the human metabolic system is optionally selected from a hepatocyte, a cholangiocyte, and a pancreatic beta cell, or a progenitor or precursor thereof.

In further embodiments, an enteric progenitor or precursor cell or an enteric cell is engineered.

Additional cell types that can be engineered herein to integrate exogenous sequences into STAPLRs are, without limitations, fibroblasts, adipose cells, muscle cells (e.g., skeletal or smooth muscle cells), bone cells, myeloid cells, myeloid progenitor cells (e.g., primitive myeloid progenitor cells).

The cells may be from established cell lines, or they may be primary cells, where “primary cells”, “primary cell lines”, and “primary cultures” are used interchangeably herein to refer to cells and cells cultures that have been derived from a subject (e.g., a human) and allowed to grow in vitro or ex vivo for a limited number of passages of the culture. For example, primary cultures include cultures that may have been passaged 0 times, 1 time, 2 times, 4 times, 5 times, 10 times, or 15 times, but not enough times to go through the crisis stage. Primary cell lines can be maintained for fewer than 10 passages in vitro or ex vivo. In some embodiments, the cells are autologous in the context of cell therapy. In some embodiments, the cells are allogeneic in the context of a cell therapy.

Primary cells may be harvested from an individual by any suitable method. For example, leukocytes may be suitably harvested by apheresis, leukocytapheresis, density gradient separation, etc., while cells from tissues such as skin, muscle, bone marrow, spleen, liver, pancreas, lung, intestine, stomach, etc. are most suitably harvested by biopsy.

Any of the foregoing differentiated cell types can be differentiated from PSCs prior to engineering them.

The present disclosure provides a pharmaceutical composition comprising the engineered cells herein and a pharmaceutically acceptable carrier.

### IV. Methods of Identifying STAPLRs

The present disclosure also provides methods of identifying STAPLRs as sites for safe genomic integration in a mammalian cell (e.g., a human cell). In these methods, the first step is to select a set of cell types for single cell RNA sequencing (“scRNAseq”). Examples of cell types, without limitation, are those referred to herein, including, without limitation, PSCs (e.g., iPSCs), cells in the immune system (e.g., T cells, NK cells, dendritic cells, macrophages/monocytes, or hematopoietic progenitor cells thereof), cells in the cardiovascular system (e.g., ventricular cardiomyocytes, nodal cells, or cardiac progenitor cells), cells in the metabolic system (e.g., hepatocytes and pancreatic beta-cells), cells in the central nervous system (e.g., sensory neurons, motor neurons, interneurons, microglial cells, oligodendrocytes, or progenitor cells thereof), muscle cells (e.g., skeletal muscle cells and smooth muscle cells), adipose cells, and cells in the ocular system (e.g., retinal pigment epithelium cells and photoreceptor cells).

The second step is to perform an scRNAseq assay wherein the sequencing analysis assigns a unique transcriptome comprising transcribed genes to each cell that passes quality criteria. To pass quality criteria, transcriptomes are filtered to exclude those with high sparsity or missingness and those that are likely derived from more than one cell.

Next, a Prevalence Score is assigned to each gene. The Prevalence Score is out of “1” and represents the fraction of cells containing at least one transcript of a given gene based on an scRNAseq database of datasets collected. In some embodiments, scRNAseq datasets are obtained from PSCs, dopaminergic neurons and/or their progenitors (e.g., those at various select differentiation states), microglia and/or their progenitors (e.g., those at various select differentiation states), cardiomyocytes and/or their progenitors (e.g., those at various select differentiation states), oligodendrocyte cell and/or their progenitors (e.g., those at various select differentiation states), or macrophages and/or their progenitors (e.g., those at various select differentiation states).

After assigning a Prevalence Score, the location of each gene in the mammalian (e.g., human) genome is determined.

The next step in identifying a STAPLR in the genome of a mammalian cell is to identify neighboring, nonoverlapping genes. By “non-overlapping genes” it is meant that the genes are separated from each other by at least 50 base pairs, at least 75 base pairs, at least 100 base pairs, at least 200 base pairs, at least 300 base pairs, at least 400 base pairs, at least 500 base pairs, at least 1000 base pairs, at least 1500 base pairs, at least 2000 base pairs, at least 2500 base pairs, at least 3000 base pairs, 3500 base pairs, at least 5000 base pairs, at least 10000 base pairs, at least 15000 base pairs, or at least 20000 base pairs on either strand. The transcripts used to calculate genetic distances for identifying non-overlapping genes may be specified by any genomic database, such as NCBI's RefSeq database and the GENCODE databases.

In some instances, different genomic databases contain non-consensus gene boundary annotations that may lead to different calculated genetic distances and contrary conclusions as to whether two genes overlap or not. In such instances, two genes are considered non-overlapping if they are determined to be non-overlapping by using at least one genomic database. For example, MLF2 is flanked downstream by its neighboring gene PTMS. As annotated in the NCBI RefSeq database, these genes are non-overlapping, with an intergenic distance of about 13 kb; however, the GENCODE V38 database reports one MLF2 transcript whose transcriptional start site is located within the first intron of PTMS encoded on the opposite strand. In this case, the RefSeq annotations are considered and the GENCODE annotations are not, and this gene pair is classified as non-overlapping.

Once two or more genes are considered non-overlapping, a Neighbor Score for the pairs of non-overlapping genes or for regions comprising three or more non-overlapping genes is determined. A Neighbor Score is the product of the individual Prevalence Scores and reflects the probability of both genes being transcriptionally active in the aggregate scRNAseq dataset. The Neighbor Score is essentially a ranking of the vicinities of transcriptionally active genes.

Neighbor Scores are then sorted to obtain a ranking of pairs of non-overlapping genes or a ranking of regions comprising three or more genes. Once the Neighbor Scores are ranked, a pair of genes or a region comprising three or more genes with the best Neighbor Scores is selected and the intergenic region between the genes of the selected pair or region is identified as a potential STAPLR.

The STAPLR may be targeted for safe genetic integration. Intergenic regions with high-ranking Neighbor Scores are then annotated in order to design homology arms for site-specific integration. In general, sequences to be avoided for integration sites include promoter regions, enhancer regions, CpG islands, epigenetic marks (e.g., H3K4Me1, H3K4Me3, and H3K27Ac), DNase I hypersensitivity peaks, conserved regions, and repetitive regions. The UCSC Genome Browser may be used with, but are not limited to, the following gene annotation tracks: GENCODE V32, RefSeq Genes, GTEx RNA-seq, EPDnew Promoters, ENCODE (transcription, H3K4Me1, H3K4Me3, H3K27Ac, and DNase Clusters), GeneHancer, CpG Islands, Conservation 100 vertebrates, and RepeatMasker.

In selecting a targetable intergenic subregion, known promoter regions and enhancer regions must be avoided. Additionally, conserved regions, repetitive regions, epigenetic marks, and DNase hypersensitivity regions are features that should be minimized in selecting a targetable region. In some embodiments, the targetable intergenic subregion comprises the sequence of an CRISPR endonuclease protospacer adjacent motif (PAM) site. A PAM site is a 2-6 base pair DNA sequence immediately following the DNA sequence targeted by a Cas (e.g., Cas9 or Cpf1) endonuclease. A short oligonucleotide known as a guide RNA (gRNA) is synthesized to perform the function of the tracrRNA-crRNA complex in a CRISPR/Cas gene editing system. A gRNA recognizes gene sequences having a PAM sequence at the 5′ or 3′ end. Different Cas proteins may recognize different PAMs. For example, Cas9 from Streptococcus pyrogenes recognizes 5′-NGG-3′ (“N”: any nucleobase); Cas9 from Staphylococcus aureus recognizes 5′-NNGRR(N)-3′; Cas9 from Neisseria meningitidis recognizes 5′-NNNNGATT-3′; Cas9 from Campylobacter jejuni recognizes 5′-NNNNRYAC-3′ (“Y”: a pyrimidine); Cas9 from Streptococcus thermophilus recognizes 5′-NNAGAAW-3′ (“W”: A or T); Cpf1 (Cas12a) from Lachnospiraceae bacterium and Acidaminococcus sp. recognizes 5′-TTTV-3′ (“V”: G, A, or C); Cas12b from Alicyclobacillus acidiphilus recognizes 5′-TTN-3′; and Cas12b v4 from Bacillus hisashii recognizes 5′-ATTN-3′, 5′-TTTN-3′, and 5′-GTTN-3′.

Finally, confirmation that the identified intergenic region will safely support an exogenous genetic payload may be carried out by inserting a transgene at a targeted location within the intergenic region using a gene editing system. The gene editing system may be, for example, a CRISPR system (e.g., those using an CRISPR endonuclease disclosed above), a Cre/Lox system, a FLP-FRT system, a TALEN system, a ZFN system, a system that utilizes homing endonucleases, a system that produces homologous recombination, or a system that utilizes non-nuclease dependent viral vectors (e.g., retroviral, AAV, or lentiviral vectors). Constitutive, inducible, tissue-specific, or lineage-specific promoters may be used to direct expression of the inserted transgene.

In some embodiments, the targeted intergenic region is at least 30, 40, 50, 75, or 100 base pairs in length. In some embodiments, the intergenic region does not comprise a promoter region or an enhancer region. While it may be better for the intergenic region not to comprise conserved regions, repetitive regions, epigenetic marks, and/or DNase hypersensitivity regions, the intergenic region may in fact contain a minimal amount of conserved regions, repetitive regions, epigenetic marks, and/or enzymatic hypersensitivity regions in some embodiments. For example, in some embodiments, the intergenic region will not comprise a CpG Island, an H3K4Me1 epigenetic mark, an H3K4Me3 epigenetic mark, an H3K27Ac epigenetic mark, a DNase I hypersensitivity region, a conserved region, or a repetitive region. However, in some embodiments, the intergenic region may comprise a CpG Island, an H3K4Me1 epigenetic mark, an H3K4Me3 epigenetic mark, an H3K27Ac epigenetic mark, a DNAseI hypersensitivity region, a conserved region, or a repetitive region. The amount of allowed conserved regions, repetitive regions, epigenetic marks, and/or DNase hypersensitivity regions depends on various factors. These factors include, for example, the size of the intergenic region; the size of the conserved, repetitive, and/or hypersensitivity regions, or epigenetic marks; the presence of gRNA binding sites; or challenges to synthesizing 5′ and 3′ homology arms for targeting.

After genomic integration, the transcription level of the integrated transgene is measured and the intergenic region between the selected pair or within the selected region is confirmed to be a STAPLR when the integrated transgene displays sustained transcription (or displays sustained transcription when an inducible promoter regulating the transgene is induced).

Unless otherwise defined herein, scientific and technical terms used in connection with the present invention shall have the meanings that are commonly understood by those of ordinary skill in the art. Exemplary methods and materials are described below, although methods and materials similar or equivalent to those described herein can also be used in the practice or testing of the present invention. In case of conflict, the present specification, including definitions, will control. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. All publications and other references mentioned herein are incorporated by reference in their entirety. Although a number of documents are cited herein, this citation does not constitute an admission that any of these documents forms part of the common general knowledge in the art. As used herein, the term “approximately” or “about” as applied to one or more values of interest refers to a value that is similar to a stated reference value. In some embodiments, the term refers to a range of values that fall within 10%, 9%, 8%, 7%, 6%, 5%, 4%, 3%, 2%, 10%, or less in either direction (greater than or less than) of the stated reference value unless otherwise stated or otherwise evident from the context.

According to the present disclosure, back-references in the dependent claims are meant as short-hand writing for a direct and unambiguous disclosure of each and every combination of claims that is indicated by the back-reference. Further, headers herein are created for ease of organization and are not intended to limit the scope of the claimed invention in any manner.

In order that this invention may be better understood, the following examples are set forth. These examples are for purposes of illustration only and are not to be construed as limiting the scope of the invention in any manner.

### EXAMPLES

In order that this invention may be better understood, the following Examples are set forth. These Examples are for purposes of illustration only and are not to be construed as limiting the scope of the invention in any manner.

### Example 1: Design for STAPLR Targeting

Selection of gRNA

We used CRISPOR to identify Cas9-based gRNAs that target near the midpoint region where STAPLR construct homology arms flanked the intended site of transgene integration. We excluded gRNAs that had perfect off-targets in the genome. We minimized the use of gRNAs that had a maximum of 3 bp off-target mismatches. Three gRNAs were selected for each STAPLR site as seen in Table 2.

TABLE 2
gRNAs for Targeting Select STAPLR Sites

| STAPLR Site | gRNA Sequence (5′→3′) |
|---|---|
| PRDX1-AKIR1A1 | CTTGCAGCACTGCCTAGGCT (SEQ ID NO: 71) |
| | TGGTTCTTGCAGCACTGCCT* (SEQ ID NO: 72) |
| | TCTTGCAGCACTGCCTAGGC (SEQ ID NO: 73) |
| ACTB-FSCN1 | GCCCACCTTGAAGATCTGTA* (SEQ ID NO: 33) |
| | TGCCCACCTTGAAGATCTGT (SEQ ID NO: 34) |
| | CACCTTGAAGATCTGTAGGG (SEQ ID NO: 35) |
| RPL34-OSTC | TTATCATGATACTATGTCCC* (SEQ ID NO: 25) |
| | AGTTACTGAGCACAGTGCCT (SEQ ID NO: 26) |
| | GAGTTACTGAGCACAGTGCC (SEQ ID NO: 27) |
| AKIRIN1-NDUFS5 | ATTAATGCCTTTAAATAGGT (SEQ ID NO: 55) |
| | TCTTAAACCTACCTATTTAA* (SEQ ID NO: 56) |
| | GCTAATTAATGCCTTTAAAT (SEQ ID NO: 57) |

*Preferred embodiments.

A list of additional Cas9- and Cpf1-based gRNAs for STAPLR targeting is listed in Table 3.

TABLE 3
Additional STAPLR-Targeting gRNAs

| STAPLR Site | Nuclease | gRNA Sequence (5′→3′) |
|---|---|---|
| PRDX1-AKIR1A1 | Cas9 | CTTGCAGCACTGCCTAGGCT (SEQ ID NO: 74) |
| Site 1 | | TGGTTCTTGCAGCACTGCCT (SEQ ID NO: 75) |
| | | TCTTGCAGCACTGCCTAGGC (SEQ ID NO: 76) |
| | | TAGGCTGGGCTCAAACTCCA (SEQ ID NO: 77) |
| | | CTAGGCTGGGCTCAAACTC (SEQ ID NO: 78) |
| | | TGAGAGGATCACTTGAGCCC (SEQ ID NO: 79) |
| | | CTGGAGTTTGAGCCCAGCCT (SEQ ID NO: 80) |
| | | TTCTTTGTTTGTTTAGAGAC (SEQ ID NO: 81) |
| | Cpf1 | AGCCCAGCCTAGGCAGTGCTG (SEQ ID NO: 82) |
| | | GAGACTGGTTCTTGCAGCACT (SEQ ID NO: 83) |
| | | TTTAGAGACTGGTTCTTGCAG (SEQ ID NO: 84) |
| | | TTTGTTTAGAGACTGGTTCTT (SEQ ID NO: 85) |
| | | TTTGTTTGTTTAGAGACTGGT (SEQ ID NO: 86) |
| ACTB-FSCN1 | Cas9 | GCCCACCTTGAAGATCTGTA (SEQ ID NO: 38) |
| | | TGCCCACCTTGAAGATCTGT (SEQ ID NO: 39) |
| | | GGGCCAAGAGGCTCAGTCAC (SEQ ID NO: 40) |
| | | GGATAAAGAGATCAGGTCCA (SEQ ID NO: 41) |
| | | CACCTTGAAGATCTGTAGGG (SEQ ID NO: 42) |
| | | TCCCTACAGATCTTCAAGGT (SEQ ID NO: 43) |
| | | TTCAAGGTGGGCAAGGAACT (SEQ ID NO: 44) |
| | | TCCCTCCCTACAGATCTTCA (SEQ ID NO: 45) |
| | | CTCCCTACAGATCTTCAAGG (SEQ ID NO: 46) |
| | | ACCTTGAAGATCTGTAGGGA (SEQ ID NO: 47) |
| | | CTTCAAGGTGGGCAAGGAAC (SEQ ID NO: 48) |
| | | ACAGATCTTCAAGGTGGGCA (SEQ ID NO: 49) |
| | | AAGAGATCAGGTCCAAGGCC (SEQ ID NO: 50) |
| | | GGGCAAGGAACTGGGCCAAG (SEQ ID NO: 51) |
| | | TAGGGAGGGATAAAGAGATC (SEQ ID NO: 52) |
| | | ATCAGGTCCAAGGCCAGGTG (SEQ ID NO: 53) |
| | | AGGTCCAAGGCCAGGTGCGG (SEQ ID NO: 54) |
| | | TGAACCACCGCACCTGGCCT (SEQ ID NO: 36) |
| | Cpf1 | TCCCTCCCTACAGATCTTCAA (SEQ ID NO: 37) |
| RPL34-OSTC | Cas9 | TTATCATGATACTATGTCCC (SEQ ID NO: 28) |
| | | AGTTACTGAGCACAGTGCCT (SEQ ID NO: 29) |
| | | GAGTTACTGAGCACAGTGCC (SEQ ID NO: 30) |
| | Cpf1 | CAAGCATTTATCATGATACTA (SEQ ID NO: 31) |
| | | TCATGATACTATGTCCCAGGC (SEQ ID NO: 32) |
| AKIRIN1- | Cas9 | ATTAATGCCTTTAAATAGGT (SEQ ID NO: 58) |
| NDUFS5 | | TCTTAAACCTACCTATTTAA (SEQ ID NO: 59) |
| | | CCACTTCCCTCCTCCATTAA (SEQ ID NO: 60) |
| | | GCTAATTAATGCCTTTAAAT (SEQ ID NO: 61) |
| | | CTTTAATGGAGGAGGGAAGT (SEQ ID NO: 62) |
| | | CCTTTAATGGAGGAGGGAAG (SEQ ID NO: 63) |
| | | TTTAATGGAGGAGGGAAGTG (SEQ ID NO: 64) |
| | Cpf1 | ATGGAGGAGGGAAGTGGGGTG (SEQ ID NO: 65) |
| | | AGAGAATCTATGTCACCCCAC (SEQ ID NO: 66) |
| | | AAGGCATTAATTAGCGTTTGC (SEQ ID NO: 67) |
| | | AATAGGTAGGTTTAAGAGAAT (SEQ ID NO: 68) |
| | | CATGCACTACTTTAAAATTTT (SEQ ID NO: 69) |
| | | AAGTAGTGCATGCAAACGCTA (SEQ ID NO: 70) |
| PRDX1-AKR1A1 | Cas9 | AAGGGCCAAGGGAGTTAGTG (SEQ ID NO: 87) |
| Site 2 | | AGCTCCCTCACTAACTCCCT (SEQ ID NO: 88) |
| | | AGGGCCAAGGGAGTTAGTGA (SEQ ID NO: 89) |
| PRDX1-AKIR1A1 | Cas9 | ATGAAAAATAAGCCCGGTAG (SEQ ID NO: 90) |
| Site 3 | | CAGGCTGAGTCACCACTACC (SEQ ID NO: 91) |
| | | ACAGGCTGAGTCACCACTAC (SEQ ID NO: 92) |

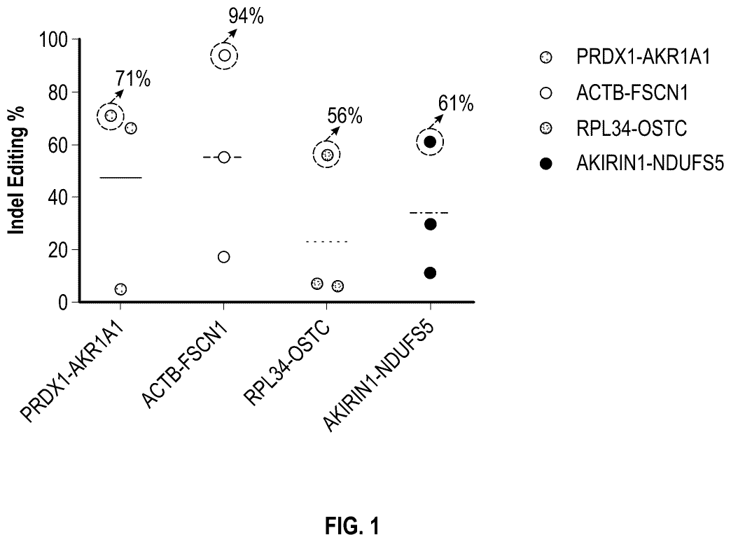

For each STAPLR site, human iPSCs were nucleofected with each individual gRNA complexed with Cas9 nuclease in the form of a ribonucleoprotein (RNP). Three days later, the nucleofected cells were harvested, genomic DNA was extracted, and PCR amplification of the genomic region flanking the intended cut site was performed. Purified PCR product was sequenced and the sequencing data were analyzed for overall cutting efficiency through Synthego's ICE Analysis Tool (available at Synthego's website) (FIG. 1). gRNAs were considered to be efficient when showing greater than 50% indel editing.

The data show that there was at least one efficient gRNA (>50% indel editing) per STAPLR site. The gRNA that had the greatest overall cutting efficiency was selected for use in future experiments to integrate transgenes at STAPLR sites.

### Design of STAPLR Homology Arms

A list of gene neighbors consisting of genes that were both highly expressed was generated. This list was filtered to remove gene pairs that contained at least one gene that is a known tumor suppressor gene or oncogene. Initially, gene pairs with less than 5 kb intergenic distance between them were discounted. However, gene pairs with only about 100 base intergenic distance between flanking genes can also be annotated and tested. Promoter regions, enhancer regions, CpG islands, and regions containing epigenetic markers were avoided in the design. Subregions that avoided regulatory elements and were capable of being synthesized in a donor plasmid were classified as potential homology arm regions and were used as the basis for a gRNA search (Table 4).

TABLE 4
Parameters for Selecting STAPLRs
STAPLR Site Parameters

Site is flanked by adjacent highly expressed genes
Neither flanking gene is a known tumor suppressor gene or oncogene
>50 base intergenic distance between flanking genes
Site avoids annotated regulatory elements and repetitive regions
Site allows for homology arms to be synthesized and cloned into donor
construct
Site has CRISPR gRNAs available that have predicted high efficiencies
and low number of off-targets

After selecting gRNAs with predicted high efficiency, homology arm sequences were finalized to center selected gRNAs within an 800 bp left homology arm and an 800 bp right homology arm that flanked the intended site of transgene integration. Table 5 indicates the intergenic distance in base pair between the two gene neighbors for each exemplary STAPLR site, along with the coordinates for each set of STAPLR left and right homology arms based on the hg38 human reference genome. Gene distances were calculated using NCBI's RefSeq database.

TABLE 5
Intergenic Distance Between STAPLR Gene Neighbors
and STAPLR Homology Arm Coordinates

| | Homology | | Distance from | Distance from |
|---|---|---|---|---|
| | Arm | | Insertion Point | Insertion Point |
| | Chromosomal | Intergenic | to Upstream | to Downstream |
| Gene | Coordinates | Distance | Gene Neighbor | Gene Neighbor |
| Neighbors | (Hg38) | (bp) | (bp) | (bp) |
| RPL34- | chr4: | 20,100 | 8,568 | 11,532 |
| OSTC | 108,638,253- | | | |
| | 108,639,852 | | | |
| ACTB- | chr7: | 62,214 | 15,223 | 46,991 |
| FSCN1 | 5,545,025- | | | |
| | 5,546,624 | | | |
| AKIRIN1- | chr1: | 20,229 | 7,533 | 12,696 |
| NDUFS5 | 39,012,798- | | | |
| | 39,014,397 | | | |
| PRDX1- | chr1: | 27,888 | 3,449 | 24,439 |
| AKR1A1 | 45,525,540- | | | |
| site 1 | 45,527,139 | | | |
| PRDX1- | chr1: | 27,888 | 2,286 | 25,649 |
| AKR1A1 | 45,524,377- | | | |
| site 2 | 45,525,976 | | | |
| PRDX1- | chr1: | 27,888 | 6,713 | 21,222 |
| AKR1A1 | 45,528,804- | | | |
| site 3 | 45,530,403 | | | |

Sequences for the left and right homology arms of the targeting constructs based on the hg38 Human Reference Genome are shown in the table below.

TABLE 6
STAPLR Left and Right Homology Arms

| STAPLR | | |
|---|---|---|
| Site | Left Homology Arm | Right Homology Arm |
| AKIRIN1- | TTCTCATTTAGCAAGAGTGGAAAACTG | TATTTAAAGGCATTAATTAGCGTTTGC |
| NDUFS5 | TTGTGGATCCCAGGAGAAGTCTGGAGC | ATGCACTACTTTAAAATTTTCCTCTCA |
| | TAGGTAGTAGGGGCTAGATAATAGAGG | ATTGCTTGAGTCCAGGAGTTCGAGGTT |
| | GCTTTGACTAATAGGAGTTTGGGCCTT | ACGGTGAACTATGATTTCACCATTGCA |
| | ATCCTGTGGAAATCTGAAGGGTTTTGG | GTCCAGCTGAGGCAACAGAGAGAGACC |
| | GAAAATGAATTCAGGGCTAGGTGCAAG | CTCTTAAAGAAAAAAAAAACTTTTTCC |
| | ACAGAAGGGCAAATACCAGACTAAGAC | TCTCAGACTCTGTCCCTAGCATTGACT |
| | ACACAGCCACTAAGAGCCTCTTTTTTT | CACCCCATCATTTTTTTTTTTTTTCTT |
| | TTTTTTGTCCAGTCAGAGGTAATGAGG | GAGATGGAGTTTCAAGAAAAAACTCCA |
| | CCTATATATTTATTAGGAAGGATGATT | GGCTGGAGGCAGTGGTATGGTCTCGGC |
| | AACCATGGCTGTAGCTTCACCTAAGAG | TCACTGCAACCTCCGCCTCCCGGGTTC |
| | GAACGTGGCTCTTGGGCCGGGTGCGGT | AAGCGATTCTCCCTGCCTCAGCCTCCC |
| | GGCTCACGCCTGTAATCCCAGCACTTT | AAATAGCTGGGATTACAGGCACCCGCC |
| | GGGAGGTAGAGGTGGGCGGATCACCTG | ACCACACCCAGCTAATTTTTTGTATTT |
| | AGGTCAGGAGTTCAAGACCAGCCTGAC | TTAGCAGAGACGGGGCTTCTCCATATT |
| | CAACATGGAGAAACCTCTTCTCTACTA | AGCCAAGCTGGTCTGGAACTCCTGACT |
| | AAAATACAAAATTAGCCGGGCGTGGTG | TCAAGTGATCCACCCACCTTGGCCTCC |
| | GTGCATGCCTGTAATCCCTGTTACTCG | CAAAGTACTGGGATTACAGGCACGAGC |
| | GGAGGCTGAGGCAGGAGAATCGCTTGA | CACCGCGCCCGGCCTTCACCTATTATA |
| | ACCTGGGAGGTGGAGGTTGCGGTGAGC | TCTATTCTTGTCTTTTAAATAGTGAGT |
| | TGAGATCACACCATTGCACTCCAGCCT | GACTTCACAGCTCAGTCTCTTTGTTCT |
| | GGACAACAAGAGCAAAACTCTATCTCA | GAACTTCCATTCTGAGTCGCAACCCAG |
| | AAAAAAAAGAGAGGAATGTGGCTCTTG | GTACACCTTAGGAAGGGCGGGATCCCG |
| | AAGAATCTTTCTAGTCTCATCTTCCCT | TTATCCTGTCCCTGAACATCAACTACT |
| | GTCCCCTTAGACACACCTTCAAAGATC | GAGACAGCTGATGAAGCTGGCTCGGTG |
| | CTTTTTCTTTTGGTTTGGCTTGTCTTT | TGTGACGGAGGCCCCCTCAGAGTCAGC |
| | CCCCCTCACTTTACCTAATCCAGCCCA | AGAGCCGAGCGTACAGCGCACATCAGG |
| | ATTCCATAGCCACTGCTGGTTCCTTTA | CAGAGCAGACCAGAGGCAGGGAAGACA |
| | ATGGAGGAGGGAAGTGGGGTGACATAG | CACTGGGGCAGGGCCAGTGGGCACATC |
| | ATTCTCTTAAACCTACC | TGGACCACCTGCGAGGG |
| | (SEQ ID NO: 17) | (SEQ ID NO: 18) |
| RPL34- | TTATTCTTCACAGAGGACAGCTCCCAA | CACAGGCACTGTGCTCAGTAACTCAAG |
| OSTC | CTCTTTACCTCTCTTCCTAATGCAACA | TCATTATAATACTGTGTGCTTGACAAT |
| | CTCACTCTCACTCTCAGCAGCCTTATA | ATGTTGTACTTACTGGCCAAAAATCAC |
| | ATATCTGTAAATGTAGAAACTATCAGT | CAATGTCTAATTGTCAAGCTAAATGTC |
| | TATGAACTTCTGTAACTCCCTTTTCAT | TTCTTTCAATTTCAATCTATCTGACAT |
| | CTTTGGCACCTCAAAACTTTAATGTAT | GCTGGAAACTTCCCTTTCCAAGGCTTC |
| | GTAAAAGCATGGGCTTTGAAATTGGTA | TTTGCTATCGCTCCCTTGGCTCTTCTG |
| | AACTGGAGCTCTATCAATCTCTGGCTG | TTCCTTTGATCTTGCTGTATTTACTTT |
| | TGTCAACTTGAGCATGTTGCCCAATTT | GTAGTATCCTCCTAATTATGGGGGTGA |
| | CTATGAGCCTCAGTTTTCTTATCTAAA | TTCCCTAGGCATCCATCTTCAGCTTTC |
| | AAATGAAACTAATAATGCCTACTCCAA | TTCTCTCTCACTCTTTTAACTTTACTC |
| | AGAATTGTTGGATGGATTAAGTAAGAC | TGTGAATTTCCTGTAATTATACACTAC |
| | AACCTAATATAATTGCTGTGCATAGTA | ATTTGGTGTGTGTGTGTGTGTGTCTGT |
| | AATCCTAACTGTTGGTTGCCTTATACC | GTGCACGTGCATTTTTCTAGGAAGGAG |
| | TCTAATCTTCTTTCTGCTCTCTTTTCT | TTCATAGCTTTTATCAGCTCTGCAAGG |
| | TTAGAAGATGGCATGCATCTTTTCCAA | AGGTCTATGACCCTCCCAATAACAACC |
| | GGTAAATTTTACCTCTGTTGAACTCTA | CTGACCAGTTCCACCTCACGTGTAGAC |
| | TCCCATACTCTCTTGCCATGGCCCTAA | ACATTCTTCCACTGTGATGACTTCCAT |
| | TTTTGTTCAGTTACTTAACTCCTTCCC | TATCCTCCACATGCTGCTGATTCCTGG |
| | TTCCCTCTGACATCCTTGGTCCCTTCC | ATCTCCAACCCTAAACCTCTTTCCCAT |
| | TCTCCACAGTTTTATTCTCTCGTCTTA | GCTCCAGCGACCATCTTCCCAACTCTG |
| | CACAAACATGCCCAAGTCTCATCATCC | CCTAGGTCCCTCCTGGAGGGTCCTTCT |
| | ATAAAAACCTCTGTCATTACCACTTCA | AGAATAATAGTTCTCATCAGGCAGGTG |
| | GCTCTCTTCTTCTGTTCATACCCAGCT | GCAGAACCTCTGTCTCCCACTCCTTGC |
| | GTGTTGGGTGCCATGAATCATTCACTA | ATGCCCCTCTGTGAGGCTTGGGTGGGT |
| | CTATTATTTTTAAAATTCCTATCACTA | TGGGGGTGATGGGAGGGGTGCGTGTAT |
| | CTTCTCCCCTCTGCAATCTGGCTTCTC | AATCAGAGTCCCTGGGGTGCAGAAGCT |
| | CTGCCAACATGCTACTGAAATTAATGA | CTGTTCTGACCTTGCCTCTTGCTATCC |
| | ATTAATTCATCAAATATTTACAAGCAT | ATGGAATTACTGCTCTATCCTGGCCAT |
| | TTATCATGATACTATGT | TTTCCCATTCTCTTTCT |
| | (SEQ ID NO: 19) | (SEQ ID NO: 20) |
| ACTB- | GAAGCCGGGTGTGGTGGTGCATGCCTG | GTAGGGAGGGATAAAGAGATCAGGTCC |
| FSCN1 | TGGTCCCAGCCACTTGGGAAACTGAGG | AAGGCCAGGTGCGGTGGTTCACGCCTG |
| | TGGGAGGATTGCTCAAGCCCAGGAGGT | TAATCCCAGCACTTTGGGAGGCCGAGC |
| | CAAGGCTGCTGTGAGCTATGATTGTGA | CAGGCAGATCGCGAGGTCAGGAGTTCG |
| | CACTGCACTCCAGCCTGGGCAACAAAG | AGACGAGCCTGGCCATCATGGTGAAAC |
| | GGAGACCCCTTAACTAACTAAATGAAT | CCCGTCTCTACTAAAAATACAAAAATT |
| | GAATGAATGAATGAATGAAGGCAACCT | GGCCAGGCATGGTGGCAGGCGCCTGTA |
| | GAGCTGCATTCCTTGGCCTGCCAACCT | ATCCCAGCTGAGGCAGGAGAATCGCTT |
| | GCCCAGCCCCATCCCTCAGCCCTCCCT | GAACCCGGGAGGTGGAGGTTGCAGTGA |
| | GAGTCTGAGGGCCCTGCAGGTCCCACA | GCCGAGAGCGAGCCACTGCACTCCTGC |
| | CAGGGCCAGGCTCCATCTTGTTTCTGC | CTGGGTAACAAAGCAAGACTCCATCTC |
| | AAATTTGCACCTTCCGTTCCATTCCCT | AAAAAAAAAAAAAAAAAAAAAAATCAA |
| | GCCACACTGCTCACGGGTCACCATGGA | GTCTGGCGCAGCCCCAGGAAGCAATCT |
| | TGCTGGATAACTGCCACCGTCTCCTTG | TGGGCTGGGCACACCGCATTTCTGTGA |
| | GAGAAGCCCTCCGTCATCTCAGCCCCT | CATGAAGGCAGCGACACCTCGTCGCTG |
| | GATGTCAGGTATTCAACCTGCAAATCC | TCACAGGGTTGCTGGGGTTGGGGGCTA |
| | TCCCTGTAGGTGCTGGGCTCTCCAGAG | GAGGAGAGGAGGAGCCTCTGTTGAGGG |
| | CCCTGGGCTGTGTGCAGGGGACTCAGG | CTCCAAGAAGGGACAGAGAGGTGGTGC |
| | GGAGAACGGGCCCCACCCAGCTCCTTC | TCATGGGTCCTGGAGACACCTTTTGGG |
| | CTCACAGAGCTTTCAGAGGCTGGGGGC | TGGTTCGTGCCCTCCATCCTGGGCCAC |
| | CTCCCTGATCCCCTCCCAGATCCCAAG | TTTGGGGAGGTGAAGGAGGGAAGCATT |
| | GCCCCTGCTGCCCCCACCCTGCAGACT | AAGGGACAAGACCCCCCGTTCCCAATT |
| | GGGACTCCGTGAGGCTGGGCTCTGACT | TCTCTCCGGAGCCAGGGTTTCTCTGAT |
| | TGGATATTGTGGTTCCAGCACACAGCA | TCGAAGAAACAGGTGTCACAACCCAGG |
| | GGCACCGTGGCTGTAGTAGGCGTGCAT | AAGTCCACTGATGGCATCTGCCCTGGG |
| | GGGAAGTCAGGAGGAGCAGACCTGTCA | GCATCAGCATTTAGGGCTGATCACTGA |
| | TCTCCCCTGCTGAGGACACAGCCCGGT | GGTCTGCACCTCCCAAGGCTGCTGTGC |
| | CAGCGTGTCTTGGCGGCCTGGGGCCAG | CCATTCCTGGGCGCCCCAAAGGGGAAG |
| | TGACTGAGCCTCTTGGCCCAGTTCCTT | AAAAACTCCTGAATGTGCACCGGGACA |
| | GCCCACCTTGAAGATCT | GGACCCATCCCATGCGG |
| | (SEQ ID NO: 21) | (SEQ ID NO: 22) |
| PRDX1- | AGAAAATTGCCACCTATTGTATTCACC | CTAGGCAGTGCTGCAAGAACCAGTCTC |
| AKR1A1 | ATATGCCAGGCATTGGGCTGGGCACTT | TAAACAAACAAAGAAAAAAAAACAGGA |
| Site 1 | GAGTTTATTTTTCTAATTCAAGAACAA | AAAAAAAAGAGTTATCCTAGCCTAGAG |
| | GGCCAGGCACGGTGGCTCATGCCTGTA | AAATGTTAACAGTCTATCCCCTTAAGA |
| | ATCCCAGCACTTTGGGAGGCTAAGGCG | TCATAAAAGTGAAATAACTGGACAGAG |
| | GGCAGATCACAAGGTCAGGAGTTTGAG | TACTGAAAAAAGAAGAAATCTGTTGAA |
| | ACCAGCCTGGCCAACATGGAGAAATCC | CAAGTCTAAGTGATTTGTAGAGTCTCT |
| | CATCTCTACTAAAAATACAAAAATTAG | CCAAAGGACAGTAAAGATTTCAGAACC |
| | CTGGGTTTCGTGGTGTGCACCTGTAGT | ATGGGGAGGAGGCTTTCTTGCAGGGTC |
| | CCCAGCTACTCAGGGGGCTGAGGCAGG | TGATGTATACCTCTAGTTCTATTTTTA |
| | AGAATCTCTTGAACCCAGGAGGCGGAG | GATATATTCCATTGTGCTCTCTTCATT |
| | GTTGCAGTGAGCCGAGATCGCGCCACT | TGCTCACCATTAGCATGGGAATTATTC |
| | GCACTCCAGCCTGAGTGACAGAGTGAG | CCCTTGTTTTATTTATATTTGTAGTAA |
| | ACTCCGTCTAAAAAAAAAGAACAAAAG | CAAGTCTAGCACAGTAACTGGCATAGA |
| | CCGGGCATGGTGGCTCAAGCCTGTAAT | AGTACTTAATAAATGTTACAATGTGTT |
| | CCCAGCACTTTGGGAGGCCAAGGTGGG | GAAGACTTTTGAGATGTAGAATTAAAT |
| | CAGATCATGAGGTCAGTAGTTCCAGAC | TATTTGAACTACGTTTCAGGAAGTTGA |
| | CAGCCTGACCAACATGGTGAAAACCTG | CTCTGGAAGCTGTGTGATGAATGAATT |
| | TTTCTACTAAAAATACAAAAATTAGCT | TAGTGACTAGGAGATTAGGCAAAGAGA |
| | GGGCGTGGTGGCAGGCACTTGCAATCC | GCAATTGGGAGGCCACGGCAATGATCC |
| | CAGCTACTCAGGAGGCTGCAGCAGGAG | AGACAAGAGAAGTCTCAGAACTGTAAT |
| | AATCACTTGAACCCGGGAGGCAGAGGT | ATGACATTAGCAGCAGGGTAGAAAGAA |
| | TTTAGTGAGCTGAGATCCCGCCACTGC | GGAAATACAAGGTATATTTCTTCAGGC |
| | ACTCCAACCTGGGCAACAGAGCAAGAC | TTTAGGATTAATTGGATTTAGGATGAA |
| | TCTGTCTCAAAAAAAAAAAAAAAGAAA | AGGAAAGAGTAAATTAAATCCAGTTTC |
| | AAAGAAAAAAAGAAAAGAAAAAAGAAC | TTGGTGACATGAGTGATGTCATTAACC |
| | TAAAAGAGAGTGGAGCACATTTGGCAT | TGTGACATAGGAAACACTGGAAGAAAA |
| | GTGACTATAGTCCCAGCTACTTGGTAG | GCAGGTTTGAGGTTTAAAATAATGTTA |
| | GCTGAGGTGAGAGGATCACTTGAGCCC | AACAGGCCGGGAGCGGTGGCTCACGCC |
| | TGGAGTTTGAGCCCAGC | TGTAATCCCAACATTTT |
| | (SEQ ID NO: 23) | (SEQ ID NO: 24) |
| PRDX1- | AATAATATTAATACTTTATTGTGATTA | TAACTCCCTTGGCCCTTATAGTGGGAT |
| AKR1A1 | TCTATGTAGTTCAGTACAGTACTTAGT | AATGAATGAGGAAGTGCTGTCTCATGC |
| Site 2 | TCTTATTATAAATTAGAAATGTTAGCT | TCATTGAGCATCTCAATTTTTGTTATT |
| | GCTATTAGTATAACTCTTCCATCCTTC | TATCATACTACTTTGCATAGACGTGAT |
| | ACACTTATAACACCCAGAACACAGTGT | TTATTTGCTTTTTCCTTCTTTGCCTGA |
| | AGTTGTTTCAAGCCTTCAGACTTTTTT | GGCTGAATAGTTAGTTGGCATTATTCC |
| | TTTTAAGATGGAATTTCGCTTTTCTTG | TTTTTTTAATTTTTATGTAACTTTTAT |
| | CCCAGGCAGGAGTGCAATGGCACGACT | TCTTTTTTATCAGGCAGCCACCCGAGC |
| | TCTGCTCACTGCACCCTCCACCTCCAG | CAGCAGCCAGCGTAGGCTCAGAGAGAC |
| | CGTTCAAGCAATTCTCCTGCCTCAGCC | TCCCCTTTCTTTCTTGCAGGAGCAAGG |
| | TCCCAAGTAGCTGGGATTACAGGCGCC | GCAGCATGAAAGTTCAGAGCTTAATAA |
| | CACCACCACGCCCGGCTAATTTTTTGT | TGGATGGTTATTGGCTATGGGCAGGCT |
| | ATTTTTAGTAGAGACGGGGTTTCAGTA | AAACCAATACACAGTACATACAGTCAT |
| | TGTCAGCCAGGCTGGTCTCAAACTCCT | ACTTCAGCCCAAAGAAAATTGCCACCT |
| | GACCTCAGTTGATCTACCTGCCTCAGC | ATTGTATTCACCATATGCCAGGCATTG |
| | CTCCCAAAGTGCTGGGATTACAGGAGT | GGCTGGGCACTTGAGTTTATTTTTCTA |
| | GAGCCACTGAGCCCGGCCGCCTTCAGA | ATTCAAGAACAAGGCCAGGCACGGTGG |
| | CTTTTGTTTGAACCTGGAAAACTCTTT | CTCATGCCTGTAATCCCAGCACTTTGG |
| | CCCTGGAGAAAACATGTCTTTCAGCAT | GAGGCTAAGGCGGGCAGATCACAAGGT |
| | TACTTTCTCTCCGGCTGTTTCTTACCT | CAGGAGTTTGAGACCAGCCTGGCCAAC |
| | TTACTCATTCCCAGGATTTACAATTCC | ATGGAGAAATCCCATCTCTACTAAAAA |
| | CCTCCTTTGTGTCCAAACAATAGCTTG | TACAAAAATTAGCTGGGTTTCGTGGTG |
| | TATAGCCAAAAAAATCTTTTAAGCAAT | TGCACCTGTAGTCCCAGCTACTCAGGG |
| | TTTAGTTTTACATTTCTTCCTCTGCCT | GGCTGAGGCAGGAGAATCTCTTGAACC |
| | GCTAGAGTGACTTTCTGGAGAGTAGGA | CAGGAGGCGGAGGTTGCAGTGAGCCGA |
| | TGTGGGTGACATCTGTCTCTGTGTCCC | GATCGCGCCACTGCACTCCAGCCTGAG |
| | CAAAGTCACAACAAAGCATCTGATATA | TGACAGAGTGAGACTCCGTCTAAAAAA |
| | AAATAGGAGCTCAGTCAGTGAGCATTC | AAAGAACAAAAGCCGGGCATGGTGGCT |
| | AATCAATAGTATCATGTTTCTGTCTCT | CAAGCCTGTAATCCCAGCACTTTGGGA |
| | GTCCCCAGCTCCCTCAC (SEQ ID | GGCCAAGGTGGGCAGAT (SEQ ID |
| | NO: 93) | NO: 94) |
| PRDX1- | TGTTCACGTGTTTGTCTGCTGACCCTC | CCGGGCTTATTTTTCATACAGATGATT |
| AKR1A1 | TCCCCACAATTGTCTTGTGACCCTGAC | TAGAATATGTTTATGTTAAATTTTTTT |
| Site 3 | ACATCCCCCTCTCCGAGAAACACCCAC | AGATTATCACTAAAAGAAAACAGTTGA |
| | AAATGATCAATAAATACTAAGGGAACT | ATTCTTCAATTTACAAGGATTCAATTT |
| | CAGAGGCTGGCGGGATCCTCCATATGC | TTAAAATTATATTTGAATGTACAGAGG |
| | TGAACGCTGGTCCCCTGGGTCCCCTTA | CCTACAAATACCAATTTCAGAAACACT |
| | TTTCTTTCTCTATACTTTGTCTCTGTG | GTTATCAAGGATATCTATTTGTTAAAA |
| | TCTTTTTCTTTTCCAAGTCTCTCGTTC | TTGAATGCAACTGAAAGTAACAGATTA |
| | CACCTAACAAGAAACACCCACAGGTGT | ACTGCAAATAACAAGCTTAACCAAATA |
| | GGAGGGGCAACCCACCCCTTCACCATC | GGGGATTAATTTTTCCCATTTAACAAG |
| | TCCACAAAAAATAAAAAATTAGCCCAG | GAGACAGGGCCGGGCACAGTGGCTCAC |
| | TGTGGTGGTGTGTGCCTGTGGTCCTAA | GCCTGTAATCCCAGCACTTTGGGAGGT |
| | CTAGTCCAGAGGCTGAGGTGGGAGGAT | CGAGGCGGGTGGATCACTTGAGGTTGG |
| | TGCTTGAGTCCCAGAGCTCGAGGCTGC | GAGCTCAAGACCAGCCTGACCAACATG |
| | AGTGAGCCGAGATGGCACCACTGTACT | GAGAAACCCCGTGTCTACGAAAAATAC |
| | CCAGCCTGGGTGACAGAGTGAGCAGAG | AAAATTAGCCGGACGTGATGGTACATG |
| | TGAGACCCTGTCTCAAAAAATAATAAA | CCTGTAATCTCAACTACTCGGGAGGCT |
| | ATAAAATAATGGGAACCCAAATCACAA | GAGGCAGGAGAATGGCTTGAACCTGGG |
| | GACAGCACTCATTTTTCTTTTATTTAT | AGGTGGAGGTTGCAGTGAGCCAAGATC |
| | TTTATTTTTTTTGAGACAGAGTCTTGC | GCGCCATTTGCATTCCAGCCTGGGCAA |
| | TCTGCTGCCCAGACTGGAGTGCAGCAG | CAAGACGAGCTCCGTCTCAAAAAAAAA |
| | CATGGTTTCGGCTCACTGCAACCTCCA | AAAAAAAAAAAAAAAGCAAGTAGACAG |
| | CCTCCTGGGTTCAAGTGATTCTTGTGC | GGCCAGGTGGAGTGGCTCATGGTTGTA |
| | CTCAGCCTCCCAAGTAGCTGGAATTAC | ATCCCAGCATTTTGGGAGGCCGAGATG |
| | AGGTATGTGCCACCATGCCTGACTGAT | AGAGGATCATTTGAGCTCAGGAGTTGG |
| | TTTTGTATTTTTAATAGAGACAGGGTT | AGACCAGCCTGGGCGACATAGGGGTAC |
| | ACGCCATGTTGTCCAGTCTGGTTTCGA | CCATCTCTACAAAAAATTAAACAAACA |
| | ACTCCTGGTCTCAAGGGATTCACCCAT | AACAAAAAATAGCCAAACATGATGACG |
| | CTCGGCCTCTCAAAGTACTGGGATTAC | CATGCCTGTAGTCCCAGCTACCCAGGA |
| | AGGCTGAGTCACCACTA (SEQ ID | GGCTGAGATGAGAGAAT (SEQ ID |
| | NO: 95) | NO: 96) |

### Example 2: Testing of Inducibility and Transgene Expression at STAPLR in a Pooled Population of Targeted iPSCs

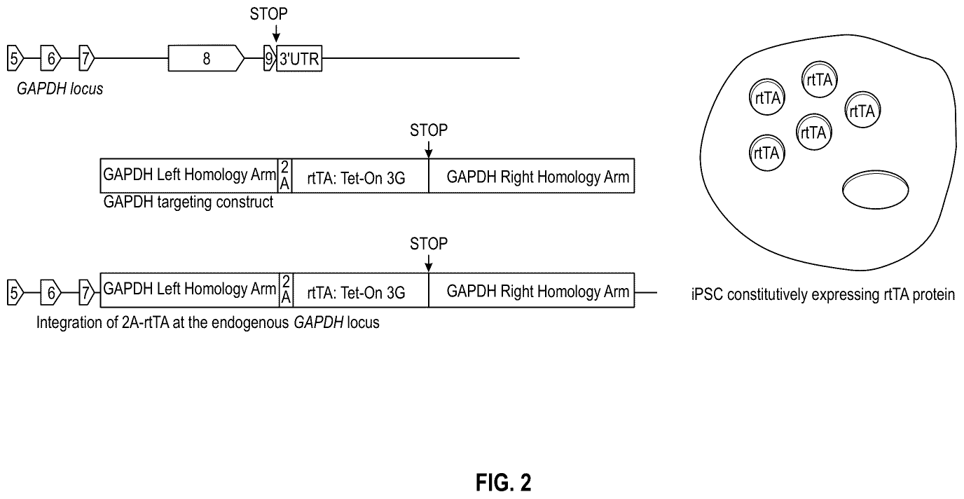

To test for robustness of inducibility and transgene expression at each of the four annotated STAPLR sites (PRDX1-AKR1A1 (Site 1), ACTB-FSCN1, RPL34-OSTC, AKIRIN1-NDUFS5), the dual component doxycycline-inducible rtTA/TRE system Tet-On 3G from Clontech/TakaRa (as described in U.S. Pat. No. 9,127,283, incorporated by reference herein in its entirety) was used. For the constitutive component, the Tet-On 3G rtTA (reverse tetracycline transactivator) was expressed biallelically from the GAPDH locus, via the inventors' “sustained transgene expression loci” (STEL) approach (FIG. 2) as described in WO 2021/072329, incorporated by reference herein in its entirety.

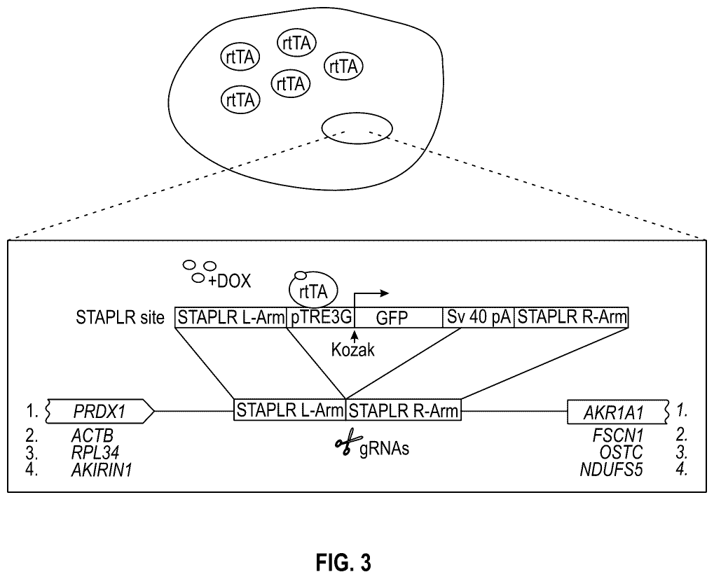

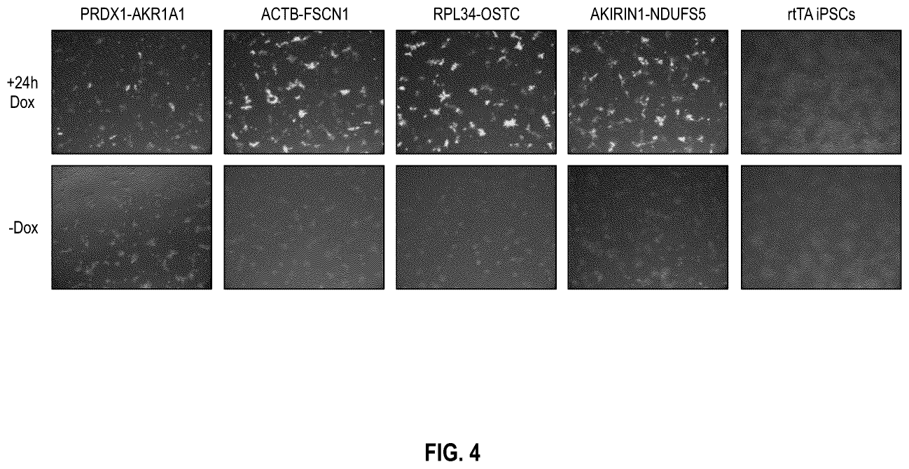

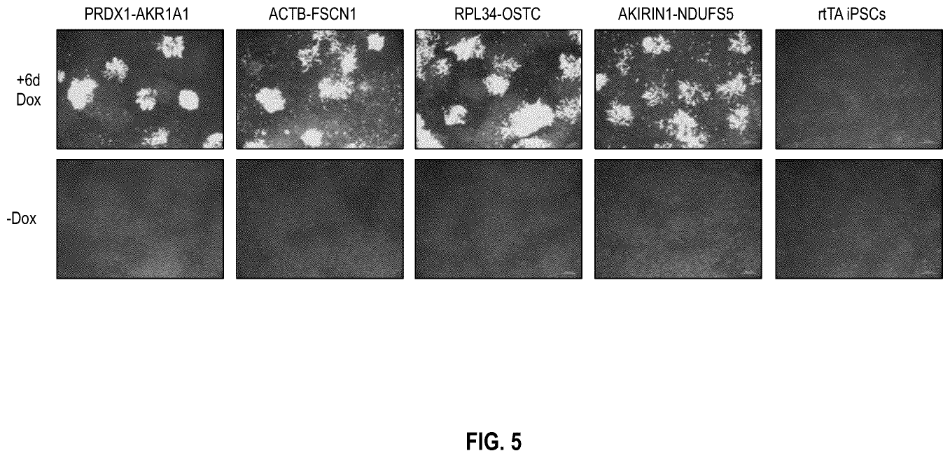

To test inducibility of transgene from a STAPLR site, the TRE3G promoter was used to test expression of an eGFP cargo. A Kozak sequence was included to enable translation initiation and an SV40 PolyA sequence was added to enable transcription termination. In the presence of doxycycline, the rtTA protein binds to and activates the tetracycline-response element (TRE) minimal promoter (FIG. 3). For each STAPLR site, a parental iPSC line with bi-allelic rtTA integration at GAPDH (GAPDH::rtTA iPSCs) was nucleofected with a selected high-efficiency RNP and the corresponding STAPLR targeting construct (STAPLR left homology arm-TRE3G promoter-eGFP-SV40-STAPLR right homology arm). Pools of cells that received both STAPLR RNP and STAPLR targeting construct were fed with media containing 2 μg/ml doxycycline starting at day one post-Nucleofection (FIG. 4) and continuing to day seven post-nucleofection (FIG. 5) in order to induce GFP expression. The parental rtTA iPSC line was also given 2 μg/ml doxycycline media as a control. GFP expression was monitored over the course of a week by fluorescent microscopy. An increase in GFP intensity was observed as cells were treated for longer duration with doxycycline. Preliminary testing of this rtTA/TRE-based transgene expression system at STAPLR indicates robust inducibility and expression of GFP in a pooled population of STAPLR site-targeted iPSCs.

### Example 3: Testing of Inducibility and Transgene Expression at STAPLR in a Clonal Population of Targeted iPSCs

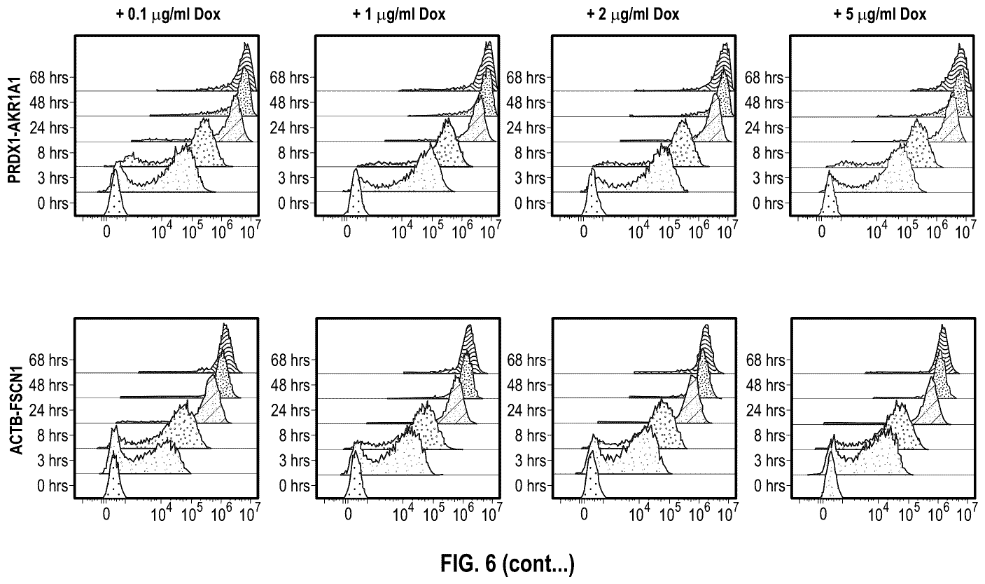

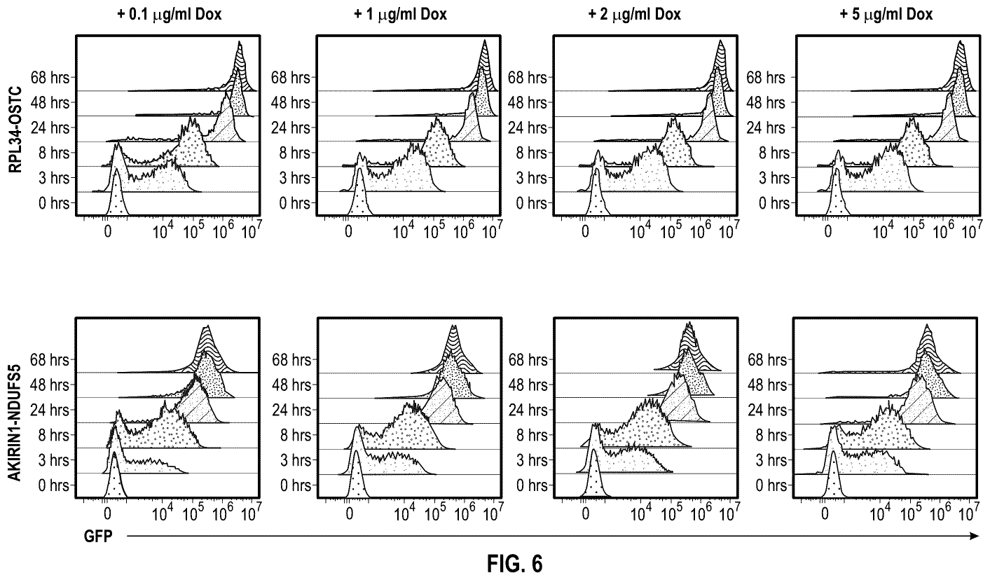

Parental GAPDH::rtTA iPSCs were nucleofected with RNP and a STAPLR targeting construct at each of the four STAPLR sites followed by plating each pooled population of STAPLR-targeted iPSCs at clonal density. Individual clones were picked and screened by PCR across the junctions of the left and right homology arms to confirm accurate integration of the TRE3G-eGFP-SV40 at each of the four STAPLR sites. Targeted iPSC clones were expanded and treated with media containing doxycycline at a range of 0.1 μg/ml to 5 μg/ml from 0 to 68 hours. Cells were collected at a time course of 0, 3, 8, 24, 48 and 68 hours over this time course of GFP induction and flow cytometric analysis was performed (FIG. 6). The results indicate that maximal GFP induction from all four STAPLR sites can be seen from administration of 0.1 μg/ml doxycycline and after 48 hours of doxycycline administration. STAPLR sites vary in their maximal expression levels of GFP, with the PRDX1-AKR1A1 site demonstrating the highest expression of GFP in doxycycline-induced iPSCs. One clonally derived line from each STAPLR-targeted site and a wildtype unedited iPSC control line was then treated with media containing 2 μg/ml doxycycline for 72 hours (FIG. 7). TheAKIRIN1-NDUFS5 STAPLR line showed slightly delayed GFP induction so treatment with media containing 2 μg/ml doxycycline was increased to 6 days (FIG. 7). The results indicate that all four treated STAPLR-targeted iPSC lines could induce high levels of GFP expression, with the PRDX1-AKR1A1 site again demonstrating the highest expression of GFP in doxycycline-induced iPSCs, while the wildtype unedited doxycycline-treated iPSC control line did not express GFP. In all instances, cells that did not receive doxycycline treatment did not express GFP.

### Example 4: Testing of Inducibility and Transgene Expression at STAPLR in iPSC-Derived Myeloid Progenitors

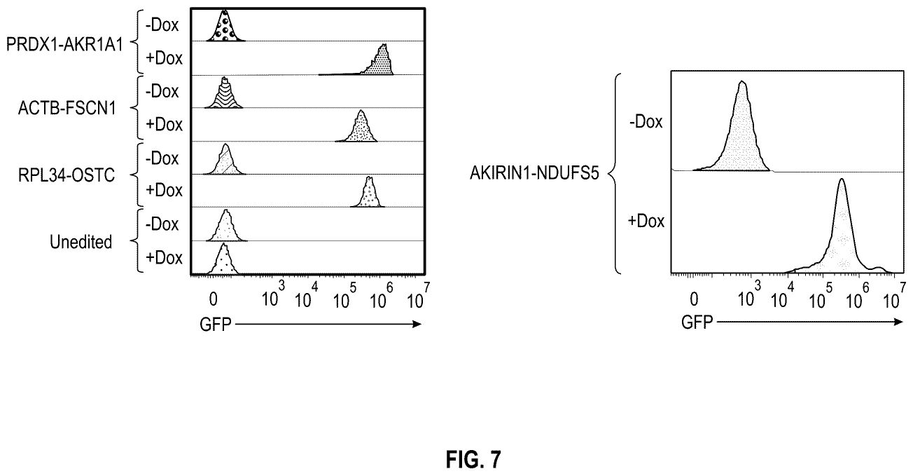

Clonally-derived STAPLR iPSC lines were differentiated into myeloid progenitor cells to demonstrate that transgene integration at STAPLR maintains sustained transgene expression in differentiated iPSCs (Douvaras et al., Stem Cell Reports (2017) 8(6):1516-24). 2 μg/ml doxycycline was added to each STAPLR-targeted clonal line at day 12 of differentiation and doxycycline was replenished daily for three days. Adherent myeloid progenitors were harvested for flow cytometric analysis of GFP induction at day 15 of differentiation. Three of the four TRE-eGFP-SV40 STAPLR lines (PRDX1-AKR1A1, ACTB-FSCN1, RPL34-OSTC) demonstrated efficient GFP induction in heterogeneous adherent myeloid progenitor cells, compared to differentiated cells that did not receive doxycycline (FIG. 8). A wildtype unedited iPSC control line differentiated using the same protocol and similarly treated with doxycycline did not show induction of GFP. One of the TRE-eGFP-SV40 STAPLR lines (AKIRIN1-NDUFS5) demonstrated delayed GFP induction under fluorescent microscopy. This cell line was replenished with doxycycline for an additional three days and adherent myeloid progenitors were harvested for flow cytometric analysis at day 18 of differentiation. FIG. 8 shows the bimodal GFP induction seen from the myeloid progenitors harvested at day 18 of differentiation. In all instances, cells that did not receive doxycycline treatment did not express GFP.

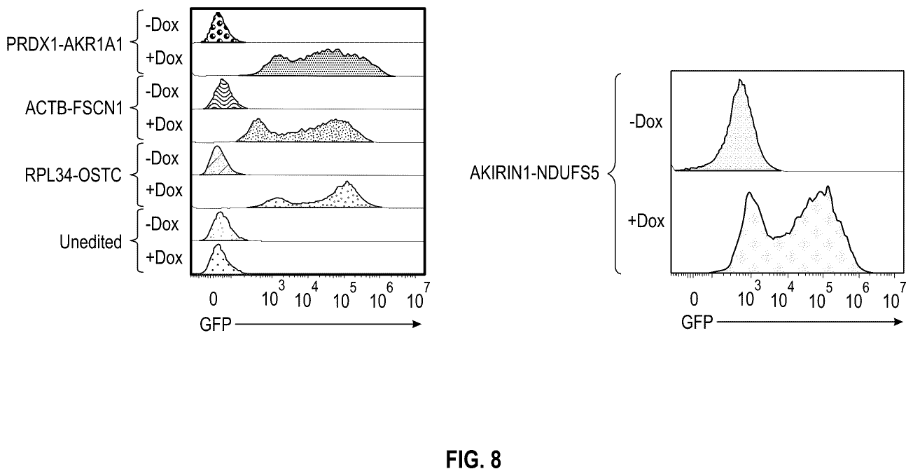

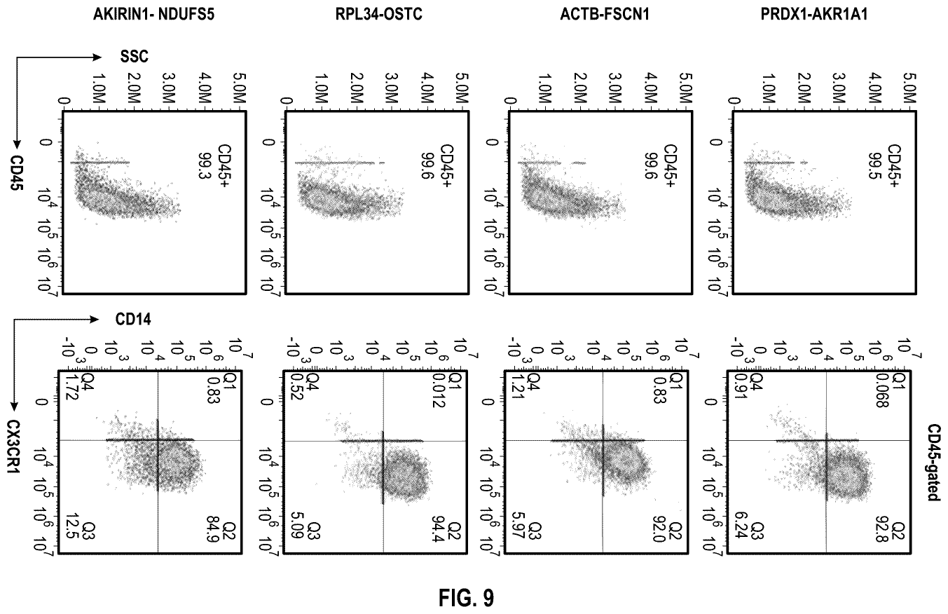

STAPLR-targeted lines were further differentiated past 30 days to the point where non-adherent myeloid progenitor cells could be collected in suspension culture. 2 μg/ml doxycycline was added for six days and the non-adherent myeloid progenitor cells were collected for flow cytometric analysis of GFP induction. All four TRE-eGFP-SV40 STAPLR lines cultured past 30 days demonstrated efficient differentiation into triple-positive myeloid progenitors as defined by >80% co-expression of the cell surface markers CD45, CD14 and CX3CR1 (FIG. 9). The doxycycline treated STAPLR lines also demonstrated efficient GFP induction in heterogeneous non-adherent myeloid progenitor cells, compared to a doxycycline treated wildtype unedited control line, with some variability in maximal GFP expression levels (FIG. 10). This data demonstrates that transgene integration at all four STAPLR sites permitted sustained expression of the transgene under external promoter control during and post-differentiation into myeloid progenitor cells.

### Example 5: Derivation of Human Induced Pluripotent Stem Cell Line with Inducible Expression of CD19t-IL12 from the PRDX1-AKR1A1 STAPLR Site

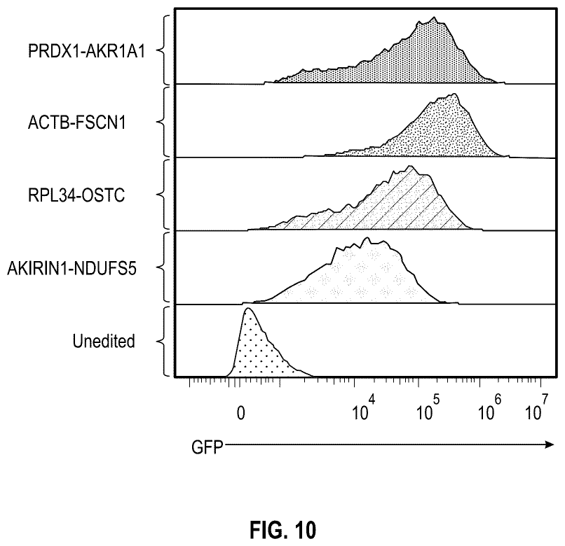

A parental iPSC line with bi-allelic rtTA integration at GAPDH (GAPDH::rtTA iPSCs) was transfected with a selected high-efficiency RNP for the PRDX1-AKR1A1 STAPLR site (Site 1) and a STAPLR targeting construct comprising a doxycycline-inducible promoter (TRE3G)-driven CD19t-IL12 cassette flanked by PRDX1-AKR1A1 left and right homology arms. CD19t was included here as a non-biologically functional cargo; it served as an epitope marker for surrogate detection of IL-12 transgene integration by flow cytometry. Two different gRNAs and their corresponding nucleases were used for targeting at the PRDX1-AKR1A1 STAPLR site. Either a Cpf1-based guide RNA with sequence 5′-GAGACTGGTTCTTGCAGCACT-3′ (SEQ ID NO: 83) or a Cas9-based guide RNA with sequence 5′-CTTGCAGCACTGCCTAGGCT-3′ (SEQ ID NO: 71) were selected to generate clonal lines. The GAPDH::rtTA constitutively expresses the reverse tetracycline transactivator (rtTA) from the GAPDH locus. In the presence of doxycycline, rtTA binds to the TRE3G promoter and induces expression of CD19t and IL-12 driven by the TRE3G promoter (FIG. 11).

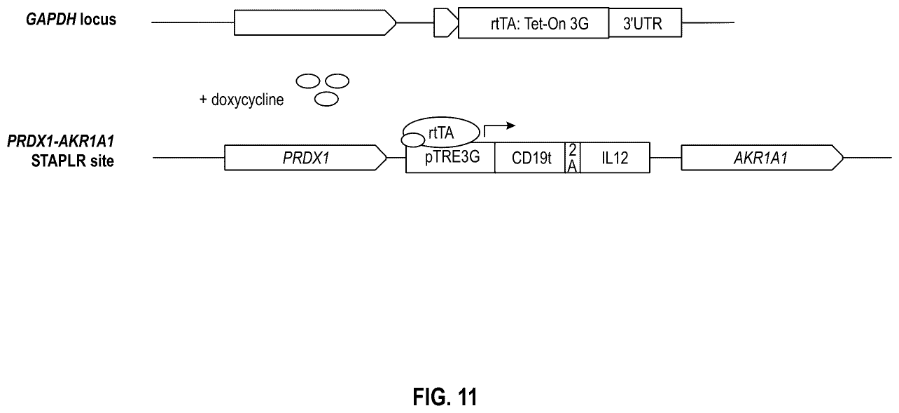

Single cell suspensions of GAPDH::rtTA iPSCs were prepared for transfection with either Cpf1 or Cas9 gRNA RNP complexes and the PRDX1-AKR1A1 targeting pTRE3G-CD19t-IL-12 DNA donor template. Two days post transfection, cells were treated with doxycycline (2 μg/mL) for 48 hours to induce CD19t-IL12 expression that was analyzed using live cell imaging of AF488 conjugated anti-CD19t antibody staining (FIG. 12, Panels A and B). Cells were then dissociated and plated at single cell clonal density. Four days after clonal density plating, growing colonies were treated with 2 μg/mL doxycycline for 48 hours to induce CD19t-IL-12 expression. Colonies were analyzed with live cell imaging using an AF488-conjugated Ab against CD19t after the 48-hour doxycycline treatment. CD19t positive colonies were identified (FIG. 12, Panels A and B, marked under “Clonal density”).

The data demonstrate that the CD19t-IL-12 expression cassette integration at the PRDX1-AKR1A1 STAPLR site permitted sustained expression of the transgene under external promoter control in both pooled and clonal populations of STAPLR-targeted iPSCs after treatment with doxycycline.

### Example 6: Induction of Reporter Transgene Expression at Various Sites Within a STAPLR Intergenic Region in Targeted iPSCs

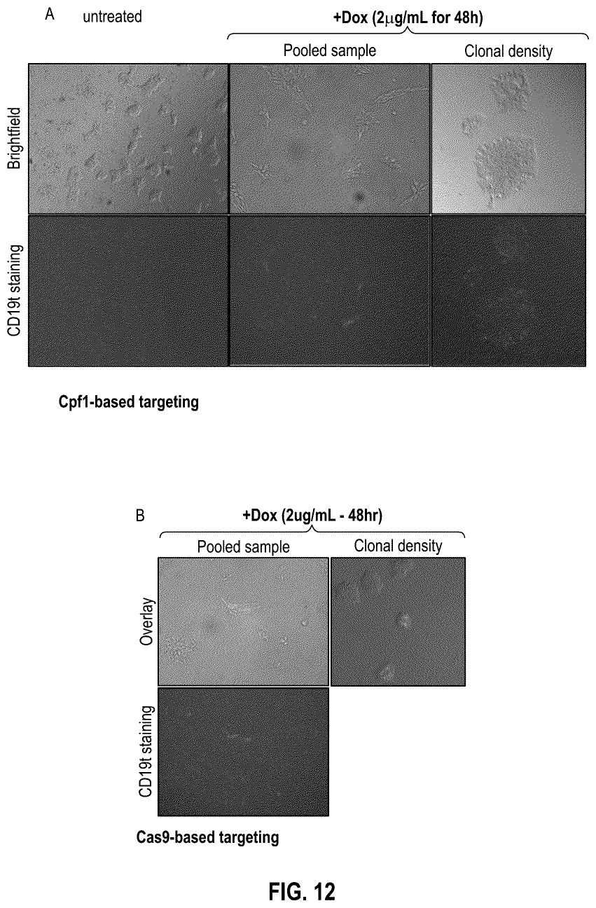

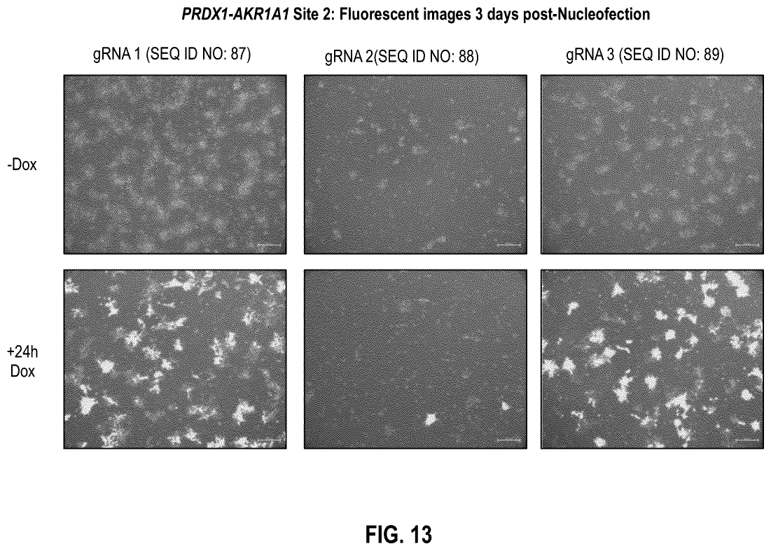

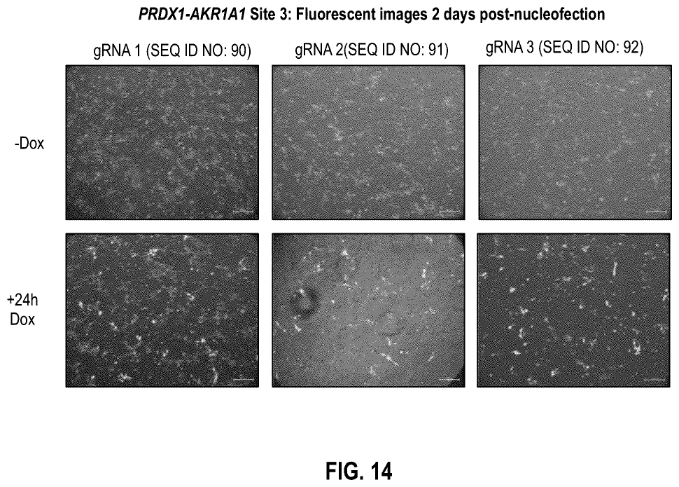

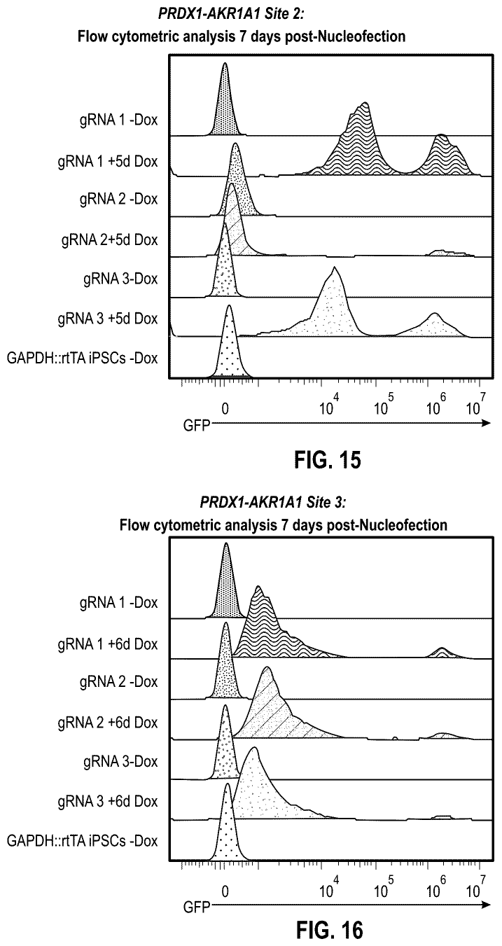

To test for robustness of inducibility and transgene expression at two alternate sites within the PRDX1-AKR1A1 intergenic region (PRDX1-AKR1A1 Site 2 and Site 3), we again utilized the dual component doxycycline-inducible rtTA/TRE system. The TRE3G promoter was used to test expression of an EGFP cargo. A Kozak sequence was included to enable translation initiation and an SV40 PolyA sequence was added to enable translation termination, as per the design of the original PRDX1-AKR1A1 targeting construct. In the presence of doxycycline, the rtTA protein binds to and activates the TRE minimal promoter. A parental iPSC line with bi-allelic rtTA integration at GAPDH (GAPDH::rtTA iPSCs) was Nucleofected with a selected high-efficiency RNP and the corresponding PRDX1-AKR1A1 targeting construct (for either Site 2 or Site 3). Three different gRNAs were tested for PRDX1-AKR1A1 Site 2 (SEQ ID NO:87-89) and three different gRNAs were tested for PRDX1-AKR1A1 Site 3 (SEQ ID NO: 90-92). Pools of cells that received both PRDX1-AKR1A1 Site 2 or Site 3 RNP and targeting construct were fed with media containing 2 μg/ml doxycycline starting at day two (Site 2; FIG. 13) or day one (Site 3, FIG. 14) post-Nucleofection and continuing up to day 7 post-Nucleofection (FIG. 15 and FIG. 16) in order to induce GFP expression. GFP expression was monitored over the course of 7 days by fluorescent microscopy or flow cytometry. GFP expression was induced from both PRDX1-AKR1A1 Site 2 and PRDX1-AKR1A1 Site 3. All three gRNAs tested for each site displayed differences in construct targeting efficiencies (different sized peaks seen in flow cytometric histograms), but all were able to induce GFP expression to similarly high intensities (similar log levels of expression) following doxycycline addition. The peak observed around 10\hat{ }6 represents edited cells that express high levels of GFP, while the peak observed around 10\hat{ }4 represents transient GFP expressed from non-integrated targeting construct. The data demonstrate that multiple sites within the PRDX1-AKR1A1 intergenic region permit robust inducibility and expression of GFP in a pooled population of STAPLR site-targeted iPSCs.

1. A genetically modified mammalian cell, comprising an exogenous nucleotide sequence integrated in a sustained transcriptionally active payload region (STAPLR) in the genome of the cell, wherein the STAPLR is selected from the group consisting of the intergenic region between the RPL34 gene and the OSTC gene;
the intergenic region between the ACTB gene and the FSCN1 gene;
the intergenic region between the AKIRIN1 gene and the NDUFS5 gene;
the intergenic region between the PRDX1 gene and the AKR1A1 gene;
the intergenic region between the PTGES3 gene and the NACA gene;
the intergenic region between the MLF2 gene and the PTMS gene;
the intergenic region between the RAB13 gene and the RPS27 gene;
the intergenic region between the JTB gene and the RAB13 gene;
the intergenic region between the AKR1A1 gene and the NASP gene;
the intergenic region between the NDUFS5 gene and the MACF1 gene;
the intergenic region between the SRSF9 gene and the DYNLL1 gene;
the intergenic region between the MYL6B gene and the MYL6 gene;
the intergenic region between the GPX1 gene and the RHOA gene;
the intergenic region between the HNRNPA2B1 gene and the CBX3 gene;
the intergenic region between the ROMO gene and the RBM39 gene;
the intergenic region between the PA2G4 gene and the RPL41 gene; and
the intergenic region between the NDUFB10 and the RPS2 gene.

2. A method for modifying a mammalian cell, comprising integrating an exogenous nucleotide sequence in a sustained transcriptionally active payload region (STAPLR) in the genome of the cell, wherein the STAPLR is selected from the group consisting of:
the intergenic region between the RPL34 gene and the OSTC gene;
the intergenic region between the ACTB gene and the FSCN1 gene;
the intergenic region between the AKIRIN1 gene and the NDUFS5 gene;
the intergenic region between the PRDX1 gene and the AKR1A1 gene;
the intergenic region between the PTGES3 gene and the NACA gene;
the intergenic region between the MLF2 gene and the PTMS gene;
the intergenic region between the RAB13 gene and the RPS27 gene;
the intergenic region between the JTB gene and the RAB13 gene;
the intergenic region between the AKR1A1 gene and the NASP gene;
the intergenic region between the NDUFS5 gene and the MACF1 gene;
the intergenic region between the SRSF9 gene and the DYNLL1 gene;
the intergenic region between the MYL6B gene and the MYL6 gene;
the intergenic region between the GPX1 gene and the RHOA gene;
the intergenic region between the HNRNPA2B1 gene and the CBX3 gene;
the intergenic region between the ROMO gene and the RBM39 gene;
the intergenic region between the PA2G4 gene and the RPL41 gene; and
the intergenic region between the NDUFB10 and the RPS2 gene.

3. The method of claim 2, wherein the integrating step is performed by using a CRISPR/Cas system; a Cre/Lox system; a FLP-FRT system; a TALEN system; a ZFN system; homing endonucleases; random integration; homologous recombination; a transposase; or a non-nuclease-dependent viral vector.

4. The method of claim 2, wherein the integrating step is performed by using a CRISPR/Cas system comprising a guide RNA, and wherein
the STAPLR is the intergenic region between the RPL34 gene and the OSTC gene and the gRNA is selected from SEQ ID NOs: 25-32,
the STAPLR is the intergenic region between the ACTB gene and the FSCN1 gene and the gRNA is selected from SEQ ID NOs: 33-54,
the STAPLR is the intergenic region between the AKIRIN1 gene and the NDUFS5 gene and the gRNA is selected from SEQ ID NOs: 55-70, or
the STAPLR is the intergenic region between the PRDX1 gene and the AKR1A1 gene and the gRNA is selected from SEQ ID NOs: 71-92.

5. The method of claim 3, wherein the CRISPR/Cas system comprises a gRNA-dependent nuclease of type I, type II, type Ill, type IV, type V, or a variant thereof.

6. The method of claim 3, wherein the CRISPR/Cas system comprises a gRNA-dependent nuclease selected from the group consisting of Cas9, Cpf1, Cas1, Cas1B, Cas2, Cas3, Cas4, Cas5, Cas6, Cas7, Cas8, Cas12, Cas13, Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, CasX, CasY, Csx3, Csx1, Csx15, Csf1, Csf2, Csf3, CasPhi, MAD7, and Csf4.

7. A DNA molecule comprising a nucleotide sequence of interest flanked by a 5′ homologous region (HR) and a 3′ HR, wherein the 5′ and 3′ HRs are at least 95% homologous to a first genomic region (GR) and a second GR, respectively, in a sustained transcriptionally active payload region (STAPLR) in the genome of a mammalian cell, wherein the STAPLR is selected from the group consisting of:
the intergenic region between the RPL34 gene and the OSTC gene;
the intergenic region between the ACTB gene and the FSCN1 gene;
the intergenic region between the AKIRIN1 gene and the NDUFS5 gene;
the intergenic region between the PRDX1 gene and the AKR1A1 gene;
the intergenic region between the PTGES3 gene and the NACA gene;
the intergenic region between the MLF2 gene and the PTMS gene;
the intergenic region between the RAB13 gene and the RPS27 gene;
the intergenic region between the JTB gene and the RAB13 gene;
the intergenic region between the AKR1A1 gene and the NASP gene;
the intergenic region between the NDUFS5 gene and the MACF1 gene;
the intergenic region between the SRSF9 gene and the DYNLL1 gene;
the intergenic region between the MYL6B gene and the MYL6 gene;
the intergenic region between the GPX1 gene and the RHOA gene;
the intergenic region between the HNRNPA2B1 gene and the CBX3 gene;
the intergenic region between the ROMO gene and the RBM39 gene;
the intergenic region between the PA2G4 gene and the RPL41 gene; and
the intergenic region between the NDUFB10 and the RPS2 gene.

8. The DNA molecule of claim 7, wherein each of the 5′ and 3′ HRs is independently about at least 50, at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1000, at least 1100, at least 1200, at least 1300, at least 1400, at least 1500, at least 1600, at least 1700, at least 1800, at least 1900, or at least 2000 base pairs long; or between 50 to 1500 base pairs long.

9. The DNA molecule of claim 7-Gr-8, wherein the 5′ and 3′ HRs are at least 95% homologous to
SEQ ID NOs: 17 and 18,
SEQ ID NOs: 19 and 20,
SEQ ID NOs: 21 and 22,
SEQ ID NOs: 23 and 24,
SEQ ID NOs: 93 and 94, or
SEQ ID NOs: 95 and 96,
respectively.

10. The genetically modified mammalian cell of claim 1, wherein:
the intergenic region between the RPL34 gene and the OSTC gene comprises a nucleotide sequence at least 95% identical to SEQ ID NO: 1;
the intergenic region between the ACTB gene and the FSCN1 gene comprises a nucleotide sequence at least 95% identical to SEQ ID NO: 2;
the intergenic region between the AKIRIN1 gene and the NDUFS5 gene comprises a nucleotide sequence at least 95% identical to SEQ ID NO: 3;
the intergenic region between the PRDX1 gene and the AKR1A1 gene comprises a nucleotide sequence at least 95% identical to SEQ ID NO: 4;
the intergenic region between the PTGES3 gene and the NACA gene comprises a nucleotide sequence at least 95% identical to SEQ ID NO: 5;
the intergenic region between the MLF2 gene and the PTMS gene comprises a nucleotide sequence at least 95% identical to SEQ ID NO: 6;
the intergenic region between the RAB13 gene and the RPS27 gene comprises a nucleotide sequence at least 95% identical to SEQ ID NO: 7;
the intergenic region between the JTB gene and the RAB13 gene comprises a nucleotide sequence at least 95% identical to SEQ ID NO: 8;
the intergenic region between the AKR1A1 gene and the NASP gene comprises a nucleotide sequence at least 95% identical to SEQ ID NO: 9;
the intergenic region between the NDUFS5 gene and the MACF1 gene comprises a nucleotide sequence at least 95% identical to SEQ ID NO: 10;
the intergenic region between the SRSF9 gene and the DYNLL1 gene comprises a nucleotide sequence at least 95% identical to SEQ ID NO: 11;
the intergenic region between the MYL6B gene and the MYL6 gene comprises a nucleotide sequence at least 95% identical to SEQ ID NO: 12;
the intergenic region between the GPX1 gene and the RHOA gene comprises a nucleotide sequence at least 95% identical to SEQ ID NO: 13;
the intergenic region between the HNRNPA2B1 gene and the CBX3 gene comprises a nucleotide sequence at least 95% identical to SEQ ID NO: 14;
the intergenic region between the ROMO gene and the RBM39 gene comprises a nucleotide sequence at least 95% identical to SEQ ID NO: 15;
the intergenic region between the PA2G4 gene and the RPL41 gene comprises a nucleotide sequence at least 95% identical to SEQ ID NO: 16; and/or
the intergenic region between the NDUFB10 and the RPS2 gene comprises a nucleotide sequence at least 95% identical to SEQ ID NO: 97.

11. The genetically modified mammalian cell of claim 1, wherein the exogenous nucleotide sequence comprises a transgene.

12. The genetically modified mammalian cell of claim 11, wherein the transgene encodes
a therapeutic protein;
a cellular marker; or
a protein that regulates the differentiation state or activity of the cell.

13. The genetically modified mammalian cell of claim 1, wherein the cell is a human cell.

14. The genetically modified mammalian cell of claim 1, wherein the cell is a pluripotent stem cell (PSC).

15. The genetically modified mammalian cell of claim 1, wherein the cell is:
a) a cell in the immune system;
b) a cell in the cardiovascular system;
c) a cell in the metabolic system;
d) a cell in the central nervous system;
e) a muscle cell;
f) an adipose cell; or
g) a cell in the ocular system.

16. The method of claim 3, wherein the non-nuclease-dependent viral vector is selected from the group consisting of: a retroviral vector, an adeno-associated viral (AAV) vector, and a lentiviral vector.

17. The genetically modified mammalian cell of claim 11, wherein the transgene comprises a constitutive or inducible promoter.

18. The genetically modified mammalian cell of claim 12, wherein the therapeutic protein is selected from the group consisting of: a protein deficient or defective in a genetic disease, a cytokine, and a recombinant antigen receptor.

19. The genetically modified mammalian cell of claim 12, wherein the transgene encodes SOX10, IL-10, IL-12, CD19t, or ThPOK.

20. The genetically modified mammalian cell of claim 14, wherein the pluripotent stem cell (PSC) is an induced PSC (iPSC).


SEQUENCE LISTING (102 sequences)

SEQ ID NO: 1 - DNA, 20100 bp, Homo sapiens
  source 1..20100: mol_type = genomic DNA
gttccatcttcttaaaaactagatgtggtaaacaactattctaaaccaggagtagaaaagctaacactatctggctggacacggtggctcacacctgtaatcccagcactttgggaggccaaggtgggtggatcatgaggtcaggagatggagaccatcctagctaacacagtgaaaccccgtctctactaaaaatacaaaaaattagccgggcatggtggcaggcgcctgtagtcacagctacccggaggctgaggcagaagaatggcgtgaacctgggaggtggagcttgcagtgagccgagattgcgccactgcactccagcctgggctacagagcgagactccgtctcaaaaaaaaaaaagaaaagaagttaacattgtctttatgaatctcatttctatctcagctagttcatgctactgtaactaccacggactgagtggcttgaacaacatttatttctcactgttctggaagctaagtccaagatcaaggcactggcagattccatgtctcttcctgtttgcagatggccatctccttgctgtatccacatgtggtggagagatcacctgtctcatgtcttttcttagaagggcacttatctcgtcatcagggctccatcctcaaaatctaataatatatacctccccaaagcctcaccatcccattggaagttagtactccaacatataaattttgagggggatgcaaacattcagtccctaacattttctaaattaaaggataagatccgcaataattctggatgaaaagatgcttttgggtttccaaaatattagctattcaataatttttaataaatcttaaatgtttctggttaacctgaaggattatgtaagtatttttgccaaacatttacattcttttataattgctttagctttcattcatttactcgataaatatttattagaaactaccatgtaccagaaactatgatgctggataattgttaatacagtagtcaacaaagcagatgagactcttcccttggcctcatggcacacaggagagacaatgttaaatggggaacatataaatacatttgttgttgtacacactgggattagactcacaaaaagaaagtccacagtgctgtgaaacgatgtggaaagggaacctcatgggggagttaagggaggctttttctgagaaagtgatgtttaagcccacaacaaagataagtaggtgttgtctgggtgacaaaaagggagaatactctggacatagaaaccaacatgtgcaaatgccgtaagtcaagaaatcgtttttatgaaaaacaaaatcaattcaggcaatccaattcactctcaatatttaaaggccaataaacctgaaaagtgtgaactaattgttaatcactaagaaggaacatccctgtcagcacatagatttactatacctgtgtggccccagggctacttttcatcctaagggacaccagcaaaagaaaagctttcctaaactgttttagttaggagcaaataaatcctcctctacattaggggatatttaaattatgcaatcccctagataatttaggcaccaaccacctaccattaaggaggaaaaggatcaaatttactgaccaaattcccttgatattcattcagcttctatattccttggcctgtgcaaatagcctgaagactttactctctcaactctctctcattctagggttgaaaataagcttacagcttgacttgcctaaagaacaaatggtcttgaagaaaattcacgtgtttattgcatcttagtgtaagtcataaaagatgtcacaggccatagggtctctctttcacaactactcaattctgccattgtagagtgtcagtagccatagacgatacataaacaaatggtcatggctatgttccaataaaattttatttacaaaaactgatgtcaggtcagatttggtctcttagctcatagtttgggaccctgctctagacaactctgcttctagccaaagctgccatctgattgcaattgcatgagagaccttaagcaagaccagtagaaaactgccttgttaagtctaatcagcccaatggatggtgggaaaaaataaaatggctgctttttaaattccattcaattttgaggtgatttgttacatagcaatataccactaagacaatggtacagtgcaatgatctttaaatatttctttttacttgagtaccccttaacattttggaaagttatgtagactctggcacattttcagattgtcatctaaaacttttcattcaaaatgtaaatccttgcaaaggatatcatttccaacagagtgaacattttaataattttaaagagaactgttttatcaatttttaagcatatcaaaatctccatagccatttttttgagtttttgtatatgtatttatttatttatttttaacttttaagttcagtggtatatgtgcagatttgttatataggtaaacttatgtcatgggagtttgttgtatagattattttatcacgcaggtattaagcctagtacccattagttatttttcctggtcctctccctcctcccaccctctaccctgcaataggccccagtgtgtgtttccctctatgtgtccatgtgtcctcatcatttagctcccactttataagtgagaacatgcagaatttggttttccgttcctgcattagtttgctaaggataatggcctccagctccatccatgttcctgcaaaggacatgatattgttcttttttatggtatagtagccattttcaaaaagacaggaacaagcttttctaacatttggaagttttatattcgttcttttttcttccattttgttttgtttttttagagacagggtcttgctctattgactgggctggagtttaatggtaccatcctggctcactgcaacatcaaacttttggggtcaagcaatcctctgcctcagccttccaagcagctgggactacaggcatgtaccaccacacgtggctaatttgtaaaatggtttttgttttaaaaaagaaaaaaaagatggagtcttgctacgttgcttaggctggtctcaaactcctggcctcaaagcgattctcctgcctcggcctcccaaagcaatggaattatagatgtgagccactgtgtcacccctttttctttttgacatatattaccattccatttcctccatagaattttaagctaatatatttttatgttgaaatattttcctgattggtaattcccacaattctctctcttttttttcttttttttctttttttgagacaagagtctcactctgttgcccaggctggaatgcaatggcgcaatcacaatctcagctcactgcaacctccacctcctgggttcaagtgattctcctgcctcaccctcccaagtagttgggattacaggcacccaccaccacggtcagctaatttttgtatttttagtagatacggggtttcgccttgttggtcaggctggtctcgaactcctgacctcaggtgatccacccgccttggcctcccaaattgctgggattacagacatgagccaccgcgcccggccctacaattctatttttaagagagaacaagcaaaagagagagtcatggagtcttgccacttgtttatcccatggataaaataaggcgttgattgctgcctcttcaaccatgctctctgcttcattctcaagggattccctcaggaagattcatttttttgacaatttgggggagatgaaagaggataaggttgctaaatgaaaatcgtcatcattctccttcactccattcgacattatctctgagttcacgccgtcccccagtgacagccaggatatcctgctccagtgtaagatgggaaagacaccaagcccctggtttttaaaatttcttcacaatcagtcaggtattatttacaatttgttgactatgtacaaaatgctagtataggcactaagatgaacaaaatagacaaaaatccctgccttactggagattaccttccagtgagggctgacagacaataaatgaatgaaggaataagtaatatgtcaataggtaagtaagtaaaaaacacgatatgaacaataaatgctatacaaaatataaagcagatgccgggcacgggggctcatgcctgtaatcccagcactttgggaggccgaggcgggcggatcacgaggtcaggagatcgagaccatcctggctaacacggtgaaaccccgcctctactaaaaaatacaaaaaaataagccagacatggtggcaggcgcctgtagtcccagctactcaggaggctgaggcaggagaacggcgtgaacccaggaggcggaggttgcagtgagccgagatcgcgccactgcactccagcctgggggacagagcgagactccatatcaaaaaaaaaaaaaaaaaaaagtatatatatatatagcagataaaggggatagaatgcagggtggaagtagagggttgcagtttgaatatcctggtgaggggagacttccccaagaaggggacatcgaagcaaagacctgaaggcagtgaggagggtggccataaggatatgtgggaaagagctttccagcagagaatagtccggaggcaggaggaggtcctgaagctgagggaaagcgcaacagaaatgaggttagggagttctccagacaccaggttgtgtgggtccttgccattcatctctaggaccagtgcggccctcagacaaggggcctctcctctgggtcccatgctttagagggcgctgctctggctttcacctgccaatatcccaccccaaaagcaaagagttcaggggaccaaggggcatgctaaccctaaatgtatgctctctttctggacataatcccagaatccctgcccgaggcacccaaacccacgcacatgctcatttcatgtgagtcaacatgttccctcctcaaggagtatttatatttttggttttgtaattgctgatggtgagttgaaagactgacttctgctgagggaattttaagcagaggaagctggtaactcaatctgtttggagtcaaggatcactctactcaatgtaacacaaagctaagaggtaatgcaaaggacaggatcggcagtgaagaaaacacgggattgccatgtcttcagaaatcgacatcagaagctcccatgaggtttgtcttctgaaatttccttttctgtttcaaaaatctgagcttaacaatattgtcagtggttctcacactttagattcacttaggattcttctgggaaacttgttaaaattcagatacccaagctccatcaaagatgtccccaatcagaacctctgaaagtcaggccaagaactttcatggtaacagatctcctagctgattttcacccagatggatcttgaactgccctttcacaacgatgtggtacaaactgtgtacggttgggcattagacaaacatgggtttaaatcataggtctaccattaaccagttaggtgactgagcaattttcttaccttctgtccatcttagtcacctcacctgtaaaaatgaagataaataatcataatttcagaggttctgagggttgctgaaataccttgtgttgtccacatcgcagagcacatggttcatggtaatgatgctcagtaagcctctttcctgcttgcagtacaagagctgccttcttacctacttacagagaaacacagaaaatgcatcaagatggccaggatgaagtcttctcatttagagtgattgaagagcagtgataatattggaaataaaccaaaaagccaactgatatcctcctgtatcatgttttacccaaaatgatttttttttttctctttttaaggaattacacacacacacccccatacacacacactggctgatagtatcaagctgtctttgattagctctgggcaaatgcatggcagggcatctgaaaaccacaactggagacatcatttctgatgggcctaatgctctgggtatttcctttcaactgaacttggggaaagtactttgatataatgtacatatggaaatattttacaacattctttaaaagttagatactttattcaggatatatgtaaaacacacttttctcttcaacgtgtatggctaaattccttgtcatttctaagccagcgtatttttgtgttgttttgttaaattgatctcagtgtgtaagaaaggaccatattgacagccctggcgacagtcttttttctgggcttctggcagacagcaggaacagctgctgcttaagcataccccaaatattttacctctggaaagatcatttctggaggtgagcaggcactttgatttctcattccctctgaaaggtgtcggtgggaatggtgggggaatagctggccatttactcggggctttggttctgaggccattgccacatttttttttttttttttttaaaacggagtctcgctctatcacacaggctggagtgcagtggcgcgatctcggctcactgcaagctccgcttcccgggttcacgccattctcctgcctcagcctcccgagtagctgggactacaggcgcccgccactgcgcccagctaatttgttgtatttttagtagagacgaggtttcaccgtgttagccaggatggtctcgatctcctgacctcgtgatccacccgcctcagcctcccaaagtgctgggattacaggcttgagccaccgcgcccagccaccattgccacatttcacatgtgcaatcaaattgcaacctgggttctggttcaaattcttttgaagtgattaaagcctgttgttttaaatgccattcctaatcttagggtaaaacattccttgggaaagtgttcttcaatccaagaatctaacagtggttactttcagggagaaggaataggggaggggagaagatgataaggggaaggtgacatttgctttgcatttttaaaattatttgtactctttgaatttataactccaaacatgtcatcttattttttaaaaaaaggaatcgtctgtataatggtgttgtagaccattttggttattaattctctttaaatttctgaattgaaaaatgaaatctagtaagtatacttttgaaagcttagctagaaattatttatttttaagattcaattaccaaaacgtgccaaatatacttttttcttttttagtttacaagcctgtatgtaaaatatttgtaagacaactagatataaaaaataccctgggtttgatattttattcatctgaaaatgatgctttcacaagattttttctttcatcattaaagaagtaaatcatttgcattttaaagatatgctcgaatgctgtgggatgcagcttttcccatttatgtaatgtttaattgcatttacttgatcacagcattgaggcaagtttctttaaaataagtttgccttttcataatgcttgcgactatagtttgttaatctcaaagtatcaagaatgcactttgatgaggataaaattatgcaggactatgattcctatgaaagagaactcctgccctactctggccacagaaaagaattacatgtgggcagctgtgagaaaaatgctgttgttatttagttgaattgcaacagctttgcaagtgaggtaaaaaacgtgtagatctacagttatttttctttaacacattttaatatctatgtgcctgtgtgagtgtttttttgttttaacctgggtccctaatgttgcaaaacaatttattcttcacagaggacagctcccaactctttacctctcttcctaatgcaacactcactctcactctcagcagccttataatatctgtaaatgtagaaactatcagttatgaacttctgtaactcccttttcatctttggcacctcaaaactttaatgtatgtaaaagcatgggctttgaaattggtaaactggagctctatcaatctctggctgtgtcaacttgagcatgttgcccaatttctatgagcctcagttttcttatctaaaaaatgaaactaataatgcctactccaaagaattgttggatggattaagtaagacaacctaatataattgctgtgcatagtaaatcctaactgttggttgccttatacctctaatcttctttctgctctcttttctttagaagatggcatgcatcttttccaaggtaaattttacctctgttgaactctatcccatactctcttgccatggccctaattttgttcagttacttaactccttcccttccctctgacatccttggtcccttcctctccacagttttattctctcgtcttacacaaacatgcccaagtctcatcatccataaaaacctctgtcattaccacttcagctctcttcttctgttcatacccagctgtgttgggtgccatgaatcattcactactattatttttaaaattcctatcactacttctcccctctgcaatctggcttctcctgccaacatgctactgaaattaatgaattaattcatcaaatatttacaagcatttatcatgatactatgtcccaggcactgtgctcagtaactcaagtcattataatactgtgtgcttgacaatatgttgtacttactggccaaaaatcaccaatgtctaattgtcaagctaaatgtcttctttcaatttcaatctatctgacatgctggaaacttccctttccaaggcttctttgctatcgctcccttggctcttctgttcctttgatcttgctgtatttactttgtagtatcctcctaattatgggggtgattccctaggcatccatcttcagctttcttctctctcactcttttaactttactctgtgaatttcctgtaattatacactacatttggtgtgtgtgtgtgtgtgtctgtgtgcacgtgcatttttctaggaaggagttcatagcttttatcagctctgcaaggaggtctatgaccctcccaataacaaccctgaccagttccacctcacgtgtagacacattcttccactgtgatgacttccattatcctccacatgctgctgattcctggatctccaaccctaaacctctttcccatgctccagcgaccatcttcccaactctgcctaggtccctcctggagggtccttctagaataatagttctcatcaggcaggtggcagaacctctgtctcccactccttgcatgcccctctgtgaggcttgggtgggttgggggtgatgggaggggtgcgtgtataatcagagtccctggggtgcagaagctctgttctgaccttgcctcttgctatccatggaattactgctctatcctggccattttcccattctctttctctggttccaggaaaagccttccctggtccagctcttggacacagctgtagtgtagcctggagccattgttacacaattcactttcatcagagccctctctttccagagtgttcaagccctatatttatttttaatcaaattctcatatttctatctgcacagaaagatcttatggtggggtgcagtggctcacacctgtaatcccagcactttgggaggccaaggggggcggatcacaaggtcaggagatcgaggccatcctggccaacatggtgaaaccccgtctctactaaaaataaaagtattagctgggtgtggtgggaggtacctgtaatcccagctactcaggaggctaaggcaggagaatcgcttgaaccagggagtcagaggttgcagtgagccgagatcgagccactgcactccagcctggcgacagagtgagacacaggctcacagaaaagaaaaaaaaaaaaaggtattttatagcttttgctttaccaccaccttccctgctctcctcactcacagtctgtctcagcccctcgctgtctctcatctggacttccacactaaggtcctaaaaggtttctctgcctcaagtctttctgtcatcccatctattcttccaaacacaatactaatcatttcaagttttaaaatccttcaatacttctttatgcctatataatgaaatctaaaagcatcatgactttgaaagaccctgtctagtcaggcttcttcttttttaggtcgcatctccacttttactcctccatgatcaccatcactctgttaccacctttggggctttcccatctctctgcctttgctcagccttgtccgaatgcctgaaagccttttcccaaccgaggaatccagctcttctatcaatgctcctgctcaaatgtgagcacttctgtaagcctgccttttccctacctgtttgagaattttttttttttttttttgagagagagtcttgctgtgtcgtccaggctggagtgcaatggtgcgatctcggctcactgcaacctctgcctcctgggttcaagcgattctcctgcctcagcctccaagtaactgggattacacacatgcgccagcatgcccagctaatttttgtatttttagtagaggcggggtttcactatgttggccaggccgttcctgacctcctattcctgacctcaagtgatccacctgcctcggcctctcaaaatgctgtgattacaggcgtgagccactgtgcactgcctacctgtgataattaatcactatttcctcaggtctctcaaagcccaattggcccaaacctccaataggcatgtgcttctcaggcagttctgccatagcgtgtattctcctcagactaggataacagtttcttggggataagagttatgcactggttatcttggcactgacagcatttaagacagcttgtttctgctacatgatccataaatgcttgatgaatgaatgaatgagtgatgaacgttgtcatcaacaacactactgcctactgtctttttgtttgtttgtttttgagacagagtctctgttgcccaggctggaatgcagtggcgaaatctcagttcacggcagcctgcacctcctgggttcaagtgattctcgtgcctcagcttcccgagtagcttggattacagatgcctactacgacgctcagctaattttgtatttttagtgagatggagttttgccatgttggccaggctggtctcgaagtcttgacctcaggttatccactggcctccaccacccaatgtgctgggattacaggcatgagccaccgtgcccggccctgcctgctgtcattgattcccatcttggcaaggaaacggagggaagtgtgtgagattaggcgtatttccacaaaacaagtattaaaatatggaaaggagttgtaacatagcaaagaaggcagtagtgaataaaaagaaccaaattttacttcctgtttagctacttacttctctctagctagcagtaaagacagcatcttgattggcataatgagatttgttcagtttcttaaacaatgcaccctggctgcaatgtagagagaaatagaggcaggacatcatgtggatctgaattaataagtggtggtggaggtagaaaggagggaacaaactaaataaacattaaggaagtacaattgctaagatgacaccttgaaggatcaaaggaaatggaagaatcgggttcatttattgattcattcaacttatttaatgagtcctactatgtgccagcactgtgagaggtggtaggaattccatggtgaaaaatattttgtccctgaatttgagacactgagtttctgatattacaattggctgtgtaatggtacatagtaataaacatccaatcactcctacttgaatgtaagcaaatatgagaagcattattatgcatctcttacagaatacagcagtggacacctgggtagagagacagatgtgctgatgtaagaccagaaagtactttgtgttcttacaaagtactattataaaagtttattttaattacaaaagtaatatatgcttattacagaaaaattaggaaatacaagtagtttcaaagaagggaaaaataatctataatcacatcacatagacacagtcatctttatgttccttcagacttttttccagtggaaaatacatacaataacaaataagccagtactctacatacaattttatggtctgattttcttcattttaagcctattgtggaggccgggcatggtggctcatgcctgtaatcccagcactttgggagaccaaggtgggtggatttcttgagcccaggagttcgagaccagcctaagcagcatagtgaaaccctatctctacaataaaaaaacacaaaaattagccaggtaaggtggcgtgtgcctgtagtcgcagctactcgggaggctgaggcaggaggattgcttgagcccaggaggtcaagaatgcagtgagccatgactgcaccactgcgctctaccctgagtgacagagtgagactctgtctcagaaaaaaacagctattttggacatctgtccattccagaaaataaatttccacagtgtgctttttaatagctgtataatattacatggtatgaatgctttacaattttttttaattagtccactatttttaattgggtatttttaatgtttcagaaataactgaagatcagttggaaagttacttcactagttagacaatatgtttcttaaaaataatgatgtctcttatggcccatactaatttagagctctgcaatgaagggattctgggagatgtagttcagcttagctaagttaacacaatacaaatccaccacagttgattccttgtcaactaaatatctatatatacctttctgaaccacatttggcttgatttttttttttttttttttgagaaggagctcactctgttgcataggctggagtgcagtggaacaatcttggctcactgcaacctccatctcctgggttcaagcagttcttctgcctcagcctcccgagtagctgggattacgggtgtgggccaccatgcccagctaatttttgtatttttagtagagacgggatttcactttgttggccaggctggtcttgaactcctgacctcaaatgatctgcccaccttggcctcccaaagtgctgggattacaggcatgagccaccgcactcagcctagcttaatttttttttttttttttttttttttttgaaatggagtttctctcttgttgcccaggctggagtgcaatggtgtgatctcagctcaccacaacctctgactcccgggttcaagcgattctcctgcctcagcctcccaagtagctgggattacaggcatgtgccaccacaccgggctacacccagctaattttgtattttaagtagaaacagggtttctccatgttggtcaggctgctcttgaactcctaacgtcaaatgatctgcccacctcggcctcccaacgtgctgggattacaggcatgagccactgtgcctggcctgcttaatttttaaataagagcaataacgacattatgcttccacctagcaatgtaactatccctcatacaattgaaacatgctgtctccccaaaacaggaaacaaactccttttatccatttttgggtgataatcatctctcttctaggtgattcaatattccctctttgatatccggttacttaaacactgagagcttaaatattatcaacaaattgtatatcacatttgactaggagaatgggagcctgaaaaaaattggttaatatacacaaaaatatatttgtatcaaaataaaatactcctaactattacagtgcttatttctaaaactgcttacatggtcatagctggtatttataactacattcttccactacccatttcatattgtctttgccctcagcaaaaatctcagctggttgtggtttcttgcctgttggagtgacctaaaccttaatccctgaaaggtctaagccactaacagtcctggctctattgggttgttgtaattttccattgaagtttatcacaggatatgggattactaaggcatctcaggggatctactgcattccagacatactcctccttactactattttgtgatgacaatctaatttcctcttgattgtcaagataaatctgcctagcccataaagtattcttttatctgtttcttgattcattggcacgaggacttgtgaagtggctagctgacagtctcatctctcaaattaatggaaccattaatgtgttccttggtggaagtattcttctttggtactaatacttctagatcagtagagccctaagttgagtaaaagcaagaggagccattcccattccctacacatgatttctggacttgtgaattctaactgtgagagaaatatgttgcttactgatttactgcatatatgtatcttagaaaacacggacctgctcccaaaggggttgccatcataactgagttttcaaaagaccattccaccatttcacatactaagactagtaagtttcatgaacatgagccaatggccacaactcatttgctgttaaatgagttccttggccagaagcaatgctgtatagtaaaccatggtgatgattatggcattccctaggcctacagaatagtgattttggtagcagcatcatgcgtaaggaaggaaactttgttctagtgtgtttacttcagtaaaaaccaagtgcctactggacctggtcctatataattaacttgccaccaggtctggctcatcctcctgtagaatagtgccatattgtgggctcattgttggtttctcctgtggcaggctgggcactcagcagtggttctagccagatcagccttagtgaatgaaaggccatgcatagcttccatccctcctgccatggccacttcatctgtaggtcctttgggcaaggtcacaaatggatatggaaagaggctgactgatgtccacagaatgggccatcttgtccacctaattataaagatcttcctatgcagagattgctctttggtaagcactcacataaggcacaaatatctttacatagatatcctgtagggatagacccctacaggaaggtctatccacatgcctctcctccagacctcctcagtaattttcctattgtgttccttcccatttttcaaccatctgactaaatgattgaactgcccatcaatctaactaaatgattgatctgcaattcttgctgttgctccttccaggcaaaatgaacaaccaggtacactgctaaagttctgcccaatgggaagatttttcctcaccactttccttcaggcctaccactctccttcatagtgaggctgttgtgcaaagccatctactttcagagagtgccagcacattgtgtggaactatctgtaaatcaggctttcttcttcagtcaactggtcataggttgcctgaaggtataatgccatagatataggttgagagaaaagagacaatgtggcaggaatgtgggccatatgcatttgaaactattttttgtacaacttacttgtaccttcaagatctacttgagcttgatcttatgcacaacgctttcatttgatgatagaatgctgctgtagacacccacgtttattggatgatagattagacaatgccaaattgtcaaatgatgggtagttcaagtttcatggtagcttgttgcccacggtcaatagtttcatctctactaaggttcagtggcaagcctagagtcatttgtcaatgactgcctgtgaaataatctcagtggcaagcctggagattatttcttgacagaggatgacataggtttgcgccaaagctctaaggctctgtgctacagtggcttgtcaaaagcttcataaagcatgcctatctaccacagacgttacaagtaatactggatctactaagttgtatggcccaagtggcagagcagtttgctattgaagaacctttgcttattcaggatcccaatcaaaactagcagcttattggattgctcagcaaatacatcaaaatggtatgaccaaatgaagtatatgttgcaagtggtccatccattgagcattgtgcagcaaatatcctttattttgaaagagctttctatgtattacagatcacttaacccctagacatttccctgaggagaccctgacattttattgtgcttatttttccatcctctggcatgcatttgtcctaacaatatatctactgtagttgctacttagatcactgtctaatgagcatattatcaatgcaatgtcatagtgtgatattctgtgaatgagaaggcaatcaaagttcctgcaaatcagattatgacatagagctggggaattgatataattctgaaatggaggtgtgttgctgactctgccagttgaaagcaaacttcttctgatgatctttaccataggcataagtagaaacagcatttgccagaacagctaacaggtgccacgagatgtgttgatgtgctccagcgacaaaaccacaagtggaatagcagcagcagttggagtcaccacatgattaaatttacaataatccactgttgttctccaaggtccatctttcttttgcaagggccaagttaattgagcgaaatgtggcagaaatcactactcagcatctttcaagtctttgatagcacatcagccactgaagtcactttagatttattatttctgtaggtaaaagcagttctactggttacacttagactttcctgccataaaagccctcataatatgggtcaggaaaccaatatggtgatttttccatttgctgagtacatttatcccaattgtacattctagaactcagtgatctcactacctctggaaggagaactaaaattagaatagtgataatgggaaactcgttccatatgtgtctctaatatttgaataaattaattattttacattgggcaggttcctatgttttacttgcataattttatttcttattttcattatttatttgtttattcatttatttttagagatggagtttctctcttgttgcccaggctggagtgcaatggcacgatctcggctcactgcaacgtccgccactcgggttcaagccattctcctgcctcagccttgcaagtagctgggattagaggcatacaccaccatgcccagctaattttgtattttttagtaaagatggggtttcatcatgttagtcaggctgggttcgaactgctgacctcaggtgaggaggcctcccaaagtgctgggattacaggcatgagccaccgtgcccagccattacttgcatataattttaaaaacaaccttcaaaatgtaaaataactacaacaataacaccaaaccccacaatattgcattagaaagagtttttatttagatcatgagacataatagtgcttcgtactcatgcttatacagaacctagcgcgaaagaacataattcaagacactgtcaaccaaactgcaaaatgtcttcataaataaaaatccaggttaatattagtattattatcctacaaaaaacaaagctactaatttcctctccaacacatatttaaataaacagaaatcaacaatatttgtagcctttgtaattctagtttgtttagaatttagttaaaaacttatttgtggagctctccctctcttttggctggcagaaccatttctgacttcaaggctttttattaaaatattctacaatgaacacagaaagcttggtagataccatctttacacttttttcttctttgaatatatatttgcttgtgcacagtaagaattattaacatttgcttttatatgtagcttaagatgacagctctactgcatattatattgtaaactgtgtttctctcaacatttttccttcttcatctataaatagaaaaccactgaatgattgattatatgtttatttaagggcatgctctaatgcccactgctatattcattatttctttggttttcagttacagagtttatgttatgtttaaatagaacatttttctgtaggaattgtgggttaggacatggatgataacagatatgcatatcttactcatgaaaggtctcttttatacgtggcttatagaatattacaaactcatgttgaaaaatatttcaaagcttaagaggttaaaaaataataatgaattggttcctggagcaaacttactttttggaaagtgatagtaataatttaacatatagatgaatgcagtgtgttctgacttggcagttttattatttatgaaattaatttttcatttaaaaattatatattaataatttatatgttttccacaatgtaggtagaaattatgtgtccctagtatacacaggaagaaggataagtatataaacgtataccattttaaacgtcttaaatctatttacttctacagcaattgccttttaggtctcatccagtcacagactatactacactttttgagtttatttcccttcccaggagccacttttgtcatatgtaaagcagttatactagtatttacctaacagaattgtcatgaagagtatataagcacaaatgtaaatcacttaacatagtacctgccaagcagtggttaattaatgttagattcctttttaagctataaaggaaatttaaaaacctttaatccctatgtttttccaaagcaaaatgttttatttttatttttttcttcataccaaagcaaaatattttaattgttccaaagtgttccttacttataaaactgtgggaatttcagaaacattctgctttacagtgcccagagaaacttcctacatataaacgctggttttggtgtaaatgcattcattattggtgttatcattggcctttacataacctaagattcgtgtggctccgttaagacataggcaatatgactctgggagagactctcactaatgaacagtaagggcagcaagcctcaactgtacttagaattggatactacaatgttttgatagtattttcattaccgtcttcaagcaatttcttaacaattgagttgttagctaattagaagtgcagaaccaatacatggcatcctgtaaaaataaccacgggcttagtaacaagcaaataaaaaaatataaaaacattggctctataatgtgtcaatatgctctggacttatgtaatcagtgactcttaactcttgctccaaaaggaagttaacatttgtagagtcttaaaagattatataaaaaagaaaagcaaaacagaaaacaaaaaatcttttagaccttctagtccatccctaatgactaattctccatcatatattttcatctatttggattgtaaatgttgcccagaccagagatggcggtggttcacactagggtgtcagtggtggaatggtgaggggcagatgaattggagatattttggtagtacagccaaagagacctactaatattttggatgcagaaggttagtaaggagagaaatccagacgactttaagatttttggccagagggattttaccttttagcgacatacagatgaccaagggaagagcaaagttgagaggatgagttattcaaaatagtattatggtcacgccaagctatcagcattaaggcatccctgtagagatgtcctgttgacatttgtgatccaggctggatgtttaaaaaaaattggtaacatattactttagcaactattattaattcttattattaccacgactacatcgggaagtaaaagctcagcagcctccggcttttcctaacccttgaaaaaacctctaatataaaaggcatttatatccaaaagttcccattcatctcccgctctgggaaccgggagcgccccctgtccaatcagctttcaaatcccctgcgtcagcgagtgggcggggctcacctagattccttggcctccgcttctccggcttggccacgttagaagtcccgcaggtccttgaaggtacacaaatcacctatatatccattagaaaaatgtagagtacattatcacaatgtaagtttaattagattaaataatttacaacactttattcatatcatttatttgttttatttagtttcttattttcttctatgagtatagtaaccacctccgagctttacggatctgcgggtcctttcctcgcgcacgcgcacttgt

SEQ ID NO: 2 - DNA, 62214 bp, Homo sapiens
  source 1..62214: mol_type = genomic DNA
ggtggcgcgtcgcgccgctgggttttatagggcgccgccgcggccgctcgagccataaaaggcaactttcggaacggcgcacgctgattggccccgcgccgctcactcaccggcttcgccgcacagtgcagcatttttttaccccctctcccctccttttgcgaaaaaaaaaaagagcgagagcgagattgaggaagaggaggagggagagttttggcgttggccgccttggggtgctgggcccgggggctgggggcgcgcgccgtggcccccgcgccccacgctgggcagtgcccggttcggccccgcatggccaggcctgcccccggcctgcccgtctctcgggccccccacccaccgcgggacatcctaggtgtggacatctcttgggcactgagcgcccaggtggggtgggccagggtctgcacgggtgccagggccctgggttctgtacgctcctgcagaaggagctcttggagggcatggagtggccaggcagtcactcccccttgccgacttcagagcaactgccctgaaagcagggcctgaggacctctggctgtggggctcagctagctaaatgtgctgggtgggtcactagggagagacctgggcttgagaggtagagtgtggtgttgggggagtcaggtggcttgcggccacttagaagtcgcaggaccacactccccaggacagggcaggggccagcggtccagtggctggaggtggcccgtgatgaaggctacaaacctacccagccgcagccctgggaaggaaggtgggctctacagggcagggcaccttttaccctggagctgcctgcttttgagggtaacagtcacgcccagccaagacaaggcctggggcgttagtgggtgacctaggcactgcggggcgggggggctgggtctacacagcctgggtctgggcccaccgtccgttgtatgtctgctatgcgcagccacagctgaactgccctcccagaccatctggaggccgctgggggactctggggaccaagactccatgtgccacagaggattgggggcggggcggtgctaggaactcaaagccagcctgggaagaccctgtccttgtcaccctttcttgccttgggtctgtccactgagtagcacacaagaccgggtgggcagggtccgttctgctccgggaatcacagactgtgtgtacccaggtggtgggcatgcagcgatcagtggcgtgggaccacagaggggcattcggctcagcctagaggaacctgccttcctcctcctccccctccttggaggtggtgacaagaagctggatttttacacaaaaataggaagagtggtgaggtgggggcacctcaggcctgcggggctctttgcagcgcgtagtaggtgttcactgagtgtgtgtcgcaataggcaaagatgagtccatataaggacatggaacctgtggccaggaaggaggctgcttgttccagttcacggtacaaggctgtgctcagccaatgggacctgctcctcccttgaaggttgcagaggccacagcctggtgggaaagatgaccaccacccagcacacagtggcagacacaggttcacagtccagaagacttgctgagcctcctccatcaccaccccacgcaagggcaaacttccccagcctgtatactccctgcctcctggggatgggactgtcctgtccctggccttgtatctttcagctgcaggcgtcttgccagctgcctcaggacctgccaacgttcacccggctggcgttgtggggcgatgggcggggggcccttcttgctgcctgttgatgagggctggttctgggggttcgtggaacgttgaactgggtggagtgctgggggggtggggtgggcagtggggatgggaccggatctgcagagcagggatgagaggttgtcaggtgggcggctccaggcagcaaggtggggtgcagagcagacctgatccccaggcaggacctgtgttgccactgggcctccattcagctggccaagagacaggagctccagccccaccccgccccgggctgcagcattcctggcctgtcctcctggcttgtggttccctctgtctgcagcggccacttcctagtgctctcctatgcacccttcaagtcccatcccagaaagcccctctttggggaagcgttttagcctctgcagccacgccttcctccctacctcagaccaaggcttgcagctggcatttagaactgggcctcagccaccaggctgcgagccctcccagggtctccgtcacccccactccttcccactacacccaccccagggctcctcgctgttgaatgagtgactgcctgggtgtcctcaggtggcaggttggaggtggccgagcagcctggggtgaggggcagggaggagctgaagagagaaagcaagcagcggagcaggaaaagaggaagtagaggtgcctggccagggggcgggccgagcaggggagaagtgaacaggttctcttctgtgcttgggaactttgccctgtaagggagctttcggagctgtcactggggcacgcacgcagcccccggagaaccctcggttgaactcagccaaggggactccacctgcctggctgggacttttgttttttcccctccaagcctttgctttggtgacacccccaccctgcaaggcctccaggcaaccaactcttacagcgcagacccagggaccactccttggccctcccgcaaaacaaccacccagccaccttgccctgggcactgcagcgccctccctatcctgccaggagggaatccaacctcctttttattattattactatttttgatacaaggtcttgctgtgatgcccaggctggagtgcgatcacagctcactgcagcctcgacctcctgggctcaaagccttcacgagtagctgggactataggcacacacttccaagcctggctaatttttaaattatctgtagaggtatggcttctcaccatgttgcccaggctggtctccagctccggggggctcaagcgatcctcccacctcagcctcccaaagtcctgggattacaggcatgagccaccgtgcccgtcctcaagctccattttgtagaggaaggaactgagacagagcttccctccaagacttgagttagctctaggaggacagggaccctgaacacggtaggtgctcagtgagaggagaatgaatgaatgagggggggcaacattccaggagacacacccccattcacagactaggaaactgagtcaggactggtgaagtgctcaagtccccaaatgcggtgggaaggactctagcctcagggtccctgaggcggcagccctgcctcctgcctcacactgcccctccctggtaaagggtcccctggaatgtccccaaggcctgagtccaaaggctgtttgaaagtcggaattcctgggccctacctccctcctcagaatttatcacctgaggcccagagaggacaccatcagcccatggtcacgtggcagcaagtggggtctctcccactggggtgtgatggcttgtacctgtaatcccagcattttgggaggccgaggagggaagatcgctcgagcccaggagttggagacaagcttgggcaacacgggagatcctatctctacaaaatcagctgggtgttgtggcacgtgcctgtactcccagctaattgggaggctggggcaggagcattgcttcagcgtggaaggtcaaggttatagtgagctatgatcaagtcactgcattccagcttggatgacagaggaagaccctgtctcaaaaaaaaaaaaaaaagaaaagaaaaattaaatttaaaaattaggtgctccaggctgggcgtggtggctcacgcctgtaatccctgcactttgggaggtcaaggcgggtggatcacctgaggtcaaaaattcaagatcagcctggcaaacatgatgaaaccttgtctctactaaaaatacaaaatattagctgggcatagtggcgcgtgcctgtaatcccagctactcaggaggctgaggcaggagaatcgcttgaacccgggaggcagaggttgcagtgggccgagattgcgccgctgcactccagcctgggtgacgagcaaaactctgtctcagaaaaacaacaacaaaaaagttgggtgctccagcttttattcatttaaaaataaaatagccgggcacagcggctcacaccgtaatcccagcattttgggaggccgaggcaggaggatctctcgagtccaggagttttgaggagcatggtgagaccctgtctctaccaaaaaaaaaaaaaaagacaaaattagccgggcatggtggcatgtgcctgtagtcccagctactggggaggctaaggtgggagaactgcctgagcctgaggggtcgaggctgcagtgaaccaagatcatgccaccgcactccagcctgggtgatagagaccctgtctccaaaaaaaaaaaaaaaaagtaaaagaaaaagaaaaagtaggtgctcccggtttatttatttaatttatttttagagataaggtctctaaaaattttagagacagggtctagctctgtggcccaggctggagtgcagtggcatgatcacagctcactgcagccttgacctcctgggctcaagcgatcctcctgtctcagcctcctgagtagctgggacggcagggatgccaccacatctgactaatatttttatattttgtggagatggggtctctctgcaatataacagggtctgtctccctgtgttgcctgggctggtctcgaactgctgagctgaagtgatcctctcacctcagcctcccaaattgctgggattagaggtgtgagccactgcacctggcctcagttttttgttgttgttgttgttcgtttttagttgttgttgttttgagatggagtcttgctctgtcgcccaggctggactgcagtggcacaatctcagctcactgcaacctctgcctcccgggttcagggttcaagcgattctcctgcctcagcctcctgagtagctgggactactggctcgcaccaccacgcccagctagttttttttttttttgtatttttagtagagatggagtttcaccatgttggccaggctggtctcaaactcctaacttcagctgatctgcccaccttagcctcccaaagtgctgagattacaggtgtgagccaccatgcctggatagtttatttatttatttatttattttccttttttttttttttttgagctggagtcttgctctgtcacccaggctggagtgcagtggcacgatctccgctcactgcaagctccacccccctgggttcgcgccattctcctgcctcagcctccccagtagctgggactacaggcgcccgccaccacgcccggctaattttttgtattttcagtagagacggggtttcactgtgttagccaggatggtctcgatctcctgaccttgtgatccgaccgcctcggcctcccaaagtgctgggattacaggcgtgagccaccgtgcgtggcctattttttccttttttgagacagagtcttgctctgtcgcccaggctggagtgcaatggcgccatcttggctcactgcaacctctgcctcccgggttcaagcgattctcctgcctcagcctcctgagtagctgggattacaggcacccaccaccccacccaggtaattttgtatttttagtagagatggggtttcaccatgttggccaggctggtctcaaactcccgacctcaggtgatccgtctgccttggcctcccaaagtggtgggattacaggtgtaagccaccacacccggcctttatttatttatttatttatttatttatttatttatttatttattttgagatggcgtctcgccctctgtcaccaggctggagtgcaatggtgccatctcagctcactgcaacctccacctcccgagttcaagcgattcccctgcctcagcctactgagtagctgggactacaggcgcgtgccaccacgcccagctaattttgtttgtattttagtagagacggggtttcaccatgttggccaggatggtctccgtctccatctcctgaccttgtgatccgcccgcctcagcctcccaaagtgctgggattacaggcatgagccaccgtgcccagcctattttattttttaaaataaaacagagtggggtctctctgcctcctggccaccttctcagccttaagggccagcttcaggctgccttctccaggaagcctgccatgacttcctccctcttctactctaacagcagaaggtttcctgctcagaaaggggcagtgacccagccaggatcacccagtaagggcacagccaggagctgaagggccttttgcctggagcagctcctggatcacactgctcctgcatgcagcacaccgtcactgaccagccggacatgcttccagagttaactggcactgttttgagaaatgagaaaagcaccctgaagaaagcaagcacaattcctaagacagatcttaaatgagagagagagcaagagaacaacaagtttaatggcatcaggaggaaaagcaacaggtttccttcagaatgaggtattagggggtgctgggtgtagtggctcatgcctgtaatcccagcactttgggaggctgaggcaggcagatcacgaggtcaagagtttaagaccagcctggccaacataatgaaaccccacctctactaaaagtacaaaaattaaccgagtatggtggtgcacacctatagtcccagctactcgggaggctggggcaggagaatcgcttgaacctgggaggcggaggtttcagtgagccaagatcgtgccaccgcactccagcctgggctatagagcgagactctgtctcaaaaaaaaaaaaaaaaaaaaaaaaaaaaaggagggtgttgggccgagtgtggtggctcacgcccataatcccaacactttgggaagacaaggtaggaggatcacttgagtccaggagttcaagaccagcctgggcaacatagcaagaccttgtctctatatttaaaaaaaaaaaaaaaaaaaaggaaggtgcttttttttatttttcgagtcagggtctcactttgtcaccaggctggagtgcagtggtggaatctcagctcactccaggctcaaccttttggcctccattgatcctcctacctcagcctcctgacttactggtactacaggtgtgtaccaccatgcccagctaatgttttgtagagacaaagttttgccatttgcccaggctggcctccaactcctgggttaaagcaatccacctgcctcagcctcccaaagtgctgggattacaggcatgagccactgcacgcagcagagagtgctgcttagaaagcagtcagggggccaggcgtggtggctcacgcctgtaatctcagcactttgggaggctgaggagggtggatcacgaggtcaggagatccagaccatcctggctaacacggtgaaaccccgtctctactaaaaatataaaaaattagccaggcatggtagcgggcacctgtagtcccagctacttgggaggctgaggcaggagaatggcatgaacttgggaggtggagtttgcggtgagacgagatcgtgccactgcactccagcctgggcgacagagcaagactccatctaaaaaaaaaaaagaaaagcagtcagggagggctcctcagaggaggtgaccagcagagccttggtccccacatacacctggcctccagacatagcctggggtggtcacttggctgccttcagtagctctgccctgggcctctgcagctccttggcagaggcaccaggctaaggggaatccctgccccagaggaagtgggaacttctaacacttcttgggagagttttaaaaaatccttaaaaactaccctctgtctccagcccgcagggtacccggagaaacccttccagccttcctagggagttcctggacttgcttatttgattgaattattcagaaaaagaaaatgcggtttcctgggcttcccccttacacacacacacacacacacacacaccacaccacacacacaccccacacacacacaccccacacacaccacacacacacaccacacacaccccacacacacacatcccacacacacaccacacacacacacaccacacacacaccccacacccacacaccacacacacaccacacacacacactgaacgctttttgctttctctggacccatctgctgtagggccagctctcccccttctccagatgagaaagagaccacatgtgcagagaggaactggatcctttggggcctgcaggcctaggctgtgggcgcttggagggggaggctgcgggcaccctcatcctccccgggactcacaaccactctcctagaggccaaggtagcccttgggactcaggacccttctggttgtcccgagtggggcctcagaagctgggtgacaagttgggccaggagagatgggggtcagtagcaagttcaagttcaggtcaagtggggttcccgaggccctggctctgacaccctcttggggtgctgcacaggccaagggtgggtccttcccatgctaaggactgtcccaggcagtggcttccctgccctgggcctcttgccctcagctgtgaaaaggggcagggggttacctagctcccaggtggcccctgagaggggccagtgaggcggggtctgcagtccaggcccagggtcagtgcgagggactcgccccttcctggctggctgggcttatccctgacgctctcctgcctggcctgggacctcagaaatgccccttggagctgggcatagtggctcatgcctataatcccagcactttggaaggctgaggcgggcagatcacctgaggtcaggagttcaagaccagtctgggcaacatggtgaaaccccatctctaccaaaaatacaaaaaattagctgggtgtggtggcgggcacctgtattcccagctactcgggaggcggaggcaggagaatcagttgaacccaggaggtggaggttgcagccagctgagatcgagatcacgccattgcactccagcctgggcgagaagagcaaaactcagtcttaaagaaaaagaaagaaagagacaaagagagaaagagaggctgggcacggtggctcacacctgtaatcccagcactttgggaggctgaggcgggcaggatcgcctgaggtcaggagttcgagaccagcctggccaacagggtgataccccatctctactaaaaatacaaaaattagctgcgtgtggtggcgggcacctgtaatcccagctactcgggaagctgaggcaggagaatcccttgaacccgggaggtggaggttgcagtgagccgagatcgcgccattgcactccagcctgggcgacaaagagcgaaattccatctcaaaaaaaaaaagagagagagagaaggaaggaaggaaggcaggcaggcaagcaggccccttgggaggccaggcacctctcagcctcagtctcccttgttctgaggtggggtgaagattcccacccgtggttctgatatgggggacacgtgattgggctgggatggagctcacatccttcagcccctcggggtgcctgaagaactaagaacggaggccgaagcacagtggctcacgcctgtaatcccagcaagcaggtaggtggctaaatacaaaaattagctgggcgtggcggcacatgccagtaatcccagctactcgggaggctaaggtgggagaattgcttggacccaggaggcagaggttgcagtgagccgacatagcgccattgcactccagcctgggcaacaagagcaaaactccatctcaaaaaaaaaaagaaaagaaaagaaaaaaagggatgattgcctgagcccagcagtttgaggctgcagtgagccatgatcgtacctctgcacatcagcctgggagacaaagcgagaccctttctctaaaaaataagttttaaaataaataaaatagtctggggcactggctcctacctgtaatcccagcactttgggaggccatggtgggtggatcacttgtggtcacgagttcaagaccagcctggacaacatgttgaaactccatctctactaaaaaaatacaaaaattacccaggtatggtggcaggtgcctgtaatcccagctactcgggaggctgaggcaggagaatcgcttgaacccaggaggtggaggttgcagtgagctgagatcacaccactgcactccagcctaggcaacagagcaagactccaccacacacacacacacacacttttttatttaaaaatttaaaaaaatgtcccttggaggtggggcagtggcggtatgggtgaggcccccttggaggggtggagaaggagagaaaagggaggtgttgggaggtgcaggcggacggggcagctgctgactgaggaggccactggctcacctgccttggggctcagcctgtcctcctggccacccccagtccagagaggagaagggaagacagaggccccaagggcctactgtgtgctgggcgcgtgtcagggtggtgcccacgtcacctcatcaaaagcaccccacacccctgtcggccaagtgggactctcccatttctcagatgagtaaaccgaggccagggaaagtgagagacttggccctatagttaacacggtttggctattaccaggatgttctggaacatgattatccaggtcctggcccagggccaccattcatggcgagcttccaagggcccaccaagtccttcagcctctatgtggttgttgttctcagccccatgtttcagatgggtaaactgaggtttggagtggccagtgggaagaggcagagccaggctcggtggctgcagttggcctcgctgccctgccgtggggcgcattcagggttcacttggtgctcctgagctggcgtgggctcctgcagtcccggggcctgcctgcccaaggggaaatccgatcacagccaccgcgcagcatcaccaggccctcctcagccagaaatctctgccactgctgggcttgccaggaacctgagccggcccgtgggggacaggcccagctgtctgggcttcctgaggtgtctcccatacgctgccccgtgccaggtgctggggacaggaggggacctccctggcatgcactgtccactggggacaggggcacagagctggccagccctgatcccatgagactggtcctgtgcaaacccagggaaagccccagacccagcctcagtgcagggagagtcccagaggcctcctggtcttcgattttgccggctcaggcaagaaggggtttggggtttctcttgggacttcggcttctgctggggacaaggcacaggcagggctccctgacctccctgaggaagccccgccgctccgtttcccaggtctctagggggtaatttttttttttagacggagtctcgctctgtcacccaggctggagtgcagtggcgcgatctaggctcactgtaagctccgcctcccgggttcacgccattctcctgcctcagccttccgagtagcctataggtgcccgccaccacgcccggctaacttttttttgtatttttagtagagatggggtttcaccatgttaaccaggatggtctcgatctcctgacctcgtgatctgcccgcctcggcctcccaaagtgctgggattacaggcttgagccactgcgcccggcctctaggcggtaatttttgctacttaagtcaccttgtctggcacggtggctcatgcctgtaatcctagcactttggaaggcctaggcgggcagttcacataaggccagaagttcaagaccagcctggccaacatggcgaaatcccatctctactaaaaatataaaaattagcagggtgtggtggcacatgtctataatcccaactactcaggaggctgaggcatgagaattgcttgaaatggggaggtggaggttgcagtgagctgagatcacaccactgcactccagcctgggcaacagtagagacaccgtctcaaataataataataataatgtcacgtgggctctccgatagccaagggccatcaaggccagagatgaccaaggcccaatgttgggacctccatacctttgtccctggggccacctgggctgcacagacatgtagcattgggggtgacagtggagaaaggcacacagatgtccctgaggctgtgacccgggacatgtaaggctggagggctggtccaggctcaggtgggacttagcagtgagccagctgtgcacaggccagggtgctaagcgttgggccctcttagctcagcaaagccggaatgaagccgggcgcggtggctcacatctgtaatcccagcactttgggaggccgaggcaggcagatcacccgagattgggagttcaagaccagcctgaccaacatggagaaacctcgtctctactaaaaatacaaaaaaattagccgggcttggtggcacatgcctgtaatcccagctattccagaggctgaggcagaagaattgcttgaacctgggaggcggaggtttcggtgagccaagattgcaccattgcactccagcctgggcaacaagagcgaaactctgtctcaaaaaaaaaaaaaaaaaaagccacagtgaagagcgccccagatccggtggggctctacagaaataggcatagcatccttcccacgctgcgagacccccagaatagtcttttgaggaacctgatctttcctactcggagccactttcaggctttatattaagtagggttcagccatgccactcacacactggatgtccttggaaagtgactcagcctcttcaagcccacaagcctcagcaaggccctgtgtgggtcaggtgcagcggctcatgcctctaatcccagtcctttgggaggctgagttgggaggttcgcttgaggccaggaatttgagactagcctgggcaacatagtgagaccccatttctacagaaaaaattttaaaagttagctgtgtgtgataacatgtgcctatggccccagctactcaggaggctgaggcaggaggatcgcttgaggccaggagttagagactagcgtggtaaacatggcaagacccctgtctctacaaaaacaaaaaaagacaaaaaaacatgaccccatgtgtatgacactgactggggaggcggggcttccaggcctttcagggacttgtcacagaccctctagaagctggacacaaaatgaggaaaccaggccaggcttggtggctcttgcctgtaaccccaacactttggggggccaaggcaggtggatcacttaaggtcaggagttcaagaccatcctggccaacatggtgaaactccatctctactgaaaatgcaaaaagtagctgggcacagtggcgcatgcctgtaatcccagctattagggaggctgaggcaggagaatcgcttgaacccaggaggcggaggttgcagtgagccaagatcacgccactgcactccagcctgggcgacagagcgagactgtctcaaattaaaaaaaggggagggccgggcgcggtggctcacgcctgtaatcccagcactttgggaggccgaggcgggcggatcacgaggtcagaagatcgagaccctcctggctaacacggtgaaaccccgtctctactaaaatataaaaaattagccgggcatggtggcgggcgcctatagtcccagctactcgggaggctgaggcaggagaatggcgtgaacctgggaggtggagcttgcagtgagccgagattgcgccactacactccaggctgggcgacagagtgaaactccgtctcaaaaaaaaaaaaaaaaaggtgggggaaaccaggctcagagatgatcgaagtctcagccagaatcacacagcacacgtggcgagccagagcccaggcctggagggccgggtagggggatgcaccttgcatgccgctgggatattgaggacaatttgaatcttctttttttatttttatttatttatttattgataacagagtcgctctgtcgcccaggctggagtgcagtggcaccatctcagctcactgcaacctctacctcccgggttcaactgattctcctgcctcagcctcccgagtagctgggactacagacgcctgccaccatgcccagctaatttttgtatttttagtagagatggggtttcaccatgttggccaggctgctcttgaactcctgacctcaggtgatctgcccacctcggcctcccaaagggctgggattacaggtgtgagccaccaagcccggccgcaatttgaatcttctgctgggccaggctggtctgtccaggggggatgcaggaccccgaagccgggtgtggtggtgcatgcctgtggtcccagccacttgggaaactgaggtgggaggattgctcaagcccaggaggtcaaggctgctgtgagctatgattgtgacactgcactccagcctgggcaacaaagggagaccccttaactaactaaatgaatgaatgaatgaatgaatgaaggcaacctgagctgcattccttggcctgccaacctgcccagccccatccctcagccctccctgagtctgagggccctgcaggtcccacacagggccaggctccatcttgtttctgcaaatttgcaccttccgttccattccctgccacactgctcacgggtcaccatggatgctggataactgccaccgtctccttggagaagccctccgtcatctcagcccctgatgtcaggtattcaacctgcaaatcctccctgtaggtgctgggctctccagagccctgggctgtgtgcaggggactcaggggagaacgggccccacccagctccttcctcacagagctttcagaggctgggggcctccctgatcccctcccagatcccaaggcccctgctgcccccaccctgcagactgggactccgtgaggctgggctctgacttggatattgtggttccagcacacagcaggcaccgtggctgtagtaggcgtgcatgggaagtcaggaggagcagacctgtcatctcccctgctgaggacacagcccggtcagcgtgtcttggcggcctggggccagtgactgagcctcttggcccagttccttgcccaccttgaagatctgtagggagggataaagagatcaggtccaaggccaggtgcggtggttcacgcctgtaatcccagcactttgggaggccgagccaggcagatcgcgaggtcaggagttcgagacgagcctggccatcatggtgaaaccccgtctctactaaaaatacaaaaattggccaggcatggtggcaggcgcctgtaatcccagctgaggcaggagaatcgcttgaacccgggaggtggaggttgcagtgagccgagagcgagccactgcactcctgcctgggtaacaaagcaagactccatctcaaaaaaaaaaaaaaaaaaaaaaatcaagtctggcgcagccccaggaagcaatcttgggctgggcacaccgcatttctgtgacatgaaggcagcgacacctcgtcgctgtcacagggttgctggggttgggggctagaggagaggaggagcctctgttgagggctccaagaagggacagagaggtggtgctcatgggtcctggagacaccttttgggtggttcgtgccctccatcctgggccactttggggaggtgaaggagggaagcattaagggacaagaccccccgttcccaatttctctccggagccagggtttctctgattcgaagaaacaggtgtcacaacccaggaagtccactgatggcatctgccctggggcatcagcatttagggctgatcactgaggtctgcacctcccaaggctgctgtgcccattcctgggcgccccaaaggggaagaaaaactcctgaatgtgcaccgggacaggacccatcccatgcggtgacagaacctcatgatagcgcccggcgctctggatattcaaactgtggagcccagcaagggagcgccggtggtcgggcagggtcagaggggttttgagggtgtgcgatggctgctgtggtgagccactggcatgcctaatacatgcaagacctgttgccaggccctgagctgcagagccacctggaccaaggtcactgtggtggcagggtgcccacatccatcagctgctggccacaggattgcgagccctggccatctctgcccaatgccaggcacctctgaagggccttccggctacagagctcctgcgggatgggccggggtgtccctggccatcatggtgtccctgggcctgcattgaagctcaacttctgttgaccccaagggcacttcttaatagatgtcctactctttttttttttttttttgaaacagaatctcactctgtcacccaggctggagtgcagtggtatgatcttggctcactgcaacctctgcctcctgggttcaggcaattctcctgccgcagcctcccgagtagctgggattacaggctcatgacaccatgcccggctaattttttgtatttttagtagagacggatcgggggtctgttgcccaggctagtgtggcactcctgggctcaagcagtccacctgccttggcctctcaaagtgctgagattacaggcatgaatcaccacacccagcctacatccatccatccatcgatccattcatccatccatccatccatccatttagacaaagtctggctctctggctgtgttgcccaggctggagcgcagtggtatgatctcagctcactgacctcctgggtttgacctcagccttgacctcctgggttcaagcaatctccctgcctcagcatcccaagtcgctgagactacaggtacacaccactatgtctgactgttttattttttgtaaagatgggatcttgctgtgttgtccaggctggtctcaggtgatactccagcctgggcctcccaaagtgctgggattacaggcgtgagccactgtgcctggtcgtctctagtccttggagcaccaaactcctgcctgccacagggcctttgcacttgctgtgcccccgacataccctcctgtcaggctctccctaaattgtctccttggtggactcctattcagacttcaaggcccagcccaaaagacccctccctctctggaaagccttgccagttctaccacacagagcttttttagctctttcttacttatccttcagggctcagtgtcagcatcacctcctacagcaagtcttccctgatccctagactgggtcgggactcttgtagtattttttgtcatactggtcacagtggataactaagtgtatctgtgtgatgatgggtttatgctctgcctcgctgctccacaggcaggaatgacatttgtttgtttcttttttgagacagggtctcactcttggcccaggctggagtgcagtggttcaatcatagctcactgcagcctcaaactccggggctcaggtgatcctcccacctcagcctccggagtagctaggactacaagtatacaacaccacacctggctaatttcttttttttttttttttttttttttttgagatggagtcttgccctgtcgcccaggctggagtgcagtggtgtaatctttggctcactgcaacctccacctccaaggttcagggcaattctcctgcctcagcctcctgaatagctgggactacaggcgtgcatcactaggcctggctaatttttgtctcaactatgagacagggtttcacaatgttggccaggctggtctcgaactcctgaccttgtgatccacccgcctcagcctcccaaagtgctgggattacaggtgttagccaccacgcccggccaaatttcttgtactttttgtagagatggggccttcccttgttgtccaggctggtctcaaactcctgggctcaagcaatccccctgcctcagcctcccaaagtctgggattacaggtacaagccaccacatctggccttgatacctgttttgatcactttgggatcatggcatgtagttaggactcagtaaactcagctcctacttgcaaataggaagaaaggaaggaaggaaggaggaagggaaggaaggaagggaaggagggaggagggaggagggaaggaataaaggaaggaaagaaggagggagggaggaaggaaggaataaaggaaggaaggagagagggaaggaagggagggagggaaggaagggagggagggaaggaaggagggagggaaggaaggagggagggaaggaaggagggagggaaggaaggagggagggaaggaagggagggagggaaggaaggagggagggaaggaaggagggagggaaggaagggagggagggaaggaagggagggagggaaggaaggagggagggaaggaagggagggagggaaggaagggagggagggaaggaagggagggagggaaggaaggagggagggaaggaagggaagaaggaaggaggaagggagggagagagggaagaaggagagggaaggagaggaaaggaaaggagaggagagagggagagagggcagaccccgttctatgtcagaagggacagaagtaacatccggaagggatgtgttatctgtcaagtgagtgagcatcctgtcccaggagtgtgtcccaggagggtgtgggcacacccttggcaggaatggtgtcccgggactgccagagctgttgaggaatagcgctagggaggaaagggctgggccggcttcgggctcacaccttccaaggatgtcagagaggagggtgtgtcacaggaagtgcctgggtctctgggctgtaactgactcaccgggactctgataatattaatactcagacgccagactgcagcctgttctccagaggcccccaggggaatgaatcaatgactgtctgatatcctgaatgtgtgttagaggctgacctggaggaggcagctaacccagcttgaggaactggcaaggctttcctgaggaagggtcttttgggctgggccttgaagtctgaataggagtccaccaaagagacaatttagggaggggttgacacggggggatgttggggaaactgcaagtatctgatgtgcctgggctctttgggaagcgtctggagagactttggtggggaggtctggactggacaccatttctatcctggccagtacccagcaaagtaagtgaagaggccgggcgcggtggctcacgcctgtaatcccagctatttgggaggctgaggcaggagaattgctccaacccgggaggccgaggttgcagtgagccaagttcgcactactgcactccagcactccagcctgggcagcagagcaagactctgtctcaaaaaaaaaaaaaaaaaaaaaaaaaaaaaaaggctgggtgccgtggctcacacctgtaatcccaccactttgggaggccgaggcgggcggatcacggggtcaggggatcgagaccatcctggctaacacagtgaaaccccgtctctactaaaaatacaaaaaattagccgggcatggtggcgggcgcctgtagtcccagctactcggcaggctgaggcaggagaatggcgtgaacccaggaggcggagcttgcagtgagccgagatcgcgcctctgcactccagcctgggtgacagagcgagactccgactcaaaaaaaaaaaaaaaaaggtgttggggagatctggagtgagaaagtctagttggacaaactccattgagaaagtctagttggatgaggaggcttgtgggggcatgggcgctgggtgagccctcttagcactgcctacccctttctctctgccctgatccatcccaagacacagcgtgggacaagaagtcagaggggaccctgggtgctcgaagcaatggaccggacccattgtacagatgggaagactgaggtgggcagggacagagcccacgcttacacacctaagctcagcttgggttctggagcccatgaaggcaagagacaggctcccctggagttaggctccacatagaggcaagttgactgggccagcttctgggcccagcgctggggcactggaagacctgccaaaaaacttagcagaaagtaggtactagaactctggatcaggcctcccagctaacagccgcgggacctctgggcttccttcccccaaagctgggggaaggtgagcttttcctccccctcagccctggcccagagaagaaaacagagcttgcaaggtcacacagcaagaatgtggcactggaactcagctaagaggtgggagagggccttggaatctagagattagccggtgtctggaatgcgtggggcaggagtgtcctcagaggaggaccgcagggctgggcatgggctcaggagcttcggctttgttccctaagggcaatggggagccatggagggtatgagagcaggagagaagcaaaagatgtttcaggaggtggctctacattataaaggacccaagaggagtccaacacagaccctgtgaccttggatttcagatttctgtccgcctctggccaagtaaaagtttgtgttagaagttgtaatcatgggccgggcgtggtggctcacacctgtaatcccagtactttgggaggccaaggcatgtggatcacgaggtcaagagatagagaccatcctggccaacttggtgaaaccccgtctctactaaaaatacaaaaattagccaggcatggtggcgcatacctctagtcccagttactcaggaggctgaggcaggagaattgtttgaatccgggaggtagaggttgcaatgagctgagattgcgccactgcactccagcctgccaatagagcaagactccgtctcaaataaaaaaaaaaaaaaaaaaaagaagttgtaataataggttgggtgcagtggctcacgcctgtaatcccggcattttgggaggccgaggaggacagatcacttgaggtcaagagttcaagaccagcctggccaacatggcaaaaccccatctctactaaaaatacaaaattagccaggggtgatggcacatgcctataatcctagctactccggaggctgaggcaggagaatcgcttgaacccgggaggcagaggttgtggtgagccaagatggtgccattgcactccagcctgggcaacacagtgaaactccatctcaaaaaaaaaaaaaaaaaataataataataatactaacaacctgtgtgtctgtgttgctttttccgttttttgatgctttataatttgattttgcccccatggcaactccgagatgtaggcagggcagctgtcatcacaccagtttattttatattttattttattctatttttatagagatggctagggggagtctcactatgttggccacgctggtcttgaactcctggccccaagcaatcctcccacctcagcctcccaaagtcatcacgtcattttatagctgcgggtccctgggccagtagttgcagtcggtgtgtgcagctcccacgccttccatcagggcgttgcccacctggatgaggggaggtggtgagcccctgcctaagaccccctgtcccctctggcccagacatccttcaaaggtcatcaggagccaggccctgtcctgctctgtccctgtcaattcccaacagaggcacaggcccctgttttctcatctgtaaaatggggtgggccgagcgcagtggctcatgcctgtaatcccagctctttgggaggctgaggtgggtggatcactagagttcaggagttcaagaccacctggtcaacatggtgaaatcctgtctctactaaaaatacaaaaaaattagccaggcacggtggcacgcgcctgtaatcccagctacttgggaggctgagacaggagaatcgcttgaacccaggaggcggaggttgcagtgagccgagatcacgccactggactgcagcctgggcgacagaatgagactctgtctcaaaaaataaaataaaataaaataaaataaaataaaatggggtggaaacctgctgtatggagttcctgggaggacaacgccagcgtgtccaggaatcagaccagcgctgcccgcagctcccgtgttagtaaaacgttagctgtcattactgcttacgtgcccggggagggaggaatcaggagccactggcctctctgtgggttccagggaaggagggcaggggaggtacagagagaaggagcagagtgttcaaggatcggatggaggctgagcttcctggccatgccttagctcacacccagcgacctgagtccagcgcagaggacaaagggcaaaaggcaaaaggcagggtctggagtctggcctctccctgtcatccaggggccactccgggcattatctaggttttttttgagacggagtctcgctctggcgcccaggctggagtacagtggcgcgatctcggctcactgcaagctctccctcccaggttcatgccattctcctgcctcagcctccggagtggctgggactacaggcgcccgccaccatgcccggctacttttttgtatttttagtagagacggggtttcaccgtgttagccaggatggtcttgatctcctgaccttgtgatccgcctgtctcggcctcccaaagtggtgggattacagccactgtgcctcgcctatctaggcttaatcatgacagccatccttccctaagggtgcaccgggtggggtgcgggccctctatctctgagcttttgcaagcaggccccctctgcccccacctgcccactgaaccatcaccctcagcctgggggcagggagcagatatgttactgtccagaaatggaggcccggaggctcccagaagagggaggtgtcaaaatgccacagtgcattccttccttggggagctgaaccagaggccacgcccagccacagccctgacaccctgacactggacatttctgggtgggtcataagaagtcacagcctgaggccgggcacggtggctcaggcctgtaatcccagcacttcgggaaggtgaggcaggagggtcacttgagcctgtaatcccagcaatttgggaagctgaggcaggaggatcacttgaacccaggagtttgagaccagcctgggcaacatggtgagaccccatctctacaaaaaaaatacaaaaattagctgggtgtggtggcacgtgcctgtagtcccagctactcgggaggctgaggtgggagaaacacctgagcccgtgaagttgaggctgcagtgagccatgatcacaccactgcactctagcctgggtgacagagcaagatcctgtctctaaaaaaaccaaacaaaaacaaacaaacaaaaattagcctgatgtggtgtcagacgcctgtagtcccagctactcaggaggctaaggtgggaagatcacttgaacccagtagtttaagaccagcctaggcaatatagcgagaccccatctctaccaaaaaaaattaaaaattacatgcacgtggtggctcacacctgtaatcccagcactttgggaggtcaaggtaggaggatcacttgagcccaagagtttgaaaccagcctgggccacaaagcgagaccctctctctatagaaaatctaaaaatctaaaaattagctgggtgtggtggcactcgcctgtggtcccagctactagggaggctgaggtgggaggatcatttgagcctgggaggtgaaggctacatgagccgtgatcagaccactgcactccagcctgggtgagagagtgagaccctgtctcaaaagaagaaaaaagcaagaaaagaaaaccaataaatcacagcctgtcaggcggctcatggggaccctccttgggaatatctagaacaacattcctggtgtctggagctgtctagtcagtccttacggcccagcagcaggagatcagaacttttgcctcatccagggttagccctgccgtgtctggcctcaaagtgcccatctgtataatggagccaacggcctcctagggccctaggtggacgctggacaacctgggtggggcggagcgggtggcatttggagcaagatttccctactgggagtccctggccccagacaagagtccttctggagtcccagagctgtgtgtgtgtgtgtgtgtgtgtgtgtgtgtgtgtgtgtacacacaagtgcactggggggggtgctcgttcagagctaattagaggtgttggccccatctgcccccaagtcggtgatccctcagatctctctgctgcccgagaagagactgtttcgcccccttccccagacgggccttctggcatggttggggttcgaacccggggccagggctagagtctcagcttggggtgcgggggccgactctgcccgggctgggtggatagttgttatcgggagctcccaggcccgcgcggctcacgtgggccgcagctcgcggcgcctcccgcgtcccagggccgcgaggcgctccgggagataagccaggccccgtgcgagtccagcgcggacccggccggcccccagcccgcccctcaccggcccccaccccgcccctcggccggcccggcccacttggcccgagttcctctccataaaaggcgaggtttgggagcgcccggggagggggcgttgggtccacgggccgcccccgcccaccgtgttcccctccctcactgcgagccctcgccggcccgggggtgcagcccagcccccctgccgcgacccccacgccgggcccgtagaccacgaccctgaccccagtccccactccggatcgggcccacagcaccccccgccggtccccgcgagtcccgcgctctgtccccattttgcgattgaagaaattgaggcccgcagcgtgcgtagcccggctggggccacggggttggggactgaacggccggaaacgccacacgtgcctccaggtggcaagccagctcctcatagacgcacacgaacccttctgcaagtggggtgcatgtgtatatttacacctatagggattgggggcctcagggatggggggccctcccccgcacttagactagaatcccccctccccgcagcgctgccacttagctcttacgtgaccctgggcacacgatacttttactcacagcctcagtttccctgtctgttaaatggggggaggtttgcatctgccgggtttgcacaacgggttgtgcaaagtacaacacccagccaagggtcagtgcaggggaacaggaggaatggggcaagggcggggcccggcgcacccccagcccccgccctgccgcacacccctctcagaagaacctgcatttagggtgagacggatcggacctaagcccgccccggcgagactgcagtgagacttagacataaggcacaccatcaagcaaacaccgggtgctccataaatgcagctgcgccctcccgccccaccccactggggagcgtgagtcagctcggtccaggcgaggctgggaagccgagccttggctctcatgtggctccacccgcctggctctggggccggggcaggtctcgcctctctctgggcctcagtttcccctctgcctgctgcaggccagggccgggcctgaccccctagggggaggagggcggcgaggagccgggcagcggggagcccgggggtccctcgggggagcgcgccccctcccgcccccggcccgcggcccctgtggctcagaggcagggtgggccctgagccggccacgtccgcggcccacgagtccgccataaaaggcaaacggcgcgggtcgctctgccaaacttagtcatccggcccgccggcagccgcagcgacttcctccgcgggcttcccgttttcggagcggggcccggagggggtccgaggtgtggccgcgactttcgaaatgcccggctgcttctccgaaggggcggcggagggggcgccctcggagccatggggggaagggggtatcggccgggtgggggaggggatctcccccgccctgccccgccctccggaatgatcccgcggctccttaacccctgagctgccgggagcgggggaaggggctgggctccggccggggtcggggcgtccctgggtccagcagggagtggcctcccggaagacgccggcgagggaggcccaagtgcctgctccgagaaatctcagcgaggcctccttgggagaaaagtgcaaggcggtgacgtccagtctgtaaaatgtgaaaaacgcccaggccgaaaccacccagaacccgttcctgcagggaggagggtgggtgggaccaacaggggatcagtcactgcgccaaggcagggactagggccatggtggacaacgggggtctacctcagtgttccccagacacagacgggttcctactgtctggccccacttatgtgaagttctagaacaagccccacattttctggtgacagcaaacaggtggcattatttatagatgtaggggcaggtatttatgggagggggcacagagaagagcatgagctgggggtggaggggggcatccactgattctgagatcctatatttctttgagaaaacaactgaataaaacatagcaaaataataattgcttcattcggggtgtattatagtaccttctgtacttttctgtatctttgaaatatttcggctgggcgcggtggctcctgcctgtaatcccagcactttgggaggccaaggtgagcggatcacctgaggtcgaaagttcgagaccaccctgactgacatggagaaaccaattagccgggcgtggcggcgcatgcctgtaatcccacctactcggaggctgaggcaggagaatcgcttgaacctgggaggcagaggttgtggtgagccgagaacaggccattgcactccagcctggacaacaagagcaaaactccgtctcaaaaaaaaaaaaaaaagatttcacactttccaaaaaagggaagaaagcgaaaggacaaaggctcagagttggacatggtggtcaaccactgtgaccctcagtttcttcttctatactcatggggcaataatctctttgggggtaattaaaatgatatgcgataatgcaggcaaagtgctaagtacgaggcttgggcatagagtaggtgctcagcaaaggcgtctggtgaggggctgccctttctccccgggggaaggggaggacttgctggggtgagggtcctggatgggaaggttctctgcccacagctctctgcacccagctcctgcctggcccgacctggcctggttgggaaggcttgtgtttctgacaaatggggccagccaatcccaggccctgcctgtgctgttccatcctgtcctgtcttgtccccggggccagggtcactccgggtgagactcagagccagtgtacccaacgggacacaggcaggcaggtcaccagacctccgattccaggcggccggatcttcaggcagcctgtctgggcagtgaggacactggggtggcaggtgccagccggtcaggcctagagccctggccggagcactgttgcgggatcttagcccagagagccccagaaggctgtgggcggccagagccggcctgggaggagggcaggagagggtggggttaggggaaggacgagaaaagagaaagtaaaaggagaacagctacttttcctgatgttatttttgaacacctgtgggtattgagccctgcacccaggctctctgagaaaagtcccccatgaccctgataggggaattaagaccccaaggcaaggcacagtggcgtgtgcctgtaatctcagcactttgggaggtcaaggcaggaggattgcttgaagctgggagtttgagaccagccttggtaatgtagcaagaccccatctctacaaaaaaatttaaaaattagccaagcatggtggtgcacatctgtagtcccagctactagggaggctacaacaggaggatcacttgagtccaggaggtcaaggctacagtgagcggtgattgtgccaccgcacttccaacctgggcaacacagtgagaccccgtctccaaaaaaagaaaagactcccaaatacagcaaggtcaagcagcttgaccaaggtgaccagctgagaagggcagtcaggggaagctgggacccagggagagaaaaagagagattgacagacacatggctggggggacttgcaagccctcgggagggggtaggaagcagctgccgggaatgggtgcccaaagaagcaggaatgaggggtggagggaagggcctcatttctggggaaggcccagggaagggcctcatttctggggaaggcccagggaagggccagggtgcagagaagccaatgacagctggagagttgggactgctgccaggacagactagacggagactgctcttgggtgccacctagtgggaaccttctttactgtcatccctggagagatggacctgtgacgtccccaccggatgccccgcccaccatgggcatccccatcccaatcatccctcaccacccccaggctcgggtcgcgtccccccaggctttgatggacaagtggccatggaaagcatcctcagctgccccagggttctgccatcacccatccactaaatggtccagggaccctgggaggtattgctttcccctttacacagatgaggaaactggttgggcacggtggctcatgcctgtaatcccagcactttgggaggctgaagcgggtggatcacctgaggtcaggagttcgagaccagactggctaacacagtgaaactccatctctattaaaattacaaaaaggagcccggcgtggtggcaggagcctctaaccccagctactcaggaagctgaggcagaagaactgcttgaacccaggagatgatggaggttgcagtgagccaagatggcaccattgcactccagcctggacgacagagcgagactctgtctcaaacaacaacaacaataaaacacagatgaggaaactgaggtctagaggggcggcagcacttgtccagggtcgtggccgacaagtcgccaagctggggagtttccatgggatccaagcctatccccaagcagcccaggggacacagacagcatgcaggcacacaatcgcacaccctgccccgccacccctgagcctgccgcatcgtcattcctgtgcaactaggcaccctgagtctccacatcggggtggctctggggtacacagacgaagcagagagccggcaccccatcctgcgtctgaggaatccagctttccctggctggaagacccggtcatggtaacgccctgtcctgcaggttccgcctctgctgtgacctccaccccttacctaaatgcagatttcagatttccgaggttagcctggaaggaaatagcttatactgtacagctgaagcatggcaaaggaagcaatctccatcaacaggagtctgtgggctgggtgtggtggctcacgcttgtaataccagtagcttgggaggttgatgcaggactgcttgaagccaggaggctgaggctgcagtgagctatgattgtaccactgcactctagcctgggaaacagagtgagactgtgtcccccaaaaacacataaattaataaaattaaactaaataagagacggggttttttttttggttttttttttgagacagagcctcactctgtcacccaggctggagtgcagtggcacgatatcggctcaccgcaaccaccacttcccgagtttaagcgattctcctacctcagtctcccaagtagctgggactacaggcgcccgccaccacgcctggctaattttttggatttttagtagagatggggtttcgccatgttggccaggctggtctcgaactctcaagctcaggcaatccacccgcctcggcctcccaaagtgctgggattacaggtgtgagccaccacgcccagccatgagacagagtcttgtcatgttgtccagactggtctcagactccccagctcaagcaatccttttacctcggcctcccgaagtgctgaggttacaggtgtgaggcactgcacccagccaggaaactttgattaaggctcccacgccttgctcagctccagccctgcaaaaatctccagccctgcagagactgagcatcgagtccctgatttctcttttccagaagtatgtgggaaagggtggttataactgcactctctccaagacggagttcctaagcttcatgaatgcagaactggctgccttcacaaagaaccagaaggaccccggggtccttcaccgcatgatgaagaaactgggcaccaacaatgacgggcagctagatttctcagaatttcttaatctgattggcggcctagctatggcttgccatgactccttcctcaaggctgtcccttcccagaagcggacctgaggaccccttgggcctagccttcaaacccacccccttttcttccaacctttctgtcatcatctccacagcccacacatcccctgagcccagcacaccaaccacctcatgcaggtcccacctgcggatggtaatacaacaatgtcacttttttaaaacatgaaaaaaaaatttttttaaaaagacaggccagaactagtggctcatgcctgtaatcccagcactttgggaggccgaggcgggcgcatcgcctggggttaggagttcaagaccagcctggccaacatggtgaaaccctgtctctactaaaaatacaaaaattagctgagcgtggtgggcacctgtaatcccagctacttgggaggctgaggcaggagaatcgcttgaacacgggaggcagaggctgcagggagccgagacagcaccactgcactccagcctgggcaacagagcaagactccatctccaaaaaaataaacaaataaacaaaatgccaagagtatgctgatgtaaaggtttaagtagaaactctggttgttttactcatgtgtgtaaaccaagcccccaagatgtgccaccaaaatacagagtttgtgggtggatcagcctgtacctgagccccctctgccacactgacatgatcctcattaaagaaagaaaagagggggcaggtggatcacttgaggtcagcagtttgagaccagaatggccaacataacgaaaacctcctctactaaaaatacgaaaaactagccgggtgtggtggcctgcacctgtaatcccagctactcgggaggctgaggcaggagaatcactggaacccgggaggcagaggttgcagtgaaccgagatcacaccactgcactccagcctgggcgacagagggagactccatctaaaaaaaaaaaaaaaatgttacaatggtaaactttatgttatgtatcttttactacaattttttttaaccttgattctgagaaggggtccctaagcgtcatcaaatgctaatcgcgtccacgcccctgaaagcttacagccctggtacttcctattggtccagcctagcaggaagctggcagccatgaattctgggaaatgtagtgtgcagcccccatgccggagtcacgcgcccgctgtgatgggtgggctcggagctggcacacaatagttctgtggccgacatccagctcagcagggaaattgtgaaaggcacactcggtgcccctattcccacgctgcccacgagcagtctcgcgcacggccacacagacctgggcgcacacctgtcccctagtaccatccagcctgtgggcgctccaggtctgcagtttgtacctggacaccgttgccaatctaagtgccaatccgagccgcctgcggatcaggctcgcagacgctcgctcgctcttccctgacgcagcctcagcgggagcaggggccttcgcagtgagggtgggaggatgtacaggctacaggtgccccggcgggaaggaggtatcaggcatgcaacacaagttactgcaaagaccctgacgtgggagcccgattggcccagggtgtggccgggggagacgataggggtgaggtggggttggccacgttgtccatgaagaggcttctaacaagagaccgtggccaagtcccccaggtgagcgctgggacgctgcctagtgaattgaatgactcgtattgttcccaaggttgcagctgtggcctccgaagtgactcagcagaattcctgggagttggggtgggggcgaggcttactccagacgggctgggggagggggcgttgcccgcgcgctagcccctcctcccagctcctgcggaccgcggagctcgcccactctcgcgttctcccctccgcccagacatctacaggcaccagcggcgacgaggcggggatgatgctggaagacttagggtgggacggccccatcctctcggaacctccgtccctgcaaccgattttagggtggggatggggtgggggctgagtctgccctcaaaacggggctcaaggctcttggcttggagcaggcagggttggccctgtccgggcccccggagtgactcagcccagtcctggccctctccgggactaactctcttcctctaaataaagggaatctttcacccagcagcctggagtgggtccgcggagataaaacaggccctccagagtgatcccttccctggtggctgatggggcatgtgtgtgttgggagagagggagagggcagccctccacctcaggctggggcaggctggagtgaaggtccaagcgctggacccgagcagcagcctcagccccaatctaaaagctcagcccgttttcccatgcatacaatgggcataactgtcccaagaatgaggcttgctggagttactggcaagctgcgcgctgtccgcctaattcacctaattcagggcaagaaccgctctctggcccctgcacctgggagaggtggggtgctggaccgagggcgaagtaacagcgtcctgagggcccatccccaaatcgccttccttagggaagccggaacaaacgatgcagcggggcggccctggatctgttccttcctaagagctggagcaggtacagccccatcctagaccccagcaacccgcgcgccctccctcgcaccatctcccctacctggttggttgggttgtggggttccaggcgaccgccaggtggcgcaaccgacgcgcctgccgcgccaagctgggtggggctggactcgtccggtgatggggccctttgggcagccagacacatttttttttcagagtttcactcttgttgcccaggctggagtgcaatggcgcgatctcggctgactgcagcctccgcctcccgggtgcaagtgattcccctgactcagcctttcgagtagctgggattacaggcatgtgccaccacgcccagctaattttgtatttttagtagagacggggtttctccatgttggccaggctggtctcgaactcccgacctcaggtgatccgcctgcctcggcctcccaaagtgctgagattacaggcgtgaagccaccgcgcccggcagccagacacatttaaaagtcctttgggccgggcgcggtggctcacgtctgtaatcccagcactttgggaggccgaggggggcggatcacgatgtcaggagatcgagaccatcctggctaacacggtgaaaccctgtctctgctaaaaatacaaaaaattagcagggcgtggtggcgggcgcctgtagtcccagctactcaggaggctgaggcaggagaatggagtgaacccaggaggcggagcttgcagtgagccgagatcgcgccactgcactccagcctgggcgacagagcaagactccgtctcaaaaaaaaaaaaaaaaaaaaaatgttatccgtgcatggtggcgggtgcctgtagccccagctactcaggaggctgaggcaggagaatcacttgaacccgggaggcagagcttgcagtgagccaagatcacgccactgcactgcagcctgggcgacagagtgagactccttctcaaaaaaaaaaaaaaaaaaaaaaaaaaaaagaaagccctttgaagtggagaaaaggtactgggttcaaaaatgcaaaataagagtccagtcgacggagtgcagtggctcacgcctgtaatcccagcactttgggaggccgagacgggtggatcacgaggtcaggagatcgagaccatcctggctaacacggtgaaaccccgtctctactaaaaatacaaaaaattagccgggcgtggtggcgggcgcctatagtcccagctactcaggaggctgaggcaggagaatggcgtgaacccgggagctggagcttgcagtgagccgagatcgtgccattgcactgtagccttgacaacaagagtgaaactctgtctcaaaaacaaacaaacaaacaattagctgagcgtggaggcacatgactgtagtcccagccactggagaggctgaggccagagggatcacttaagcccaggaattcgaggctgcagtgagctatgattgcattactgcactccagcctgggcaatatagtgagacccccatcttagaaagaaagaaagagagaaaggaaggaaggaaggtagaaagaaaaaaagaaaggaaaggagggaggaaggaaggaagggaatgaaggaaggaagattcctaaattcaattcagaaacacaaaacccaacaacctgactaatgtcctttctgcaatcaagagtaactttttgtgttatcttttgtctcagatacaaatccacaagttaacctgttaacttcactgagcctcagcccttaatcaaaagcagatagtaaagtcaaatcaaagtttcctggaaagaattaaataatccttaggcgggctccttatgcaaagcagccctccccttggcctcctgtccaatattcgaatgttgttttcttttatttatttatttactttatttatttatatatgacaggtctcgctctgtgcccaggctagagtgcagtggcatgatcttggctcactgcaacctccacctcccaggctcaagccatcctcccacctcagcctcctgtgtagctgggactataggcacacaccagcacgcccggctaattttttttttttttttgacatggagtctcactctgtcacctaggctggagtgcagtggcaagatctccactcactgcaacctccgcctcccgggttcaagcaattatcctgcctcagcctcccgagtagctgagattacagatgcgcaccaccaagcctggctaatttttgtattttttttttagtagagatggcgtttcaccatgttggtcagactggtttcaaaatcctgacctcgtgatccacccacctcggcctcctaaagtgctgggattataggcgtgaaccactgtgcccggccttttttttttttgagacggagtctcactcttgttgcccgggctggagtgcagtgacatgatgacatgatctggcctcaccacagccttaacctctcaggctcaagcgagtctcctgcctcggcctcctgagcagctgggattataggcgtgtgccactaccacctggctaatctttgtttttgttgttgttgttgtttttttgagacggagtcttgctctgtcacccagggtggagtgcagtggcatgatctgggctcactgcaacctctgcctcccgggttcaagtgattctcctgcctcagcctcctgagtagttgggattacaggcgtgcgccactatgcccggctaatttttttttttttttttttttgatggagtcttgctctgttgccaggctggagtgcagtggcgcgctcttggcctcctgggttaaaccgattctcctgcctcaccttcccgagtgctgggattacaggtgtgcaccatcatgcccagctaatttttgtatttttagtagagagggggtttcaacatgttggccaggatggtctcaatttcttgaccttgtgatctgcccacctcagcctcccaaagtgctgggattacaggcgtgagccaccacgcccggcaatttttgtatttttttagtagcgacggggtttcaccatgttggtcaggctggacttgaactcctgaccttgtgatccgcccacctcggcctcccaaagtgctgagattacaggcatgagccactccgcccggcctaatctttgtatttttagtagagatggggtttcaccatgttgaccagactggtgttgaactcctgacctcaaatgatccaccctcctcggcctcccaaagtgctgggattacaggcctgagccactgcacctggcctaatttttgtatttttactaaagacaaggtttcaccatgttggccaggctggtctcaaacacttctgacctcaagtgatacccaccccggccttggcctcccaaattgctgggattacggatgtgagccactgtgtctggctgcagctaacttattttaatagttttattttttgagacagggtctcattctgtcacccaggctggcatgcagtagcatgatcacagctcgctgcagcctcaacctcctaggttcaagtgatcctctttttttttttttgagatagagtctcgctctgtcgcctaggctggagtgcagtggtgtgatctcagctcactgcaagctccgcctcctgggttcacgccgttctcctgcctcagcctcccgagcagctgggactacaggcacccaccaccacgcccagctaattttttgtattttcagtagagacggggtttcactgtggtctcgatctcctaacctcgtgatccgcccgcctcggcctcccgaagtgctgggattacaggcatgagccactgtgcccggcctcaagtgatcctcctacctcagcctccccagtagctgggactacaggcgcgtgccaccttgcccagttagtttttgtagttttagtagagatggggttttgacatgttgcccaggctagtcttcaactcctgagttcaagcaatctgcccacctcggcctcccaaagtgctgggattacaggcatgagccaccatgcctgacccctctaattttttttaaagaaggaattggagcagccacacttttgagctctgccccccgggttttgtgtgattccaggaaatcttggcccctctctgggtctctatgtcttgtctccagttggagactctggggaggaggggagattgtggctgcccccatacagcttgcagctgagcagaggcccttggatttgggagtttgggaatcctcttttcccttgattctactggattgcattcagtccagatggaatgaggttgtgggaaggggagccaggatgaactcttaatggcttggaaacagatctgatctcctaagaggacaggaagtcagactccttttgtgggttacaggggaaatcattcggtagttgattggtaatgtctgccatggtgcaaaattgagattcagctgtgtatgttgttaccaccttggtctagtaaatacagcacctagtagagagtcaggaaagcccagactctggtcccaatttaccactgcctttctgaggtatcctgaataaattagctccttagagccttggtttcctaaactctaaagtaagacataatcttcccagggttgatttgaagatgcagtgaacatattgtcagttaggatcttcgtgttgcaagaaatagaaaatacgatgggtgctgtggctcatgcctgtaatccaaggactttgataggtcaaggcaggaggatcacttgagcccaggagtttgagaccaggctgggcaacatactgagacctcgtctccaccaaaaattaaaaaattaaaaattagcaagccaggtgtagtggctcacacctgtaatcccagcactttgggaggctgaggcaggcagatcacctgaggtcagaagttcgagacccacctggccaacatggtgaaaccctgtctctactaaaaatataaaaagttagccgggcgtggtggtgcgtgcctgaaatcccagctactcgggaggctgaggcacaagaatcgcctgaacccaggaggcggaagttgcagtgagccacgctcaagctactaaactccagcctgggtgacagaatgagactctaaaaaaaaaaaaccccaaacaaaaagttaaaaattagctgagagcggccgggcgcggtggctcacgcctgtaatcccagcactttgggaggccgaggcaggcggatcacaaggtcaggagattgagaccatcctggccaacatggtgaaaccctgtctctactaaaaatacaaaaattcgctgggcatggtggcccacacctgtagtcccagctactcaggaggctgagtcaggagaatcacttgaacccaggaggtggaggttgcagtgagcccagatctcgccactgcactccagcttgggcaagagagcgagactccttctcaaaaaaaaaaaattacctgagagcagtggcacaggaggctgaggtgggaggatcggttgagcccaggcagtggaggctgcagtgagccataattgcaccactgcactaccctagcctgggtgagagaggaagaccctgtctcaaaaaaaaaaaaaaaaaaaaaaggataagaagtggaaaataccagtgcatcctggctagaactgtcgtaagggcttctgtaactgtgtagaggtgacaggacttcaggtatgtatgatgaaggctccagctgcatatccctgtgacttccttagcagtgtctttcccatgggtcagctttgtctgtattcataggtggccacatgcctgcttgaggtcctggtgactccaggtgtttcctacagagcagggttctgcggcagcagttccaagcaacagccctgagatcgctgctgatggggcaattcaggttcacatgcccatccctagagcgatgactgcgtgggaagagggtggccccctaggcaccagaatgaagatctcagctgtcctgaaatgtgtggcagtgcacagcgtgggggggaaagagagatttacaaggaaatccctaggcagtcagagagggcttggtagaggatgtggctcggggcgggggttggaggggcgggcgctgtcgggcgcagcggaagacacagggagggtggcaggagcatgggatgtgggtgtggcaggagaccttagggaagggagcctgcacccagggatttcgcagactctccaggaagccatcgggatctaggccagaaacctctgcatcccccatcccccatcccccaccccagagctcctgctgagcgcccctctgcctttgccagcaggaaaccagtggaggggcggtgagggctgtgatccagattcaccggagagggaaacaaccgggaggggtcggcaggcagggcgtggccaagggtgtgcgctcttcaaggcctcgtgaaccccagggcgccccctctccagctgcgcccacaattgggggctgacctccactgcaggaccccaggccttgctcccccgggacgcctctctcgggtgtcccaccgcaccactgaggccgcgcacgaccctcagccaccgcagcccgcttgagcccagctgcccatctcttgccagcaccgcctactccccgcccgccaccccaccattggcttcccagagcaaagacctcctatgattggtccatttccgcgaggccacgcctcctggtcggcacaaagagccggaccagcacagcccgccccaactccgcgtgctcctagcccctggaggcgcggatgagccgcggccgccacgggggggtcgtagtagtgactgcgaccatggtggctgtgagcaccgaactcgagcacttgaggaaggcgctggcttagggcctgtcgcgctgccatagcctcggatcccacctgagaggggaaaattcggagcctgtctacccagcccccagccccggggctctaatccctcccagggactgacccccgcccccttgttcccagaccttccctattttctctccctgcccaggggagggggatggtggacttattaggtaacggaactgttacagttaagaaaactgtagctcagagagggtaaactaccccgagtcatacagcagtcggggggagcccagaaataatactgagggatgggaaggaaagggagaagctgtggctggtgtaatcctctacatcccactcctccccacccctgatacacagttatgagctaagccagggagggctccctgagcacacatcaagcctgcaggctgacggatttttttttttttttttttttgagacggaatcccttctgtcgcacaggctggagtgcagtggcgcgatctcggctcactgcaatctccgcctcctgggttcaagcgattctcctgcctcagcctcccgagtagctgggactacaagcgcccaccaccacacctggctaatttttatatttaatagagacggggtttcaccatattggccaggctggtctcgaactcctgaccttgtgatctgcccgcctcctcggtctcccaaagtgctgggattacaggcgtgagccaccgtgcccggcctttttttttttttttttttttttttttaaggagtctcacgctgtcgcccaggctggagtacagtggcgctatctcggttcactgcaacctccacctcttgggttcaagggattctcctgcctcagcctctcgagtagctgggattacaggtgcccgccagcacgcccggctaatttttgtatttttagtagagatggggtttcaccatgttggccaggctggtctcaaactcctgacctcagatgatctgcccaccttggcctcccaaaatactaggattataggcgtgagtcactgtgcccggccaatttctttcaaaattttgaaaaattttgaaacaatcacacacttaaagctacaaatacagtgcgaaatttttttcagccaggtgtggtggcctgtgcctgcagtcccagctattggagaggctgagatgggaggattgcttgagcccaggaggtcaaggtgggtgagctatgatcaatctactgcactccaacctgggtgacagagtcacaccctatctctgaaataaaaacaaaataaaaaacaaactaatttttttcctagaaaaaaactgaaaataagttactagcctgatgccacaatcaccccttgtatttccttcaaacagggttattctgctatataaatggccaggaagttaatacattacttttggggttttttttgtttttttttttttgagacggaatctcactctgtcatccaggctggaatgcagtcacacgatctccgctcactgcaagctccgcctcccgggttcatgtgccattctcctgcctcagcctcctgagtagctgggactacacgtgcccaccaccacgcccggctaattttttgtatttttttagtggagacggggtttcacctgctagccaggatggtctcgatctcctgacctcgtgatccgcccgcctcggccttccaaagtgttgggattacaggcgtgagccaccgcgcccagcctgttagttttttgagatgaagtctcactgtcacccagcctggagtgcagtggtgcaatctccgttcactgcaaactctgcctcccgggtgcaatcgatccacctgcctcagccttctgagtagctgcgaccacaggtgctcacaaccaagcccagctaatttttcatatttttggtagagacggggtttcgccatgttgcccaggctggtctcgaactcctaacctcaagcgatcagtcggcctcagcctcccaaagtgctgggattacaggtgtgagccactgtgtcggccaacatatgatacatcactgccatctaaacctcagacccatttgagttcctgaagatgtccccagaatgtccttgcagcagaaaaatccagcccatgtgttatttcgttcttgcatacctttaatttccttcgggatggaacacttctcagtctgtgtttggctttcataacgttgactcttttttttttttttttgagactgaatcactctgtcgcccaggcaggaatgatgcagtggtgcgatcttggctcgctcactgcaacctctgccgcccgggttcaagcaatcctcccgccacagcctttggagtagctgggattacaagcgcccaccaccacaactgactaatttttgtattttcaatagagatggggtttcaccatgttggtcaggctgatctcgaactcctgacctcatgatccacccgccacagcctcccaaagtgctgggattacaggcgtgagcaactgcacccaggcattgttttgtatttttagtagagctgggatttcactatgttggccaggatggtctcaaactcccgacctcaggtgatctgcctgcctcagcctctcaaggtgctgggattacagaaatgagtcactgcacctggactattttattttatttcatttatttattgttgagacggagtttcacttttgttgcccaggctggagtgcaatggcaagatctcggcttactgcaacttctacctcctggattcaagcgattctcctgcctcagccttccgagtagctgggattacaggcatccaccaccatgcccggctaggaaaatgtatttaggtgcatctgtatgaattcctatacctcttatgtatattgaaagccaagagtttacattgatagatcctggggtgcactccagtttacaactcccttctctgcctttgagaacctggctacgattattgtgatacatctgcttctttttttttttttttttgagacagagtttcactctgtcgcccaggcttgagtgcagtggtgcgatcttggccggctgcaacctccacctcctgggttcaagcgattctcctgcctcccaagtagctgcgattacaggtgcccgccaccatgcctggctaacttttgtatttttttttttctttttgagatggagtctccctctgtcgcccaggctggagtgcagtggcacgatctcggctcactgcaagctccgcctcccaggttcatgccattctcctgcctcagcctcccgagtagctgggactacaagtgcccgccaccatacgcggctaattttttgtatttttagtagagatggggtttcactgtgttagccaggatggtctcgatctcctgacctcgtgatctgcccatctcagccttccaaagtgctgtgattacaggcgtcagcccccatgcccggcctaattttgtgtttttagtagcaatggggttttgccatgtcttccaggttggtctcgaactcctgacctcgggtgatctgcccacctcggcctcccaaagtgctgggattacaggcgggagccaccatgcctgaccgtatctgcttcttttttttgagttggagtctcactctgtcgcccaggctgcagtgcagtggtgtgatctcggctcactgcaatctcagcctcccggattgaagcggttctcgtgcaccagccttctgaggagctgggatcacaggcacgggccaccacacgcagctaattgtttgtatttttagtagagatgaggatccaccatgttggccaggctgatctcgaactcctgacctcaggtgatccgcccgccttggcctcccaaagtgctgggattacaagcgggagccaccatgcctggccacatctgcttcttttgtcaacccctccgggtgtagcacgtgtctcctgggcactgtcacctttcaccttgtagacctgtcctccagccttgctgtcctttggcagggatatctcctagccccaggacagcttagtcacccctcccaggtcaccggccccgcctaattgagctccctaccctcaatgagatctcatcctcccccattttgactgtccaccaagcagccagctctctcctaccccaccacccccgagccctctccaccccattgacgctgctcagagccaccaccctcccccaccccactgggacgcccacctggctctgctccagcaaatggctccgggactgattattctggaaggaaacggaaaggggaaggaactaagggaagagaaggaaggaggctttctccggttcttgttttagcttaaaaattcatgccccgtgtgcacttcagtctagaacacacttccttttgttttaacgtgatacagcgttttcactatacaacttctggtaaaattgctgcctggttatccgggggctgtgtctggtcagagagggggcctggcaggctgggaaggacagggaggggtgggggagggggaagacgggtgggggaaggtggacggggctggccggcctcctgtcgtcttccttccctggacaatgggctggcggctgccgcagagataaggagtcttgtcagggctgggatcacagccgccggccgactgcctgctggggaaggtcccttcctcgcagacctggccaaaccacccccaccaagccccgccgcctcaagtgagatctcctttcattgtaccttcctgagagcgaggtcctcagagccatgccccagggtcaccaggaggctggctgtgagcttggcgtctggggaggccctttcccaggccgagacacctcccctggctccaggacacagtcgcacaaagataattgttgtctttttgtttctttattcttttttattattttttcttttgagatagggtctcatttcatcaccctggctggagcgcagtggtgtgatcatgtctcgctgcagccttgacttccccagctcaagtgatccttgtgcctcagcctgagtagctgggactatggatgtgcaccaccaggctcggctgatttttttatttcttttgagagatggagtctcactatgttgcccaggctggtctggaactcctggactgaagtaatcctcccccatcctggctaacacggtgaaaccccgtctctactaaaaacacaaaaaaattagccgggcatagtagcgggcacctgtagtcccagctactcgagaggctgaggccggagaatggcgtgaacccgggaggcggagcttgcagtgagccgagattgcgccactgcactccagcctgggcgacagagagagactccgtctcaaaaaaaaaaaaaaaaaaaaaaaaaaaattcctccctcctgggcctctgaaagcgctgggattacaggtgtgacccactgtgttcagtttaagattaaatttatgtgaagtaacttatgttactttccagcccggccaacatggcgaaatcccatctgtactaaaaatagaaaaatttggctgggcgcgatggctcacgcctgtaatcccagcactttgggaggccaaggcaggcgaatcacgaggtcaggagatcgagaccatcctggctaacatggtgaaaccctgtctctactaaaaacacaaaaaattagccaggtgtggtggcacccgtctgtaatcccagctactcaggagtctgaggcaggagaatcacttgaacccgggaggcagaggttgcagtgagctgagatcgtgccattgcactccagcctgggtgacagagcgagattccatctcaaaacaacaacaacaaaaataaaaattagcggggcacgcttgtagtcccagctacttgagaggttgaggcaggagaatcgcttgtacctgggaggcggaggttatagtgagccgagattgcaccactgcgctccagcgcctgggtgacagagcaagaccctgtctcaaaaaaaaaaaacaaaaacttaccacactcaaagtacataaacctgcagtacatctcctggcatggttatgggtgtctaggtccgaatcgcagcctgtggaagcccagggggacagctccggccccagtggttagaaagtgtagccctgccaagtggggatgaacctgatgtcgtagtatcgggggaagaaaaggacctaaaatttctcataagtttcatgttaattacatattgaaatgatagtatttgggatataggagttaaataacatacattaagaaatttgggccgggcgcggtagctcatacctgtaatcccagcactttgggaggccgaggtgggcggatcacgaggtcaggagatcgagaccatcctggctaacacggtgaaaccccgtctctactaaaaatacaaaaaaaattagctgggtgtggtggcggacgccctagtcccagctactcgggaggctgaggcaggagaatgaagtgaacccaggaggcggagcttgcagtgagtcgagatcgcgccactgcactccagcctgggcgacggagcgagactccatctcaaaaaaaaaagaaagaaatttgatgtcacttgtttatctttacttaaaaatttttttaacttttaggctgggcttggtggctcacacctgtaataacagcactttgggaggccgaggcaggtgcatcacttgtgggcaggagttcgagaccagcctggctaacatggtgaaaccctgtctctattaaaaacacaaaaattaactggatggggtttcaccatgttgggcaggctggtctcgaactcctctcagatgattctccctcctcggtctcccaaagtgttgggactacaggtgtgagccaccatggcgagctcttgttcttttttttaagatgaagttttgctctgtcacccaggctagagtgcagtggcacgatctcggctcactgaagcctccacctcctgagttccagcaattcttgtgcctcagcctctcgagtagctgggattacaggcatgtgccaccatgcccagctaattttcgtatttttagtagagatgtcgttttgccatgttggccaggctggtctcaaactcctgacctcaagtgatccactcaccttggcctcccaagtgggaggcccacataagccactgtgcccggccccaattaggttgttttttttgttttgttttgttttgttttttgagatggagtctctttgtcgccaggctgaagtgcagtggcttgattttggctcactgcaacctctgcctctcagattcaaagtgattctcctgcctcagcctcccgggtagctgggactacaggcgcgtgccatcatgcctggctaatttttgtatttttatttattttattttattttattttattttattttgagacggagtcttcctctgttgcccaggctggagtgcagtggcgcgatctcggctcactgcaagctccgcctcccaggttcacgccattctcctgcctcagcctcccaagtagctgggactacaggcacccgccaccacgcccggctaatttttcgtatttttaatagagacagggtttcaccgtgttagccaggatggtcttgatctcctgacctcgtgatccacccgcctcagtatcccaaagtgctgggattacaggcgtgagccaccgcacccagccggttaatttttgtatttttagtaaagatggggtttcaccattttgaccaggctggtctcgaactcctgatctcgttatttgcccgcctcggcctccaaaagccaagtgggtttttacagttccatctgtttgctctattaggtcagaagattgagaatggtaactaaaatggaaattatgtgaaactggccaaaataggttgcagtgagccaagatcgtgccactgcactccagcccgggtgacagagtgagacccagtcacacacaaacacacacaccaacacacacaagccagaatcagtacttacaggttttcctgaaataaacttagggcaggggccactggtcaaataagcattttttgcagcttttttcttttttcttctttttttgtttcatttttttttgcagcttttaaatgctgcccaccaatattgtcttataaaagaaatcaatttgttggttacccgatgagtcaaactgtgttccgcggttaactgagtatttcactgaatttcaaagaactggtttgttatcagatccaaaccacagctctcttttatcgtcacatcagcagccatgatctttccagttttcttttttggcctctggtgccccatctttggtaattatttgtggttcatcttttgaagtttctttgagtggggccgcaagagagatttgattacacggagttgattaacggcagccttcgtagcagtattatcagcagggttgttccctctagcttccatagcgtctaaccttgaatgtctgagaattttgataaaagtcaaggcctggtcactggacggcctccagtaaattctgtacgcaggatccatgtttcatttgatctccagtggaagtgaggaacgcctttgtttctttgtttcttttcttttctttttttgtttttttgagatggagtctcactctgtcaccaggctggagtgcggtggcgcgatcgcagctcgccacaacctccacctcctgggttcaagcgattctcctgcctcagcctcaggagtagttgggacttcaggtgcccgccaccacacccggctaatttttttgtattcttagtagagatggggcttcgccatgttggccaggctggtctcgaactcctggcctcaagtgatctgccttggccgcccatagtgccgggattacaggcgtgagccaccatgcccagccaaaaatgaaattttctgtgcccatattccagccgaggaagcccaagttcccaccgggagcgtctacacgtgcagccgctgttatctgtacccaggtaacatcctccagctcttggggagatccagctgacctccagtgtccccgtgcccacacacgtggccaggagcagctgtcaacaaatggctcttgggttgaggcaggagtcctccagatcctttggccctagcatgggctgactgggaggcacggcacctggagtctcccacatgtccctggcggaatgaacgttcagtgggattcctcatggtaactggcttgataatgcaccctctgggatctgtcccttccctgtcctatgtccctgtcctctacctgaaccttttgtgtggaatggcacctcaggcccggcctctgggggatgaagatggtgttaactgcattcaaagcctccctcttgtcacccacgatacactttctgccctggcagggggacaggaggttccttgacggctagtccaggccatgacgggttgatcctggattaaaataagtcaagcttcaggctggtgcagtggctcacgcctgtagtcccagcactttgggaggccgaagaaggcagatcacctgaggtcaggagttcaagaccaatctgggcaacatgttgaaaccccatctctactaaaaacacaaatatggccgggcgcggtggctcacgcttgtaatcccagcactttgggaggccgaggcgggcggatcacaaggtcaggagatcgagaccatcctggctaacatggtgaaaccctgtctctactaaaaatacaaaaattagccgggcatggtggcgggtgcctatagtcccagctactcaggaggctgaggcaggaaaatggcgtgaacccgggaggcggaacttgcagtgagccgagatcgcaccactgcgctccagcctgggcgacagaagcaagactctgtctcaaaaaaaaaacaaacaaaaaaaacccacaaatattagctgggcatggtggtgcatgtctgtaatcccacctactggggaggctgaggcaggagaatctcttgaatccaggaggtggaggttgcagtgagctgagattgtacctctgcactccagcctgggtaacagaacaagagtctgtctcaaaaaaaaaaaaagaagtcagtcgggcacggtggctcacgcctgtaatcccagcactttgggaggctgaggcaggtgaattgcctgagctcaggagttcgagaccagcctgggcaacacggtgaaaccctgtctctgctaaaatacaaaaaataagctgagtgtcacggtgtgcacctgtactcgagctacttgggaggctgaggcaggagaattgcttgaacccgggaggcagaggttgcagtgagccgagatcgtgccactgcactgcaacctgggcaacagaacagagtgagactccatctccaaaaaaaaaggagtcaatcgtgtcaatcgttagggagctggtggatttatagcctgtagtgaccacatcagtgaggcactggtgctgggaggcacctcagaggtcatcccattcaaccccttcactgactcacagctcagagagggcaagagacttacccaagggcacacagcaagtcgagggcaccagcaaggtcagcacccaggtctcccgcacctgggcccacgtggttttcagtctggaaactggaaatggatatgaagccttggaaaatcccagcagggcaggatgggggtggcccagctgtttcactatggaaacaaaagtcttgttttcctggctactccagcggggccgggacggaggcagatgtttctcttcacctgccaggaaagtttggaaattaagccctccttgcagggaggaagggacccagtgctctggattttcaatttaatgggctacccaggtcttcggagtgcagccagggaagcacagaggaagcgccctggagaggaagccagagatttcctggaggcgccagggatgcaaggagtggcctggagagggagtgagggaaaagcagcagacgcccctaacttcctcctcctccttccaacagaaacctggtttggggttttagtggagacgtctcgccatggccgccagtgtagacattaattggtctaatgcaggcaccaggtccccggcttcttgcagggaggggcatggttttaggacaaggcatggctttaaaataaaaaattaattgttgtaaaatatgcatagcatgacccggcatggtagctcattcctttaaccccagcactttgggaggctgaggtgggaggatcaggtgagctcaggggttcgagaccagcctgggcaacatggtgagaccccgtctctaccaaaaatacaaaatttagcccggtgtggtggtgcatgcctgtagtcccacctagtcgggaggatgaggtgggagaatcacttgaacctgggaagttgaggcgattttttaaaaaattaatactctaggccaggcatggtggctcacgcctgtaatcccagcactttgggagaggccgaggcgggcggatcgcctgaggtcaggagttcgagaccagcctgggcaacatggtgaaacctggtctctactaataatacaaaaattagccgagcatggtggcatgtgcctgtaatcccagctacttgggaggctgaggtaggagaatcacttgagcctgggaagtggaggttgtagtgagccgagatcgcaccattgcactccagcctggggcgacaagagcgaaactctgtctcaaaaaaaaaaaaaaaatcataatactctttttttttcttttgagatggagtcttgctctgtcacccaggctggaatgcagttgcaaaatcttggctcactgaaacctccgcctcctgggttcaagtgattcttctgcctcagctgcccaagtagctgggattacaggcgcccaccaccacgcccagctaatttttgtatttttttaagtagagatggggtttcaccatgttggtctcgaactcctgatctcagataatccgctggccttggtctcccaaagtgctgggattacaggcatgagccaccatgcctggctgaggcaattactttttgcagagacagggtcttgctatgttgcccagccttgtcttgaactcctagactcattcaatctgtccattttggcctcccaaagtgctgagatcgaccacgaccacacctggtcataactggctttctttatattcatatgcattttatcttgtgcatttaaaaacttgatactgtgagaaggttcataatttctcaaaagtctgttttagatagtgctggggagatgtgatgtctggagccctggcagctgccttgagattctgaggacctgagcctgaaggcagagttcacctgacagggaaggcagagtgagaagtgggggaagttccagggtcctttccagctctgttacagtgaaccaaccctggaaactcccctctttggggcttcttgtttcttgctataatgaaacatcttgctggttagccagttgagttagagttttctctttcttgcatttgaagctaccatgactgcaagagggagttcccgaaggggtctgggaatggcccagggaaataggggtgaggtgagactccactgccccgacagtgcccatgtatgaattagaaaaagtgggccaggcgtggtggctcacacctgtaatcccagcattttgggaggctgagtgggcagatcatgaggtcaggagttcgaaaccagcctggccaacatggtgaaaccccatctctactaaaaatacaaaaattagctgggcttggtggcacacatctgtagtcccagctactcaggaggctgaggcagaagaatcgcttgaacctgggaggtggaggttgcagtgagccgaggtcatgccactgcactccagcctgggtgacagagtgagactttgtctcaaaaaaaaaaaaaaaaaaaaaaagaaaagaaagaaagaaaaagaaaaaaagtgggccaggtgtggtggctcatacctgtaatcccagcactttgggaggctgaggtgggaggattgcttacagccaggagtttgagaccaggttggacaacatggtgagaccttgtctttacaaaaaaatacaaaaacctatctgggcatggtgttgcattcctttagtcccagctacttaggaggttgaggtggaaggatcacttgagttaagggaggagaccacccctcatattgtcttatgcccaatttctgcctccaaagaaagaaaaagtaaaaactaaaaggcagaaatgaaaaccacaggcagacagcccagcgccacaccctgggcctcgtagttaaagatcgacccctgatctaatcggtgatgttatctatagactacagacattgtatagaaatgcactgtgaaaatccctatctggttttgttctgatctaattaccggtgcatgcagcccccagtcacgtaccccctgcttgctcaatcacgaccctctcacgtgcacccccttagagttgtgagcccttaaaagggacaggaattgctcactcggggagcttggctcttgagacaggagtcttgctgatgcccctggccaaataaaccccttccttctttaactcggtgtctgagttttgtctgcggctcatcctgctacagagtctaggaggcagaggttgcagtaagccaaattcacgccactgcactccagcctgggtgacagagcaagaccccaccaaaaaaagaaaagaggccaggcgcagtggctcacgcccagctaatttttgtacttttagtagagacggggtttcaccatgttggccaggctgatctcaaactcctgtcctcaggtgatccgcccaccttggcctcccaaagtgctgcgataacaggagtggaggctcaactttttaaagaagaaaaggacgaatcaggacaggagacaattacaagctctgttcattcggaattctcattggcttacagaaataactttggctagtgattggttatatgttgtagacagacccacagggtggatggcgtctgtggccacttgggcattagctggtccagagcccagagtccatgtagcaagtagcttcaggacgtaattatttagctcaatagaaagtgagatgtgactgctgttacttttttttttttttgagacggagtttcactcttgttgcccaagctggagtgcaatggcgcgatctcggctcactgcaacctctgtctcccaggttcaagtgattctcctgcctcagcctcccgagtagctgggattacaggcgtgtgccaccaagcccggctaatttttgtatttttagtagagacagggtttcactatgttagccaggatggtctcgaactcctgaccttgtcatccaccggcctcggcctctcaaggtgctaggattacaggcatgagccaccacgcccggccttttaactgctgttacttttttttttttaatcatttttattctgctagttccataaaagcaatgtaaacaccagcattctatacatcttgctggtgaactcacataatgcttagttccctgatcctttgacctccttgtcttctccagttattttctgtttggaccactggccacaggaatggagtggggttggggcttaggagagagcaatgtggctttttggtatgacttgtttcattgactgctgttacattttaaatgcctttctgggcctgataatttaagggggctggcatttctcacatcaaagggcaaagggtttttttgtttgtttctcactattgctcaaaagccacatgtgtcaaggacagctcttgttaacgggcagagaagcttatagaacctttccagggctggcgcggttgttcatgtctgggggacaagagcgaaactccatctcaaaaagaaaaaaaaaaaaaagaacatttccaggccaggcacagtggctcacacctgtaagtcccagcactttgggagattgaggtgggaggatcacttgaggccaggagtttgagaccagcataggccacatggcaagaacccagtttctacaaaaacattttttttttaaattagctgggctcggtggtgcacacttgtggtcccagctactcgggaggctgaggtgggagaatcgcttgagcctgggaggcggaggttgcagtgagctgtgatcgcaccactgcatttcagcctgggcaacagagggagactctgtctcaaaaaaaatcaaataaagaaaaaagaacatttccattattgcagaatgttctattggacggcgctgggccagatgcctgccccaagccctgggacacccaaacctggtgaaagtgccaagctcccatctgggggtgctcacggaaggggccgggagctgggattacaagcgtggaggcaggcgccaccccagaggaggtgggtgatgtctgagccagagtctttgggatgagcagggctttggcaggcagctttgttgggcaggcaacaggcacagcaggtgcaatggcatcgaggttggagaggtggactggcggggagaagagaagggagggtggcaggagagatgggcagaggccccccaggagcccagagccttcagggctttgggccctcttggggcactggggagccacgggagaagtgtggggtggagaggggcgccctggatttgaccaccttcaggaacctaccttggctgcccgggtgggtgggatggaggtgatgaaggtggaggtcgtagcattgtcactgagagcgatgcttattctgatttttgcccccgtgagcctcagaaatcggtcagaacagtgctcgggctgggcgtggtggctcacgcctggaatcccagcactttgggaggccgaggcgggcggatcacctgaggtcaggagtttgagaccagcctggccaacatggcaaaaccccgtctctactaaaaacacaaaaattagctgggtgtagtggtgcatgcctgtaatccagctactccagaggctgaggcctccctgggaggcggagtgcagtgagccaagattgtaccaccgcccctccagcctgggtaacagagtgagactctgtctcaaaacaaacaaaaaagaacaaaccaaaaccagtgctccaattcaccttgctgaattggagaagaacaggtgcctgggtcctcccggctgacggtgacgtcactatgagccaccacgcctggcccggtgggtgtttacagcagaggtggttcagggaggagtgagtggcagatggtgcaggctcagggaggagtgagtggcaggtggggcctgaggccagaggcaggaggaccgttcaagactgaggtgtcttgggcactggtgccggggccggggagagccctggcatgccgggccccggccctcagtgaaagcaggatgaggtggcggccaagcagggaggccaaggcctggcagcagccggcagcccctctcctggcagggacatcttggcaggagccgggctctgcccagggcgctaatgtcctcttaattagccaggcacttgccaagcacttgccgctgcggcctcactttgcagaacgcacttgggtttggagcaggaccagcatcggccaccgggtgagtaggggggatggccaggtgagtaccatggccagaaggtgcggcctctgagtccccactgaagccaatccaggccccgacctctggtcatcatcaacagaagagtgacagaaacagaaaagatatcaaggaacacgttttcagggccagattccaggtcccccacctattttcctttgctcctgccttgcctggctctgcccccttgaactcttattcacccagtgtaggcatcacctcctcctgaaggccctcctggactgcacagttaactctggagcctcccctgcacagggcagctcatgtatggatccacaggtgagttcctgtctttgaagggttacagtttaggtcctggagcagtggtggggaggtcacacctccacccacatccctctttgcaaatcccatgggaactatgagcctgggtcccccaaactgcatgttccccacttaaagctgggccagatcctgtttgctgtatccaggcctcagtttccctgcttgtaaccagaagtccatgcagccagaaattgtcagggagctgggcacggtggctcatgcctgtaatcccagcactttcagaggctgaggcaggaggttcacttgtgatcaggagtttgggaccccagcctgggcaatacaatgagatccctgcctctaccaaaaaattaaaaacaattagccaggcctggaggcatacacttgtagtcctagctattcaagggaggctgaggtgggaggatcacttgagtccaggagtttgtggctggggtgagctctgattgtaccactgcactccagcctgggtaagagaatgagaccttgttttattttattttatttttatttattttgagatggagtctcactctgtcacccaggctggagtgcagtggtgcaatgtcggttcactgcaacctctgcttcccggcttcaagagattctcccgcctcagcctcccaagtagctgggattacaggcgtgtgccaccacgcctggctaattttttgtgtttttagtagagacagggtttcaccatgtttgccaggctggacttgaactcctgaccttaggtgatccatgcccccttggcctcctaaattgctgggattataggcatgagccaccgggcctggccgagaccctgttttaaaaaaattaatcaggtccagtgcggtggctcacgcctgtaatcccagcactttgggaggccgaggagggcagatcacctgagatcgggagttcgagaccagcctgaccaacatgcagaaaccccgtctctactaaaaatacaaaattagcagggcgtggtggcgcacacctgtaatcccagctacttgggaggctgaggcaggagaatcacttggacctgggaagcagagcttgtggtgagccgagattgcaccattccactccagcctgggcaaaaagagtgaaactccatctcaaataataataataataataataataataataataataataattggtagagatgttggtagaggcaggacagtgggagtgacagcctggtcctgtcctcccgggctctggctgaccatgctgtagtgtggctcagggccctggagggcacagctgtcctgtggccctgtggtcagccgggaagccaggcctggcaggactcctgccctagaatcagctcctcccactccccagggtcccatgcagggcgctggccgtaccaggcctcctcgcgggtgccacagagccagcggctgcacagcaacagagacgccattcacaggctctcggccacgaggccgctcatttacagagggctgagccgctgacatctgtgcgtttgtaacatggacagcatttctggtcccatacagacgcctgggccagccacatttatctccccttgagagaagggaaacgaggctcgggagggaatgagggattggcttggggtcacacaactttaatgatgaccctgggcaggaagctcccaggatggcaacagagactcagggcaggctgggcatggtggctcacacttgtcatcccagccctttgggaggcagaggtgcaaggatcactcgagcccaggagctcaaggccagcctggggaatatagtgagaacccatctctaaaaaaaaaaaaaataggccggtcgcggtgactcacgcctgtaatcccagcactttgggaggctgaggcgggtggatcacgaggtcaggagattgagaccatcctggctaacacggtgaaaccccgtctctactaaaaatacaaaaaataggccgggcgtggtggctcatgcctgtaatcccagcactttgggaggctgaggccggcggatcacgaggtcaggagttcgagatcagcctgacaaacatgacgaaaccctgtctctactaaaaatacaaaaattagccgggcgtggtgacacgcgcctgtagtcccagctactcaggaggctgaggcaggagaatcgcttgaaccatcgaggtggaggttgcggtgagccgagatcgcactactgcactccagtctgggagacagagtgagactccgtctcaaaaaaaaaaaaaaaaattagccgggcgtggtggcgggcgcctgtagtcccagctactcgggaggctgaggtaggagaatggcgtgaacccgggaggcggagcttgcagtgagccgagatcgcgccactgcactaaagcctgggcaacagagcgagactctatctcaaaaaaaaaaaaaaaaatttagccgggcatggtggtgcatgtctgtacccccagcaatttgggaagctgaggtgggaggattgcttaagcccaggagtttgaggcgggagtgagctgtgatggcaccactgcactccagcctgggtgacagagtgagaccccgtctctccctatcccccgcaaaaaaaaaaaaacaactcagggctttcccacccctgtccttgcacagatgaagaaccgaagcttctagaaggggtatattttgcccccaggccccaaatcctggtctttggactacagtcaagacctagcacagggctcaggcctaaaaaaaagctgttgaggagggtgtgaggctgcaacgttgcggtgagaagggggtccccagggagggcgcagcaggaagccccagggaagtgcctgggagagggaggttgtgcagccagaaggagcagcccgcagcctttggttgttgactcctgctttgtaagtggcagtctggttgggtaggacagggtccgagtcctcactcagggaactgagtccagggtgagctgagcagcccttcctggtggcctcactttcccctgcagagcctgctgcatggtgtccagtggccagcctgggaggagctcaggactggcctcacctcgtgccacgccctcgtaccaaggtggctcagatggcacctgggttcccgaagggcccaggaacacagcgtccatgtccccatccttccccggagggacttggggtggggcctgcaggaaggatcatgtgacttgttcaagggcagcttgtcctgccccggacacagaggtgcccatcgtaagcgagatgcaggcaaggtgaggacagggcatggtgccgagcaggaatgattttcgaaaatgctactctgtggtcgggcacggtggctcactcctgtaatcccagtactttgggaggcccaggcgggcagatcatgaggtcaggagttcaagaccagcctggccaacatggtgaaaccccttctctactgaaaatacaaaaaactacccgggcgtggtggtggatgcctgtaatcccagctactcaggaggctgaggcaggagaatcgcttgaaccctggaggtgaaggttgcaatgagccgagattgcaccactgcactccgacctgggcgacagagaaagactccgtctgaaaaacaaacaaacaaaaccccgagattctcttttcccccgtccggagctctatggccatctggagctcgttctgtccacaaggacacatttcctggcagcagctgtggaccagggctcactcactcacatctgtacccaatctagagcaggacaaacacctatcacctgcactggcaatggacagaggacagtggcagccccatcaccagaggcattcaagccaaggcattttctatcgtctatttatctatctatctatctatctatctatctatctatctatctatctagatgagatcttgctatgttgcccaggttgaactcctggcctcaagcgatcctcctgcttcagcctcccaaagtgttgggatttcaggtgtgaaccactgtacctggctggagtgcagtggcactgtcatagctcactgcagcctccacctcctgggctcaaggaatcctctttcctcagcctcctgagtagctgggaccacaggcatgcaccatcacacccagctaaaccttgattttttccatagtatatggctcagggcagagcaaagaagcacaaaaacatgttggcttgcggttgagggctgatgggagggggttctggccaaagcccaaaattaacagccacaacgtcagtgtctgtgggaggggttgccaggggcgtgcgggttctggggctcaaggccctgccggtaaacccatttgaagcaggatggcaagaaggtgacaccatcttcccccgcgcactgaaagcccctggctgggattgcctggggcagacacaggctcggaccagccccagcaatcccagtttatcagcgagccggctgagggcccggagttatctcagtgcccggcctgagaccttgtgggcagcctgtggtcatgctggcattccaggggccttttggcatgtggggaatgtccaggaaaagcctcagccttcggtgaggcgcagaaaagggaagtgtccctagagggggtgggtgagggcgtgggaggtggtgtctgcagggaatgtcccctttgggggaggaggatggagggttgggattctgaggatgggggggggggctgtagccagcaccatgtccctcctgtgtgaccagctcagagtcccatgaaattggggcttgggaggggaagggacactggcctgggaaccagagacctgggctggtctggctcacagttgctggacctctgtgatccgtgtcaaaaaacgagaacaccaattcctgtcctgcccactcaccaccaggtgggacctgaaccctggcatcgccagcattgggaatgtcggccactgactcaaccactctcccggagacctatttgggccacccgaggcgggtgcctgggccacacggaggggtcctggtggtcttcagggcagcggctgtggggctgaagcctcaaggaaccacatctctgcataggagggccaggctgcagggcctcggagacaacctagttgggcgtgttggggtctgtgggtcccaggtcctggcctcaccgggtccccaccgcgctgtcagctcccagcctctttccctcgtctgcctctgggcttctgtaaggctatgtgctccaaggccacctcctccaggcagccctcagacccccaccttcgcccagtaccgatctgcaccgtggtctctgaagtctccatcgtgacctcaaacctcgctcgtccttgctcctagcgaggcttggggtcggggtgtccgaggtgggggacatccgggggggttaggtggctggcgcggggagccggggttgtgaggggtgatgtcctcaggcggcggcgctgcggggtgcggcgaggacaccggtggggtgagagcaccggcggggcagcagcgggggccgcagcgccgggtccctcggcccggggcccctcccgcgcggagccaggggcgggacaggggggcgtggcctggtggcgctgacgtcacctcgcctataaaatgtccggggcgccgctagctgggcttt

SEQ ID NO: 3 - DNA, 20229 bp, Homo sapiens
  source 1..20229: mol_type = genomic DNA
gggtgatttgtttcatctcttcttttttcagggtagtcacttgttcctaaaagaggaacatagcagtggctgtaatgggaatggctctgcagtgggctagtcacaccaaaataaagaaagggcagctgtgggagccatcagaagctgtggaggctggactggtaatggggaatgctgccccactgaagtcttgttttagagatcagtgtgatgtgctcacttaactataaagtgtcagtccccattccaactaagtggctcctggctgggaagtagagtaggtttctctaaggaaaatgacacctgctcaccacccgccccactataccacattgttgttaggctatcatataatgataaggtagccctctgaggccacaaggtaagaccaaaccaccagggctgggttttgggatcccctctaaagagaccacacatgtttgtttagtgtttataatctagagaaaagacaactgggacagaaaatggggacctggaaggtaggtctcagactggtgaatgaatttttccctagaccttgatttccagagcttgaatttaagggcactcccaggtagagaaatgtctttgtcccatggaaattatagaccaaggaagacgtcaacctataaaaacgtataagggtttctcaggaacagtgttttcagaatatctggtgagctagctaatttttttctctattttaattccagttattatccttgataaagtcttaagcttaagaggaaaaagtaagtgtccatccttgggtcagaagttaaaaggaagatcagagctcctaaggcagtgataattgttaagatcttccaaaggggaagaccatcagagagtacaagaatttaagccagcgggtggcgcacgcctgtaatcccagcactttggaaggctgaggggcagatcacccgaggtctggagtttgagaccagcttgaccaacatggtgaaaccccatctctactaaaatacaaaaattagctgggcgtagtggcgggcgcctgtaatctcagctacttggaaggctgaggcgggagaatcaacctgaaaccatgaggcggaggttgcagtgagccgagattgtgccattgcactccagcctggcaacagagttaagactatgtctcaaaaaaaaaaaaggatttaagccaatctttcaaacctggtgaatcttcttaccatggaggattggttggctagcatagtaaaggactgactgcgtggttgtagctctgtagtttggtttcttctctgccactaatttggttatgtcagccttgccagattactaaacttgctgaaacatggcttgcttacctgtaaaatcgaggttaatagtaggttcatattgggttttgttttcaaatgcaagaaatcatccatgtaaagaaagtatttagcacagcttagggcatagactatgcactttacaatgttagagcaggtttctttatagtagtcttcgtcagacctggagacccaaacttttgtgtcccaagtagtgattctcagctttccaatatgaatagccccttgttaacataaagatctcactttttcttattagaatttgtacctttgtttgattaaggaaataaaagatgaatgacaaatgaatttggtaatgcctttctgtgaatctgtattcagtatgttttgtatgttaatatagcagggtcatacaaaaactggcctatttttgtaaagttttgtagaaacactgctacacctattcatttgtctgtggcagttttcacactaactccagacttgagtagagtaggtgctacaaagaccaaagcatgggccacagagctgaaaatacttactctgtgtccttttacagaaaagtgtgctggcctttgcactaaagcatgaagaaacatttgggaaaaagcacacaggctgggcacggtggcccatgcctgtaatcccagcactttgggaggccgaggcgggcagatcacctgagaatgggagttcaagaccagcctggccaacatggagaaatcccatctctactaaaaatgcaaaattagccgggcgtggtggtgcattcctgtaatcccagctacttggaaggctgaggcaggagaatcacttgaacttgggaggtggaggttgaggtgagccgagatcacaccattgcactccagcctgggcaacaagagcaaaactctgtctcaattaaaaaaaagaaaaaagagcaacaaacaaggcaaagttacttcatttataatccttgttgcatcatgagttggtcaacacttaccctaatttgtcattcttaaggctgttataccttaagttgaggtgtgatgaaaataccctttttgcttacaaataaaaactgattgccaaagtaagtatgtcacagaaaaagctattgaacaactgtttagagcaatgattctcaaggatggggacacagagcatactatttggggagggaaaaagatttgaaatgaccaattttatttcaagcatttttaattttggaattttaggtacaaaaatgggtgtgagaattttgtttaacagaaaatagcatggctttaaaatagtttgtaaaatactgttcttaaggaacagagtgtaatagtgcccatgggctattaaaaaggaacagaaacttctctactactctggagtcctttgttgaaaacagattgagggagtgctgtagtcttccagagctacagttaaactggataaggttggacacctggaaaaaagcgtacattaacccaccctaaaccatactttttttttttcttttttttgagatgaagtttcattcttgttgcccaggctggaatgcactggtgcaatctcggctcactgcaacttccgtctcccgggttcaagtgattctcctgcctcagcctcccaagtaagtgggattacaggcatgcaccaccacgcccagctaattttgtatttttagtagagacggggttttaccatgttggttgggtgggtcttgaacttctgacctcgtgatccactcgccttagcctcccaaagtgctgggattacaggtgtgagccaccgcacctggccaccaaaccatactttcaagtaaataattagcactttattgtactgaaggtgtgatattttgacaatttaaagttctggtcttcatgggtctttgtttcgttttacttattgctgtatctaatatctcctttttttttttcttttttgagacagagtctcagtctgtcacccaggctggagtgcagtggcgcaatttcagctcaccgcatcctccgccgcctgggttcaagcagttctgcctcagtctcctgaatagcttggattacaggcgcccgccaccacgcctggctattgtatttttcaatagagaaggggtttcatcatattggccagactggtctcaaacttctgacctcgtgatccacccgcctcaacctctgaaagagctgggattacagacgtgagccaccgcgccctgtgtagtatctcattttatgcctggttatctgtgagctggacagtatatttgaaaaattacggtgggcatggtggctcaggctgggtgcggtagctcacgcctgtaatcccagcactttcggaggccaaggtgggcagatcatgaggtcagaagtttgagaccagcctggccaatataatgaaaccccgtctctactaaaaatacaaaaaatcagccgggcgtggtggcgggcgcctgtaatcccagctactagggaggctgagccaggagaatcacttgaacccaggaggaggaggttgcagtgattcaagattgcataccattgcactccagcctaggcaacaagcaaatctccatctcaaaaaaaaaaaaaattattatttgcaggagtgaacaggacaagaatgagggtatctatctctaagaaggatttatgttgcctgtggctgaggatactaccaaatcatctgatgcaggtttcagaaggctgaaagtacctcaggtcatccagattaaatataacctggtggcaagtccacttgagaactggtttactttttttgtttggttttgttgttgttttttgagacggagtctcactcttgtcacccaggctggagtgcggtggcacgatctcggctcactacaatctctgcctcccgggttcaaaggattcccctgcctcagcctccggaatagctaggattacaggcatgtgccatcaagcacagctaatttttgtatttttaatagagacagggtttcaccatgttggccaggctggtcttgaactcctgacgtcaggtgatctgcctgcctcggcctcccaaagtactgagattacaggagtgagccaccacgcctggccgagaaatggtttacttttgattcacctttatcttaaaggtgtaattctttgggatcccaatttaaggtgtctgtggtgagaattcccaccattagctagccttaggttttgacttttgtccagtcctttgaggctgctgaaagacttctaaaagtgcagctcaggtgtccaagaacttcttccagattggcaaatgccttaaaggaaaaagcagctctgactgttaggcttagctctgctttctcctcacttagactttggttcagttcttccttattagctggttagctttttgatgcctttaagagaatgacgggctgggcatggtggctcacgcctgtaatcccaacactttgggaggctgaggtgggaggatcatttgagaccagcctgggcaacatcagaagaacctgcctttgcaaaaaataaactcaaaaaatcagccaggtgtggtggcacacacctgtggtctcagcttctctggaggctgaggtggaagaatcacaagcctgggaggttgaggctgctgtgagccatgattgtgccatagcactccagcttgggtgacagagcaagactcttgtctcaaaaaactatatatatatatataatttttttttaactgcttttttttttgaggtggagttttgctgttgtcgcccaggctggagtgcagtggcatgatctctgctcactgcaacctccgcctcccgggttcaagtgattctcctgcctcagcttccccaagtagctgggattacaggcacctgccaccacacctggctaatttttgtatttttaatagagacgacatttcaccatgttggccaggctggtctcgaactcctgacctgaggcgatccacccgccttggcctcccaaagtgctgggattacaggtgtgagccatcgcgcccagccattttcattgctttttagttggctttgtcagtaggtttttctatttattttttagagatggtctcgctctgttgcccttgttggagtgtggtggtgcaatcatagctcactgcagctcaagcgactcccccacccttggcctcccaaagtgctgagattacaggtgtgatctcccagattttatttttttaaatttagggtgaccgtcacccatggcatggccctcatgaggtcatgtgccctcaggtggatccagattttctttttcttttttttttttttttttttttgagacggagtctcgctctgtcacccaggctggagtgcagtggcgcaatctcagttcactgcaagctccacctcctgggttcacgccattctcccgcctcagcctccccagtagctgggactatagatgcccgccaccacgcctggctaatttttttttgtatttttagtagagacggggtttcaccgtgatagccaggatggtctcgatctggcctcgtgatccgcctgcctcggcctcccaaagtgctaggattacaggcgtgagccaccgcgcccggccaagtggatccagattttctaatgctccatttctaggagtgaaattacctatatttcttgtctcatcagggctttcattagttgatgaaggctgttaacctgccaacctgccttgctacccaacatgccttcttgttagttaaacataagtaggaaaagacatgtttatgttggctttgagtgttaatcattcagcaagtattactggtgtcttctgtgttctttatttctggaggaatgatatttgagtttttaaagcctgacaagttctgtaaatggccctagcccttctgtcttataacgctagtacccaatttagagtttaacataaataaatagtcaactgaatctactgagaagggactcggtctagcaagagacataagcaggcaaggtaaaagttggcaagtgttgtgtgactggtacaggtagtagactgaggagagggcgattctgatcagggaaggcttcattggaggaggtagggttgaaacgttcaaagaatgaatgaggccgggtgtggtggctcacacctgtaatcccagcactttgggaggccgaggtgggcagatcacaaggtcaggaggtcgagaccatcctggctaagatggtgaaacccccatctcaactaaaaatacaaaaaattagccaggcgtggtggcgggcgcctgtagtcccagctactcgggaggctgaggcaggagaatggcatgaacccaggaggcggagcttgcagtgagccgagatcacgccactgcactccagcctgggcgacagagcgagactctgtctcaaaaaaaaaaaaaaaaaaaagaatgggctcaggtaggtgaaagtagggatagtaatgggaaaccgtggtggtatgcagagaggacattctcatttagcaagagtggaaaactgttgtggatcccaggagaagtctggagctaggtagtaggggctagataatagagggctttgactaataggagtttgggccttatcctgtggaaatctgaagggttttgggaaaatgaattcagggctaggtgcaagacagaagggcaaataccagactaagacacacagccactaagagcctctttttttttttttgtccagtcagaggtaatgaggcctatatatttattaggaaggatgattaaccatggctgtagcttcacctaagaggaacgtggctcttgggccgggtgcggtggctcacgcctgtaatcccagcactttgggaggtagaggtgggcggatcacctgaggtcaggagttcaagaccagcctgaccaacatggagaaacctcttctctactaaaaatacaaaattagccgggcgtggtggtgcatgcctgtaatccctgttactcgggaggctgaggcaggagaatcgcttgaacctgggaggtggaggttgcggtgagctgagatcacaccattgcactccagcctggacaacaagagcaaaactctatctcaaaaaaaaagagaggaatgtggctcttgaagaatctttctagtctcatcttccctgtccccttagacacaccttcaaagatcctttttcttttggtttggcttgtctttccccctcactttacctaatccagcccaattccatagccactgctggttcctttaatggaggagggaagtggggtgacatagattctcttaaacctacctatttaaaggcattaattagcgtttgcatgcactactttaaaattttcctctcaattgcttgagtccaggagttcgaggttacggtgaactatgatttcaccattgcagtccagctgaggcaacagagagagaccctcttaaagaaaaaaaaaactttttcctctcagactctgtccctagcattgactcaccccatcattttttttttttttcttgagatggagtttcaagaaaaaactccaggctggaggcagtggtatggtctcggctcactgcaacctccgcctcccgggttcaagcgattctccctgcctcagcctcccaaatagctgggattacaggcacccgccaccacacccagctaattttttgtatttttagcagagacggggcttctccatattagccaagctggtctggaactcctgacttcaagtgatccacccaccttggcctcccaaagtactgggattacaggcacgagccaccgcgcccggccttcacctattatatctattcttgtcttttaaatagtgagtgacttcacagctcagtctctttgttctgaacttccattctgagtcgcaacccaggtacaccttaggaagggcgggatcccgttatcctgtccctgaacatcaactactgagacagctgatgaagctggctcggtgtgtgacggaggccccctcagagtcagcagagccgagcgtacagcgcacatcaggcagagcagaccagaggcagggaagacacactggggcagggccagtgggcacatctggaccacctgcgagggctctgcagggaaccgtgactacaggccagttgtgagtagtcccctgcagaggctgcccacccaaggtttggttggggtggaacgtgatgacagcacatacactaagaaggtgggaaccgggggctgggtgcagtggctcatgccagtaattccagcacttagggaggccaaggcgagtggatcacctgaggtcaggagttcgagaccagcctggccaatatcgtgaaaccccgtctctacttaaaattgaatttaaaaaaactgggggccaggcccggtggctcatgcctgtaatcccagcactttgggaggccaaggcgggtggatcatgaggtcaagagatcgagaccatcctggccaacatggtgaaatcccgtctctactaaaaatacaaaaactagccagacgtggtggtgggcacctgtagtccagctactcaggaggctaaggcaggagaatcgcttgaacccgggaggcggagatcacggtgagcagagatagcgccactgtactccagcctggcgacagagcgagactgtatctcaaaaaacaaaacaaagcaaaacactgggaacagggttgtcacctacctagcagggctctgagagagctgggcaggcccaaagtggcttgagaaggtaagggaaaaatcctagcctgcgctcggggtcacagagtgggggcccagggggagttcctgctgagggcagggccttgccaggcttcggtctccatccagtgccaaaggtgggagctcccaggctgtcagcatctccagatgagggcagaagggagaggcttaaactcaagtcacatgtccagcgatggagtcctatggccacaccgtctgggaagtgaggagcgcctctgcccggctgctgtgcaaccctccaagtgtgaagtgacagccttgtgtgtgatctttctgccctccccaagtttgcattttcgacattaaagtttactttttaattatgttttaaattggagaatttaaaaaagtgacttaatgaattatgtggctgctcttcctcttccccaattttcagtattctacctgaaactctttcctcttaactcccagatcaaataattttaagccttctgtaggctgtctctctgcagccaaaatcagcgattcagcagtcctcaacctagagaagctcacaatctagtgggcaggagactccttactttggtaagtgttctttgggtctggacagagagagggcagggtgtttcctggggaaggttattcagctgccctggcaggttccctccaggtagtgtaattaccctcatctgtgttcaggaatctgactcactaataaagctatattgatctataaaaccagactcctggccaggcgcggtggctcacgcctgtaatcccagcactttgggaggccgagacgggtgatcacgaggtcaggagttcaagaccagcctggccaaaatggtgaaacctcatctctactaaaaatacaaaaaaattagcggggcgtggtggcacgcgcctgtaatcccagctactccagaggctgaggcagagaattgcttaaacctggagaggcagaggttgcagtgagctgagatcacaccactgcactccagcctgggcgacagagtgagactccgtctcaaaaaataaataaataaaaataaaaaaataaaaaaccaaactccagtgtgttaatttatgatgaaaatatatgtcaggtagactggtcctttggccttactagtttattttcttcatgtaatcatactgcttgggaagcttagttaaagttaaaaggcagctctataaatcaaagtgagaggagttagttgttctagggatgggatgtttaccaaaatcaagttacccctcccaccataagcctggctcagtctctccccaattcattttgactcagtgattcctgattgtttcacctgttcaacttgatatttcctccagccccaaatggcacaatcttagatgtcacttagatgatcgctctggtttccattatattccagcccttaagacccgtttgtttttcacattactttgcttctctgctctgttgtgtctgtcctacagccgaattaccaggttaggagtagggagactctccaaacaccatcttccccaaatgtgctacccagcttattcaaatctgtgtggcagtggtaccaggatacagatctaaataaagttcccaatgtgctaaattatagatggcctatctgtagaagggtactgtgagagcttttctcaggttatgactaagaggtgtgtaaacatgagggaaccttacaggtcaaggacagaaatcccgtgaatgcagacccaggaaacctgtagacggctctgcctacaaccttgcagacaccctgtgcatgcagcagctgaggcttgttcagaggggtgggactcagggccttgcattagcacttccagccccgctttgttaccctgggagcaaggctatgttgggaagaatagctagtcctattccactgggtgtcggggcctcgggatccatgaaacggaaaacagcaatagcgtgggcttgcctctctacacatgaaggcctcctttgatttcagtataaaaacacccatgaagcaatggatatgcagatccaggcaaagaaaaggtaaaggtagggaaggggggaggggacagcaacattacctcgcttgtgacatagggagcaacaaaatgcaggcagccgccagtggcacgcaggctcagttctagcagggggtactctgggatttttagtttctcagcctttgcagccaaggaagactgacacctacatccttgcacccctatcatgcatacagcaagactgtatgcagggcctggtggaggaggctggggaatcatcatttaacaatatctataatagacatttacagggctggtgggatctcaaaaaggcctgtaaatgcctgaggaattcaggaagttttcaccagaaaatgatgtgataaactggatctgaatggattaggcattcgccagggagacagtgggaaggtagcagcaagtgggtgggatcattccaggcgaagaaactgctccaacgagcttggagcgtgaaagcaattgtggatggctgagaacacgagtttgagtgggggcagtaagcaatgggatctgaggatttagaaatgggtgaatgatcttgaatgccaaacccaagaatttttaccttttcttgaagcattgacacacagccagggcagaatgaatggctcccttctttgtgtttcattcatctttctgtaacagaagatcactggctacaacattttgattgtttcttcatcccaagaacccaagggccatggaagtgttgctgttttcataagttattagggtacaggtggtatttggttacatgagtaagttctttagtggtgatttgtgagattttgatgcacccatcacccaagcagtatacaccgtaccctatttgtagtcttttatccctcgcccccctcccgccctttcctccaggtccccaaaatccactgtatcatccttaagccttcgcgtcctcatagcttagctcgcacatatcagtgagaacatacgatgtttgattttccattcctgagttacttcacttagaatattagtctccaatctcatccaggtcactgcaaatgccattaattcattcctttttatggctgagtagtattccatcgtatatatatatacaccacagattcttttttttttttctttgagacgggagtcttgctctgttgcccaggctggagtgcagtggtgtgatctcagctcactgcaacctctaagcctcctgggttcaagcgattctcctgcctcagcctcctgagtagctgggattacaggcacatgccaccacgcccggctaatttttcatatttttagtagagacggggtttcaccgtgttagccagggtggtctccatctcctgaactcatgatccgcccaccttggcctcccaaagtgctgggattacaggcatgagccactgcgcccggaccacagtttctttatccactcgttgattgatgggcatttgggttggttccacatttttgcaactgagaattgtgctgctataaacatgtgtgtacaagtatctttttcgtataatgacttcttttcctctggatagatacccagtagtgagactgctggatcaaatggtagttctacttttaattctttaaggaatctccacatagttttccatagtggctgtactagtttacatgcccaccagcagtgtagaattgttccctgatcactgcatccatgccaacatctactgcttttttattttatttatttttttagagacaagagtcttgcactatcccccaggctggagtgcagtggcgtgaccttggctcactgcaacctccaactcccaggtttaagcgattctcatgctttagcttcccgagtagctgggattacaggcgcctgccaccacgcatagctaatttttgtacttttagtagagacaggctttcatcacgttggccaggctggtctcaaactcctggcctcaggtgatctgcctgccttggcctcccaaagtgctgggattacaggcatgagccaccatgcctgttttttgatttatttatttatttatttatttatttatttatttatttatttattttttgagacggagtctgcctgtgtagcccaggctggagtgcagtggcgcgactcggctcactgcaagctccgcctcccgggttgacaccattctcctgcctcagcctcccctgtagctgtgattacaggcgcccgccatcacgcccggctaattttttgtatttttagtagcgacggggtttcaccgtgttagccaggatggccttgatttcctgacctcgtgatccacccgcttccgcctcccaaagtgctgggattacaggcatgagccaccgcgcccggccttttttgatttttttactgcggccattcttgcagcagtataatggtatcgcattgtggttttgatttgcatttccctgatcattagtgatgttgagcttttttaaatatgtttgttggtcatttgtatatcttcttttgagaattgtgtattcatgtccttagcccactttttgatgggatttttttttcttactgatttgtttgagtttgtttttgttatagattctggatattaatcctttgtcagatatagattgtgaagattttctctcactctgtgggttgtctgtttactctgctggctgttacttttgccatgcaaaatctctttagtttaattaagtcccaactatttgtctttgtttttattgcatttgcttttggcttcttcgtcatgaaattcttgcctaagccaatgtctaagttattttctagactttttatagtttcaggtcttagatttaagtccttaatccatcttgagttgatttttatttaaggtgagagatgaggatccagtttcattctcctacacatggctagccaattgtccaggcaccatttgttgaaaaggtgtcctttcctcactttatgtttttgtttgctttgtcaaagatcagttgggtgtaaatatttgggtttatttctgggttctctattctgttccattggtctatgtgcctatttatttattttattattattattattattattattattattttgagatggagtctcgctcctgtcgagcaggctggagtgcagtggcgtgatcttggctcgctgcaacctccaccttccaggttcaagcaattctccttcctcagcctcttgagtagctgggattacaggtgtgcaccaccacgccgggctaatttttttatttttagtagagatggggtttggccatgttggccaggctggtctcaaactcctgacctcaggtgatccgcctgtctcagcctcccaaagtgctaggattacaggtgtgagccactgcacccggcctatgtgcctatttttatatcagtactatgctgttttgtgactatggccttatagtatagtttaaaatcaggtaatgtgatgcctccagatttgttctttttgcttagccttgctttggctatgcaagctcttttttggttccatataaattttataatttttttttttagaattgttttttctaattctgtgaagaatggtggtggtattttgatggggattgcgttgaatttgtagattgcttttggcagtatggtcattttcacaatattgatttctacccatccatgaacatgggatgtgtttccatttgtttgtgtcatctatcatttctttcagcagtgttttgtaattttccttgtagaggtcttttgcctccttggttaggtatattcctaaatattttattttattttttgcagctattgtaaaaggtgttgagttttttttgtttgtttttgtggttttttgtctttgttttgtttttgtttgttttgagacagagtcccactccattgcccaggctggagtgcaatggcgtgatcttgacttgctgcaatttccgcctcccaggttcaagtgattctcctgcctcagcctcccaagtatttgggattacaggcacctgccaccatgcccagcaaatttttatatttttagtagagacggggcttcaccaagttggccaggctggtttcaaactcctgacctcaggtgatccacccaccttgccttcccaaagtgttgggattacaggtgtgagccaccacacccagcctccctccctccctccctccctcccttccttccttccttccttccttccctccctccctccctccctccttccttccattctctctgtctctctgtctctctctctttttgacagggtcttgctctgtcacccagactggagttcagtagctcaatcatggcttactgcagcctggagttcagtagctcaatcatggctcactgcagcctcaaccttttgggctcaagtgatcctcctgcctcagcctcctgagtagctgggactacaagcaacacaccaccatgcccagctaatttttttttaattttttttgttgttgttgtattgacaagatctcactatgttgctcaggctggtctcgaactcctgggctcaagcaatccacttcagtctcctaaagtgctgggattacaggcatgagccatcatgcttaaccctgcatggtatttctgaggaatgatatgatgaaatctgtattttaggatgaccagtcatgcagaacagattggagtgaggagttgggagacaagaaggaagagataaattggaaatttgttgaaggaatatttgacagaacttgtgctgtaattaaatactaaatgaaagaaaaggaaaaggcagaatacaactccagagtttgctgcctaagtggctgggaagatgacaccatcaacaaacaagaaaaggggcaggggactggaaaaggtagtgataactcgccactagcactttgtgggcaagggccatgagttaccagttatataatcctgcagtattcagctggacatacaagaggtcttcacaaagtttatgaaaaatgtgtattatgaaaaaactatgcatagatttcaaaaaatttttgcaccaaaataaatttgtactaacttgttataacatgtctgagcaggatctactttgaggcactatgaaggataagacattagtttgaaaagagcccctataagagtaacatgaattctgctaaaattgaagcaagaacaaatatcaagtttatggttaagcttgggtggaagaatggtgagatcattgaagctttacgaaaagcttatggggacaatgcctcaaagaaatcagcagtttacaaatagataaattgttttttgttgttgttttttgagagggagtctcactctgtcacccaggctggagtgcagtggcatgatctaggctcgctgcagcctctgcctcccacgttccagtgattctcctgcctcagcctccctgggtagctgggattacaggcgcctgccaccacgcccggctaatttttgtatttttagtagagatggggttttgccatgttggccaggctggtcttgaactcctgacctcaggtgatacacctgcctcggcctcccaaagtgctgggattacaagcatgagccaccgcacgtggccagataatttgttttaagaagggatgagattaggccgggctgtgcctctactacagactcctagtatttattttctttaagaatacttggccaggcgtggtggctcatgcctgtaatctcagcactttgagatgctgaggcaggcgaatcacctgaggtcaggagttcaagaccagcctggccaacatggtgaaaccccgtctccaccaaaaatacaaaaattagctgggcattgtggtgggctcttataatcccagctacctgggaaggctgaggcaggagaactgcttgaacccaagaggcggaggttgcagtgagccgagaccacgccattgcactccagcctgggcgacagagcaagactccatctccaaaaagaaacaaaataaagaagggatgaaatgatgttgaagatgaaacccacagtggcagaccatccacatcaatttatgagaaaaacattttgtgtcctaactgaagtggattgacaacagcagaaataataatcaataccataggcatctcacttggttcggcttacataattctgactgaaaaactaaagttgagcaaacattccacttggcaggtgccaaaccatcatgcccagatcagctgcagagctttcaatgggaattttaaacaagcaggatgaagatcctgaagcatttctttgaagaattgtaataggagatgaaacatggctttaccagtacaatcctaaagacaatgcacaatcaaagcaatggctaccaagaggtggaagtgtctggtcaaagcaaaagtggaccagtcaagagcaaaggtcatgtcattcgtttttttttttttaatgttcaaggcattttgtttattgactttctggagggccaaagaacaataacatctgcttattatgagagtgttttgagaaaactagcaaaagttttagcagaaaaatgcctgggaaagcttcaccagagtcattctccgctataacaatgttcctgctcatttctctcagcaaacaagggcaattttgcaagagttttgatgggaaatcattaggcatccaccttacagtactgatctggctccttctgacttccttttgccttttttttttttttttttggagacagagttttgcttttgttgcccaggctggagtgcaatggtgcgatctcggctccctgcaacctctgcctctccagttcaagcgatgctcctgcctcagcctcctaagtagctgggattacaggtgcctgccaccacacctggctaattttttgtatttttagtagagacagggtatcaccatgttggtcaggctggtcttgaactcctgaactcaggtgatctgcctgcctcagcctcccaaaatgctgggattacaggcattagccactgcacctggcccttgttttctaatcttaaaaaactctttcaagggcaccccattttttcttaggttaataatgtataaaagaggctgccttatccttacaagcagcctgccctcaactctctctcagagtgttaaaaaaaaatgtataaaagggccaggcatggtggctcatgcctgtaatctcagcacattgggaggcccaggcaggaggatcacttgagcccaggagctggagaccagcctgggaaacataaagagatactgatctccacaaaaaatttttttaaagaattagcttggccgggcgcagtggttcacgcctgtaatcccagcactttgggaggctgaggcgggcagatcacgaagtcaacagatctagaccatcctagccaatatggtgaaacctcgtctctactaaaaaaaaatacaaaaatcagctgggagtggtggcgcacacctgtagtcccagctattcaggaggctgaggcaggagaatcgcttgaacctgggaggtggaggttgcagtgagccaagattgcaccactgcatttcagcctggtgacagagtgaagctctgtctcaaaaaaaaaaaaaaaaaaagaattagctgggcacagtggcatgtctgtagtcccagctacttgggaggctaagcaggaggattgcttgagccctggagtttgaggctgcagtgatctatgatagcactactgcactccagcctgggtgatggagtgagaccctatctcaaataaaataataattataataaagactgccaggcacatggtggcatgcacatgtggtcccagctatttgggaggctgaggtgggaggatcaccagagcccagggaggttgaggctgcagtgaactgtgattgcaccactgcactccagcggggcaatgggagtgagaatctgtctcaaaagtaataattatatatatatatatttatatatatatatatatatatatatatatatatatatatatatatgttctagtattttcctcccatacctccaatgtataatatctcatgcaccctgtggtccaccccccaaagacatacacacaacactttagagtctctgatgtagtcaaagggagtcattggaggttctggaacagatgacaggcatggtagcagttgattcacaaactgaaagctgagacatcaaagaactgcgactgccttggaatgtttcccatgtgctagccaccgcctcagtgctttatacataaatcagttaatccctatagcaagcctccattgtgaataccataattatcttaacagacgaggagactgaggcacaatgaagttaagaaactttcctagagtcacacgaaattggtgggggcctacttttttttttttttttttttttttttttttttttttttgagactgaatctagctctgtcgccaggctggagtccagtggcgcgatctcacctcactgcaacctccgcctcccggattcaagcgattctcctgcctcggcctccccccactagctgggattacaggcactcgctgccacacccagctaatttttgtatttttagtagagacagggtttcaccatattggccaggatggtctcgatctcctgagcttgtgatccgcccacctcggcctcccatagtgctgggattacaggcctgagccactgcgcccggctgggggcctacatttgaccccacgtggtggtttgggctcatagtccatgtgcttaaccactagtgggtaggcagggagccagtgttctttctctcctgtgactttggtgaagtctctcaccactcagtgttgtcgtgagcatgctaggcagagtgcaagaaaggagcaagaactcactaatggctaggccttccctgtgtctctgtaattgatgggtttgggttttttttcctttctctctctctacctcctctctctcttcttcatcctctctctctcactctccccctgagataaggtcttgctctgttgccagggctggaatgcaatggaatggtgtgatcatggctcactacagcctcaatctcctgggctcaagggatcctcccacctcaacctcctgagtagctggaaccacaggcatgcaccacctcgcccggctaattttttattttttgtagagaggggagtttcgccatgttgcccaggctgttttcgaactcctgggctgaagtgatcttcctccctcagccttccaaggtgctgggattgcagacgtcagccaccgtaccaggccaacgggctcggtttctatggttttgtggggagacggctgagggaaggtcggcttccggcgtcgc

SEQ ID NO: 4 - DNA, 27888 bp, Homo sapiens
  source 1..27888: mol_type = genomic DNA
gcaccaccacgcccagctaattttttgtatttttagtagagacgaggtttcatcatgttggctaggctggtctccaacgcctgacctcaggtgatccgcccgccttggcctcccaaagtgctaggaatgtaagcgcgagccaccgcacctggcctctattccacaaattccataaatgttcactactagattacctttctactagactactttctacctttctagtagtaccttactactagattaccctgagtccaccacataattgttttcttcctccattccctttttcttcaaacattcaccttatcttatgtaaaaatgtagatttaggccacctaggagggcggatcataaggtcaggagatcgagaccatcctggccaacatggtgaaaccctgtctctgctaaaatacaaaaacttagccgagtgtggtggcgcgcctgtagtcctagctacttgggaggctgaggcaggggaatcgcttgaacccggaggcagaggttgcagtgagccgagagcaccactgcactccagcctggcaacagagcaagactccgtctcaaaaaacaaaacgtagatttcctgagcattagctaaagtctttgcctcaatctttcccacccccacttttcaaggaaaatgtataaatactaaaaacctcctgagaacctcttgggaagaaacagccacaaaagtgtctgtgacatggtttagtcagtttagtcactcatttcaattaacactatctcttatccatcccttccactgcagctgaaggaaatctttctgaagtctgatcatgtcactgttattcgtggtttaaaagctggggaagaaacagccacaaaagtgtctgtgacatggtttagtcagtttagtcactcatttcaattaacactatctcttatccatcccttccactgcagctgaaggaaatctttctgaagtctgatcatgtcactgttattcgtggtttaaaagctggggctaaagaaagaaagaaagtaaaatttaaaaagaaaagaaaaataaaaaataaaataaaagctgaggctctgtagtcaatttactgagttaaaactcctcactgtcagggccgggcgcggtggttcatgtctgtaatcccagcactttgggaggctgaggagggcagatcacctgaggtcaggagttcgagaccagcctaacgtggagaaaccccatctctactaaaaatacaaaattagccacgtgtggtggcgcatgtctgtaatcccagctactctggaggctgaggcaggtgaatcgcttgaatcagggaggcagaggttgcagtgagctgagatagcgccattgcactccagcctgggcaacaagagcgaaactctgtctcaaaaaaaaacacacacacacaaacacaaaaactcctcactgtaattttaggtgtgaacttgggcaagtcttaaactcattaaaccttacttttctcaactgtaaaatgggaataatattaatactttattgtgattatctatgtagttcagtacagtacttagttcttattataaattagaaatgttagctgctattagtataactcttccatccttcacacttataacacccagaacacagtgtagttgtttcaagccttcagacttttttttttaagatggaatttcgcttttcttgcccaggcaggagtgcaatggcacgacttctgctcactgcaccctccacctccagcgttcaagcaattctcctgcctcagcctcccaagtagctgggattacaggcgcccaccaccacgcccggctaattttttgtatttttagtagagacggggtttcagtatgtcagccaggctggtctcaaactcctgacctcagttgatctacctgcctcagcctcccaaagtgctgggattacaggagtgagccactgagcccggccgccttcagacttttgtttgaacctggaaaactctttccctggagaaaacatgtctttcagcattactttctctccggctgtttcttacctttactcattcccaggatttacaattcccctcctttgtgtccaaacaatagcttgtatagccaaaaaaatcttttaagcaattttagttttacatttcttcctctgcctgctagagtgactttctggagagtaggatgtgggtgacatctgtctctgtgtccccaaagtcacaacaaagcatctgatataaaataggagctcagtcagtgagcattcaatcaatagtatcatgtttctgtctctgtccccagctccctcactaactcccttggcccttatagtgggataatgaatgaggaagtgctgtctcatgctcattgagcatctcaatttttgttatttatcatactactttgcatagacgtgatttatttgctttttccttctttgcctgaggctgaatagttagttggcattattcctttttttaatttttatgtaacttttattcttttttatcaggcagccacccgagccagcagccagcgtaggctcagagagactcccctttctttcttgcaggagcaagggcagcatgaaagttcagagcttaataatggatggttattggctatgggcaggctaaaccaatacacagtacatacagtcatacttcagcccaaagaaaattgccacctattgtattcaccatatgccaggcattgggctgggcacttgagtttatttttctaattcaagaacaaggccaggcacggtggctcatgcctgtaatcccagcactttgggaggctaaggcgggcagatcacaaggtcaggagtttgagaccagcctggccaacatggagaaatcccatctctactaaaaatacaaaaattagctgggtttcgtggtgtgcacctgtagtcccagctactcagggggctgaggcaggagaatctcttgaacccaggaggcggaggttgcagtgagccgagatcgcgccactgcactccagcctgagtgacagagtgagactccgtctaaaaaaaaagaacaaaagccgggcatggtggctcaagcctgtaatcccagcactttgggaggccaaggtgggcagatcatgaggtcagtagttccagaccagcctgaccaacatggtgaaaacctgtttctactaaaaatacaaaaattagctgggcgtggtggcaggcacttgcaatcccagctactcaggaggctgcagcaggagaatcacttgaacccgggaggcagaggttttagtgagctgagatcccgccactgcactccaacctgggcaacagagcaagactctgtctcaaaaaaaaaaaaaaagaaaaaagaaaaaaagaaaagaaaaaagaactaaaagagagtggagcacatttggcatgtgactatagtcccagctacttggtaggctgaggtgagaggatcacttgagccctggagtttgagcccagcctaggcagtgctgcaagaaccagtctctaaacaaacaaagaaaaaaaaacaggaaaaaaaaagagttatcctagcctagagaaatgttaacagtctatccccttaagatcataaaagtgaaataactggacagagtactgaaaaaagaagaaatctgttgaacaagtctaagtgatttgtagagtctctccaaaggacagtaaagatttcagaaccatggggaggaggctttcttgcagggtctgatgtatacctctagttctatttttagatatattccattgtgctctcttcatttgctcaccattagcatgggaattattccccttgttttatttatatttgtagtaacaagtctagcacagtaactggcatagaagtacttaataaatgttacaatgtgttgaagacttttgagatgtagaattaaattatttgaactacgtttcaggaagttgactctggaagctgtgtgatgaatgaatttagtgactaggagattaggcaaagagagcaattgggaggccacggcaatgatccagacaagagaagtctcagaactgtaatatgacattagcagcagggtagaaagaaggaaatacaaggtatatttcttcaggctttaggattaattggatttaggatgaaaggaaagagtaaattaaatccagtttcttggtgacatgagtgatgtcattaacctgtgacataggaaacactggaagaaaagcaggtttgaggtttaaaataatgttaaacaggccgggagcggtggctcacgcctgtaatcccaacattttgggaggccaaggcaggaggatcatctgaggtcaggagttcgatatcagcctggccaacatggtgaaaccccgtctccactaaaaacacaaaaatcagctgggcatggtggtgggcacctgtagtcccagctacttgggaggctgaggcaggacaagtgattgaacccgggaggcggaggttgcagtgagccaagatggcaccactgcactccagcctggatgacaagagcgagactccatctcaaaaaaaaaaaaaaaaatgccgggtgtggtggctcatgcctgtaatctcagcactttgggaggccgaggcaggtagattgtctgagctcaggagttcaagaccagcctgggcaacatggtgaaaccccgtctctgctaaaatacaaaaaaaaaaaaaaaaattaaccgggtgtggcagtgtgcccctgtaatcccagctacttgggaggctgaggcaggagaattgcttgaacccaggagcagaggttgcaatgagccaagatcgcaccactacactccagcctgggcaacagaacaagactccgtctcaaaaaaaaaaaaaagcagtagttacatcactgcactaatttgctgtgggagacagttacctgactcatcaacagccattgtctcttcttcacagccaactaaatgtaaaaggtaaggcaattcaatgtaagtataaagcccatgtcccaggacagcaatcattttaacagtaggacttgctaacctgatagcagcctgctataaacttataaaccattaaaattaaacctataaaccatgaaactaaatgctctcagataattgtaacaacaaatgcaaaataaaataatggacgccgggcatggtggtgcatgcctgtaaagccagcactttgggagaccgaggtgggcagatcacttgagcccaggaatttgagatcaacctggacaacatggcgaaactccatctgtgggggaaaagcaagagagatcagattgttactgtgtctgtgtagaaagaagtagacataggagactccattttgttctgtactaagaaaaattcttctgccttgagattctgttaatctatgaccttacccctaaccccgtgctctctggaacatgtgctgtgtcaaactcagggttaaatggattaagggctgtgcaagatgtgctttgttaaacagatgcttgaaggcagcatgctccttaagagtcatcaccactccctaatctcaagtacccagggacacaaacactgcggaaggctgcagggacctctgcctaggaaagccaggtattgtccaaggtttctccccatgtgatagtctgaaatatggcctcgtgggaagggaaagacctgaccgtcccccagcccgacacccgtaaagggtctgtgctgaggaggattagtataagaggaaggcatgcctcttgcagttgagacaagaggaaggcatctgtctcctgcccgtccctgggcgatggaatgtctcggtataaaacccgattgtacgttccatctactgagatagggaaaaacctccttaaggctggaggtgggacatgcgggcagcaatgctgctttgtaaagcattgagatgtttatgtgtatgcatatctaaaagcacagcacttgattctttaccttgtctatgatgcaaagacctttgttcacgtgtttgtctgctgaccctctccccacaattgtcttgtgaccctgacacatccccctctccgagaaacacccacaaatgatcaataaatactaagggaactcagaggctggcgggatcctccatatgctgaacgctggtcccctgggtccccttatttctttctctatactttgtctctgtgtctttttcttttccaagtctctcgttccacctaacaagaaacacccacaggtgtggaggggcaacccaccccttcaccatctccacaaaaaataaaaaattagcccagtgtggtggtgtgtgcctgtggtcctaactagtccagaggctgaggtgggaggattgcttgagtcccagagctcgaggctgcagtgagccgagatggcaccactgtactccagcctgggtgacagagtgagcagagtgagaccctgtctcaaaaaataataaaataaaataatgggaacccaaatcacaagacagcactcatttttcttttatttattttattttttttgagacagagtcttgctctgctgcccagactggagtgcagcagcatggtttcggctcactgcaacctccacctcctgggttcaagtgattcttgtgcctcagcctcccaagtagctggaattacaggtatgtgccaccatgcctgactgatttttgtatttttaatagagacagggttacgccatgttgtccagtctggtttcgaactcctggtctcaagggattcacccatctcggcctctcaaagtactgggattacaggctgagtcaccactaccgggcttatttttcatacagatgatttagaatatgtttatgttaaatttttttagattatcactaaaagaaaacagttgaattcttcaatttacaaggattcaatttttaaaattatatttgaatgtacagaggcctacaaataccaatttcagaaacactgttatcaaggatatctatttgttaaaattgaatgcaactgaaagtaacagattaactgcaaataacaagcttaaccaaataggggattaatttttcccatttaacaaggagacagggccgggcacagtggctcacgcctgtaatcccagcactttgggaggtcgaggcgggtggatcacttgaggttgggagctcaagaccagcctgaccaacatggagaaaccccgtgtctacgaaaaatacaaaattagccggacgtgatggtacatgcctgtaatctcaactactcgggaggctgaggcaggagaatggcttgaacctgggaggtggaggttgcagtgagccaagatcgcgccatttgcattccagcctgggcaacaagacgagctccgtctcaaaaaaaaaaaaaaaaaaaaaaaagcaagtagacagggccaggtggagtggctcatggttgtaatcccagcattttgggaggccgagatgagaggatcatttgagctcaggagttggagaccagcctgggcgacataggggtacccatctctacaaaaaattaaacaaacaaacaaaaaatagccaaacatgatgacgcatgcctgtagtcccagctacccaggaggctgagatgagagaattgattgaatctagggggtttaggccacagtgagccatgaatgcaccactgcactccagcctgagcaacagagcaagaccctgcctcaacaaaaaaaaagcaaaacaaaatgataaaaagcaagtaaggcagggcacagtggctcacgcctgtaatcctagcactttgggaggccgaggcaggcagattacttgaggtcaggagttctaaaccagcgtggccaacatggtgaaacttcgtctctactaaaaatacaaaaaaaatagccaggcgtggtggtggacacctgtaatcccagctactctggaggctgaggcaggataatcgcttaaacccgggaggcgatcatgccactgcactccagactgggctacagagcaaggctctgtctcaaaaaaaaaaaaaaaaaaaagaagtagacagctgttacagtatgttaagctccatagagacagcaccagggcaagtgagagcctgacaggcacagggtgactctgtgcctcactgaggaaaaataactagacatgagcaaaggagatcctcagaaacctagaggcaaaatgtcatcatatgcatttttcgtgcaaaacttgtcgggaggaacataagcacccagaggcttcagtcagcttctcagagtttttttttttttttttttagatggagtcttgctcccatcgcgcaggctgaagtgcagtggcgcgatcttggctcactgcaacctctgcctcccgggttcaagtgattctcctgcctcagcctcctgagtagctgggattacaggtgtgcgccaccatgcctggctaatttttgtatttttagtagagacagggtttttgccatgttggccaggctggtctcgaactcctgacctcaggtgatccacccgactcagcctcccaaagtgctaggattataggcgtgagccaccacgcccagccagcattttctaagagtgctcagagaggtagaaaactgtatctgctaaagagaaaggaaaatttgaagacatggcaaaggtggacaaggcccgttataaaaaagaaatgaaaacctatgtcccccgtaaaggggagataaaaagaagttcaaagatcccagtgcatccaaaaggcctcctgtagcctttttgtctttctgttctgagtattgcccaaaaatcaaagaacacccaggcctattcattggtgatgttgcaaagaaattgggagagatgtggaataacactgctgcagtcgggcatggtgtctcacgcctgtaatcccagtactttgggaggccgaggtgggcagatcacgaggtcaggagttcgagaccatcctggccaacatggtgaagccctgtctgtactaaaaatacaaaaattggccactcgtggtggtgcacgcctgtaatcccaactactcgggaggctgaggcaggagaatcgcttgaacccgagaggcggaggttgcagtgagcagagatcgcaccattgcactccagcctgggcaatagagtgagactctgtctcaaaaaacaatgaccaaaaaaacactgctgtggatggcaagtagccttatgaaaagaaggctgagaagctaaaggaaaaataggaaatggaaattgttgcatatcaagctaaaggaaagcctgatatagcaaaaaagggagtcgtcaaggctgaaaaaacgaaagagaaagaaggaagaggagaaagatgaggaagatgaggaggaggaagatgaagatgattaataagttggttctatcacatttttttttctgtctaaaaagcatttaacccttctgtatacaaattactccctttaaagaaaaaattgatgccgggcgcggtggctcatgcctgtctgtaatcccaacactttgggaggccaaagtgggcagatcacctgaggtcaggagttcaagaccagcctggccaaaatggcgaaaccccatctctacaaaaatacaaaaattagctgggcatggtggtgtgtgcctgcaatcccaggtactcgggaggctgagtcatgagaatcacttgaatccgggaggcagagattgcagtgagctgagatcgcgccattgcactccagcgtaggcgacagagcgagacttcatctcaaaaaaaaaaaaaaaaagaaaaaaattgaaaagtaaggctgtgtaggatttgtttttaaactatacagtgtcttttttgtatagttaacacactactaaatgtgtctttacacattatcttttagtttttctagagacggagtcttgctctgttgcccaggctggagtgcagtggcaccatcatagctcactgcagcctggaattcttgggctcaagcaatcctcttacctcaggctcctgagtagctgggactatagatgggtgccaccacgcctggctaatttgaaaaaaaaattttttttttttgagatggagttttgctcttgttgcccgggctggagtccagtgtcaccatctctgctcactgcaacctccgcctccagagttcaagcaatcctcctgtctcaaccttctgagaaattgggattacaggagctcgccaccacaccaggctaattttttgtatctttagtagagatggggtttcaccatattggccaggctggtctcgaactcctgacctcaggtggtccgcctgccttggtctcccaaagtgctgggcaccacgcccgacctaaaacatttttttttgttggccaggagcggtggctcacacctgtaatcccagcactttgggaggctgaggtgggtggatcgtgaggtcaggagatggagaccatcctggctaacacagtgaaaccccgtctctactaaaaatacaaaaaattagcttggcgtgatggcaggcgcctgtagtcccagctactcgggaggctgaggcaggagaatggcgtgaacccgggaggcggatcttgcagtgagctgagattgcgccactgcactccagcctgggtgacagagtgagactctgtctcaaaaaaaaaaaaaaaatttttttttgttgtttagacagagactctttatgttgcccagacttgtcttcaattcctgggctcaagcagtcctcctcctttggcctcccaaagtgctgtgactatagttttttgattagaaaaaaaaaaaatacaagttaaaaactggtatgatcactccacactgtatcaggaactcagctcactgcaacctccaactccctggttcaagcgattctccggcctcagcctcccaagtagctgggactacaggcacgcactgccatgagcagctaatttttgtatatttagtatagatggggtttcaccatgttggtcaggatggtctcgatctcctgacctcatgatctgcccgcctcggcctcccaaagttctgggattacagccgtgagtctggcctacttttattcttatgataaccctatggtcacaaaatgtctgctctacttccagcctggacaacatagtaagacccagtctccaaaaaaaaaattttttaattcatttaaaaaataaaaaataaatgtatttaagttgctctttacctgtctttttttttaagagatgaggtctcactatattgcccaggctggtcttgaaatcctgggctcaagagattctcccaccttggcctcccaaagtgctgggattacaggtgtgaactgctactcccagcctacctaatctttatttgaacttttcttttcttttctttttgagatggcatcttgctctgttgaccaggccagagtgcagtggcgcaatctcagctcactgcaacttctgcctgccaggttcaagcaattctcctgcctcggcctcccgagtagctgggatcacaggcatgcaccaccacgcctggctaatttttgtatttttagttgagacagggtttcgccatgttggccaggctggtctctaattcctgacctcaggtggggacctcttggcctcccaaagtgctaggattacatgcatgagccaccacgcctggccttttttttgagacggagtcttgctctgtcacccaggctggagtgcagtggctcaatcttggttcactgtaaacctctgcctcccaggttcaagcaattctccaacctcagcctcccgagtagctggaatgataggtgcctgccgccactcctggctaatgtatttttagtagagacagggtttcaccatactggtcaggcttgtctcgaactcctgagctcaggtgattcacccgcctcgacctcccaaagtgctgggattataagccaccgcacccggcttttttttttttttttgagagatgaagtgggctgtgagtgctggctcatgcctatagttgcagcattttcggggtgctaaggcagtagaatcacttgagcctaggaattcaagaccagcctgggcaacatggtaagaccccatctctacaaaaattaaaaattagctatgtgtggtggtaggtacctgtagtcccaggaacttgggaggctgaggtgggaggattgcttgagccttggagggtgaggctttagtgagctgtgatcatgccactgcagtccagcctgggcaacagagtgagaccctgtctccaaaaacaaccaaaaaaaggagagatggggtcttgctctgtcatcctggctggagtgcagtggctgatgatgggtcactgccgccttaaactccccagcccaagtgaattctcccgcctaagcctcctgaatagctgagattacaggagtatgccaccatgccaggcttgaacttgtttctgggtttacattgacttccactcattattttattttttatttttacaaatctgacatttcaattttttttttttttttgaggcagaattttgctctgttgccaggctgaagtgcagtggcacaatcttggttcactgccacctccgcctctggggttcaagcgattctcctgcctcagcctcccgagtagctgggactacaggcatgtgtcaccatgcccagctaatttttgtatttttagtggagacagggtttcaccatgttggccaagatggtctcgatctcttgacttcgtgatctgcccacctcggcctcccaaagtgctaggattacaggcgtgagccactgcacccagcctcaattcttatattttctatcagatatcaaccttaaattcaagtatgatttgataattgttcttctattgctgtcctggttaatactatgctgcttttcattattaaataaggcacccatttaggatattgaacgctatggattttagcttcaagttttcattttcccagaagatgaaactagagacataaaatattgatttttcttcttttcttccctcttctttcccctccaattttaggggatcaagaatgaatgtagagatatagtccagaccagagcagtaaagggaacatgtccaaattggatcaatcatgaagccaacttggaatactaaagttggtttttatctctagctacctgatgcttcagcaactgattaattctcagattcttttttttagaaccaagtctctgtaagtagaagcacttttcggttatttcttaatctcaacactattggcattttaggtttgataattctttgttatggagggaccgtcctatgtgtaataggatgtttagcagtatcctggcctcttccctctagatgccagtagcaccccccacacacggtgccaactgtctccccaggttgagaaccgctgagttagagaaggtcaaaaaaatgactagaggactcctagtttatctaaccttctgacctatttgtcctgtttgtcctggctggcagggtcagctctgcctagaggcaggggaaaaatcttttacttctttcatgtattcatacgttacttctagctatccatcctggccaaagcttccagaaattaggaagcttgcttgccaaaagcactgcaattaggagagtgatgatgtcgggactagaaagactcacacccactgtaatctgtcttcatattgctcttgagtgccactctgcccaaaatttatcatgttgacaaatgacaaaaggctctgaaaaagcatacacatgaaaaaggagagctttacctctaggcaagaatgcttcactctgattaattcctacccccaaccacacccctcaccacaagatagaatcttgtcttttgtgaggtcttagtcatgttcctttggcccactgcaggccataattctgcaactagagacatctctaattctctccttccaccttcccctagagaaatgtcaggcctgacagatggaccctagtttgagatctctatgaccagagtgggttctgaggcaggaccaacccttacggttgagcagtctgagcatctgacaaaagggaatagtgggtggtctggtaacctgcccctaacctgctcatcagagatggtttgctgaaccaggagaaagcttggccctttctaattataaaataaaacaatttcactcagtatatgtttcaaaccagaagatttatctcccctcttttcttctttaacttaaacagccccatttccccagctgctccttaactgacctgctttccaaacccctgctgatatcttcctatagcatttagcttagtatcaagttcaacctccttccttagcttgacacacgaggctctctaccctctgaccccttgtcactcctccctcatatacacctcctgccgctctagccgtaaagcactatttgcaatccccggagtgtatgttgcagatctcagtgctctccccaattctctccacttactctttgcctaaatcctgcttgaaaaaattgggcttaagtgttaccagttcagtggtagctttcctgacaacctccttattcccagtctgattttcagtgccccttcctctgtatttccaccataccctgtgcatatttctatcatatgcttacatagttccataattgtttataatttatgtttcttttccccctttggaccatgatctttgtgagtcaaggttatggtggttgtaagtatctcagagcattgaatagaaaccgatacaaccattgcattgaactatgcattccttgagggcagggccatatcttcacctctgcataccaacatctagcatatcaccagtaaatagttggtgttcagtgaatgaagaatgaatacatgaatgcttcaggcagaaggttgctctcatgtggctagctacacatttggtgatctttttttgtttgttggtttgcttgttttttgttgctgttgtttttgttttttgagacagagtcttgctctgtccaccaggatggagtgcagtggtgtgatctcggctcactgcaatctccgcctgccgggtttaagctattctcctgcctcagcctcggagtagctgggactacaggtgtgcgccaccacacacagctaattattgtatttttagtagagacagtgtttcaccatgttggccaggatggtctcaatctcctgacctcgtgatccacccaccttggcctcccaaagtgctaggattacaggcatgagccaccgcacccggccttttttattttataaagacagagtcttgctcttgtcacccaggttggagtgcaatggtgtgattttggctcactgcaacctctgcctcctgggttcaagtgattctcctgcctcagcctcccgagtagctgggactacagacacccaccaccacacccagctaatttttgtatttttagtagagacagcattttaccatattgaccaggctggtctcgaactcctgacctcaggtgatccacctgccttggcctcccaaagtgctgggattacaggtgtgagccaccgcacctggccctttttgtttttttcagtctgaatctttttcattcagcctgctgcccaggctggagtgcagtggcgtgatctcaattcactgcaacctccacctcccgggttcaagcaactctcctgccttagctccctgagtagctgggactatgggcgtgcaccaccaagcccggctagtttttgtattttctgtagaaacggggtttcgccatgttggtcagactggtctcgaactcctgacctcaagtgacccacccgcctcagcctcccaaagtgctgggattacaggaataagccaccatgctcggccatatttggtgatcttatttttgagtgtcatgacgatgcaaagaggacatgttggagccaggagactgatggaatcctgccctcacccaatttattggtatctgtcataagtgtacaagtcccatattcatgtcagcagagaaagtgctgaacagggccgggcgcggtggctcacgcctatgatcccagcactttgggaggccgaggcgggcggatcacaaggtcaggaaatcaagaccatcctgctaacatggtgaaaccccgtctctactaaaaaatacaaaaaattagccgggcgtcgtggcgggcgcctgtagtcccagctactcgggaggctgaggcaggagaatggcgtgaaccggagaggcggagcttgcagtgagcccagatcacgccactgcactccagcctgggcgacagagcgagactccgcctcaaaaaaaaaaaagaaaagaaaagaaaagaaaagaaaagaaagtgctgaacagatatttgctgaattaatcagtctcaatttccccattttcaaagatcttagtacctgcttccttctaccatatgttagcaagaagattaaatataaaattaatatacttattactattttctgagtttgttggaagcagagagtgtggtaagtcagcctcctctaaaacggagccaaacacagccactttggatggtggcgtggaactgctgacaggagtgagctttacttatggagaactaatctaagcctgagaattgcactgtaacttctatccttctccttcctggagggggagggggggaaatacaggcatttttctttttctttttcttttgagatggaatcttgctctgttgcccagactggagtgtggtagtgcaatctcggctcactgcaacctccgcctcctggtttcaagcaattctcttgcctcagcctcctgagtagctgggattacaggcacctgccaccacacccggctaattttttttttttttgagacggagtctcgctctgtcgcccagactggagtgcagtggcacgatctcggctcactgcaagctccgccccctgggttcatgccattctcctgcctcagcttcccaagtagctaggaatacaggtgcccgccactatgaccggctaattttttgtatatttagtagagatgggatttcaccgtgttagccaggatggtctcgaactcctgacctcatcatccgcctgcctcggccttccaaagtgctgggattacaggcatgagccaccacatctggccccagagtagcttttttttttttgagacggagttttgctcttgttgcccagactggagtgcggtggcatgatctcggctcaccgtaacctctgcctcccgggttcaagcgattctcctgcctcaggctcccgagtagctgggattacaggcatgcaccaccatgcccagctaattttgtatttttagtagagatggggtttcttcatgttggtcaggctggtcttgaactccccacctcaggtgatccacctgcctcagccttccaaagtgctgggattacagttgtgagccactgtgcccagcccagagtagtaatttttgtttgtttgttttttgagatggagtcttgctttgttgccaggctggagtgcagtggtgtgatcttggctcactgcaacctctacctcccaggttcaagtagctgggaatacaggtgcgtgccaccacggccagctaattttttgtatttttcagtagagatggggttttaccgtgtaagccaggatggtctggatctcctgaccttgtgatccacccgtctcggcctccccaagtgctgggattacaggcgtgagccaccacacccagcccagagtagctattttttaagtgtgtaagaattgggcccctactgccttaggtgggacaaaccagtgtggggattttcactggagatgtttttgatcagtgctataaactggatttgctgcttcaatctcaagggggttttcagacagggtctggccagggaactccagagttctaattaacttctaattggccatttaaataataatatctcggtctgtccaatgctcagagatcctgaaggattttatctaatgaagaaggtattttgcttcttactagtgtcccaggcttatgtggagcttaattaatgttcattgagtgccatttgcttctggtgaccctgaaatcctgttgcctctccgaaatagactcaaatatcttgtctccaactggacaagaacacagtcaagccaagtagcttggctataattggtctatgactaacatggcagccttatatcaaaggcttattgctagaatccctagaaaaagaaatgaggtaaaagtacagcaggcgccatgtctggccacaaagacggcaagaagcccctgaaacagcacaaggagcaggccgaggagatgaatgactaagatgacgcttttaagcagaaacaaaaagagaagcaggccaggccaggcatggttgctcatggttgtagtcccagcactttgggaggctgaggctagaagatcacttgagtccaggagttcgagactagcctggccaaaatggcaaaaccctgtctctaccaaaaattttaaaaattagccaagtgtgctggtgtacacctgtagtcccaggacacctatagtcaggaggctgaggtgggagtatcacttgagcccaggagatcaaggctgcaatgaaccatgatcacaccattgtgagatccatgatctagcctgggtgacagagtgagatcctgtctcaaaaaaaaaaaaaaaaaaaagggaagcaaaaaaccaaaccaaaacaaaacaaaagataacataaaaacaaaacaaaaggaagcaaaaagccaggcatggtggctcacatctgtaatcccagcactttggaaagccaaggcaagcagatcacctgaggtcagcagttcgagaccagccagaccaacatggagaaaccctgtttatactaaaaaaaaatatatatatatatatacaaatacaaaaattagccaggcgtggtggtgcatgcctgtaatcctagctattctggaggctgaggcaggagaatcgcttgaacccgaggggcagaggttgtggtgagccaagatcgcactattgcactccagcctgggcaacaagagcaaaactccgtctcaaaaagaaaaaaaaaaaaaaaggaagcaaaagaaactcaagagctaaaaaatggaggtctcagggacgggcctggctgaggtgggaagatcacttgaggcgggaagttcaagaccagcctgggcaacatagagagacactgtctctacaaaaaagtgaaaaaattagcaacgcatagtggtgcgtgcctatagccctagttactcagaagactgaggtaagaggatcccttgaggcctggagtttgagattacattgagctatgattataccactgcactccagcctgcacaatagagcaagaataaacttttggctgggcattgtagctcatgcctgtaatcccagcactttgggaggctgaggtgagtagatcatgaggtcaggagttcaagaccagcctgatcaatatggtgaaaccccgtctctaataaaaatacaaaaaaaattagctgagccttgtgtcaggcacctgtaatcccagctactcaggaggctgaggtaggagaattgcttgaacctgggaggcagagtttgcagtgagccaagatcgcgccattgcactctgggtgtgcaaaggagagtctctggagcaagactccggagcaagactccatgttaaaaaaaaaaaaaaaaaaaagaataaacctttaaagaaaaaaaagaaaaatcatacagtggcttatcatctcccagcccagaatcctaccagagttggccctttggtctttgttctactctaaattatgtatcacctggaattaaaccaactgatttaaaaaaagaagaagaaaaaaagaaaggaaatcaggttcttaaagcccagcactagtagagctcagacaagttacctttttatcctaatgaagtaggaagggcaccactatgtacctagcacccaggaatgtttaacctttactcacgttctgcctggagaaggaagccaggctgcaatgcagggatctggctcctgaactctgtcaaggctgaggcagggctggataagaaatctctacccccatgtcaggcttgtgaccagcctcctccctcatcttgcacaaccttgatccaggggcaggaactgatacactggcctttggaagccgtaggactttgaacttcgttgaacttcggagctgaagggctgaacagagctggtgccaaacccacttttagtaaccccatagtttttttttccccccacttaacaacatatcaaagacattttttccacaacaatacttacatatttactttatccttgttgtgtctgtatttatcacatggatgcacaacaatttattttggatattcccttcctacaaatatttacgtaatatgttttaatttttttatattaaaaatatattttaaaaatagagacagggtctcgctatgttgcccgggctggtctcaaactgctggccccaagcaatcctccaatcttggcctcccaaagtgctgggattacaggcgtgagccaccacgctcagctgatttgggtaatctaaacaacacagtaaacagccttgtcccttttctattatttcctaaaactaagttccttacaattgctgaatcagaagggatggacatttaacattttcatatatattatcaaattgccttatatatatgttttctttttctttctcctacttgtattttgaagtatatatatttaaatttgtaatactttttttttttttttttttttttttttagagacagggtcttgctttgtcatccaggctggagtgcagtggcacaatcatagctaactgtaacctcaaactcctgggcttcagtgatcctcccacctcagcctccattgtagctaggactacaggtgcacaacacctctatttttgtagagacagggtctcagtatgctgccctggcctcatgtgatcctcccacccagcctatgctcccttcttatcaaaccacatgtagttcctggaatgtaacatcctgcttcattcctccacaacttggcacatatcaattcctctgctcagactaccattctccagcttcctttaactcccttcccaacaacagtctatttttccaaaaaactcctggcacatggtagatatcattaaatattggtcagtcgaatgaataaatcaatcacttctacccacttttagcacagagcagcctctgtgggtgacatgtgcaacagaagaccgtaaaattaggaaatgcagttgacaggggaaatccgcaccgccatatgacaggctgtctgcttccgtcagcggttattcccttgcctgcctgccctagcccacaccacttcctcctcttccccccacccccgactttccagtcccagtccattggtttcagttggctgcagcctcctttccaccacttcactgcagcattgatctttgtaactgccataatagaactgattcctaccctgatataactatagcttctatgccaacagctgtagctctacataatcatgtgtgtgcatgtacaggtgtaagtgtgcttgcatgggtgcatgagtgtatatgtgcgcatgcatgagagagaaagagactgcaaccctgtgcagagtcctcctcctctattctggagttctgtctctgtcagggatgggtggcagtttgtagtagtttcacttgaaggggtaggagaagagggaacagttttcctatttcaacctagcttgcagcagcctattttgaatataaataataatcattaccactcattaaatctctatgtaccaccaggcactttacatccattgtttcatttaatccccaaacaactcaaaaaagtaagcatggccagcttggtggctcatgcctataactgcagcacttttgggaggccaaggtgggaggatcgcttgagcccaggagtttgagatcacctgggcaaatatagtaagacctcgtctctacaaaaaataaacaaaattagctgagcacggtggtggacagttgtagtcccagctacttgggaggctgaggtgggaggattgtttgagcctgggaagtcaacactgcagtgagccgtgatcatgccactgcactctagcctagatgatagagtaagactctgtctcaaaaaataaataacatatatttaagaaattaagaattttgctttggtgcataatcaggaaaaaagcaataaatggttctgaggaccaagtcactctggtgggattggggattggagctcagatcccaaaagatgaggtagctgactgcataatgcagtccccagggcctggtgccagccttgctggctctcagcagccacttgaatgggaccacaggatttgaggggcctgagagggagaataatgcctcagtgcacaggcccagggccatttccagtagctacctgtgtagggactttcactacttgggggaaatgagtcaggtcttgtgaagttggtttcagagctggccagagtttaggaatgattcggccctaataggggattagaaggaacttaaatatgttccttaaatgtgcgctcattacaatgtcactagaaggtccacgcataaagtcaagatcttgttcaggcccccttatcgccctgcttcgtatatgggtcctctgggaacctgaactctgttcttcattctcattcagacagcctagcctacagtaagcaggaagtgccagcaaataagtcactggggtagctccgggacaaactcagagacttggacagcaggcatttatttcttcatctcttctacctggccacattcctcttagggtctagaaacccctgccaaatgctaaaacgttcagtgaggggtgctgagaacttaacctttccctccctcctgcctagttgtccagaaagggatgctcccttctccttcctagacagccattctcactttctagctcttcatctgtttcagcctcctctgtcgccacacaaggccaaccctgcacacacacataggtatgcattctgcctgttctacattagcccctctggactgtgaaccttgaaggcagagaacaaggctggtcctttttttttttttaaatttttgtgatgaaatctcactctgtcactcagactggagtgcagtgaacaatcttggctcattgcaacctgcacctcctgggttcaagcacttctcctgagtagctgggattacaggtgcctgccatcacacccggctaatttttgtattttttgtttgtttgttttgatttttgtttttccgtcggatttatttcctcacattacagcatacttttcatatgagcctacacatatcagatggtaactctggaatgggtgagtctgtgacactgacccagcacagctgtgagggtgatgacaggcaggttagcaggtgcgcacagaggggctttcttctaaattggaggtcaggttagatgattcagctccgcagagctcattcagtgctccgtcttctggtggcttcccttgatttctaataaaagctgcagatggagtcactcagttccacatggctggggaagcctcagcaaacttacaatcaaggtggaaggcacctcttcacagggtggcaggaaagagaatgagtgcctaatttttctatttttagtagagatggggttccaccatgttggccagtctcgtctcaaactcctaacctcaagtgatcttcccaccttggctcccaaagtgctgggattgcaggtgtgagccaccgtgcccggccaaggctggtccatttttgtgcccagtgaaaggactaggacatagtatatgctaagtaaatgttagctaaatgtatgatccatgatcccccccacaattatcccattattttttttctttttctttttcttttttttttgagacaagagtctcgctctgtcacccaggctggagtgcagtggcatgatcttgacccactgcaaccttcaccatccaagttcaagggattctcatgcctcagcctccaaagtagctgagattacaggcgtgcaccaccatgcccagttaattttttgtatttttactagagatggggtttcaccatgttggccaggcagatcttaaactcctaatctcaagtgatccacccgtcttggcctcccaaagtgctgggattacaagcttgagccaccgcgcccagcttccctccatcattttctccactttcatatctcctgtctcttccactggacccttccccgctgcttagcacgtggtcatgctcgcatttcccacatagtgaaaaactctccctccatctgcatcaagtcactattaacccataacttttcttcctcttacttttttttttttttttgagacagggtctcactcagttgcctaggctttgtagtggtgggtgcgatcacagctcactgcagccttggcctcccaggctcaggtgatcctcccacctcagcttcccaagtagctgagaacacaggtgcatgctaccatacccagctaatttttgtattttttgtagagatgggatttcaccatgttgcccaggctgatcttgagaacccctgggttcaagtgatctgcctgccttggcctcccaaagtgctgggattacaggcatgagccactgtccctggcctttactgtatccttgagaagtccttcctctgattctccatgtcttcatctcctgttcattcctgaattcaggtcatctggctcaggattaaccattccactgaaggagctctccacattggtcaccaatgatcttcatgtccaccaaatccaaaagtttctgctgtggatatcagacaacatgaatataaagactatggtaagtagtggatgctcaataaagtggttgttcttatattcttatttgctcacttctgtttttttgttttttaaaaattattatttatttttgagatggagtcttgctctgtcgcccagactggagtgcagtggcaccatctcggctcactgcaacctctgcctcccaggttcaagcaattctcctgcctcagcctctcaagtagctgggattacaggcacctgccaccatgcccagctaatttttgtatttttagtattgatgggatttcaccatgttggccaggctggcctcgaactcctgacctcaggtgattggcccgcctcggtctcccaaagtcctgggattacgggtgtgagccaccacacctggcctgtttgtttttagacagggtccttggtaaaaagaatttgaggggaaaaaaaaaaagagacaaggttttgctctgttgtctaggctggagtagaatggcacaatcatgacacactacagcctcgaagacctgggctccagcaatcctcccacttcagtctcctaaacacctgggactataggtgtgcaccaccgtgcccagctaatttttatattttttgtagagatgaggtttcactatattgcccaggctggtcttgaactactaggctcaagtgatcctcccttcttagtcttccacagtgccgggattacagccatgagctactgtgcctggcctgtaggttcttcttttacgtatttttttttttttgagacggagacttgctctgtcacccaggctggagtgtagtggtgcggctcactgcaacctctgcctcctgggttcaggcgattctcctccctcaacctcctgagtagctgggactgcaggcgcgtgccaccatgcccagctaatttttgtatttttagtaaagacggggtttcaccatattggccaggctggtctcgaactcttgacctcgtgatctgcccgccttgtgctgggattacaggcgtgagctactgcgcccggcccctttttttttgagacagagttttgctcttgctgcccaggctagagtgcaatggcacaatcttgccaaacgcaacctctgcctcctgggttcaagtgattcttctgcctcagcctcctgagtagctgggattacagggatgtgccacctcacctggctaattttgtatttttagtagagatgggggttctccatgttggtcaggctggtctcgaactcctcacctcaggtgatctgcctacctcggcctcccaaagtgctgggattacaggcgtgagccaccatgcctggcctattttatttttttttgaatacaggctggagtgcagtggcgcgatctcagctcactgcaacctccgcctccctggttcaagtgattctgttgcctcaggctcccaagtagctgggattacaggctcgcatcaatacatccagctaatttttttttttttttttttttttttttttttttgaggggcagtctcgctttgtcaagaggctagagtgcagtggctcgatctcggctcactgaaacctccgcctcccgggttcaagcgattctcctgcctcagcgtcctgagtatctgggactacaggtgcgcgccacgacgcctagctgaattttttttttttttttttttgagatggagtttcactcttgttgcccaggctggagcgcaatggcgcaatctcagctcaccacaacctccgcctcccaggttcaagcaattctcctgcctcagcctcccgagtagctgggattacaggcatgtgccaccacgcccggctaattttgtatttttaatagagacggggattctccatgttggtcaggctggtctctaactcctgacctcaggtgatccgcctgcctcggcctcccagagtgctggaattacaggcgtgagccaccgcacccggccaagtctgattgtttttatgaggatggcctgatacattagacatttggtccaccaggacatcacttatgttggggagacagtgggggataacgggggtggggaaatgggaagagttgctctcagggagtaaaggtgtggggaaaagcagagccaaaccgagataaccctgtgtcttgcccatccctgccaaggaaaacacatacatgcaagacacctctccactcaaacagcacagactatttcaaatcgcagatgtgtcatttaattaatacctgtgtgactttgggaaaattataaatagccttaaccttgctgagtctgtccaatctgtaaaatggattatgaatactttcctcacaggcttatggtgtgctggtaatatcttcctctggatggcagttacattgcaaaaaataatttaaaaaaaagaactgtatacatttaagatttatacattttctttcttcctggatgtagaataggaaaaaaagaaaaagaaaaaaaaaaagactggtgtattttctgtatatatgctatactacgatttttaaaaaggcttgtacgaaataaaagaatattccttttcggccaggcgcagtggctcactcctgtaatcccagcactttgggagtccagggcgggtggatcacgaggtcaggagttcaagaccagcctggccaagatggcgaaaccccgtctctactaaaaaatacaaaaaaattagccaggcgtggtatcgggcgcctgtaatcccagctactcgggaggctgaggcagagaattgcttgaaccagggaggcggaggttgcagtgagccaagatcgcgccactgcactccagcctgggcgagagcgagattccgtctcaaaaaaaaaaaaaaaaaaggaatattccttttccaggattattatgaagattcaataaaaccatgtttattaagtgttaagcacagtgcctggcacataacgtgctgggcggggtctatggtccagacagggggaccaggcactttccagcgcctggatctgcagacgcgaggtcttctgtattctggccaatcttggtgttgcagctgctctctgggcctcagtttgcttgaactaaatgtaacggggccaacttaggtgaactttgggaatccagccaacctgactttagggagagtatggagccacggatggcattgtgaatccggagggccgacaccagaagaacctgcaacgtggcatctgctaccttacttcccccggaaaagcgcctgcggcggcgcctaggcgcgcggtgcaatgtgggc

SEQ ID NO: 5 - DNA, 24142 bp, Homo sapiens
  source 1..24142: mol_type = genomic DNA
gccgggcggcggctggtgacgcagcaaaatgcccgggccttgccgccgcgcgtgcgcagtccttgtttttgggctccggcgcggctttctagagatatctacggaaagggcggggcctcccagctccttcgcgatccttcctggcgccggccccgcccttcgtccgccaccccacccccactccgccccctgctagtccctacgcgttcccgcctctcctcccgctctgtcttctcgcgttagaaaaccgttagtggcccaactgccctgccatgcgccttcggcgatttctctgagaaacggttgcgttgacagtgggaggcaaggctgaggcggagggacacggagtcgggatcctggagaactgaaatggcgcgttggttactcttgagggagtaaatcagctcactgagtcatcattccaccacaaaaacctgtaaaaagcctgtgctgtggaaaagggctcacttcagaatcaagggcctgggtccacctcgccatttttctgaaggttggaggctctgtccagggacagcggaaattcgaggttcttggcggaggaggagcatttgccaattggggcgggcttttgcccctaccttgagaccttagtgctgtagatgagccaggaaaggtatttgaggggagagtctaggtttctgggaatggagagagtaggacattgtgtgtttccacgcacatcgtttgggggagaaggggttccccgtcgctttgtgcaacatattagtagggtgtagtaggggtagcaaccaagagacccaaaacagtcctgcttaagaagaagccgcttaacggtggattggggaaaaatactctagtgcgctgcattttgaacaggaaggaggtttaacgaaagtgctataaaacctttctgaagaacggggcttcataataagcactaaggtatttagtgtctgtgttgtgcaggacaggtagtgggagctacgaaacctggaccctagctcaaatgccattaaaaagggaaattgggcggggcgcccgtggctcacgcctataatcccagtactttgggaagttcggtgggcggatcacctgaggtcaggagttcaggaccagcccacccaacatggtgaaaccccatctctactaaaaatactaattagcagggcgtggtggcacttgcctgtaatgccaaatactagctagtatgaggcgggaggatagctcgaaccccgaacccgggaggcggaggttgcagtgagcactccaacttgggcgacagagtgagaccctgtgtcaaaaaaaaaaaaaaaaagggaaggggccgggcgcggtggctcacgcctgtaatcccagcacttcgggaggcagaggcgggcagatcacgaggtcaggagattgagcccatcctggctaacatggtgaaaccccgtctctactaaaaataaaaaaaaatcagccgggtgtggtggcacacgcctctagtcccagctactcgggagagcgagacaggagaatcgcttgaacccgggaggcggaggttgcagtgagccgagatcgcgccactgcacttcagcctgggcgacagagtgagtctgggtctcaaaaaaaaaaaaaaaaaaaaaaggaagttaacttggcgcactggttcacgcctgtaatcccaacactttgggaggccgaggccagtggattactggagatcaggagttcaagaccagcttggctaacatggtgaaacccccgtccctactaaaaatacaaaatatttggccaggagcagtagctgacgcctgtaatcccaatactttgggaggccgaggcgggcggatcacgaggtcaggagttcaagaccagactggccaacatggcaaaaccccatctctactaaaaatacaaaaattagctgggcgtggtgtgcctataatcccaggtactcaggagtcttaggtgggagaatctcttgaacccggaagacgcaggttgcagcgagtctagattgtaccactgtactccagcttggtgacagtgagactctgtgtaaaaaaaaaaaaaaaaaaaaaaaaaagaaaagaaaaaataggtcacaaccaaataatggtggcactgaatgtcaggctaaatttttttttttttttatgtttttgagacagggtctcactttaactcaggctggagtgcagtagtggtatcacaggtcactgcagtcttgacttctgggtccaattgatcctcctgtaacgactgaatttaaatatatgtccagagtcacaaattgcaaaagagaactttatagactgatttgagcaattaaaaaaattgttaattgaaggtgagaacaatcctattttataagggggctttctaataagataacaacttagaagttgttaattttgtattaggaagtcaaagggaaagggtgcaaaatggtaaaaatgtctcctggaggccgggcgcggtggctcacgcctgtaatcccagcattttgggaggccgaggcgggcgaatcacctgaggttgggagttcgagaccagcctgaccaacatggagaaaccccatctctactaaaaaacacaaaattagccgggcgtggtggcgcatgcctggaatcccagctaccagggaggctgaggcaggagaattgcttgaacccaggaggtggaggttgcagtgagccgagatcatgccattgcactacagcctgggcgacaagagcaaaactctgtctcaaaaaaagaaaagtctcctgggtatagggaataaaaattgggccgggcgcggtgtctcatgcctgtaatcccagcactttgggaggccaaggcaggtggatcacctgaggtcaggagctcaagaccagcctggccaacatggcaaaaccccatctctactaaaaatacaaaaattagctgggcgtggtggcaggcgcctgtaatcccagctacttgggaggcggaggcagaattgcttgaacctgggcggcagaggttgcagtgagccgagatcatgccattgcactccagcctgggtgacaagagcgaaactccatctcaaaaaaaaaaaaaaaaaaaaaaaaagacggggcacgcgtggctcacgcctgtaatcccagcactttgggaggctgagatgggtggatcatgaggtcaggagttcaagaccagcctggccaagacggtgaaaccctgtctctactaaaaatacaaaaattagccaggcttggtggtgggtgcttgtaatcccagctacttgggaggctgtggcagaacctgtgaggtggaggtggcagtgagccgagatctccccactgcactccagcctgggtgacgagactctgtctcaaaaaaaaaaaaaaattggccataaaccttaaaagagatattatgacccagcttaatataaacattactagtagccacctgtagtcccagctacttgagaggctgaggtgggaggattgcttaagcccaagagttcaagtccagcctgggcaacgtagggagaccctgtctttgaaaaaataaattttttttttttttagatggagtctctgtcgcccaggctggaatgcagtggcacgatctctttttagtagagacggggtttcaccatgttcaccaggctggtcttgaactcctgacctcaggtgatctgcctgcctcggcctcccaaagtgctgggattacagacgtgagccactgcgcccagcaaataaaatcatttttaaaattacttatgttttaggccaagcatggtggctcatgcctgtaatgccaacattttgggaggccaaggtgggaggatgtcttgaggccaggagttcaaaacctgcctgggtggccatgagcagtgattcacacctgtaatcccagcactttgggaggctgaggcaggtagatcatctgaggtcaggagtcaagaccagcctggccaacatggtgaaacctcatatttactaaaaatacaaaaattagctgggtgtggtggtacatgcctgcagtcccagctatttgggaggctgaggcaggagaatggcttgcacccaggaggcagaggttgcagtgagccaagaatgcaccactgcacttgccactgtactccagccgaaatgacagagcgagactctgtctcaaaacagaaacaggccaggtgtggtggctcatgcctgtaagcccagcactttgggaggccgaggcgggtggatcacgaggtcaggagattgagaccatcctggctaacatggtgaaaccccatctctactaaaaatacaaaaaattagccaggcgtggtggcttcgcctgtagtcccaactactcgggaggctgaggcaggagaatggtgtagacccaggagacggagcttgcagtgagccgagatcacgccactgcacttcagcctgggcgacagagcgagactccgtctcaaaacaaaacaaaaaacagaaacaaaaacaaaaacctggccgggtgtggtggctcctgcctgtaattccagcactttgggaggccaaggtgggcagatcacctgaagtcaggagttcgagaccagcctggccaacatggtgaaaccttgtctctactaaaaatacaaaaattagctgggcgtggtggcaggcgcctgtaattccagctacttgggaggctgaggcaggagaatcgcttgaacctgggaggtggaggttgcagtgagccaagatggcaccactgcactccagcctgggcggcagcgagactccgtctcaaaaaaaaaaaaaaaaaaaaaaaaaagctatttacccgctgggcgctgtaatcccagcactttgggagaccgaggcgggtggatcacaaggtcaggagttcaagaccagcctggccaatatggtaaaaccccatctctactaaaaatacaaaaattagccaggcgtggtggcgcgtgcctgtagtcccagctactcaggaggcagaggcaggagaaccacttgaagccgggaggcagaggttgcagtgagccgaaatagcaccactgcactccagcctgggcaacagagtgagagtctgtctcaaaaaaaaaaggtcagaaggtatttacctgtaaaaatgacatgcattgaatgaagtatttttaaaatggtggccgggcgcagtggctgaagcctgtaatcccagcactttgggaggctgaggcgggtggatcacaaggtcaggagatcgagaccatcctggctaacatggtgaaaccccatctctactaaaaatacaagaaattagctgggcgtcgtggcccatgcctgtagtcccagctactcgggaggctgaggcaggagagtcacttgaacccgggaggtggagttgcagtgagccaagattgcaccactgcactccagcctgggcaacagagcgagactctgtctcaaaaaataagtaaataatataaaaaataaaaatataaatggccttctgctgggcacggtggcttatgcctgtaaacccgggactttgggaggctgaggtgggtggatcacttgaggtcaggagtttgagacagcctggccaacatggcaaaacctgtttttcctaaaaatacaaaaattaggcccggcgcagtggctcacacctgtaatcccagcacttagggaggccaaggcaggtggatcatgaggtcaggagttcaagaccagcctgcgcaagatggtgaaacctcacctgtactaaaaatacaaaaattagctgggtgcagtggcaggtgcctggaatcccagctactcaggaggctgagacaggagaattgcttggacccaggcagcagaggttgcagtgagccgagatcacacgattgcactccagcctaggtgatagaggagtaaaactccatctcaaaaaaaaaattaaaaaaattagctgggcatggtgatacatgcctgtagttccagctacttgggaggctaaggcaggagaattacttgaacctgggaggtgtaggttgcagtgagctgagatcgtgctgttgcactgtcagtctgggcaacagagcaggactctgtctccaaaaaaaaaaaaaagatcttctaccccacgtctccaacctccaggctgtggcaatccccaggctgtggataggtactggtccatggcctattaggaaagcagccacacagcaagagatgaacgctccatgagccagcattactgcctgagctctacctcctgtcagatcagtgatggcattagattctcataggtatgcgaaccctattgtgaactgcacattcaagggatctaggttgtgctcgccttatgagaatctttttttttttttttttttttgatacaggatctctgtcacccagggtggagtgccgtggcgagatcttggctcactgcaacctccgcctcctgggttcaagcgattctcctgcctcagcctcctgagtagctgggattacaggtgcttgccaccatgcccggctaattttttatttattttttttagacggagtgtcgctctatcaccaggctggagtgcagtggtgcgatctcagctcactgcaagcttcgcctcccaggttgacgccattctccttcctcagcctccctagtagctgggactacaggtgcctgccaccatgtctggctaatttttgtatttttagtagagatggggtttcactgtgttagccaggatggtctcgatcacctctacttcgtgatctgcctgcctcggcctcccaaagtgctgggattacgggcgtgagccactgcgccctgctgtgcccggctaatttttgtatttttagtagagatggggttttgtgatgttggccaggctggtcttgtactcccaacctcaggtgatccaccagccttggcctcccaaagtgctaagatgacaggcgtgagccactgcgcctggccacttcgagaatctaatgcttgatggaacggtttcattccaaaactattccctcaccatggaaaaacttgtcttgcacaaaacccatttctggtgccaaaaactttgaggaccactgttctaccccaaacatcaccttatcttatgtttacatctgaagtttatattatagaaaataagaccattgagtgggcagggtggctcattcctgtgatcccagtacttagggggccaagatgggaggtttgcttgagcccaggagtttgagaccagcctggactacatagggagagcctgtcactacaaaaaataaaaaattagccagatgtggtatatggtgatgctgggattacagacatgagtcactgtgcctggcctggtggacacatgtcttgagtgagaccactgccgtcatgcttttctcactattctgagtccctggcacatagtagatgctcaaaaatgtttgttaaatatattaataatttagctgtaatggagaaaaactatggacaaaccagtcatggtagtttaggcaagagattagacccagaactaaagcagtggcagtaggagtggacagataaagctaacccagaattttgatgtatttctaactttgaagaccagttagaaaaggaatcagataaggaggtttctagtttgggagactgggtgaaatgcgacaccattagctgagactgaaagggacagatggaggagtagaaatataatttaatatagtttatatttagcagttgaggaaagggttttttttgttgcttgtttaggaatgggatgttttatcatgtttgatgggtgagaaaagaagccagtggaaagagaggaggggtttaagaagacaggataacagggagaacaaggtctggtatgaggcaggagaggataaaagccaaaggaaacaaagagaaggcataggaagaaggaaggagatgaaagtctaagggaagggcttttaaacttgattctggctctcgtgctctctctctcttgctctgtatctgtgtgtatacacacacaaaaagaatatacatacatacatacacacacacacacacacatatatatacattttttttgagacacatcctctgttgctcagcctggagtgtagtgatgtgattatggctcactgcagccttgacctccccaggctcaggtgatcctcccacctcagtctcccaagtagctgatactacaggtgtgcactaccacacccagctaatttaaaaaagtattttgtagagatggggttttgccatgttgctcaggctggtctccaactcctgggttcaagtgatccatctatctcggcctcccaaagtgctgggattacaggcttgagccactgtgcctgcccactcaatatgtatttgttgaatgaatgaattaggtgccagatccatgacagaaatatattttacagcaaaggaactcagatcacttaagcttttcgtttgcaggtttgttgggtactacaacctggaagttaatatctaactcccatgtagtatctaacttgtgaaatcttgttttgcttccatctctaaagaagtgtccttctggaggttgcagggagccgagatcatgacactgcccttcagcctggacaatagattcaaatcttgtctcaattaaaaaaataataataataaatttttttaaaaagtgtcctacttcaagcacaggatgaccttttctgcgtgtgttctctttttttgagacggagttttgctcttgttgtccaggctggagtgcagtggcacgatctcagcttgctgcaacctccgcctctcacattcaagcagttcttctgccttagcctcccgcatagctgggattataggcatgcaccaccacacctggctaattttttgtatttttagtagagacagggttttgacatgttgggcaggctggtctcgaactcctgacctcaggtgatctgcccacctcagcctcccaaaatgctgggattacagtgtgagccaccgtgcctggccatctgcatgtgttcttaatgtcattgtctctggccttttccaggagtatgaatcttcagtttattcccttaatctttccattttgtctacaaatatgcttaggtgttccattctaaaaagcaagacaaaacaaacctttcctcaggcctccttttttttttttttgagatggagtctcgctctgtcgcccaggctagagtacagtggcgcgatcttggctcactgcaagctctgcctcccgggttcaggccattctcctgcctcagcctcccgagtagctgggactacaggtgcccgccacaatgcccggcaaactttttgtacttttagtagagacggggtttcactgtgttagccaggatggtctcaatctcctcacctcatgatccgcccacctcgctcctaaaatgctgggattacaggtgtgagccaccgcgcctggccacctcaggcctcctttttatgctgtggtctgttaaactttttccacatttctaagattactccataaactattctcactaaaggtacttctttgctgtccattcattggttcccactcccccaagctaccaaagtggatacactgaaagccactcaattgtcaaattcaaaggcccttttcttcaggctgggcactgtggcccatgcctataatcctattattttgggtaggcagatcgcctgagcccaggagttcaagatcagcctgggcaacatggtgaaaacctgtctctaccaaacccccacccccccaaaattagccatgtggcctggtgtggtggctcacgcctgtaatcccagcactttgggaggccgaggtgggcagatcacctgaggtcaggagttcgagaccagcctgaccaatatggtgaaaccttgtctctactaaaaatacaaaaattagctgggcgtggtggcgggcgcctgtagtcccagctacttgggaggctgagacagaagaattgcctgaccctgggaggcagaggttgcagtgagctgagatcatgccactgcactccagcactccagcctgggcaacagagtgagactccgtctcaaaaaaaaaaaaaaaaattagctgggcatggtgactctcatctgtaatcccaactacttgggaggctgaggcaggagaatagcttgaaaccaggaggtggaggttgcagtgagctgagatcgcaccactgcactccagcctgggcaacagagtgagactccgtttcacaaaaaaaacaaaaacaaaaacagttagccgtgtgtggtgcagtcctagctacttaggaggctgaggtgggtggatcacttgagcccaggaggtcaaggtggaaatgagctgagatcatgccatgtactccagcttgggtgacagagacactgtgtcaaaaaaaaaaaaaaaaaaaaggccgtttttttcagtctctgttcttttttttttttttttgagacagagtttagctgttgttacccaggctggaatacagtggttcactgcatcctccgcctcctgggttctgggttcaagtgattcttctgtctcagcctcctgtgtagatgggattacaggtgtgtgccaccatgctgagctaattgttgtatttttagtagagacgaggtttcaccatgttggccaggctggtctcgaactcctgacctcaagtgatccacccgccttggtgtcccaaagtgctgggattacaggcatgagccactgcacccaacctgttcttggtctctttgaagtgttaccacagagggatccctattcagaccccaggagagggttcttgagcctcaagcaagaaagaatttggacacatccatatggtaaagtgaaagcaagtttattaggaaataaagaaataaaagaatggctacttcatagccagagcagccccgagggctactggttgactacttttatgattatttattgattatatgctaaataaggggtggattatttatgagctttctgggaaaggcatggcaattccctgaactgaaggctcctcccctttttagacttattgggtaacttcctgactttgccatgacatctgtaaaccttcatggtgctggtgggtgtgtcttttggcatgctaatgaattataagcatatataatgaacagtgaggatgacgagggggtcatttttcatcaccatcttcgttttggtggtttttggccagcttctttaccacatgctggttttatcagcaaagtctttgtgacctgtaccttgggccaacctcctatttcatcctgtgacatagaatgcctgacatcctggcaatgcagcccaggaggtctaagcctcattttaccctgctcctattcaagatggagttgctctgtttcaaaggcctctgacagaagtttttgacatagttgaccagccattcctttttcttaaaaaggctgtcttctgtttctttgttatttattttttgagacagggtctcactctgtcacccaggatggagtgcagtggtgccatcttggctcactgcaacctccacctcatgggttcaagcgattctcctgcctgaacctcccaagtagctgtgagtacaggtgcgcaccgccacacctggctaatttttgtatttttagtagagacagggtttcatcatcttggccaagttggtctcgaactcctggcctaaggtgatccacctgcctcggcctcccaaagtgctgggattacagaggtgagccacggcacccggccttatttattttatctttttgagacagggtttcactctgtcacctgggctggagtgcagtggcacgatttctgctcaccatagccttgacctctgggcttgagtgatcctcccgccccagcctcccaagtaggtgggactacaggcgggcgccatcacacctggctaattttttttttgaaaaggagtttcgctcttgttgcccaggctggagttcaatggccgatctcggctcactgcaacctccgcctcccgggttcaagtgattctcctgcctcagcctccggagtagctgggattacaggggcgcaccaccacgcttggtaaatcaaggtcaaaaattattcttttttttttttttttttttgagatggagtctcgctgtattgcccaggctggagtgcagtggcgccatcttggctcactgcaagctccgcctcccgggttcacgccattctcctgcctcagcctcctgagtagctgggactacaggcacctgccaccacgcccggctaattttttgtatttttagtagagacggggtttcactgtgttagccaggatggtctggatctcctgacctcgtgatccgcctgcctcggcctcccaaagtgctgggattacaggcgtgagccactgccccccgccttttgtttttttttttttttttgagagggagttttgctttgtcgcccaggcgggtgtgcaatgacgcgatctcggcttgctgcaacctctgcctcccggcttcaagcgattctcttgcctcagccttcagagtagctgagattacaggtgcctgacaccaggctcggctaatttttgtttttttagtagagacaaggattcaccatattggccaggctggtctcaagctcctgacctcaagtgatccgcccgcctcagcctccaaaagtgctgctattacaggcatgagccaccgcgccggccccaagtgactctcatgggctggagacacaggctattatgaattgctaaccacagtctcttagtctcccttttaaataacaaaagaggaaatttcttcacttcctgacacaactctccatcatacagttggccaagagcagttagaaactttcatccttaatttgtgtcctttgggagttgctgctttcttagagtcattaaatgcatgtaggcagtgtctgcttcattaagccagatttataccaaatgaaaaggcagagaaagatctaatttaatgaatatttcttttttttttttttgagacggagtttcactcgtcacccaagctggagtgtaattcgtggtctccggtcactgcaacctccgcctcctgggttcaagcgattctccagcctcagccttccagcctcagcctcccaagtagctgggattacaggtgcccgccaccacacccagctaatatttgtatttttagtagagacaggggtttcaccatgttgggcaggctggtctcgaacccctgacctcaggtgatccaccctccttggcatcccaaagtactgggattgtaggcatgagccatcgcaccgggtcttaattaagatttctaagaactagatcaacaaatcagtgggactgtaaaggcaattcttgggacacaggtcaggaaactctgtgcttaattcagaaatcaaaaatccagcaatctcacttctgggtgttagaactgtagaaaataaaattttagaccgggcacggtggctcacgcctgtcatcccaggccgaggcgggtggatcacgaggtcaggagatcgagaccatcctggccaacatggtgaaaccccgtctttactaaaaatacaaaaattagcctggtgtggtggcgtgcgcctgtagtcccagctactccggaggctgagacaagagaatcacttgaacccgagaggcagaggttgtggtgagccgagattgggccattgaactccaggcttggcaacaagagcgaaactctgtctaaataaataaataaataaataaaaatataaaattttaagccccccaactgactgaatggatccccatttggccaagaggacccccaaactcctgaaaaagtagttcaggcaatgtggggaatgggaggcattgaatatgcctcattatactctcttccctttggagttctggcacaactgaccagcattaacattaaaacagagatcttggctgggcactatggctcatgcttgtaatcccagcactttgggaggccgagtcaggtggatcacctgaggtcaggagttcgagaccagcctggccaacatgatgaaaccccgtctctactaaaaatacaaaaaaatacaaaaaaataaaataaaaattagctgggtgtggtagtgcatgtctgtaatcccagctacttgggaggctgagacagaagaatcgcttgaatctgggaggcggaggttgcagtgaacagagatcacactaccgcacgccagcctgggcaacaagagcgaaactccgtctcagaaaaacagaaacaacaacaacaaaaagagttcgagactagcctggccaacatagcaaaaccccgtctctactaaaaatacaaatgttagcgggctgtggtggcgtgtgtctgtaatcccagctactcaggaggctgaggcaggagaattgcttgaaccagggagatggaggttgcagtgagtcaagatcctgccactgcactccagcccaagggacagagtgagagtctgtctcaaaaaaataaaataaataaaatgaataaaataaaacaagagattttaagactgacaaacgaggcggggtgcggtggctcaagcctgtaatcctagctctttgggaggtcaaggcaggccgatcaggggtttgagaccagcctggccaacatggtgaaaccctgtctctactaaaaatacaaaaatcagctgggcgtggtagtgcacacctgtaatccccgctactcaggagactgaggcaggagaatctcttgaacccgggcgacccaggttgctgtgagctgaaatcgaaccactgcactttagcctgggcaacagagtgagactctctcaaaaaaaaaaggggcggggggaaggacaaaagagactagcaaaattccaacatgatgctagtataacatcacataaaagataaaaaaaaggaaatcaaaatattttaccccaaaatatgtttctttgctatattttgaaatggtgggcacagtggctcttgcctgtaatcccagtactgggaggcagaggcaggagtattgcttgaagtcaggagttagagaccagcctgagcaacaaagacagactctgtctctataaaaataattaaattaaatataaataaataggccaggtgcggtggctcacgcctgtaatcccaccactttgggaggccgaggcgggcagatcacgaggtcaggagatcgagaccatccttgctaacacggtgaaaccccgtctctactaaaaatacaaaaaaaaaaaaaaaaaaaaattagccaggcgtggtggcaggcgcctgtagtcccagctactcaggaggctgaggcaggagaatggcgtgaacccaggaggcagagcttgcaataagccaagattgcaccactgcactccagcctgggcaagaatgagactgtctcaaaaaataaaaataaataaataaggcttggtgtggtggctcacgcctgtaatcccagcactttgggaggccgaggtgggcagatcctgaggtcaggagctcgagaccagcctaacatggtgaaaccccgtctctactaaaaatacaaaaattagccaggtgtggtggtgtgtgcctataatcccagctactcaggaggctgaggcagaagaatcacttgaacccaggaagcggaggttgccgtgagctgagatcgcgccactgcactccagcctgggcaacagagagagacttcctctaaaaaaaaaattaataaaaataaataaagtccctgccaagccatcttttgtgggggaaaatttgcatctgtaaataatctgtacgaacataactagatctttctcctaccaggtccacccaattctgaagagattaactgacagtctagttttttttgtttgtttgtttgtttttgagatggattcttgctctgttgcccaggctggagtgcagtggcaggatctcggctgactgcaatctctgcctccccggttcaagcagttcttctgcctcagcctccctagtagccaggacgacaggagcacgccacaacgtctggctaattttttttttttttttttttgaggagtctcgctttgtcgcccagtttggagtgcagtggtgcaatctcagctcactgcaacctctgcttcctgggttcaagcaattcttctgccttagcctccagagtagctgggactacaggcatgcatcaccaagcctggctaattttttttttttttttttgtatttttagtagagacggggtttctccatgttggtcaggctggtctcgaactcctgacctcaggtgatccacctgcctcagcctcccaaagtgctgggattacaggcatgagccaccatgcctggcctgtttgtctgtttttttgagatggaggtcccactctgtcacccaggctggagtgcaatggtgtgatctcagctcactgcaacctccacctcctgggttcaagagattctcccccatcagcctcctgagtagctgggattacaggcacctgccatcatgcctggctaattgtatttttgtagagaaggtatttcaccatgttggtcaggctggtgggagtgtagcagtgaggaggaccagaagtcactctggtggccatcttggttttggtgggatttagccggctcctttactgcaacctgtttcttttgtttgtttgtttttcgagatggagtcttactctgtctcccaggctggagtgcagcagggtgatctcggctcattgcaacctcagcctcctgggttccagcgattctcctgcctcagcctccggagtagctgggattacaggcgtgtgctaccacaactggctaatgtttgtatttttagtagagacagggttttgccatgttggccaggctgctctctaacttttgacctcaggtgatccattcgcctggacctcccaaagtgctgggattacaggcatgaaccgatgacaagcatgagccaatgtgtctggcctggtttaaacacttctgacatttaccccatcccttttataagagaacccttaatctttttttttttttgagacggagtctcactctttcacccaggctggagtgcagtggcacaatctccgctcactgcaacctccgcctcctgggttgaagcaattctcctgcctcagcctccagagtatctgggattacaggtgcccccaaccatgcctagctaatttttttgtatttttagtagacacgggctttcactatgttggccaggctggtcttgaactcctgactttgtgatccacctgccttggcctcccaaagtgctgggattacaggcgtgagccaccgtgcctggccttttttttttttcttttaattttaagcagagtcttgctctgttgcccaggcttgagtccagtggcacgatcttggctcactccaacctccgcctcctgagttcaagtgattctcctgcctcaacctcccgagtagctgggattacaagcatgcgccactacacccggctcatttttatatttttagtagagacggggattctccatgttggccagggtggtctcaaactcctgacctcaggtgatccacccacctcggctttccaaagtggtgagattgcaggcatgagccagtgcgcccagctttctgtaacttcttcaggatgaataagggcgatgatattcctgcctaactattaggatctcttgtgttcagggtagataggagctcagttagaaagcatcggtatggtgagggccatttataactctggagttccaacaaaaggtgatgtctggaagattagtaaatattcatttaagaaaacattcaggggtcgggcatggtggctcatgcctgtaatcccagcactttgggaggccaaggcgggcggatcacctgaggtcaggagtttgagaccagcctggccaacatggtgaagccccatctctactaaaaatgcaaaaattagctgggcatggtggcacaggtctgtagtcccagctactcgggaggtgaatcgcttgaacctgggaggcagaggttgcagtgagctgagattgcgccactacactccagcctgagtgacagagggagactccatctcaaaaaaaaaaaaaaaaaaaaaaagaggaatcatacaatatttgcccttttgtggatggctcatttcatttggcataatgccaacaatttatgtttcagcatgtgacagaatttctttcctttttaaggctgaataatatatatatatacacatatatataaaataatattttatacacacacacatactgtttttttttgagatggagttttggagttttgctcttgtcatccaggctggtgttcaatggcatgatcttggctcactgcaacctcagcctcccgggatcaaaggactctcctgcctcagcctccgaagtagctgggattaggggcataggcaaccacacctggctaattttgttttttttttttgagacagagtctcgctctgtcgcccaggctggagtgcagtggtgctgtgtcagctcactgcaacctccacctcccaggttgaagtgattcttgtgcctcagccacctggctaattttgttctgtatttttgtagagacagggtttcaccatgttggccaggctggtcttgaattcctgacctcaggtgatctgcctgcctcggcctgccagagtgttgggattacaggcgtgagccaccatgcccagtcttatatatttttttgacacaggatctcactttgtcacccagggtggagtgtagtggtgtgatcatggctcactacaacctctgcctcccagggtcaagggatcctcccacctccgcctccctagtagctgggacgtcccaccatgcctggctaattttgtaattttgtgtgtgtgtgtgtgtgtgtgtgtgtgtgtgtgtgtgtgtgtgttttagatggagtttcgctcttattgcccaggctggagtgcaatggtatgatcttggctcactgcaacctctgcctcctgggtttaagtgattcttctgcctcagcttcccaagtagcgggattatagacgtccacaaccatgcctggctcatttttgtatttttagtagtgaccagggtttcatcgtgttggccaggctggtcttgaactcctgacctcaggtgatccatccgccttggtctcccaaagtgctgggattataggcgtgagccccagtgcctggcgtgtgtggttttttttgtagagacagtgttttgccttttttttttttgggggggggatggagtctaactctgtctccctggctggagtgagtagtacagtggtgtgatcttggctcactgcaacctctgcctcccaggttcaagtgattctactacctcagactcctgagtagctgggactacaggcatgtgccaccacacttggctaattgttgtatttttagtagagaaggggtgtcactacgttggccaggcaggtctcgaactcctgacctcaggcgatctgcccgcctcggcctcccaaagtgctgagattcgccattttgcccaggctagtctcaaaagtcctgagctcaaacgatcctttatactcccaggccaagcgatcctggcttcccaaagtgctgggattacaggcatgagccactgcgcccggccgaataatattctcctgttgctatataccacatcttatttaaccattcaactgttgatggacacttgggtttctgctgcctttgtctattgtgaataatgctgttatgagtatggttgtaacactgagtccctgattgtaattcttttgggtttaaaaccagaagtggaattgcgggatcatatggtaaatctatttttaattttttgaggatctgccataccattccatagtggctgcaccatttaacattcccaccaacagtgcacaagtgttccaatttcttcacatccttgctaacactttttttttttgataagtagtcagcctaatgggttgaggtgacatctcattttttgttttaattacatttttttctttcttttttttttttgaggtgcagtttcgctctcgttgcccaggctggagtgcaatggtgcgatctcgactcatcacaacctccacttcctgggttcaagcgattctcctgccacagcctccctggtagctgggattacagacatgcgccaccacacccagctaattttttatttttagtagagatgggctttctccatgttggtcaggctggtctcgaactcccaacctcaggtgctccgcccaccttggcctcccaaagtgctgggattataggcatgagccaccgtgcctggccttttctcctttttttgagatggagttttgctcttgttgccctggctggagtgcaatggtgtgatctcggctcactgcaacctctgcctcccaggttcaagccattctcctgcctcagcctcctgagtagctgggattataggcataggccaccatgcccggctaattttgtatttttagtagagatggggtttctccatgttggtcaggttgttctcgaactcctgacctcagatgatccgcctgcctcagcctcccaaagtgctgaaattacaggtgtgagccactgcgcccggccctagcaccttttcatgtgctattggacatttatctatcttctttggataaatgtcttcatggccctttcacattttgaatcagagtttttgttgagttgtaggagttctttatatagtctggatattatccccttatcagatacatcatttgcaaatatttcctctaattccatgggctgtctttcttactgttttgttctttaatacacaagttttaaattttgatgaagtccaatttatctttttttcttttgttgcttgttttcttgcttttggtgtcatctaatagttttttattttttctgtcacccaggctacagtgcagtggtgtgatcttggctcactgtaacctccgcctcccaggttcaagcaattcttgtgcctcagcctcccaagtagctgggactacaggtgtgcgccaacatgttcagctaatttttgtatttcttggtttcgtcatgtcggccagggtcgttttgaactcttagccaggcatgagccactctgcttgactccaagaaccaaaaatttaatcattctagtaatagaggaagttatctgcatttcaattgtggtgaagcaaaagactgagaggaaggcccgttgcggtagctcaggcctataatctcagcactttgggaggtcgaggcaggaggatcatttgaggtcaggagtttgagaccagcctggacaacatggtgaagccttgtctctattaaaaatacaaaaatttgctgggcgtgtggtgtgggcctctaatctcagctacttgggaggctgaggcagaattgcttgaacctgggaggtagacgtggcagtgagctgagattgtgccactgcattccagcctgagcgacagagcaagtctgcatctcaaaaaattaaataaataagaaagactgagaggtaggaattaatagcagaagaaaagggggaactgatgttgtgagattcttgaggtgggaagtagtgaacactactgcagtgatgcaaaaagggtcttgaacaagatagttgtttttaaaaaaattgggaatgagtatctaaatttcttcctgaaataattattggattgactcagattggctgttaggcgccttttttctcatgagattttgttcatcaaataatcaactgttttgaaatgagatgaaaggtcttcattctgagccaatcttcagatttctatttgtgctcccacagtacagcctactacttcctagctgggggaaacaaaaggccctgctgtttttagtggctttgagaaagtcataaactccttagtcctcagctttcactcacatctactaaaataatgcctttttttttctttttttttttgagatggcatcttgtggcccaggttggaatgtagtggcgcgatcatggctcactgtagccctgaccaccgagggtcaagggagcagggtcaagggagcaggctcaagtgatcctcccacctcagcctccttagtagctggaaccaaagtgcacaatatcacacctagctagtatttttagtttttgtagaggcaaggtctctgtatgttgcccaagctggttttgaactcgtgggctcaaaggatcttcctgcctgtactcccaaagtgttgggatttcaggcagaagccaccctccttggcctctgttttttttttttttttttgagatgggagtcttgctttgtcacctaggctggagtgcaatggtgccatctcggctcactgcaacctctacctcctgggttcaagcgattctcctgcctcagcctcccgagtagctgggactacagacgcatgccaccatgcccagctaattttttttgtatttttttttttttttttgagacggagtctcgctgtcgcccaggctggagtgtagtggcgcgatcttgactcactgcaacctctgcctcccgggttcaagcaattctcctgccttagcctcctgagtagctgggattataggcgcccgccaccacgcccagctaatttttctgtttttagtagagacggggtttcaccatgttggtcaggctggtctcaaacccctgacctcgtgatccacccgcctcggcctcccaaactgctgggattacaggcatgagccaccgcgctgcccttttttttgttgttatttttagtagagatggggtttcactgtgttagccagaatggtttggatctcctgaccttgtgacccgcctgccctgcctgcctcagcctcccaagttggtgggattacaggtgtgagccactgtacccggccggcctcattttttatggagaccaaaagactgaattctataaaaatgtcttaactcctttagttctaaagcccagttttagatgttagccacatttattcccattctaacatattccttctaggtgtattttcacatgttcatttcaggattcttgcctctacatttacttgtaactttcccatttctaaattgtagccctttgatttcctccaaaccattctctgacaacccaaactgagtttttcttggaattcctatgtagctatctgtaaactaactgttatgttttagttagcctgggagagtcctagttttcatttattgtcctaatatgtaattatggctgtcctaatcagaagctactttgtagattaaaatgtctgtagagaatatgcttaagagcagaaagtttacaatatactcaaaatcttccccttcaggaggtagctttttcatgtacctccaactttctcttcaaccctgtaccaagctcttacatgtcgatatgtaaccaatgtaaactcctcagaggccacaaaagccccagtgtgcattcagaaagattggagttcagtgaaggggctgattactagatacagaagaatcgggatctgctaacagttaacatctgggaaagaaagtggaaatgagaaactatttttttgagacagtgtctcactctgtcgcccaggctggagtgcagtggtgtgatctcagggattactcagtgatcctattttttgtagaggcggggttttgccatgtttcccaggctggtgtcaaattcctgggctaaagagatccacctgccttggcctcccaaagtgttgggattacaggtgtgagccgtcgcgcctggtcagaaatgagaaactttatagtcaagttgtatttccttaagttaagaaggtcctaacagcctagtatagtaacttttttttttttttttttttttttgagatggagtcttgctctgtctgtcgcccaggctggagtgcagtggcgtgatctgggctcactgcaagctctgcctcccaggttcacaccattctcctgcctcagcctcccaagcagctgggactacaggtgcccgccaccacgcccggctaattttttgtatttttagtggagacggggtttcaacgtgttagccaggatggtctcaatctcctgacctcgtgatctgccccctccttggcctcccaaagtgccaggattacaggcgtgagccaccacgcctggccagtaacttcttaaaagaggtataaagtctgagattctaagtgggaaatctgtggcactaaaccttatcaaatgagggaggccttaggagtaattaataaattactagaacctttttaagttttttttttttttggtgtgggggggtggcggggaagagaccagtcaccgac

SEQ ID NO: 6 - DNA, 13221 bp, Homo sapiens
  source 1..13221: mol_type = genomic DNA
gacgtcacgatagacccgcccacccggggccgggccgtgggagccccgccccctggcgcgggtggtgagatggctggggccgagtcccaggggcgccggggccatgggccaagctcaggggagcagagcctgaggccgagaggggtcccctgggcacgaagaaggggagctcattagtaacctcgatttgggttctgtggactggaagaggctcccataaggcaggggaggagaaggagaagaggaatccattcttcgggagggaccgggttggaggttgaacctgggggcgcagtgcagcacgttctctctccctcctcaaggggaaactggaggacgaggggcagatgccccttggggagaggcgggcgctctgtgggcgacgaggcggggcggatccctggggggagctttaagccgactctactggagaagcagtctaccctagagggaggatgggcagttttcctgcgggggaggcgcttgggcctgtccccgcttgaggaggagcgttcctggcagggcggagtccccggagtgacgcagaggaggtgaaactctccagcccagatctagttcgggcttcagaaagtcggatggggcagaggatccaggttggggggttggccgccagatggcgctcctcggccaggctggaaccggaggcccttgagaactcgcgttggagagggacctggaccctcccccgttacccatctgtgaagggttggactggggaaaggctacagttctgaatctattagtttaatgccactctccccttccttttactctaattcttgtgtcctcaagagctcccagtcctgcactgacctttcttggttgctttcttaggtcaaaggtctaaagtttgggggaacaaaggagtggagagctcagttaacctggatcacccaggggaggagctaaaacggggcccagaagacctgcctccaaggccagagactgatactttgacatactaaatgtcttgcataaaatctggctctgctggagagaagggccttcccctttggggtcagggcatttggactgtctaggatggactttaaccagactcatccaaactccagctttccctggttctttactctggcttttagccatattagtgctgtgtggagtctgacccaatgagaaagacttgttcccagtaattccctgggggagaaagagggggtccctggtgactcaggaaaaggtgctaggaacagggcttctcacgtttctttggacttgatcattattaaagaggaaaaaacaataatagagacagggagcagagatagataattaagatataagctgacagactggcaagtcaaaggaaaacagacagaatacaagtaaatttgtttttcattgctatttctgggggaagagacttagtaaaatcttctgtcttccagatctctgcacttcccaccatgcaacttccccttatcctcttgccccaaatgaaagcagtttgacatgtgatcaagagttgtcccagtaaaggttctgtggctgaaacttaactaaagagatcctatccattttttttcttttgtcctctcctccttctaaagcaagttctttattgtccagccaatcaacaaatgtttaaggcctggcgcagtggctcacgcctgtaatcccaacactttgggaggctgaggcgggcacatcacaagatcaggagttcgagaccagcctgaccaacaaagtgaaatccagtctctactaaaaatacaaaaattagctgggcgtggtggcacatgtctgtaatcccagctactcaggaagctgaggcaggagaatcgcttgaacccaggagatggagattgcagtgagccaagattgtgccactgcactccagcctgggcgacagtgagactccgtctcaaaacaacaactacaacaaaaacgaaaattagccgggcatggtagcaagcacctgtaatcccacaagtaggctgaggcaggagaatcacttgagcctgggaagtggaggttgcagtgagccgagatcacgccattgcactccagcctgggggatggagcgagactctgttccaaaaaaaataaaaataaaaataaaaagcaaatgtttaatgaggcctggaactgtcctaggtacattgtattaagcatacatacaaaatataagacacggtctctgccaattgaaggcatacagtcttgtagccacctttttcttttttagttacctgttatccgtttttttgttttttttttaattaaattgtagagatggggagtttcatcatgttgcccaggctggtctcaaactcctgagctcaagtgattcgcccaaacttggcctcccaaagtgctgggattacagacatgaaccactgtgctcagccacctgctatcctttacgtaccgcctttaggtgcctagagtcctttctcttcttccttccaaaacttaagaatctgctggccaggcgcggtagctcacgcctgtaatcccagcactttgggaggccgaggtggttggatcacttgtggtcaggagttcgagactagcctggccaacctggccaacatggcaaaaccctgtctctactaaaaatacaaaaaaattagccaggcatagtggcacgtgcctgtactcccagctacttgggaggctgaggcatgagaatcgcttgaacccagcagcagaggttgcaatgagccgagatcttgccactgcactccagcctgggcgacagagcaaggctccgtctcaataaaaaaaagaaaaaaaaaaaaggaaattgttggccaggcacagtgactcacacctgtaattccaacactttgggaagccaaggcaggaggctcgtttgagccctggagttggagaccagcctgagcaacatagtgagacgttgtctcttcagtaaaaaaaattagccaggtgtggtggtgtgtgcctgtagtcccagctacttgggaggctgcggtgggaggattgcttgagccataatcacaccactgcactccagcctgggagacagagcaaggccctgtctctaaaaaagaaaaaaagaaaaaaaaaaaacctcttgaacaggtgaggatgaaagagcaaatgcaagagcccattttcagagttcttggagacataccatataggcagttgtctactccaggaattaaagggatgtaattttgcaagtaaccagagtttgtgtctgtcttccttctccgggtctctccctctccttcatttcctttcctctcttctccttcactctcttcctctctcccttcctccatccctcttgtcttttccctctctccctccctcttttttttttttttttttttgagacggagtctcactctgtcgcccaggctggagtgcagtggcacgatctcggctcactgcaagctccgcctcccgggttcacgccattctcctacctcagcctcccgagtagctgggactacaggtgcccgcacacctggctaatttttttatatttttagtagagacagggtttcaccgtgttagccaggatggtcttgatctcctgacctcgtgatctgcccgcctcagcctcccaaagtgctgggattacaggcatgagccacagtgcccggccctttctttctttatctttctttctctctctctctctttttccttcctccctctcctctcctctttttctttctttctttttctttctttcttttcttctccttccttcctctttttttttttttcagagttttgctcttgttgcccaggctgcagtgcaaaggcatgatctcggcgcaccacaacctccgcctcccagcttgggttcaagtgattctcctgcctcagcctccggagtagctgggattacaggcatgtgccaccacgcctgactaattttgcatttttagtagagacaggatttctccatgttggtcaggctggtctcgaactccctacttcaggtgatctgcccgccttggcctcccaaagtgttgggattacaggcatgagccactgcacctggcctgccctccctcccttcctttttcttttccttccttctttccttcctttctttcttccttccttcctttcctcttttcttttctcttttctttcctttctgtccagtgaactgtggtgaacagtgtcccataggtaagatgtagaagggagagcaggccctgccaggggtgaggctcaatcataaggcagataaggattgttggcaaggaagagaaacaaagttccagcattcactgttctatctgatggagccaaagtcatgaagtccttgagagggtacagccgggccagaaaaggggtcgtctcttcagatgtgaggtcatgcctctggagcaatcccagggctgtgagacattagagtggagggagggtggaggagtgatgagggaatgaggggagactgagttttttgcctgagcggcatgagcgggtgtggctgagtgtgtgtggatgtgatgtgcatttgtgttgggggagggagataaactataaaaaggagcgctaaggtgccccaggtagagagaggggggaatgccaactcagtgagagagaaaagtgacttgagagcattttttaaaagtcaccttttcagctttaaaaaaataaataaataaataaataaatatatatatatatagccaaatatgtactgaatgcctactctgtgtcaagtactgttcatggttctggagatacagtaatgatagaaaaataaagcaaggtagaatggggatggagaacaggcaggcaagctgggaggcaggcagttgttgcttttgatgtaggtgactagggaagacctcactgatgaagtgacatttaagcagagacctgaacaaggtgagagaaatagctgggatgagagcattctgggcagaagtagcaagagcaggccaggtgcagtggctcaagcctgtaatcccagcactttgggaggctgaggtgggtggatcacttgaatccaggagttcgagaccagcctgaccaacatggtgaaaccctgtctctgctaaaaaatacaaaaattaggctgggcgtggtggctcacgcctgtaatcccagcactttgggaggccaaggcaggtggatcatgaggtcaggagatcgagaccatcctggctaacacagtgaaaccctgtctctactaaaaaataaaaaaaataaaaaaattagctgggtgtggtggcgggcacctgcaatcccagctactcaggaggctgaggcaggaaaattgcttgaacccgggaggcagagcttgcagtgagccgagatcacaccactgcactccagcctgggcaacagagcgagactctgtctcaaaaaataaacaacaaaaaagggtagcaagcacaaaggccctcaagtatgcaggaaggcagaagcatgcttagtgtggtcaaggaacagcaagaagccaatgtgcctagacacgtgtgaaagaaggaagagcagtggtgagagaccaggtcacagaggaagaaggaagccagtcatgtgggactttctaagcctttgtaaaacctgcgaaaggatcactctggctgttgtatggaggacagactgttgggggaggggcaacagcagaaacagaaagccctattagcgtgctgtagccataatccagatgagacctgatggtggcttagagcaggttagaagcagtagaggcaatgagaaggggtcggtcgtattctggatgtattttgaagatagagctagcagaatttgctgatggatggatgcagtgtgagagaggagtcaaggatgtttccaggatttctggcctaggaactgcttacactgccaagtactgtactgaaaatgtacaaatagtggtttgagaatatgttaaagcggtgactgtttggaaactcgcgactgtgtggctatccgggataactgacctggacactcatttcttgaagctagcctaaggtaggaggagatgtggacaacatgaatgagggacttccacacctagtttgactataagatattggatattagaccgtcttgcgtagatatgctaatggtacctgttggcattagcactggagtttcaaattattctttaatcatttcaagaagacatcctggccgggcacggtggctcacgcctgtaatcccaacactttgggaggctgaggcaagtggatcacctgaggtcaggagttcgagactagcctggccaacatggagaaaccccgtttctactaaaaatacgaaattagctgggcatagtggcgcatgcctgtaatctcagcactttgggaggccgagacaggtggatcacgaggtcaggagtttgagaccagcctggccaacatggtgaaaccccgtttctattaaaggcggaggttgcagtgagctgagatggcgccattgcactccagcctggatgatagggcgagactccatctcaaaaaaaaaaaaaaaattgctgtagcaaaactttgagggagtagtggtgtgaggtaaggctacagaggttaaaaggaaccagaccttgaagggccgtatttgcttgtagaatttgtcctgtagggaaaattacaatagtctgtttatattttaggtagaaaggcagtttatgcatttcaggaaacatctttgagcttctacaatattccaggtatggaaataggtgctgaagatgtaaaaaaaaaaaaattctaaaatcatgattgaaaaccctaaggagcttaagcttaaagtcagcgagtgagcctttgagcagaggcagggcggggattagacagcagaggaggcactcaaggaatgagggatctggggagggaaagaaaaggtggctgacccctgggcgcgaatgtatgggtggaggacagtcccatcaactgaaagaggaaacccaagggaatttcagtgggaacaagttaggtttgaggagctgtgttgcatccggggagaactgaacagaacagaactggaatcatcactctggaactccggagagaagctagtgccagagagttgaaagtgtgtttgaggtgtgtgttggcagtgggacttaaacccattgcagtacaacatggaatagtttagggagtctgtgaagagagaacaggacctctgagattgaagtcacctgggagggttcagtggacactgtcatcgtctccttctggtcatgaaatcctcccttcctcagtcttctgggcaaaacaagtctttagctgttactccttgtcctattttcccttgccttttcatttttactggtcccctggatcagccacacagtctgtttaatctatgaaaatgtgaacactctgaacaaacacacacctatggtaaagtatgtgccaacagtggccctgatgacctgaaacacagcagccagagttgctccctgactttctacttaaggattactggtgatggaggttgaaaaggtggaaaaagaggcagtttagggccaaggtctaaagtgttgctatcccatatgggaaaatagccactagccacatgtggctatttaaatttaattaatggccggtagcggtggctcacacctgtaatcccagcactttgggaggccgaagtgggcggatcacgaggtcaagagatcgagaccattcctggccaacatggtgaaacctcatctctactaaaaatacaaaaattagccaggcatggtggcgcgtacctgtagtcccagctactcgggaggctgaggcaggataattgcttgaatcctggagatggaggttgcaatgagccgagatcgtgccactgcactccagcctgccaatagagcgagactccgtctcaataaataaataaataaataagtttaattaaaaggccgtgcgcggtggctcatgcctgtaatcctagcactttgggaggccgaggtgggcagattgcctgagctcaggagttcgagaccagcctgggcaacatggtgaaaccccgtctctactaaaatacaaaagaaattagcaaggtgtggtggcgtgtgcctgtagtcccagctacctgggaggctgaggcaggggaattgcttgaacttgggaggcagaggttgcagtgagccgagatcatgccactgaattccagcactccagcctgggtgacagagtgagactctgtctctacaaaataataataataataataattaattaaaagtaaaaaaattcagttcctcagttgcgctagccacattttcaagtgttcagtaaccacacgactagtggctactatgttggaaagtgcagataaagaacatttccatcatcacagaaagttttgttggacagagctggaaggcacccataaatgttgccttccttctggactttgttgaaggcccatgaagacatgttgaagagtgaatgtgggggagaaattgaaggcaggacagagagagtcaggcaatagcaaactattctaggtgtaaaagcaaaggcgaggccgggtgcggtggcttacacctgtaatcccagcactttgggaggccaacacaggtgaatcacgaggtcaggaattttagactagccgggccaagatggtgaaaccctgtctctactaaaaatacaaaaattaactgggcgcagtggcgggtgcctgtaatcccagctactcaggaggctgaggcaggaggagaacttgaacccgggagacagaggttgcagtgagccgagattgcgccactgcactccagcctggggaatagagcaagactctctctcggaaaaaaaaaaaaaaaaggcaaaggcattcccgaaggagggcagggggaataagaaaagccaatcaagattctgaagaagggcccacgtggtggttcacgcctataatggaggctgaggtgggtgtatggcttgagcccaggggttccagaccagtctgagcaacatggtgaaacccgatctctatgaaaaataaaaaaattagccaggtgtggtggggcgtctccagtcctagctactgaggctgaggtgggaggatcacttgagcccctggaggcagaggttgcagcgagctaagatcgtgctactgtgctccagcctgggtgacagagtgagaccctgtcccaaaaaaaaaaaaaaagaatctgaaggagaggtacaaaggtgaaacccattctttttttactggggagaaatttcatggagaaaagtaggaaaaccacaaaaactttggttgtgatgttcaaaacataaaagagacttccaaccaggacatcttcccagtagttactgggaagatctcaagtcagacggagggaagccagtatcctggcagcatggtgtgttttcccagcttccttgaagaaggttgagagagctgtgagattgagtccaaaatggtgccactgggctaggaaatagatggtttattgagctttcttgtttgtccctggtgtggtgttagatactatgccacaggagacattagtatgggtggaagaccaaggaaggcgattgggtgttgagaacaacatatgggaaaaagggatacgggtctgcaaagtcaggaggaatcagggaggcactgcagaaaaggagtacccacggagacgggacgcatgatggatgtgccttaatgttgcctggaagatcatctctccttatttttaacacccatggaggtaacacactcaatcatgatccggccaggtgcgatgactcatgcccgtactcccagcactttgggaggccgaggtgggcagatcacctgaggtcaagagttcgacaccagcttggccaacatagtgaaaccccgtctctactgaaaatacaaaattatccaggtgtggtggcacatgcctgtaatcccagctgcttgggaggctgaggcatgagagttgcttgaacccaggaggcagaggttgtagtgagtggagattgtgccactgcactgcagcctgggtgacagagtgagactctgtctcaagacaacaataataaaaaaacatgatcgaagttaggctaatttaggtcttcctctaggactccatctcctctcaactctccactgactttcgttttgaaaagttttagattgaaaataaaaaaagcaaaggaaaatggggtaaatgggggatagaaaatgaggtcactgggaatggaatggggataaagtaaaacgaggagtggagatagcccccccaactttcaggtctctgtcttccaaggccttgaatgataactaggccttccttaccccttaaccaacaggagaaataggcacactgaaaaaatccaggaagctggagagggggaagaattcaccaagaatgagatggaaataactagaattgaacacttcccacacatggtatcaaggtaaatttctaggatgtcaggccagaagtcatcgtttccagggggctcagaccttccttgttctcacctggtcacctttttcccgctttcagaaaatgtgtaagaagcacatgcttcccccattcaactagtgggagaaattctcaattggactgtagagtaaggtaattctaggaataggcaagcctccacaaagcctcagaaatagttccacagagaaaagcaaacacaggccatttagcaccgttggccccagaggctctgaggctaacccctaggactgtcgggtggctttcagatcccactcactccacattcatcccaagtctcacttccagcttctttctcatgaagccaagacttcagaagactggtttgttgtccaaccagagagagatggctaaaggatgcttcatccaccaggaaaggaattcgatccaggcctccagctttctagatcttttccctggggtccctcggaccctagtgttcaagctcttagattctcaaggcactcccttgggctttggggcaggattcccattctctgcctctccatctctctggaattttccactgcagctgcctctgctcccagacacccctccctgggcaaaattcccggctccctccgcctccccttccctaatcacacctcattcccacccccagccctgaggcccagcccacagtccggcccacagcccacgctaaccctggtggtcccagttagagaagtgggaggccaggtcccttatggaagggtgagagggagtgtcccagcctctctttagggcggggagaagggacactggaggggctggtcccctgtggggctggagttgggggagtggccctcctcacatccccatataaggcagggcaagcagttaggcttctggattggctgattcaaatgagcattccacctggcaacagggtttctattggctatcaggaaaaataggaggaaggtggatggaggttgttggtggcggtgattggaggggggagaggagtggaggtgaccggagtggggggcggttaccagggcaacagggtaagagcccagcagggtgtccgagacagcaggcggggctgggaggtggagtggagggaagaggaaaggaaggcgggagggagaggaaagagcctgatggggagggagcgagactgggagactgggttgttggttgggcgggggggggagtagcaaaggatcgaggagagggagataaatgaagcaagaggagtacagagatggagacggtgagagacggctgaagacatcagccagtgagtcagagaaacagattgagagagggacatggaaagatctcagaaaagatggacagagtccggcaaggaaaaatcctgggtggggccccaagatttttgaggagactgagactgaaatattgatagaaaagtcagaaagagaaactgggaggcgtgtacatcaagggaccaaaagatggtgtatttaagcactcaaacacaaaataatgataatgatcatcaacgtagttttttattaataaaatgaaaataataagatgaggaataatgataatgacagacaaatatgggagtgcacgcacaccaaaggtctttttccaaaccccagccttccactctgtccaccttcctgacctttaatacacaacccagcccctgttcccttctgaaatcccaagcttttcaaaatgactcaaagataccgagattgagggcgaggagctgaggatgtgtggaatagggtggggctggaactccagacctggacctgtattatatttgcaaaggaggcaaatataacccagaaaggctagggaagatggcagcagaggaatgagaaaagctgaaggatggaaaatgtaaaaaatgaggacagaaagaggagttgaggaaagccagccaagaatagagggccaggtgcctgactcagataagacaagagcagggttgtctgggaaagatagggaaagagaccagaggtggaagggcagaagagagtcagtgaggagtggagaagaggtgagagaagaatgggagcgattcgagttataattggttgtgaaggaggggtctaggtacagatgactgaggacggatgaagagaaatagaaggaccaggcgaataaaagtgaggtgagacgggcgaataagagaggtagaagatggaagagagctggaagacagcaaaggagaccagggggcctgactccccccggctcacagcacggggccaggaagggaggcacccggcaagggtcacaagtgtcaatctggcgggacagaaggctgggccgccctttctgccctcccatccctcctcaaggccatccagagaatgtggatggaggatgagagggggaaatccggcgtggtgtggaaggagcgcccgaaccctcttcctcttctttaagcttttggtttctcaaatacctgccctatcttcccatctgctcccctcaccccaccccttggcaaaatcccagggctttttttggggtcagtaggattctggccacctctccagtccgcccccaaaagccagactccacattcgcaggcagtaggttgggatttggaaaggtgcctccccgcaggaaccccaccccttcccacctcctcgccccactctttctccgcggccgtcagtctctccatttccccctcctggtctcgtcctccctcccgactccctagctctccctcccctcgctttcatcctcctatccccacctctccaacctcctctttcatccccctcccctctcctctcctccccgctcccgcctgccccacccctgggaagcccctcccgtcgggcagcgccgccttataagcgggttcccttcccgggggcggc

SEQ ID NO: 7 - DNA, 4422 bp, Homo sapiens
  source 1..4422: mol_type = genomic DNA
aagttttcctccctctccgcccaggctccgggaaagaggagaggcggcacccctccgggctccaccccctgcccgcccacccttttgagcaggccaaggctgccagcccggcctctgttctctcggttttcccttcctagcttaggccggcctgtctctctcgctattttccacagcctttcggtaattgtagctcccctcttaggaccttactcttccattctgaggccagagaggctggggtgaagggaagtagaggtttaaccacgacgaccagaaattttagaagagaaattttagagggattaggggaagagaaccaagagttttgggaataagggtgaaggtagggagagcactagaaattgaaaaaaaaaaatggggctgagttttggagatgctattcaacgaagctagaagatgagttatcatcggtttctggggctgagtaaaaggcagtcctagaattttactgttagttgtccaagcccttagttctatgggtcccaattcactttgaaaaaagcgggatgcgcagaagtaggttatttgttgagtcagtctgccccctggtggaacagggtgaaaacgtaaattagttatacagaatcggccccaccaccccaacacacacagcctacctagacacatcctggggcttcagttggaacgatctctcttatggggacccatgtgtgatttctctgaccaaatagcttaggaaaacagagatttgaactaaaaatgtcattctttacgtgaatcttttaatatggcccacttaaggtgcagtactcctgaaatctcactcaataggccatgaatgtgtagccttggtaaccaggaagctgaaatatgaacagcacgggtataaagaaacaagtttgccgggcgcggtggctcacgcctgtaatcccagcactttgggaggccgaggcgggcggatcacgaggtcaggagatcgagaccatcctggctaacatggtgaaaccccatctctactaaaaatacacacacacacacaaaattagccgggcgtggtggcaggcgactgtagtcctagccacttgggaggctgaggcaggagaatggcgtgaacccgggaggcagagcttgcagtgagccgagattgcgccactgcactccagcttgggcgacagagccagactccgtctcaaaaaaaaaaaaaaaagaaagaaagaaagagagtaaaagagaaacaagtttatctctgcctttagaaaaaaattattcttgctatatgtgtgctgggggtggaaaaaaatacgaaacacgaaagaaagaaaatattatctaagacctgttgcggtggctcacacttgtaatcccagcactttgggaggccaacgtgagaggatcccttaagcccaggagttcaagaccagcttgggcaacatagtgagactgcttcgacaaaaaaaaaaaaaaaaaaaaaaaaaaattagccatacatggtggtgtggcctgtagtcccagccgcagctacttgggggacagaagtgggcagaacacttgagcccaagagtccaaggctgtagtgagccacgattatgatgccactgctctccagcctcggtgatagagtaagatcctgtttcaaaaaaattttttatttattttttaattaattaattaatttttttgagacagagtcttgctctgtcgccaggctggagtgcagtggcgcaatctcggctcactgcaacctccgcctcccgaattcaagcgattcttgtgcctcagcctctggagtacctgtaatcctctgggattacaggtgcccgacaccacgcccagctaatttttgtatttttagtagagatggggtttcaccatgttggccagatggtctcgatctcttgaccttctgatccgcccgcctcggcctcccaaagtgctgggattacagtcgtgagccaccacgcctggccctattttttttttttttttgagacggagtcttgctctgtcgacctaggctggggtgcagtggcgcgatctcggttcactgcaagctccgcctcctgggttcacgccattctcccgcctcagcctcctgagtagctgggactacaggcgcccgccaccacccggctaattttttgtattttagggagagacggggtttcaccgtgttagccaggatggttttgatctcctgacctcgtgatctgcccgccttggcctcccaaagtgctgggtttacaggcgtaagccaccgcgcccggcctatttttatttttatttttttaatttttattgtttattttgaggtggagtttcgctcttgttgcgtaggctggggtgcaatggtgtgatctgggctaaccgcagcttccgcctccctggttcaagcgattctcctgcctcagcctcgcgagtagctgaaattacagacacccgcactaccccctgctaattttgtatttttagtagagacgagctttctccatgttggtcaggctgctctcgaactcccgacctcaggtgatccacccacctctgcctcccaaaggcctgggattacaggcgtgagccactgcgcccaacctttgtttttacttttattttattttttgagacaaagttttgctcgtcacccaggcagaagtgcaatggctcaatctctggtcactgcaacctccgcctcttgggttcaagcgattctccagcctcagcttcccgagtagctgggattacaggcgcctgccacgacgcccagctaaattttttttttttttttttttttttagtcgagacggggtttcaccatgttggccacgctggtctcgaactcccgacgtcacgtgatccgcccgcctcagcctcccaaagtgctgggattacaggcctgagccaccgcgaccggcaaagaaaaaattattattattattattattattatttgagacggagtctcgctctgtcgcccaggctgcagtgcagcggcgcgatctccgctcactgcaagctccgcctcccgggttcacgccattctcctgtctcagcctcccgagtagctgggactacaggcgcccgccaccgcgcccggctaattttttgtatttttagtagaggcggggtttcaccgtgctagccaggatggtctcgatctcctaacctcgtgatccacccgcctcggcctcccaaagtgctgggattacaggcgtgagccacggcgcccgggcaagaaaaaattattcaagggagtctctctcgagcctattctggctgcctataaaaaattaaaggccggacgcggtgtctcacgcctgtattctcagcactttgggagaccgaggcaggtggatcacgtgaggtcgggagttcaataccagcttggccaacatggtgaaaccccgtctctactaaaaatacaaaattagccgggcatggtgccgcatgcctgtaatcccagctactcgggaggctgaggcaggagaatcgcttgaacgcgggaggcggaggttgtggtgagccgagatggcgccatcacactacagcctgggcaacaagagcaaaactgtgtctaaaaaaaaaataaaaataaaaataaaaaaaaagaaggcatctttcacttatccccatcaccttccgtgtccatgcttgtcccttctcaagctaaactattcaatctctttttgtccattctctcacctctcattatctccttgagtcttccctatttcttttcactgaccaaaccatgtctcccctgtttatttatttatttaagacagagtcttgttctgtcacccaggctggagtggatctcggctcactgtacttccgcctcccaggttcatgcgattctcctgcctcagcctccagagtagctgggattacaggcacacgccaccacgcctggctaattttcgtatttttagtagagacggggtttcactatgttggccaggctggtcttgaactgctgacctcaggtgatccacccgccttggcctccctaagtgctgggattacaggcatgagccactgcgcccggcctaaactgttaaattaaaccaatagaaaaagagttaagaatataggtatttaaaaaataggacggaagtcctggatatggctccttctccctggtgttttcctcaacctttcctatttattaggctccaactgtcaaggtatggcacaaaattttaaaagtgtccatttatccctggatagtgatcaaacgctgacctcaaattagcaaggtaaccctctcaacatggtgacgcatatgtgtcacaactaacagttaaagaccttccgaaaatggcagctcccttgtctccccactgcggcggatcaaagtaagacgttccaaacatggtggcacaagatccttgcgtcatttcctgtagtgtgctctatataaggggcaggatttccgctttcgct

SEQ ID NO: 8 - DNA, 3975 bp, Homo sapiens
  source 1..3975: mol_type = genomic DNA
ggcgcctcgcctgctgctcactgggaattagctcggcgccccgttgcattgtggtcttggtagtctttgcgagaacggcaaatggcggttgcagaggctcgtccgaattctcggctcgctgctccctggctggcgcgtgtttgttcattctgggctaggtgagcagggacaatacgtcgccctctcaggactttgctctctccttaaaacccgaggattcaccgtcaagcttctggtttctctgacatccttccccagccgccccttcatcgccaccgatgatccagtggagaagctgtgttggaggtcaagggatggaggcgaaactgcgcgttctcgtccctgcagaaaccggaatatccgagacacatgacgaacatgaaactttctccttgtctcttggctcccactccgctacacagatgaacggtgttaccttccctcatgttactttgctaagcctactcctcagagctggtcctttgcataacctcttcaaatgctctcaactctcccttgctcctacataatggagtaatcataagtgcaatgagaaaagcatgagatttgaagttccataagtattgagtctaggttctggtggttattaactccatgatcttaggaaagtatctgtcagtgttctgtgtaaaacggaaatgctaatgtgtatttaccaggactttttcttgcgagaggcagatgaaatgtatctccgtttcatgagtaagttcagagaatataagtgacttgtccaaggtcagcaagctaatgagtgggaaagctgaggcttgagcctagactgtcaacacccaggccaaagattcattgcctgcttttcatcttatggagttaaaccttgatacacactcagatctgcagtgacttgaccagcactgtgggttcatgctttactgtctgataacgaattttcatttaattttattttttttcttagacagggtctagctctgtcacctaaacctgatgagggctgagccttcatttcgccgttttttgtttgtttgtttttgtttttgaacgagatggactctggctatgttgccagactggagtgcagtggcaagtcacaggagccatcatagcgcattacagcctggaacttctcgtcttcagggatcctcccaagtagctgggatttcaggcatgtgccaccatacctggctctaattttgaatttgtttctttaaattattatttgtgtttgctgaaattgtgtgtgtattatttttgtgtatgtgtgtgagtagttatatgttacataaagtaggttgtggggttttttccagtaatgtttagctataattgaagtttgggccgaaatctgctagtatttgttactcctcaccaaatcctaccttatctcacaacaaactgtaagatcttgtgatatttcacttccaataaccttaccaggcatcagagtggggtctctactctgaaattaaatatctgccataaggtatgttgaagacgttagtctctttcccaggaggcacaaagtgcaagtcttttcctcatcgcttgagcaaattaaacctaccggacagtttactacagaactcaaacacagctgggcgcagtggctcacgcctgtaatcccagcaatttgggaggccgaggtgggcggatcacctgaggtcgggagttcaagaccagcctgaccaacatggagaaaccccgtctccactaaaaatacagaattagctgggcatggtgccatatgcctgtaatcccaggtactcgtgaggctgaggcaggagaatcacttgaacccgggaggtagaggttgcggtgagccgagatcacaccattgtactccagcctgggcaacaagagcaagaattcatctcaaaagaaaaaaaaaagaacttaagcataagccacaaataaggtttatcattacctcatcaatgcttggatacttacatttatcaagttattacattttgctgtgtaaaaaatattatgtatggtgaaagggggatatatatatattatgtattaaaatatgtattatatattatatattaaacataatataattatatataattatatattaaacataatatattatatattaaatatataattataatatatatttaatatatatgtataatatataattatatattatatataatatgttatatgtttataaacatatacatgttatatatattatatatttcctatatatttaatatataatatatataattatacattgtaatatataatataatataatatatatatacttaaggatgctgtgcatcaagcctgaattttttttttttttttgagatggagtttcgctctgtctccaggctggagtgtaatggtgcgaactctgctcacagtaacctctgcctcctaggttcaagcgattctcctccctcagcctcccaagtagctggtattacaggcacccaccaccatgcccagctaattttttttgtatttttagtagagatggggtttcgccatgttggccaggctggtctcaaactcccaacctgaagtgatccacccacctcggcctcccaaagtgctgggactacaggcatgaaacaacgtgctcggcccaagcctgaattttcttttgcatttccagtagttttgccatctttgttattcagcaaagagatgtttttgttttcgtttttgtttttttgagacagagtctggctgtgtcacccaggctggagtgcagtggtgtgatctcaactcactgcaacctctgcctcccaggttcaagtgagtctcctgcctcagcctcccaagtagctgggactacaggcgccctccaccactcctgtctaatttttttttttttttgtatttttagtagagacggggtttcactatgctggccaggctggtcttgaactcctgttgtgatccaccacgcccagccaaagagctgtttttgttaaagtttgcttgtcccaggctgggcgcagtggctcacgcctgtaaccccagcactttgggagactgaggcagactgatcactcgagcccaggcgttcaagaccagcctggccaacatgacaaaaccctgtctccactaaaaacacaaaagttagccaggtgtggtggcacgtgcctgtaatcccagctactcgggaggctgaggcaggagaatcgcttgaacccgggaggcagaggttgcagtgagctgggatcgtgccactgcactccagtctgggcgacagagccacactcggtcttaaaaaagaaaaaaagtccttgtccctctgcttgtaagtaccaccaaagggctatattaactgactattggccaggcatggtggctcacgcctgtaatcccagctacttgagaggctgaggcaggaggcagaggttgcagtgagctgagatcgcactattgcactccagcctcggagacagagcaagactctgtccctaaaaataaatgaataaataaataaaataactgaccattgtgcctggcacttagtagatgctcaatatacatatatgaataaatgaacttctcattttcctcactaataaaagcagatattaatgcctatcaaataactaaatggattgctggcagaatcaaagaagcttgcacaaatcactggataaatcactaggtgcagttatttccctgaggctaggactgttagagttcttctcgctgggaatttcctgtgctcaggacaaggctctgcccacaagggcatccggaccccatggcggaaagaggatatccagaccttcagatcactctacctcctgctctttgctattttccttaccaacagaacagctagggaagagacagcagtaggattagtcctggttccacagaaaccgagcctgatttgggtaaatcaatcactttgaagacacaataattggatttgagtggagaacaattctgacaataatccaagttacccaaaacaggaac

SEQ ID NO: 9 - DNA, 13991 bp, Homo sapiens
  source 1..13991: mol_type = genomic DNA
tattgcttgcttgtcttatttgcctggtcaggcctgagatggaggctgtttccctcagggatgacagacctcgggctgctggatgtgacctgcccttgaagcccaaatattcatatcctggttctggaggaacctgtgaggaggctgagtaattctgtatccttgcattaggcacgttgggaagtagcgccatctactggcacatcttaagcactgcatgcttccctggagtcttctcaagaatgcctcaaattggccacctcaggagtgagcgatcagggcaatgattgtccgttggtttcaaggcagtgctggacctgggccttaagtttccatccccctgtcctgaccagtgcctaggtttagtaatttgaagagtccaagcgacttacattaagaagttagggaatcctagcttgcaaaatcacaaaaggtggggacaggggctttctccatcagacactatagtcagtgtgcataggtgacctgcaggaggcaacagtcctgcccagaccttgtcactcggcccctaaggccctatcttatgcactttttattggtgggcaccagagaccactgataaacaggttttaggaaggtttatggcaaatctctctaacctccagtcacacgaaactctttactttcccaaaccattgctctttgttcaagaagtatttattgggtacttatgggttaggcatgatgctaagccctggggatatagctataaccaagacacataagtccctacagcctacttgaggagacagactaagtaattgcataattagtttaattacagtcggaagaagtgttatgagaagtgcagggcgagtagcctacttatttaatcagctaagatctgaaagatgaatagggattaattagttaaaggaggtaagagtgggaacagcattccatacaaattggacagcttagggaaaagtcttgaaactggaagacgctgctggaaaagcctaaagaagacttgtaggacagcaactaaaagtaagggggacctggtggtagaggcgatggcataaaacaatgctggcattggtaggaactgatgattggaaggcctaaaagactgttgagatttctattatttattttttgaattgaggtctctgtcaccgaggctgtagtatacaaaaattttaaatttttatagagattaggtctccttatgttgcccaggctgttttttgtttgtttgttttgtgatggagtcttgctctgtcacccaggctggagtgcagtggcgccatctcggctcactgcaacctccgcctccttggttcaagcgattctcctgcctcagcctcctgagtagctgggattacaagtccccgccatgacacctggctaattttttgtatttttggtagggatggggtttccccgtgttggccaggctggtcttgaactcctgacctcaagtgatctgcccgcctcagcctcccaaagtactgggattataggcgtcagccctcccaggctgattttgaactcctaggttcaagactcctaaattcttggcctcccaaagtgttgggattacacgtgtgagccactacacccagccgcttccaaatctttgtgaacagtgctgcaacaaacatggaagtgcagatctctctgatacactcatttcctttcttttgggtatatactcagcagtgagattgctggatcatatcgtagctctatttttagttttttgagaaacctctaaaatgttctccatagtggttgcactaatttacattcccaccaacagtgtttgagggttctcttttctccacatcctctccagcatttgttattgcctgtcttttggataaaagccgtttgaactggggtgagatgatatctcattgtagctttaacttgcatttttctgatgatccgtgatgttgagcaccttttcatatgcctgttttccatttgtatgtcttcttttgagaaatgtctattcaaatgttttgtgtattttcactggattatattttttcccatagagttgtttgacttccttatatactctggttattaatcctttgtcagagagatagtttgcaaatattttctcccattctgtgggttatctcttcactttaatgattgtttcctttgctgtacagaagctttttaactagatgtgatcccatttgtccatttttactttggttgcctgtgcttgtggggtattactcaagaaatttttacacagatcaatgtcctggagagtttctccggtgttttcttgcagttgtttcatatttcgaggtcttagatttaagtctttaattcattttgatttaatttttgtatatggtgagagataggggtctagtttcattcttctgcatatggatatcctgttttcccagcaccatttattgaagagactgtcttttccccagtatgtgttcttggcacgtttgttgacaatgagttcactgcaggtgtgtggatttgtttctgagtcctctattctgttccattggtctatatgtctgtttttatgccagtaccatgctgttttggttactatagctctgtaacaaaatttgaagtaagataatgtgattccttcagttttgttctttttgcttagtatagctttggctattctgggtcttttgtggttccatataaattttatgattgtcttttccatttctgtaaagaatgtcattgatattttgatagtgattgcattgaatctgtagattgctttcagtagtatggacattttaacaatattgattcttccaattcatgaacatggaatatctttccatttttttgtgtcctcttcaatttattttgtcagtgttttatagttttcattatagagattttttctttggttaagctcattcctagatatttggttttatttgtagctgttgtaaatgggattactttatttcgtttccagactgttcagaaattagtcttgacttaaacatctcaccctgcctaacattaacattctctatagcaaatgtctattttgttgttgttaacaacttacctgaaaaattagcaacagacatctttgtctaatcactgtggttgtaggaaaatccaggttcttgtcacatgaacaggaaagattaggctcgcagaaactttgaggggtgagggggatggaatttattggggaaaaaggaaaaacaacacagcaaagcaagagaggggttcctgttaacaggccctcatctcacagattgaatcccaggttccacacaggaacaggaggggccaggctcctccccactgcaaacagcatgaacttccatggctccaccccattctcccagtgcgcaggccagtcagatgttctctgggaacccctttatacttggctgtctcattcccccgtctaaagaagtacatctaactgctcttagagtgaggctaaggacaaagaccaatcttaactgcttcctgctgacagggggtgctgttttgggaagacggcagtcagatctccctcagaggcctatattagggtcccctgcagaaggggccattgtccaaggctctggttgcatgtctgtttggagtttgatggcctgaagctgagaagagacaaactgggttattagaaaacatgtatcaaaccaaaacaagggtaaggacaggtgaaaaatcatgaggccttttaccagtttgtatggggagagggaggccaaaagcccaattggtaaaaaaaacttttacccttttgctggcatgtcaggcttctgggttcccttcctctgagcccaattctaagttagccagtttaaggttcgggaaattaactctttctcaatttggaggattatctgaggggagaaaaaaaaggtgttttttcaaaggagtcccaggagttcaagatgcattcaaaaggagtacaaactgaaggtgaatggctactcatctagaaaggggcaagcaggcgcccctagttcctttctcttcctaccaaatacctggggtatgtgagggagggaaagtgaggcattcctctttctttccaccatccttgtatccctgagtcctggcgaatgcaacagcatgccacccatgggtgttaaagaggcttttacccatgttaactagcggggctgggggagggaattattcactctactcacatacgccttatctcccctgctgtaagtagccgtgaattccctagacttcatttacgccatggatactagcatgacttttatccatgaaacagaaagcttagcttaattggcaggagtcagccacgctcacctgtgctgtgccttttaacttccattatcttctgcctctggatccctcagatccagtcttctttcctagggctttgacccaaagcttggaattgagtttgagacaaaaatgtgtcttggagggagttgcacggactccttatcataagccaaatgctaaagtgaagctgtggaattgagtcctcccccaacaagggaaagaaaaggttgtcttgtgacacacccagataactggtggctatagttatgcttgctaggatttgggtgcatggtgcttggctttggttagctcccttggtcttgctttcccaaaaaggaaacccccaggtgatgggcaccctatttattcccattgcctggcaggatttgatgtataattgctcagaacaagaatattgatccagatttctacattacccatcccttttgttctttctgagctgtagctggagattgctggttggttcacaggaacaagcagggttagtctaaaatgcaggcaaagacttaaaaacaactaattagtttagaatttaatgacaaatgaagtcgggtgcggtggctcacgcctgtaatcccagcactttgggaggccaaggcaggtggatcatgaggtcaagagattgagaccatcctagtcaacatggtgaaaccccatctctactaaaaatacaaaaattagctatggtggtgtacgcctgtagtcccagctactcaggaggctgaggcaggaggattgcttgaaccctggaggaggaggttgcagtgagccaagatcgcaccactgcactccagcctggcaacagagcgagactgtctcaaaaaacaaacaaacaaacaaacagaatttaatgacaaatgtatacgttttgaaacataatttctttctctgcagttctcatttatttttatttttatttttcaatttttcttttgttaccagttctcatttctgttaaaaaatcatgataggactgagttgtttgcaaaatagactttagtcttatacttggcctgattagttgcataaagtgcagcaagaataactatttctacataggccttttagattggctttgatgcaactcttttccacaaggactcttagagaagaccttttaaagccgagcccagccatgggtctgtatccttaaatacctgtgagttgagtgctcctcttctcttaaggtcccaagataaacttggaactctgggccagttagaaagtgacactcttggctgggcatggtggctcacacctgtaatcccagcactttgggaggccaaggcaggcggatcacgaggtcaggagatccagaccatcctggctaacatggtgaaaccccatctctgccaaaaatacaaaaaaaaaaaaaattagccgggtgtggtgccacatgcctgtagtcccagctactcaggaggctgaggcaggagaatagcttgaacctgggaggcagaggctgcagtgagccaagatcatgccactgcacgccagtctgggcgacagagcaagactctgtctcaaaaaaaaaaaggaaagtgacattctttactgaccacaggtcaggaaccctgtacagggactgtgtaggcaagggtatgaagccagttttctccccaaggggatttttttggctctgcaactcgagcttgactgcttaaagggaagcatacccttgcactcaaagccttggtaaaacaaccagtttctccagttgcatcctgttgcaaaagaaaatggattcttactgcactgatgcaaacaactatattaccacaagttaaaaatactcacagaggctgggtgcagtggctcatgcctgtaattccagcactttgggaggccgaggtgggcagatcacctgaggtcaggagttcaagatcagcctggccaacatggtgaaacgccatctctactaaaaatacaaaaattagccaggcgtggtggcaggcacctgtaatcccagctacttgggaggatgaggcaggagaattgcttgaacccgggaggtggaggttgcagcgagctgagatcatgccattgcactccagcctgtgttgtgggaagtcaggaaccccgaatggagggaccggctgaagccacggcagaagaacataaattgtgaagatttcatggacatttattagttccccaaattaatacttttataatttcttatgcctgtctttacttcaatctctgaacataaattgtgaagatttcatggacacttttcacttccccaatcaatacccttgtgatttcctatgcctgtctttaatctcttaattccgtcatcttcgtaagctgagaaggatgtatgttgcctcaggaccctgtgatgattgcgttaactgcacaaattgtttgtagagcacgtgtgtttgaacaatatgaaatctgggcacctccgaaaaaagaacaggataacagcaatgttcagggaacaagagagataaccttaaactctgactgccagtgagctgggcagaacagagccacatttctcttctttcaaaagcaaatgggagaaatatcactgaattctttttctcagcaaggaacatccctgagaaagagaatgcgtccctgagggtaggcctctgaaatggccactttggggggcagctgtcttttttggtcgaagctgtagggatgaaataagccccagtctcccgtagcgctcccaggcttattaggacaaggaaattcccacctaataaattttggtcagatcggttgtctgctctcaaaccctgtctcctgataagatgttatcaatgacaatgtgtgccaaaacttcattagcaattttaattttgccctggtcctgtggtcctgtgatctcgtcctgcctccatttgccttgtgatattctattaccttgtgaagcacgtgatctctgtgacccacaccctattcgtacactccctccccttttgaaaatcactaataaaaacttgctggttttacggcttggggggcatcacggaacctgccgacatgtgatgtctcccccgaacacccagctttaaaatttctctcttttgtactctttccctttatttctcagactggccgacacttagggaaaatagaaaagaacttacatgactatcgggggcaggttcccccgataagcctgggggacaagaatgagacttcatctcaaaaaacaaacaaacaaacacaaaagcaaaaaagaatactcacagatagtttccaaattctagaggaacaaggcagagagaaacaaaaaacatgctccaggccgggcgcggtggctcacgcctgtaatcccagcactttgggaggccgaggcgggcggatcacgaggtcaggagattgagaccatcccggctaaaacggtgaaaccccgtctctactaaaaatacaaaaaattagccgggcgtagtggcgggcgcctgtagtcccagctacttgggaggctgaggcaggagaatggcgtgaacccgggaggcggagcttgcagtgagccgagatcccgccactgcactccagcctgggcgacagagcgagacttcgtctcaaaaacaaaacaaaacaaaacaaaacaaaaaaaaaacatgctccagattttgttcagaagagtataccttactcctcatgcctgtaatcccagcactttgggaggccgaggctggcggatcacaaggtcaggagattgagaccatcctggctaacacagtgaaaccccgtctctactgaaaatacaaaaaaaatagccaggcctggtggcgggtgcctgtagtcccagctactcgggaggctgaggcaggagaacagtgtgaacctgggaggcggagcttgcagtgagctgagattgtgccactgcactccagcctgggagacagagcgagactccatctcaaaaaaaaaaaaaaaaagaggataccttactcaatttattaaaggacataaatagctcaaagtaagtttccttgactctgaaaaacaaaataaggatcagcaatattccaagcaaaagttaaaaagtttgcttcagtttcttgagtacattcagttaactcttgttttacttgatatttgtgaacattttagctcttcatgagtcctgtacgtttttcctttattccaatgtcacaatctccaaagttatcagaaacctgtatttgagagcacctgtcaaagttctatagcttattataaaccttgttttgaaaaggattaaaacgaaacaacaattgtctgtgaataacaaaatgttcagtgtagttacagttagaagcacaattgacaaagaagtttgattatctccgtggtttacaaaaacttaacaaccttaattatgattgatagcatatactcagacattagaattttagaaattccatacaattttggaacatgtattattcaccaaaatgtaacctaaagaagattgaacacagtatcaagtacctaaatgtttcaaataatcctgtttagctctcttttctggacactctaggggcccttctgaagtattcgaaaagccaggtgtcaggaaagacaattttgaaactgaagttttattttggaaagcctgttaaatatgttagaggtttgaaacacttaaaatagaattccagattacaataaattatttattttgccaaaataatgactcagaaacataaaaagaaaaaaaaaaaaccttttataagcctttacaaattttgccaaagagcagattagtgccttaagagtaccttattgtgcttttatttcaatgttcaatttacagaaaaatcatacaatacacttttgaatttagtcaatatgttcacacagataatttctttgcaagattaatttttttttttttgagatggagatggagtctcactctatcacccaggctagagtgcagtggcatgattccagctcactgcaacctctgtctcacggttcaagcaattcttgtgtctcagagtcccgagtagctgggactacaggtgcctgccacaatgccaagctaatttttgtatttttagtagagaccaaaaagcagtttataaccataaaacatttagccaacctggtatctgacctgcataatttagtccaactattttttttttttttaagacaaagttttgctttgttgcccaggatgcagtgcagtggcacgatctcgactcactgcaacctctgcctcctggattcaagcaattctcctgcctcagcctcctaagtagctgggactacaggagcacgccaccatgcccagctaattttttgtattttagtagagatggggtttcaccctgatgcccaggctggtcttgaactcctgagctcaggcaatccgctcacctcagcctcccaaagtgctgggattacaggcgtgagccactgcactcggcccagtccacttatttatattttgatgacattggctttttaccaataatctttaaggctgttttcatttctcaaagattaaagtcaggtgaactgaaaagtatcacaggttttatctttccttttattttttttgagatggagtttcgctcttgttatccaggcttgagtgcaatggcacaatctcagctcaccgcaacctccacctcccaggttcaactgagtctcctgcctcagcttcccaagtagctgggattacaggcatgggccaccaagcctggctaattttgtatttttagtagagatggggtatgtccatgttggtcaggctggtctcgaactcctgacttcaggtgatccgcccgccttggcttcccaaagtgctgggattacaggcgtgagccaccacacccagcctatcttccctttaataaatatttgatccaagtgcttatcttcctttaggccaattaattagagctatttctatagacgtcacacacaacacgtatataactccacggacaggcagaagaaaacccagcccccacaagatccttcatttgccaatctcctatttggaccattggcctttgaggacttttaattcctggggttacatgaggaaaacaggccgggcatggtggcttatgcctgtaatcctagcactttgggaggccaaggtgggtggatcagctgaggtcgggagttcaagaccagcctggccaacgtggtgaaaccccatctctacgaaaactacaaaaattagctgtgcacggtggtgctcgcctgtaatgctagctactcaggaggctgaggcaggataatcacttgaacccaggaggtggagattgcagtgagctgagatcatgacagcacactccagcctgggcgacagagtgagactctgtctcaaaaaaaatttaaaaagtacatggatttaaaatatacttattttttaaaatttttattttttgtttttttgagatggaatctcactctgttgcccaggctggagtgcagtggtgcaatctcgactcactgcaacctccgcctcctaggttcaagcgattctcctgccttagcctcctgagtagctaggattataggcgcccaccaccatgcccggctaatttttgtacttttagtagagacggggtttcaccatgttggccaggctggtctcaaactcctgacctcaggtgatctgcctgcctcggcctctcaaagtgctgggattacaggcgtgagccaccctgcctggccttatttttatttatttattatttatttatttttgagacagagtctcactctgtcacccaggctggagtacagtggtgcgatctcagctcactgcaacctccacctcctgggtttaagtgattctcctgcctcagcctcctgagtagctgggactacaggcccccaccaccatgtccggctaatttttgaatttttagtagagatgggtttttaccatgttggctaggatggtcttgatctcctgaccgtgtgatgcacccgccttggcctcccaaagtgttgggattacaggcgtgagccactgtgcccagctattcattttattttatttatttattttttgagatgaagtctcgctctgttgccaggctggagtgcagttgcacgatgtcggctcactgcaaactgcctcccaggttcaagcgattctcctgcctcagcctcccgaagtagctgagatttcaggcgcctgccaccacgcctgcctaatttttgtatttttagtagagatggggtttcaccaggggatccaggctggtctcaaactcctgacctcaggtgatctgcccgcctcggcctcccaaagtgctgggattacaggagtgagccaccgcgcccggcctgcatccagctatttatttttttttgagatggagactcaccatcgcccaggctggagtgtagtagtgcaatctcagctcactgcaacctccaactcctgggttcaagtgggtctcttgcctcagcctcccaagtagctgggattacaggagcctgccaccactcccggctaatttttgtatttttagtagagatggtgtttcactatgttcgctaagctggtctcgaactcctgacttcaagtgatccgcctgcctcagcctcccaaagtgctgggataacaggcacctgctaccacgcccagcctaaaatatattttaaaatatatttctctgagttcctcaaggtagagtccatgtcttttgtccccagaacctagtacaaagtcttgtacctcaaagttgctcagtgaatattaatttaatggatgaatcggtcacaccagtaattgcagaggttttctcaggccctagccctggctgcaaggactttggagagatctcccctcccaacaccctccatttaccacatcatcagactgtggtagccattcaatgagtgcttagcgtgaattctacacagtgctaattacctgtaattctcacaatgtccttaagcggtggctactaatatgtcttcattttacacttaaccttaatcccagctactcgattacaatccgggttacgtgacctggctcacgtcacctagctaataatggaagagcccatgtatgtttgccataggaattggcattttcacgtccagctctctagccccaaagatttcaacctcttcgagccttgacagattggaataagaatcctctccaggaatctttcccaatatgctggaacctaaagggaaataaaaacccctactccgatcagcgtagactgggccatggtgaaaaggaatggatgatactccaggtgtgaggaattgcagctttttagcctgccagttatttttcactcagccatttgcaggaaatgaggcacactgctttccataactgatttgtcttgcaactttctgaacaagtcacagcttccccttttttttttttttgagacggagtctcgctctgttgcccaggctgcagtgcagtggcgagatctcggctcactgcaacctttgcctcccgggttcaagtgattctcctgccttagcctcccgagtagctgggattacaggagcctgccaccacacctggctaattttcgtatttttagtagagacggggtttcaccatgttggccaggttggaaccttggcctatttatttatttatttttgagacggagtttcgctctgttgcccaggctggagtgcagtggcgagctctcggctcactgcaacctctgccttccgggttctagtattctcctacctcagcctcccgagtagctgggattacaggcgcccgccaccacgcccggctaatttttgtattttttagtagagacgggggggtctcaccatcttggccaggctggtcttgaactcctgacctcgtgatccaccagcctaggcctcccaaagtgctgggattacaggcgtgagccaccgcgtccggccccttggtttatttttctaagaaacagactaacactgatctcactagtagcattgttaatattaaacaagatagctaatagctttaaagcgtctaatcacggtacccccacgagtagtagtcacacagcaaatcataggcaggagtggacaaacagcgctgggagcattaattctggtagatttggagtctgggcagggcctaagaggatgggatcagcggattgggccagccaaagggggcgggttcagagggaggagccagctgcctcccgctgcaagccccgagctttgaatcaagcgcggtagtttcagctgtagccaatcctagaacaaaatctttttgcgtcagttgttgctgaggcctagatctcacccagatcagttctagtcgctgctgcttgtacccgtcctcggcccttgcgtgtccgtccgttacgactcagaggacggagagaaagtctaagtcaccttcctcagtatccaggccggaaggagactctcagctactcgccctgaacatgcagagcagcactggcttccctgtctctggatgccgcaggggcgtcctgtcaggagttgtagtgtcaagattccagttcttcttatgcgacttgctggagaagcaggaagttgctgtttattggagaagtcgggctagggagggaaacaatcccgcggagccatatggagagggcaggaggtgtctagatagtggtaggccggcgggggacggagggtatgggtggtgagtcctggccctttaaacccgagggggaggatccggaagtggatgggcggggcgaagggtcctcgtatataaaagggccccggccgcgcggggt

SEQ ID NO: 10 - DNA, 49551 bp, Homo sapiens
  source 1..49551: mol_type = genomic DNA
tttgttctattttctcttggatcatgactttcttttcttttctttttttgtttttgagacagagtcttgctctgttggccaggctagagtgcattggcacgatcttggctcactgcaaccaccatctcccagattcaagtggttctcctgcctcagcctcccaagtagctgggactacaggcatgcaccaccacacccagctaatttttgtatttttggtagaggtggggtttcaccatgttggctaggctggtcttgaactcctgacctcatgtggtctacccacatcggcttcccaaagtgctgggattacaggcacgagccactgtgcccagccatatcatgcctttctatactgagattataacctattttttaaatttgattttaaaaacagttgaaacttttagacagttcctaaaaaattagcagctatggaataataactaacatttaattaacaatgtgtacaagatactttaaatatgttaactcatttaagaaatgatagtgttttcattctggtttttcttatatatttttctctctctctttttttttttttttcaagatagagtctcattcagtcgcccaggctggagtgcagtggcgtgatcttggctcactgtaaagtccgcctcctgggttcaagcgattcttctgcctcagcctaccaagtagctgggactataggtatgcaccaccacgcccagctaatttttgtatttttagtagagacggggtttcactatattggccaggctggtctcgaactgaccttgtgatctgcccgcctctgcctctcaaagtgctgggattacaggcttaagccaccacgcccagcctttttttttttttttttttgagactgtgtcttactctttcacccagcctgagtgcagtgatgccatcactgctcactggagcctcagcctcctgggctcaggctgtccttcctcctcagcctcgcgagtagctgggactacaggtgtgcaccaccacttctgcctgtaacttatttttcataaaacatagacttgagctgggagagggaggcagtggagataggaattgtgtgttgactgtaaggtctgtgaagtctaaggctaagaaaatgccaagttcagataatttaattttgaagaagtaataacattgttttaatttgccactttttgagatggagccttgctctgtcacccaggctggagtgcagtgtcacgatctcggctcactgcaacctccacctcccaggttcaagcgattcttctgcctcagcctcctgagtagctgggattacaggcgcgtgctgccacacctggctaatttttgtatttttagtagaaacagggtttcaccatgttggtcaggctggtctcgaactcctgatgtcatgataacacctcggcctcccaacatgctgggattacaggcgttataacagtggcatattagcactagttaacttttttttttctttttaaaatttattttattttgaaatggaatttcgctctgttgtgcaggctggagtgcaatggcatgatctcagctcactgcaacctccatctcccaggttcaagtgattctctcacctctgcctcccgagtagctgggactacagtcaggtgccaccacgcccagctaattttcatatttttagtacagacagggtttcaccatgttgaccaggctggtctcgaactcctgacctcaagtgatccgtgccccctccccgccccggcctcccaaagtgctgggattacaggtgtgagccaccatgcccagcctattttattctttaatattaattttttgagacagggtgtcactgagacctaagtgcagtggcatgatgatggctcactgcagcctcgacttcccgggctgcaatgatcctgccagctcagcctcccaagtttctgggaccacaggtatataccgccatgcgtggctaatttttgtattttttgtagagacggggtattgccatgttgtccaggctggtctcaaactgctgtgctcaagcaatccacccacctcggcctcccaaagtgctgggattacaggtctgagccatagttaacatgtttagaagcttctggtttttgtttttgagatggagtctcacccaggctggagtgcagttaacaccatcttggctcactgcaacctccacctccaaggtaaaagagattctcttgcctcacttcctgagtagctgggattacaggcatgcaccaccgtgctcaactaggttttgtatttttagtagagacagggtttcaccatactggatgttggccaggctggtctcgaacttgtgacctgaaatgatcttcccacctcagcctcccgaagtgctgggatatcaggcatgagccaccgtgcctggctagaaacatgtttttaaaagaaacaggccaggtgtagtggctaacgcttacaatcccagcactttgggaggccgaggagggtggatcacttgaggttaggagttgtaggccagcctgggcaacatgatgaaaccatgtctctactaaaaatgcaaaaattagccatgtgtggtggcgggtgcctgtaatcccagctactcaggaggctgagacagtagaatcacttgaacctgggaggcagaggttgcagcaacccgagatggtgccgctgcgctccagcctggacaacaagagtgaaactccatctcaagaagaaaaaaaaaagacccagatatagaaagtatgaaactgcctgggcatggtggttcatgcctgtaatctcagcactttgggaggctgaagcaggtggatcacttaagctcaggagtttaagacccgcctggggaatgtggcgaaaccccgtctctacaaaaattacctagtgtggtggtgtgcacctgtagtcccagctactcgggaggctgaggtgggaggattgcttgagcccaggaggcagaggttgcagtgagctgagattgtgccactgcactccagcctgggcaacagagccagaccttgtctaagaaaaaaaaattatatggggttataaggaacccagaatatgaaaaaataatcttgaaaatgacgagcatatttagaggactcaacactttccaattatttcagaacttactacaaagcagtaatagtcaagactgtggtgttagcataaggatagacatatagatagaattgcaagttcagaaataaacacatctatggtcaattgattttttttattaaaaaatcacaataggcaagcatatggtcaattgtttttttgttttaatttttttttttttcagatggagtctcactctgttgcccaggctggagtggtacaatcttggttcactacaacctccaccttgtgggttcaagtgattctcctgcctcagcctccctgtagctgggattacaggtgcatgccaccacgcctggctaatttttgtatttttagtagagatggggtttcaccatgttagccagattggtctcaatctcctgacctcagcctcccaaagtgctgggattacaggcgtgagccactgcaccttactggtcagttgttttttgacaagagtgccaagactattgaatggtcaacaagtggtgctgggataaccaaatagctacatgcaaaagaatgaacttggacctcctagttatactgtatataagttgaaaggccttttcctagacccagcctaacattggataaaatcaattctccataaactcaaggagaattgcctgccagaacaaaaaccaacactttccagtaaataacaacagaagccagagtctacaacttttgtcattcacaatatttagtatacaacagacgtgtatactgttaatgggaaatgggacacacagtaaaggagaaaagcagtcaacagaaacagagaaacagacccaaagatgactcagaagttgaatcagcagagaaggacttaaaaatatctatcaaaaatatatttgggccgtgtgtggtggctcacgcctgtaatcccagcacttgcagagactgaggtgggtggatcacctgaggtcaggagttcaagaccagcctggccaacatggtgaaaccccgtctctactaataacacaaaaagtagccaggtgtggtggcatgtgcctgtaatcccagctacttcagaggctaaggcaggagaatcacttgaacccaggaggtggaggttgcagtgagctgagatcatgccattgcactccggcctgggtaacagcgtgagactatctcaaaaaaaaaaaaaagttcaaagacttaatagtcataatgagtgaacagaccggcaatcttagaagagaaacagaaacagaaaaagaccttaatgaaagtcttacaaagaacacctgcaatatctaaaatgaataatttactacctaggcttaacaacaaattagaggccgggcacggtggctcatgcctgtaatcccagcactttgggaggccgagacgggtggatcacgaggttaggagatcgagaccatcctggctaacacggtgaaaccccgcctctactaaaaatacaaaaaattagccgggcatggtggtgggcgcctgtagtcccagctactcgggaggctgaggcaggagaatggcgtgaacctgggaggcagagcttgcagtgagccgagaccgtgccactgcactccagcctgggggacagagcaagactccatctcaaaaaaaaaaaaaaaaaaaaaaaaaagaggggaggccaggtgcagtggctcacgcctgtaatcccagcactttgggaggccgaggtgggtggattgcctgaggtcaggagttcgagaccagcctggctaacatggtgaaaccttgtctctactaaaaatacaaaaatcaggtgggcgtggtggcgcacacctgtagtcccagctacttgggaggctgaggcaggagaatcgcttgaacctgggagtcagaggtttcagtgagccaagatcacgccactgcactccagcctgggacagagcaagactccatcacaaaaaaaaaaaaaaagaaaaaaaaaaggaaacgttcagtgaagttgaaggtagatcaaagcatacaaagagagatggcaaaaaaaaaaaaaaaaaaaaaaaaaaaaaaaaaaaaaaaagccaatacaggaaatctcaaaatcaggctgctttaaagaagccaaaagaaatatatatatacaacccgtatagtgtgtatgcacaataagatacacacatacacaccataagattctactcccaagaaattgaaattcaagaacaggcaaactaatctctgaggatagaaagtaaaacaacagttgcctaaagaggggatagggattgaatgggagaggacataaggaactttctagggtgatggaagtgttctatatcctgaatatagtggtgattaaatggatatatgcaatcatggagtctcatgaaagataccttttttttttttttttttttgagacagagtttcattctttttgcccaggctggagggcaatagtgccatctgggctcactgcaacctccacctcctgggttcaagtgattcttctgcttcaccctcccgtgtagctggtattacaggcatgtgccaccatgcccggcgaattttgtatttttagtagagacggggtttctccatgttggtcagggtggtcttgcactcccagcctcaggtgatctgcctgcctcagcctcccaaagtgttggggatataggcatgagccactgcacctggccctccaaagaggattttagaagagaggaaaccggtcaaagggattctttaagcctgtatatgaagtcctaggtagagccctgattgacccatacgtcaaactgaatagaattaacaacaacaacaaactcagcaattagaatatataaaaatagaagtagaaaatgttaaagtgggccggatgcagtggctcacgcctgtaatcccaatactttgggaggccaaggcaggcagatcacttgaggccaggagttggaaacagcctggccaacatggcaaaaccccacctctactaaaaatacaaaaaattagccaggcagggtggtgcatgcctgtaatcccagctactcagggtcaggcgcaagaatcacttgaactcaggaggcagaggttacattgtgctgagatcgtgccactgcactccagcctgggggacagagtaaaactcggtttcaaaaaaacaaaacaaaccttttgttcatcaaaggacactatgaagaaaatgaataggcaagccaaaattgagagaaatgttcaaaatacatatatctgacgaaagtcttgtgtctaaaatatgtctgtgtgtttacaccggtgtatattactcgtatctgcaaatctgtaattaaaaggctcacaatacaattttctttaatatgatggagttttgctcttgttgcccaggctggagtgcgacggcatgatctcagcttaccgcaacctccgcctcccgggttgaagcgattctcctgcctcagctgctgagtagctgggattacaggtatgtgccaccacgcccagctagttttgtatttttttaaatagagatggagtttctccatgttggtcaggctggtcttgaactcccgacctcaggtgatctgcccgcctcggcctcccaaagtgctgagattacaggtatgaaccactgcggtcagccaatacaactttttaaaaatgacaaaatacttgaacagatattttcctaaaggaagatatacaaatagccaataagcacataaaaagataatatcattagtaatgtggaaaatgcaaattacaaccatgctgagataccaccttacatccactggaatgtccaaaatttttgaaaaactgacatcgccaaatgttgctgaagatgtagagcgtctggaactctcttacattgccagtaggaatataacatgatacagccaatttggaaaactgttggcctgttttttatgaagtttaacatacacttaccacatgactaagaaccttcattcctagatattttctcaagataaatgaaaatgtatgtttaaatagacacttgtacgtgaatgttcatagcagcttataatagcaataataattattattgtcgttattttttcagatggagtcttgctctgtcacccaggctggagtacagtggcacaatcttggctcactgcaacctccacctcctggttcaagcgattctcctgcctcagccttccaagtacctgggattacaggcacacgccaccatgcccagctaatttttgtatttttagtagagtcgagatgtcaccctgttggccaggctggtcttgaactcctgacctcaaatgatctgcccgccttgggcttccaaagtgctggtattacaggcatgagtcactgcacccagcccaaaagtttcttatttaattgacaaaatcttatatatttatcatgtacaacacactgttttgaaatatgtatatattgtagaatggctaagtcaagctaattaatatatgcattatcccatatactttcatttttgtggtgagaacacaaaatcttagtaatttttaggaatacaatacactggccaggtgcggtggctcatgcctgtaatcccatcactttgggaggccggggaggggtggatcacttgaggtcaggagtttgagaccagcctggccaatatggtgaaaccccgtctctactaaaaatataaaaattagccgagcgtggtggcacacacctgtaatcccaactactcaggaggctgaggcaagagaattacttgaacctgggaggcggaggttgcagtgagctgaggttgcaccactgcactccagcctgggtgacagagcgagactccatctcaaaaaaaaaaaaagaatgtaatacattgttattaactagcatcaccatgttgtacaacagttctcttgaacatattcctccagtctaattgaaattttgtatctttagaccaacatctctccgtctaccctccccaggagatagaaattccttatccacaaggatggagacaatgttggtcaacccactttcatttacttaacattacttttattctaggtgtggcatctggcatgaaaccaaggacaagacactcagttgagctttgtgaaagagtgacagatgatggatagagtcatgggcctgcctcttatggagctaagaaatgaaatctggatacgggtaatcagtttgtggtagaaagtagtcactcatttccgtgagggaagcacaagtcctgtgtaggcccagagaagggaaaaagcctttttgggcataagagtgaatgttgagggttggagagaggaatggtctggaggctttgtagcattcatgttgtgccctaaaggacagataacatttggacatgagaagctgtagtgagccatgactgcaccactgcactccaggctgggcgacaggttgagaccctgtctcaaaaaaaaaaaaaaaaaaaaaaaagaagaagaagaaagaagatttggacatgaagagatgccaggcattacagacagagaatacagtatgagcaaaagcatggaggtttttttgttttttgttttttgtttttgagatggagatttttgctctttttgcccagactggagtgcaatggcatgatattggctcactgcaacctccacctcctgggttcaagcaattctcctgcctcagcttctagagtagctgggattacaggtgcacactaccacgcccagctaatttttgtatttttattagagatggggtttcatcatgttggtcaggctggtctcgaactcctgacctcaggtgattgtcctgcctcagcatcccaaagtgctgggattacaggcatgaggcaccgcgcccagccagcatggaggtatttgagagcaacagtgatcagaaccatttggttcaagcagcggttttaaaacggaagtggagaaggaattagcagatccctgacatcctcttcaatcagagttcctccattgtgaactggtttacatgtcagcattatggattttggtgcaacacctgcccccaacaggaagaaaagaagaaaaagaaagaagaggaaggaagaagagaaagacaaagaagaagaaggaggaggaggcggcaggaggagtaggagggaggaagaaggaggaggaagaaaaagaggaagaagaaaggaggaaggaagaagaaagaagaattgggaggctgagacaggcagatcacaaggtcaggagttcaagaccagcctggccaacatggtgaaaccccatcacttctaaaaatacaaaaattagctgggcgtgttggcacatgcctataatcccagctacttgggaggctgaggcaggagaattgcttgaacctgggaggcggaggttgcagtgagcagagagctcgccactgcactccagtctgggcaatgagcgagactgtcttgaaaaaaaaaaaaggaaggaaagaaggaggaggaggagaagaaagaagaagttttattattgttattttttgagatggagtcttgctctgtagcccaggctggaatacagtggcacactcttgactccctgccacctctgactgctgggttcaaaggagaaggcggaagaagaaggaagaagaagaaagaaggagaaaggctgggtgcagtggctcacacctgtaatctcagcactttgggaggccgaggcaggtgaatcacaaggtaaggagttcgagaccagcctggccaactggtgaaaccctgcctctactaaaagtacaaaaattagccgggcgtggtctgaggcaggagaatcgctttaacctgggaggaggaggttgcagtcagtcaagatggcgccactgcactccagcctgggtgacagagtgagactttgtctcaaaaaaaaaaaaggaaagaaggagaagaaagaaggaggaggaggaggaggaggagagaaggaggagaaaagtagttgaggcccaaacaccaagagggagcaaagattgaaaagatgagatgagccatgaaagcaagtacaggagttactgatggtactggggagcccgtgtaggtttaggatctgagcttttggaagattgattgggtagcctttgagccacctgataagtggaaagaacaagagaggctggatctgtgttttcaggaagcatatgttggcccagcaattgctggcttgtagtgaggaggcacacaactggcctaggacagtggtcatgaaaatgcagaggaggtaaagtccctgcactcctagggagactagtcctgatgtcagtctggagtcagtcagaatggtgtcctctccctccctgcactacccagcccagtcagtgggaggacttcctcaattccagtagccattcaagtccctggaattggtggctgtcacttgcaaactatagccacttgagcagaaatgggccaggattacttatctttaatctgcatatcattgggaggcacttacctgctagctctggctaaaaactagagcaaccctggcctgccgtagctcctgctgcccagacaactcctccaatatgaaagggatgaggggaactcaaagttacaatgtcctacttggagcagtaagttcagtagacatatcacttgcctcattaacatcaagcatcccaaaacccagtctgggtcagttttgcccagagtggggtttgtagaacacgggttctcctgggatcctatacctagcccagaatcagttgcaaaagccaggccatagcgaattgtcctgccagccagatagcagagaatctgacggcagcaggcagaaggagccgctccattgcagtaagccaagatcgcgccacttgcctcattacatcaagcatcccaaaacccagtctgggtcagttttgcccagagtgaggtttgtagaacacgggttctcctgggatctatacctagcccagaatcagttccaaaagccacgcttttggactttctctctctctctctctctatatatatatgtgtgtgtgtgtgtgtgtgtgtgtgtgtgtgtatacacacacatacatacaatgttgtatatattgtatatgtgtgtgtgtgtatatatatatatatatatatttttttttttttttttttttgagacagagtttcactcttgttgcacaagctggagtgcaatggcacgatttcggctcactgcaacctccgcctcctgggttcaagtgattctcctgcctcagcctcctgagtagctgggagtacaggcgtgcaccaccgcgcccggctaatttttgcatttttagtagagatggggtttcaccatgttagccaggatggtctcgatctcctgacctcatgatctgcccacctcggcctcccaaagtactgggattacaggcatgagccaccatacccggccttcatttttatatttttagtagagacaggatttcaccatgttggccaggctggtctcgaacttctgacctcaggtgatctgcccacttcagcagcctcccaagtgctggaattacaggcgtgagccaccgcctggccaattttttgtatttttagtagagacagggtttcaccatgttggccaggcgggtcttgaactcctgatctcagatgattaatccacctcagcctcccaaaatgctgaaattacagtgagccaccatgcccagcctctgagtcaaagtttctttaattgcaaagtagagatcattgttttttttttgctttttttgttttgagatggagtctcgctgtgtcacccaggctggagtgcagtggtgtgatctcagctcactgcaacctccacctcccaggttcaagtgattctcctgcctcagcctcccgagtagctgggactacagatgtgcgccaccatgcccggctaattttttgtatttttagtagagatggggtttcaccgtgttagccaggatggtctcgatctcttgacctcgtgatctgcccgcctaggcctcccaaagtgctgggattacaggcacaagccaccgtgcccagcctgagatcattcttacttcacaggatttccctgataattaagtgaacacacacacacacagcatctaacacaaaatactttgtacacagtagatactagacaaaagataaccatggctactgctgtttcttttttctcctcctcctgcctacccttccccctcatccttttatctatggccaagccctcctgaagggtctctttgaatcgcgttacctcctagtttgacaccagtgctcttatttccctgtggcattggagggctagtcacatggcactgtggccaaggagaaggatgatgttccaggccaaacacatctgcaaatgtgttgagctggtgtccttgatgacaaatcaaattgttttgtgtttggttccccagaggcctgagtacctcagagaagccaaggttcctgcaggaacctcgtgcctgctgtccacgttgggcttcattttatcaaaatacagcaaagagctcatcatttctcagctcctgtcggaagcaacagtggctgttcctggacacagagctgcagacaaggagtggtgaggattcaggcaagagaagatgggcagcttccccttcatgcttccctctcaggaagaagccagggtgcctgggatcaaagaggctaggaggctggtgaggcctcctgtccctgaaagcggtcagtgaacccaaaagttgaaaagcctctgctttgagagtggttctgctgccctactggtgctgacagttttagaaactgacatggcagagctgaccaggcagactatatttttaggactctgctggggcctattgaaacgtccacattactcagatcaccaatctggctggagctaccaacacagcattctttgctgttttagaatacaaagctacctcccctcggggcttcactttgcagccactgagaatccaaggcttcgctaaattacacgtttccatggtcccactggcttgctttgcagggcttccacaagaatgggctgggagcgtaactgagtaactgagtcatgattaggggttaattaagaggagaaatgggctagctgtttcctaagataagggaggggaagtttttttaaaaactgtaactgctcagaacagtatgtgctaaggcagactttctgtgctgggagacagaacacttctgcagtgatgtagcagaatccttaaaggatcctgccaatgactctttttttctggtgtcacatccaatctttgctgcagccaacacttcctggagatgaaacacaacttcaatacagagatagcaagttcacactctggcagtcatgcaaggatgagtgatgcgggccttttctaagtgagtatgaacgggtgggacctacggtaaattcaatgcagtataatccgtctacggggcagtgggcacccagcaacagccaactgttaccaaatcaaagtgcaggcccaggatgactcatcttccagcatttcatgagcaaccagaagtctacatttctttgagaaaaaaaatttttttgagacgaggtcttactctgttgcccaggctggagtgtgtagcttctacctcccggggcttaggtgattctcccacctcaagtctcccaagtagctgagactgcaggcatgtgccaccacgctggctaattttttttttttttttgagtcggagtctcgctctgtcccccaggctggagcgcagtggcgcgatctcggctaactgcgagctccgcctcccggggttcgtgccattctcctgcttcagcctcccaagtagctgggactacaggcgcccgccaccacgcccggctaatttttttgtattttgtagtagaaacggggtttcaccatgttagccaggatggtctcgatctcctgacctcgtgatcctcccgcctcggcctcccaaagtgctgggattacaggcgtgagccaccgctcccgccccatgccagctaatttttttgtagagacagggcttcgccatgttgtccaggctggtctccaacccctaggctccccgccttgccctcccaaaatgtggagattacaggtgtgagccacaatgcctggccattttgaggaatttttaaacactggctttaatttttcaaagtatctggagcaacttctgcttctgagagatagagtagatattcttttccccagtcctcctgctaagtacagctaaaaaccccaaaaacactctatattatacaaaatgaacataagatgactctgaacaagaagaaagcagattggttagggacctccagatccaaggaatgactcagcagtgacctccctaagttttctttttgcctcatatatgtcagaatgggtactggagaagccagcaacccaaaactgccaacgattgcagacaaaagccccaagaaaagcttgtcttttttttttttttttttttgaaagagtgtcttgctctatcgcccaggctggagtgcagtggtgcaatcatggctcactgcagccttgatctcccaggcttaagctgcttagactacagatctgcgccaccatgcctgactaatcaatacagttgttttttttttttgaaacggagtttcactcttgttgcccaggctggagtgcactggcgcgatctcagctcactgctgcaacctctgcctcctgggctcaagcagttctcctgcctcacctcctgagtagctgggattacaggcatgcaccatcacgccctgctattttttgtatttttagaagtagagatggggtttcaccatgttggtcaggctggtctcaaactcctgatctcaggtgatccgcccgactcggcctcccaaagtgctgggattacaggcatgagccatcgtgcccggcctttttgttttttttgtttgtttgtttgttatgtttttttgtagagatgggggtctcactgtttcccaggctgatctcaaattcctgggctcaagcaatcctctggcctcagcctcccaaagtgctggaattataagcgtgaaccatggtgtcctgcaagtctgctttctgagtcaaaggacaagaaaataaacagcctagaaagacagacagccttgatttttttatttttagttttcatttggtttgatttttcttgagacagagtctcactctgtcgcccaggctggagtgcagtggcacgatctcgactcactgcaacctctgcctctcgggttcaagcagttctcctgcctgcctcagcctttcgttcggcctcaaatgatccgcctgcctcagcctcccagagtgctgggattacagccatgagccactgcatccagccgacagacagcctttagactatagcccctctactccagccccaccctcacccccatcagcaaaggccaagtggaaagcctagatctccaaaccttgttaggctgtaatgaagtactccttcccttccccaccagggtgatgtagaagaggcctagttatccactcaggcctttcatccataatcaaggccacctctcccaacaatgtcggtggaggcccactccctctcccagccggtgtggtatcagtgaatgcctaatagagaaactggactcccacccctgcccagcagtaagaaggagccctgccaacaccggtgtcaatggaggccaagtggaaaacctggacttccacaccctcctggtaaaaataaggtagcatccgcactttccccacagaagcaatgttagaggaggcctgctgaaacacaagctataaagtaagatccagaggctgggcgcagtggctcatgcctgtaatccctgcactttgggaggctgaggtgggaggattgcttaagcccaggagttcaagaccagcttgggcaacatacggagaccttgctgctacaaaaaaacaaaaaacaaaaaaattagctgggcatgctgatgcctgcctgtggtcctagctacttgggaggctgagatgggaggattgcttgaggccagaaggttgaggctgcagtgagtcatgaccccactgcactccagcctgagtgacaaggtgagaccctatcccaaaacattaaataagatccagagtcttataacatgatgtgcaaaatgtccaggtcttaaagtcatttgaaccggaaccagaaaattttgaataagaaaggataatctatatacactaacaccaaaatgacacagatattagaattatctgaaaaagattttgaagtagccatcataaaaatgcttcaatgtatataacgtgttcaaatacaaacatacttggaacaaatgaaaaaatggaaagtctggaaaatataatgtctcaacagatcgatacataaagaagaaccaaatggaaattttaggaataaaaaatacaatgattgaaatttaaaactcaatgaataagctcagcaacagaatggagaggacagaggaaagaatgagtttgaagacagaaaactacaaattatccaatctgaacactagggaaaaaaaatagaatggaaaaaaaaaaaaacagagcctcagggccatgtgagacaataacataagatctaacattcatgtcattggagtcacagaaggagaagaaatattaaaagaacaaatagccgaaatttgcccaaattttgcaaaagacaaactgacagattcacatagctgagcaaacctcaaacaggatatgcccatgagaaaacacatcataaacttctcaacatatactttttttttttttttttttttgagatggagtctccctctgctgtccaggctggagcacagtggcacaatctcagttcgctgcaacctccgcctcctgggttcaagcgattctcttgcctcagcctcccaagtagctgggactatagacgtgtaccaccatgcctggctaattttttgtatttttagtagagatggggtttcaccatgttggccaggctggtctcgaaatcctgacctcaggtgatctgtccgcctcagactcccaaagtgttgggattacaggtgtgagccacccgcacccagccacaaattcttgaaagcagccagagagaaatgatacattacctatagggggaaaacaattcaaatgatgctgaatttctcgtaaggcaccatggaggccaggaggaagtggaacaacatttttttttttagacaagagtcttactctttcacccagggtggagtgcagtggcgcgatcttggctcactgcaacctccgcctcccaggttcaagcaattcttctgcctcagcctcccaagtagctggggctacaggcgcgccaccatgcctggctaatttttgtatttttagtaaagacaggagtttcaccatattggccaggttggtctcgaactcatgacctcgttatctgcccacctcggcctcccaaagtgctgggattacaggcctgagccactgcgcccagctggaacatttttgaaatgctgaaagaaaagaactatcaacttggaatcccatacctggtgaaacaatccttcaggaattggggggaagggctgtgggggaggaaatcatgacattcttacatgagggaaaaactaagggaatttgtcactggcagatttactttatagggattactaaaggcaattctctaaacaaagggagtggtaacaaaaggaatcttggaacatcaggcaggaaaaaaggacaaaaagagcaaacatacaggtcaatacaatagattgtctttttcctcttgagtattctaaattatgttcaacagttgaagcaaaaattacgcttgtctaagtggctgtccatatatgtagaggaaatacttaagatagttacattataatgggagagtaaaaggacacagaaaaaggtttctttctttatttgtaatctggtagaactttccagaggtatagaggtaatgtttctacctttcactcaaactggtaaaatgtcaacaccagtagactgtgataagttactgcactccagcctgggcgacagagcgagaccttgcctcaaaaaggaaagaaaaaaaagaaaaaaagaaaagaaaaaggaacacataccaaacttaaatgtgtacgtaccaaacaacagacttgaaaaataagtgaacctggccaggcgcggtggctcatgcctgtaatcacagaagtttgggaggccaaggcaggtggatcacctgagatcaggagttcgagaccagcctggccaacatggtgaaaccccgtctctactaaaaatacaaaaattaggctgggcgtggtggctcacggctgtaaacccagcactatgggaggccgaggtgggtggatcataaggtcaagagattgagaccatcctggccaacatggtgaaaccccgcctctactaaaaaaatacaaaaattagctgggtgtggtggtgcacggctgtaaacccagcacttggtaggctgaggcaagagaatcgcttgaagctggaaggcagaggttgcaatgagccgagatcatgccattgcactccagcctgggcaataagagtgaaactccatctcaaaaaaaaaacaaaaaccccaaaaaacaaaacaagaaaaactattagacagaaagtcaacagggctgagtgtgtggctcacacctgtaataggatatagaatttaacactaccattagccaacaggatgtaatcagcacttatagtacatcaacaacaaaaaaaacacattctttttaggcacccatgaaacatgtatcaagatcaaccatatcttgggttataaaacaaacctcaacaaatttaaaagaattgaaatcatacagagtgtatgaccaaaatggactcaaacttagaattaataacacaaagacaatctccaggaagatcttcaaacacttgaaaactaaataacacatttctaagtaatctatgaaccaaagagcaatcctcaagagaaatacaaaattcattaacctgaatgaaaatgaaaatacaatgtatcaaaatttgtaagatacagctaaatcacaactaggaaggaaattaatagcactaaatgcttacattagaaaagaggaaaaaaacctcaaatcaataatacaagcttccacctcaggaaattagaaaagaacaaaataaaccctaaacaaacagaaagaaggaaaagaagagaaatctatataattgaaaacagaaaaacagtagagaataccaacaaaacagaaaactggttctttgaaaaaatcaataaaattgacaaatcttttattttcttttaattgagacagcctctcgctctgttacccaggctagagtgcgatcacagctcactgcagcctcaacctgccgggctcaagcagttctcctgcctcagcctcctaagtagctgtgactacaggtgcatgccaatatgcctgtctaatttttgtattttttttagagacagggtttcaccatgttgctcagactggtctcgaacttctgggctcaagctgtccacccacctcaacctcccaaagtgctcagattacagacgtgagccattgcctgaggccaaattgacaaatctttagcaaggctgacctaaaaagagagagagaggctgggtgcgatggctcatgcctgtaatcctagcactttgggaggccgaggcagatggatcacttgagctcaggagttcaagaccagcttgggcaacatatcaaaaacccaaccctgcctctacaaaaaatacaaaaattaaccaggcacggtggcacacgtgtagtcctggctacttgggaggctgaggtgggaggatcccataaacctgggaggcggaggttgcagtgagccgagatcatgccactgcactccagcctgggcagcagagtaagacttcatctcaaaaaaaaaaaaagttatttttatagagacagggtcttgctatgttgcccatgctatccttgaactcctgggcccaagtgatcctccagcctcagctctcccaaagtgctgggattacaggcatgaaccactgtgcccaccccatacattcttcatatcaggaataaaacagaagctatcactacagactctgcagataacgataggataataagggaattttattgtattgtttatttatgtatttattttttgagacagagtctcgctctgtcaccaggctggagtatggtggcgtgatctcggctcactacaatctctgactccctggttcaagtgatactcctgcctcagcctcccaagtagctgggattacagccacgcgccaccacgcccagctaacttttgtatttttagtagagacgggtttcaccatgttggccaggatggtcttgaactcctgacttcgtgatctggctgcttggactcccaaagtactgagattacaggcatgagccaccgtgcctggccctggaaattttataaacacacatttgttttatatgttactaacacacatttgacaacttagatgaaatggactaattccttaaaaaataaaaactactacaacatacccgatatgaagtagataatttcagtagctttataactattaaggacattgaatttgtaatttaaaacttcacccccaagaaatctttactccagatgatttcagtggagaattctatcaaacgtctaaagaattaacaccgattctacaaatctgatccagaaaatagaagagatgaagcatttcctaagtcattttatgaggcccatcttacccatatcaaaaccagacaaggaaaatacaaaaaagatgactacagaccaatgacctttaataagaacacagacacaaaaatccttaacaaaacatcaacaaataggccaggcacggtgcctcatgcctgtaatcccagcactttgggaggctgaagtgggtggatcacctcaggtcaggagttggagaccagcctgaccaacatggtgaaaccctgtctctactaaaaattaaaaaaaatgagctgggcgtggtggtgcgtgcctgtaatcccagctactccggaggctgaggcaggagaatcacttagaactcgggaggtggaggttgcagagagccaagatcgtgccactgcactccagcctgactctgtctccataaataaataaataaataaataaataacaaatagaggccgggcacggtggctcacgcctgtaatcccagcactttgggaggccaaggcaggcggatcacctgaggttgggagttcgagaccagcctgaccaatatggagaaaccccatccctactaaaaatacaaaattggccaggcacagtggctcatgcctgtaatcccagcactttgggaggcctaggtgggcggatcacgaggtcagaagttcaaaaccagcctggccaacatggcaaaaccccgtctctactaaaaatacaaaaattaaccaggcatggtggcaaacacctgtaatcccagctacacaggaggctgaggcaggagaattgcttgaacccgggagggggaggttgcagtgggccaagatcgcaccattgcactccagcctgggtgacagagcgagactccatctcaaaaataaaataaaataaaaaatacaaatacaaaattagccgggcatggtggtgcatgcctgtaatcccagctactcgggagactgaggcaggagaattgcttgaacctgggaggcacaggttgccgtgagcggagattgtgctattgcactctagcctgggcaacaagagggaaactccgtctccaaagaaaaaaaaatgaatagaattcagcaatatatgaaaagaattatatactatgaccaggtggggattattccagggatgcaaggctaagtcaatattcaaaaatcaatcaatgaaaaaaaatcaatcaatgtattccaccctgttaacaggctaaagaaaaatcaattgacagagagaaagcacttgacaaaattccgtacccattcataataaaaactctcaggaaactagaaataaaagggaattttctcaatttgatagagtatctacaaaaaacctacaactagcatcatacttcacagtgagagactgaatgctttccccctgactgaacacaaggcaagcatgtccactctcaccaatcttattcaacacagtgctggaaattgtagtctgtgtaataagaataggaaaagaattttaaaagcatacatattggaaagaaagatatcaggcggtccttataatggcatgatggcctttatagaaaatttcatgaaatctaaaaaaaaaaaaaaagctagaaccagtaagtgaattcagcaaggtcacaatacaaaatcaacatacaaatatcaacatatatatattagcaatgaaaacacggacatcaacatttaaaacataatatttataatcacagaaaaaggaaatatttaggtatgaatccagtaaaacatgtacaagactcatatgatgaaaactagaaaatggtgttgaaagaaatcaaagaagatctaaatagacagatatactgttttcatgacttcaaagactcatcacagtaaggatgtcaattctccctaaattaatatacaagtttagtgcaattcctatccaaatcccagcatgatttttgtaaataaagattattataaaatgtatgaggaaaggcaaataaactagaatagctgaaacaattttttttttttttttttttgagccagagtctcactctgtcacccagactggaccacagtggcgtgatcacagctcattgcagccttgacatcctgggctcaggtaatcctcctacctcggtctcccaagtaactgggaccacaggtgcgtgccaccacgcctagctaatttccttttttttttttttgagacggagtcttcctctgtggcccaggctggagtgcagtggcatgacctcggctcactgcaagctccgcctcccaggttcacgtcattctcctgtttcagcctcccgagtagctgggactacaggcactcgccaccacgcctggctaattatttttgtatttttagtagagacgaggtttcaccgtgttagccaggatggtctccatctcctgaccttgtgatccgcccatctcggcctcccaaagtgctgggattacaggcgtgagccaccgagcccggcccaggcccagctaatttttaaaaattattttttatacagacaaggccttcctgtgttgcccagggtgcttgaattcctggactcaagcaatcctcccacctcagcctcccaaagtgctaggattacaggcatgagccactgtgcccagcataaaataattttcattaaaaaaactttttttgagacaaggtcttgctctgtcacccaggctggagtgcagtggcacaatcttggcccactgcagcctcccgagtagcttcaactgtagacatgtgccaccacacccagctaatttttcaattttttgtagagacggggttttgccatgttgcccaggctggtcttgaactcctgagctcaagcaattgtcccaacttggcctcccaaagtactgggactacaggcgtgcactaccacacgtggcctaaaatatatttttaaaaagaagattaagccgggcgcagtggctcacgcctgtaatcccagcactttgggaggctgaggcgggtggatcacttcaggagtttgagaccagcttggccaacatggtgaaccaccatctctactaaaaatacaaaaaattagccaggtgtgatggcaggtgcctgtaattccagctactcggtaggctgaggcaggagagttgcttgaatctgggaagcagaggttgcagtgagctgaggtcgtgccattgcactccatgcactccagcctgggcaacaagagcgagattccatctcaaaaataaaaaaattaaaaaggctgggcatggtggctcacgcctgtaatcccagcactttgggaggctgaggcaggcagatcacgaggtcaggagttcaagaccagcctgaccaacatggtgaaaccccatctctactaaaaataaaaaaattagcccggcatggtggcgtgcacctgtaatcccagctacttaggaggctgaggcaggagaatagcttgaaccggggaggtggaggttgcagtgaaccgagactgcaccattgcacaccagcctgggcgacagagcgagactccatctcaaaaaaaaaaaaaaaagaagattaaagtgggaggaatcactgcctaatttcaagaatacagctatagtaattatgactgtggggtattggtggatggaaagacacatcaatcaatggaagacaatagaggacccagaaataccccatgcaatacagtggaggaaggatagttttgtcaacaaatggtactgaagcaaattgatgttcataggaaaaaacaaaaaacaactccacagaaattggctttctgtgatctgatacagcctgcctagaagcagaagttctggctgcccatcacactgtttttagtaaacctagagttttatttgaaatattgagtctggcccaggcgcggtggctcacgcctgtaatcccagcactttgggaggccgaggcaggtggatcatgcggtcaggagatcgagaccatcctggctaacacggtgaaacaccctctctactaaaaatacaaaaaattagccaagtgtggtggcgggcgcctgtagtcccagctacttgggaggctgaggcaggagaatcacttgaacccgggaggcggagggtgcagtgagccaagatcgtaccactgcactccagcctggataacagagcaagattctgttaaaaaaaaaaaattataaaagatggaaaagatggagagccaccaaggaagcaataattctgcatctagactgccttcagacttgttcagacttgaactgcaacatcaactcttccctgggtctttagcttgctggccagttctgcagattcagatttgccagcccccacaataagacaattcctttaattgccttagttctgatgaatacagagatctaggctgggtgcagcggctcatgcttgtaatcctagcactttgggaggctgagacaggcggatcacttgaggccaggagtccaagaccagcctgaccaacatgacaataccccatctctgctaaaaatacaaaaaaattagccagatatggtggcatgcaactgtaatcccagctattcttgaggctgaggtacaagaatcatttgaacccaggaggcagaggttgcatgaatgctgctgaaaaaaaaaaaaagtaaaataaaaatgtaaaagtgaccattaaaaaaaaaagcaaccaacctaaacctcatgccttatacaaaaattaacttaaaatggaacaggtttaaatgtaaactgtaaagttataagatgtttagaagacaagatagaagaaaatcatcaggacctaggagtctttccccagggctcagggaaaagttctcagacatgatgccaaaagcaagaaccataaaagaaaaaatatatacataattggacttcatcaaaattacaaacttttgctctgtgaaagaccccattaagaggatgaaaagacaaactacaaagtgagagaaaatatttgcaaaccatatatctgacaaaggacttatatttagaatatatataaagaacactcaaggccgggcattgtggctcactcctgtaataccagcacattgggaggccaaggcaggcagatcacttgaggccgggagtttgagaccagcctggccaatatggtaaaacccaatctctgctaaaaatacaaaaattagctgggcacggttgtgcatgcctgtaatcccagctattcaagaggctgaggcacgagaatcacctgaacccaggaggcagaggttgcagggagccgagattgcaccactgcactccagcctgggctacagagctagactctgtctcaaaaaaaaaaaaaaaaaaaaaggttgggcgtggtggctcaggcctggaatcccagcattttgggaggccgaggcgggcggatcacgaggtcaggagattgagaccatcctggctaacacggtgaaaccccatctctactaaaaatacaaaaaaattagctgggtgtggtggcaggtgcttgtggtcccagctactcaggaggctgaggcgggagaatgacatgaatctgggaggcggagcttgcagcgagagccactgcattccagtccagcctgggtggcagagcaagactctgtctcaaaaaaaaaacaacaaaacaaaacaaaacaaaacactcaaatctcaacagtaagaaaaacaccagataatcccattagaaaatgtgcaaaaaaaaaaaaaacttaagatatttccccaaagagaatatacagaaggcaaataaacacgtgaaagaatgttaacataactatccattaggaaaattaagaccatgatgagataccattacatacttgtgagaatagctaaaataaaaaaatagggccaggcgtggtggctcgtgcctgtaatcccagcactttgggaggctgaggcaggcagatcacttgagtccaggagttggagaccagtctggccaacatagtgaaaccctgtctctactaaaaatacaaaaattagcttggcatgatggtggtgcacacctgtaataccagctacttgggaggctgagataggagaattgcttgaacctgggaggtggaggctgcagtgagccgagatcatgacactgtactgcagccggggtgacagagtgagactctgtctcaaaaaataataataaaataaaaaaaataataaaacagaataccacatgctagaggatgtggacaattgaatctttttttttttttttttttttctgagatagagtcttgctctgtcacccaggctggagtgcagtggcacgatcttggctcactgcaacctccgcctcctgggttgaagcaattctcctgcctcagcctcccaactagctgggattacaggcatgcgccaccacacttggctaatttttgtatttttagtagagatgggtttcaccatattggccaggctggtctcgaactcatgacctcgtgatctgcccgcctcagccttccaaagtgctggtattacaggcgtgagccaccatgcccggccaacaattgaatctttcatacattactgttgggaatgtaaaatggaatagccactctgtaagctaattttgtattttcatttatttatttttaagagacaaaaacttggagtgcactggtgccatcatagcttactgcagcctccaactcctgggctccagcaatccttccacctcaccccaccgagtagctgggactacaggtgtgcaccccaaagtctggctaattttttaatttttaatttttgtacacatagggcctaattacgttgcccaggctggtcttgaactcctgagctcaaatgatcctcctgccttggcctcccagtgtgctgggattacaggcatgaatcaccatgcccggccctactatttgctttaaacactaaacattcccttaccatatgacccagaaattttcctacttggcatttatcccagataaatgaaaacttatgttcacacgaaagcctgtacatgatttttcataacacatttatttgtaatagccaaaacctggaaacaaccaaaatgaccatcaaatagatgaatggctaaataaaccatgatatatccatatcacatgtatcgagttaaatgggagccccccaaaagacatgtacatatattaactcctgaacctatgaatgtgacgttcgaaaatagcgtctttgaagccgggcgcggtggctcacgcttgtaatcccagcactttgggaggcctaggtgggcagatcacctgaggtcgggagttcgagaccagcctgaccaacttggagaaaccccgtctccactaaaaatacaaaattagcccggtgttatggcacatgcctgtaatcccagctactcaggctgggcaggagaatcacttgaacccaggaggcaaaggttgtgatgagccgagatcgcgccattgcactccagcctgtgcaacaagagcaaaactccatctcgaaaaaaaaaaaattaaaaatagggtctttgcagctgtgattaagttaaggatctcgagatgatatcaacctagattatctgggtgggccctaaatccaatgacaagtgtctttttttccccaagcaatcaatcctcttgttgctgggaccacaggcatctgccaccacacctggctgaatttttttttttttttgagacaaagtctccctgacatccaggctggagtccagtggtgctatctgggctcattgcaacctccgcctcccaggttcaagcaattctcatgcctcagcctcccgagtagctgggattacaggtgttccccaccatgcccggctaatttttgtatttttagtagagacggggttttgccatgttggccatgctggtcttgaactcctggccagaaattattcgctgccctcagcctcccaaagtgctgggattacaggcgtgggccaccatgcccagccaacatttgactaattttttaaaaaattttttgtagggacaggtctcactatgttgcccagactgttctcaaactcctgggctcaaatgatcctcccgccttttcctcccaaagtgctgggattacaaaggtgagccacgtcactcagccaataagtgtccttttaagagacaggagaggagaaggcacagacatagggaagaccatgtgattggagcgatgcccttacaagccaaggaatggctggaaccaacaaaacagaacctagcaaggaagcattctcccctcgaggcttcagggggagcatggccccgctgacacctgggtttcagacttctggcttccagaactatcagagaataaatgtccgttgctttaagccacagaccgagtttgttgtgattggttatggcagttctaggaaacaaatataccatgggatcctacttagtaataaaaaggaacaaacttgatacagcagcaacttagctggatctcaagggctgagtgagaaaagccatctgaaaaggtcacatactgtatagttccacttacgaaacattcttaaaaagacaaaattataaagatggaaaatagtgcttgccaggtgttagggatggtgggggacagtgtaactataaaggaatatctccctgcacaaagaagggagatctttgtggtgatatatttatcttggttgcagtggtgcttacaggaatataaacgtgataaaatgacgtagaactatgtattcatatcagaccagagtacatttcctgcttttgatattgtgttgtaattatggaagaggcagccatcggaggaaactgggtgaaggctatgtggaacttttctgcactatctttgcacttcctgtgcatttacaattatttcaaaataagaagtcaaaagttaatttgaataagggagctgggtgcagtcgctcatgcgtgtaattccagcactttgggaggttcaggtgggaggatcgtttgagtccaggagttcaagaccagcctgggcaataaagtgagaacccatttctatttaaaaaaaaaaaaaaaaagaccgggcaccgtggctcacgcctataatcccagcactttgggaggctgaggcgggtggatcacaaggtcaggagtttgagaccagcctggccaatatggtgaaaccccatctgtactgaaaaacaaaaaattaactgggcatggtggggcgcgcctgtagtcccagctactcaggaggctgaagcaggagaattgcttgaacctgggaggtggaggttgcagtgagccaagatcacaccactgcactccagcctgtgcaacagagtcagactccatcccctttaaaagacaaaaaaaaaaaaggccacagtggctcacacctgtaatcccagcactttgggaggccgagggaggcagatcatgaggtcaagagttcgagaccagcctggccaacgtggtgaaaccccgtctctactaagaatacaaaaactcggcagggtgccgtggctcacacctgtaatcccaacactttgggaggccaaggcaggcggatcacgaggtcaggagaccgagaccatcctggccaacatggtgaaaccctgtctctactaaaaatacaaaaaaattagctgtgcgtggtggcgtgtgcctgtaatcccagctagtcaggaggctgaggcaggagaattgcttaaaccaggaagtcagaagttgcagtaagccgagattgcgccattacactccagcctggtgacagagtgagactctgtctcaaaaaaaaaaaaaaaaaaatacaaaaattagccgtgtgtggtggtggcgcatgcctgtaatcccagctactcaggaggctgaggcaggagaattgcttgaacccgggaggcggaggttgcagtgagcccagatcatgccactgcactccagcctggagaacagagcaagactctatcttgaaaggaaaaaaaaaaaagaaggaactttttgggatgatgaatatgttctatgtttcatgggtaaatacaactctcaaaactcatccaactagaccgttaatgtgggtgcatgttcttgcatgtaaattacacctcagtaaaaaaaaattttttatacaaaaattagccaggcacggtggggcacctgtaatcccagctacttgggaggctgaggcaggagaatcgcctgaacccagaaggcggaggttgcagtgagccaagatcgctccactgcaccctagcctggccgacagagcaatactccgtctcaaaaaaaaaaaaaatttagttaaaataaaaacaacaacaaaagtaagaggagcaaacaaaatgtatcaatttcagccgaggttttctagggcaaacactagaatattgtaccttttgctacctgataccttatcaatcaatatatattactgagcacttcgatgcaaggattccatcacctccccaacatgtgatatagaatagaagcaggtaaatgtttactaaatgaaggtacacagcgggcttttggagaggaaatagactctggcctccagccatggaataatttatactgtctcttgctaacatacctggagccgtttcctcattttgtgatccaaagagtaaacatgtaaaaccagccaatcttaggttatattcttgccatcctagagagtaagtgctccaggacatcagagtaagaagtctggaccaggggcaattctctttgtataatgtggattattgtttatgttcctatcttgtttccctgttagactgtgagctggagagtttgcacttttatgtgtcgttctaacactctcagtccttacagttcactgaacccagcagccagtcaaaaatctttgccactgttgaattgtaatgctgtacgttcaggggaatacattgctatttattaaatgaaggagaactttcaaatttaagaagagattgttttgcatgctgaagacagtaggtattataaaatacttaaatgggagcctttcccaccccagaacaacctacttagcattagataaatttactatttaaagtagccaaagaccacttctagaaatagaacctaaagaaataatagcaaatgcatgtagagaaaaaaaagctaaaagaaatctttgtaaaaaaacaaaacaacaacaacaaaaaagcgcatttagaagatttgttgcagcattgtttgtttgttttgagacagggtctcactctggcgtccaggcgggagtgcagtggtgaggtctcggctcactgcaacctctgcctcctgggttccagagattctcgagcctcagcgtcccaagtagctgggattacaggcatgcgccaccaagcccagctaatttttgaatttatttatttatttatttttattcttatttttttttgagacagactcttgctctgtcacccaggctggagtgcagtggcgtgatcttggctcactgcaacctctgcctcttgggttcaagtgattctcctgcctcagcctcctgagtaactgtgattacaggcgccccctatcacgcccagctaatttttgtatttttagtagagatggggtttcaccatattgggcaggctggtctcaaactcctgaccttgtaatccatccgcctcagcctcccaaagtgctgggattacaggcataagccactgagcctggcgtaatttttgtatttatagtagagatggggtttcaccatgttggccaggctggcctggaactcctgacctcaagcgatccgccctcctcggcctcccaaagtgctgggattacaggtgtgagccactacagctgtccaaaatgcacaccagattttgagaattttgttcatgaaaaagtacttaatttttttttttttttttgagacagagtctcgctctgtcgcccaggcgggagtgcagtggcttgatctcggctcactgccagctccgcctccggggtccatgccattctcctgcctcagccccccgagtagctgggactacaggtgcccaccaccacgcccagctaattttttgtatttttagtagagatggggtttcaccattagccagcatggtctccgtctcctgacctcatgatccgccctcctcagactcccaaagtgctgggattacaggcgtgagccactgtgcccagccaaaactatttaaatttttaaaatattggttacattttgagatgataatatcttggatatgttggacacatagggttgttgtgaggactgaatatgctgagagttcttatacatatcctgccctcataaacacccaagcttcctactgtcagcatcccacactacagtggtacatttgttaccgtcaatgacagatcatttccatccaaacttcatagttaatgttaaggttccctcttagtgtcaagcattctatgggttttggcaaatgcataacgatatctattcaccatgatgctatcatacagtgtaatttcattgccctgaaaatcgtctgtgctctgcctgttcattcctctttcccctcattccttggcagccactgatctttccttttttatacatatactcctagtttaggagctgaactcttttacttttttcttttttttttttcttgagacagagtctcaccctgtcacccaggctggagtgcagtggcgcgatctcggctcactgcaagctccgcctcccaggttcacgccactctgctgcctcagcctcccgagcagctgggactacaggcacccgccaccatgcccggctaattttttgtatttttagtagagacggggtttcaccatgttagccaggatggtttcaatctcctgaccgcgtgatccacccgccttggcctcccaaagtgctgggattacaggcgtgagccaccgtgcctggcctcttttacttttttaaatgtgaattttagaaaattttaaattacacatgtgggccggggacagtggctcatgcctgtaatcctagcactttgggaggtcaaggtgggtggatcacttgaagtcaggggtttgaaagcagcgtggccaacatgacaaaaccccgtctctactgaaaatacaaaaattagctgggtgtggtagcgggtgtctgtaatcccagctactcgggagactgaggcaggagaatcgcttgaacccaggagacggaggttgcagtgagccgagattgctccactgcactccagtctggatgacagagagagactccatgtcaaaataaataaataaataaataaataaataaataaataaataaataaaatagtagctctggttgagggcagtggctaatgcctgtaatcccagcactttgagaggccaagacaggagcatcgcttgagcccaggagttcaagaccagtttgggcaacatagggagaccccatctctacaaaaataaaaaaattagccaagtgtagtggcacatgtccatggtcccaactccttgggaggtgggaggatcacttgggcctgggaggtcgaggctgtggtgtgtcatgattgcatcactgtattccagcctgggcaacagagtgagaccctgtctcaaaaaataaaataaaatagtaactcccagtgtcatgtttttctattttattcttcttcctaacacttattaataactgcattagacagaaaacaaacaggtaaacaaatgagctacatgatgacaaagtgatcaaagttatgaagaaaataaagcaaggcttaaaggagggggctactttggatgggaggtcaaaaagggcctctttgagaaggtgacttctaagatgaattttaggtgatgaaaatggcaccaatcatgcaatgattttagggaattccaagcagccagaacagctttgcagagtcccaaagtgggacggaaattcacttattagaggaacagaaagaaggtttgatttgctagaatccagtgagcaagggggtcctggagcacgagtcagatcatttgaacattatggtccattgaaaagagtgtatattttagtgtaagtgcatattggaactgtgtaggtgacatggtttatggtttttttttttttttttttttgagatggagtttccctcttgttgcccaggctggagtgcaatggcttgatctcggctcaccgcaacctctgcctcctgggttcaagtgattctcctgcctctgccttccaagtagctgggattatgggcatgcatcaccactcccagctaattttgtatttttagtagaggtggggtttcttcatgttggtcaggctggttgcgaactcctggccttaggtgatccgcctgcctcagcctcccaaagtgctgggattacaggcatgagccacggcgcccggccctaccttaggtaatccgcccgcctcagtctcccaaaatgctgggattacaagtgtgagccaccgcacccagccactgatctatgttttaaagaattcactcttggctgcagtatggattgaaatgggggagggagaaagaatggaaataggccattcagaaggctggtgcagttgtccaggtgattgatggcagtgcttggaccaagatggtgacagcaagatagaagaaatgggtgaattcaggatacattatggaggcagtttgtcagaactggccgatagggataagaggaatgagagaaagaaaaatcaagaatgacttctggctgggtgcggtggctcacgcatgtaatcccagcactttgggaggctgaagcaggtggatcacctgaggtcagaagtttgagaccagcctgaccaatatgatgaaacctcatctctagtaaaaatacaaaaattagctgggcatggtggcaagcacctgtaatcccagctactcgggaggctgagacaggagaatcgcttgaacccagggggtggaggttgtagtgagccgaggttgcgccattgcactccagcttgggcaacaagagcaaaactccatctcaaaaaaagaaaaaaaaaaagacttctaaatcttgtacttgagtaactgggtaggtggtcaagccattttctgagatggcaaaaactgggagaacagttctgtgggtgttgtgggtgttagaagccagatatcaaaagtgtgtgggacatttagactttgagatgcctattagacaagcaggtggggaaggaggcagttgaatatgagtctggagctgggaggagagagatgagagctgtaggtacggatagaagtcaaatctggatgttgccaggcgcggcggctcacacctgtagtcccagcactttgggaggccgaggcaggccaattgcctgaggtcaggagtttgcgaccagcctggtcaatatggtgaaacactgcctctactaaaaatacaaaaaattagccaggcatggtggcaggcacttgtagtcccagctacttgggaggctgagacacaagaatcgcttgaacccggaaggcagaggttgcagtgagctgacatcgcgccactgcactccagcctgggtgacagagtgagactccatctcaaaaaaataaaaaataaataaaaataaaaacaaatctggatgtcatcagtgtatggatggaagggatcaactaggaaaaggtgggaggaagaatgttctaggcagagcctggagatcttcgaagtccctcccaatttgaggattctgtgctatttcttctgtttgacagtgctacctgcacagagttttagtctctactcaggttcatatttcaccttcaacctgtcacctgtcttgagtccagaggatccaatggaacaagttgagatggagggcaatcatggaaggaaaagccagtattgaagtgtgacccctgaaaatttgagacaggtctcagttttttttgctttttttgtttttttgttttttgttttttgaaactgagtctggctctgtcaccaggctggggtgcagtggtggatctcagctcactgcaacctccaactccctgatttgtatttataatagagatggggtttcaccatgttggcctggatagtcttgatctcttgacctcgtaatccgcctgactcggcctcccaaagtgctgggattacaggcgtgagccacgacgcctggctaagaataattatttttttacataggctttaaattggctttgatggaactttgtccatagaaggaatcccagataagactttttttaaagctgatactggccatggatttgtgccatcaaatatctatgagttgggtgaattttctctccccttgaggttccaagatgaacctggggcttctgtgcctgttagaaagtgacactctttacttaccacaggtcagaaaccctgtacatgaactgtgtacacaaaatatgaggccagtttttccaacggctttattgggtccataagtcaagttttattccttaaaggaaagcagaccattccagtcaaagccttggttaaaataaccagtttctccaatagtgtcctgttacaaatgaaaacagattcttattgcatttatgcaaataactgtattgccataagttaagaatactcacaaatactttctaaattctggagaaatcaggtagagagaaacaaatattctccaaattttgttcatgggagtatactaaattgttaaaacctatcaatagctcaagaaaaaagttttaagactctgaaaaacaaaacaaaggatcagcaacattttaagtcaaaagtcaaaagattagttcagtccatgcagttaatttctgttctgcttgatactcatcaacattttagctctccatgagtcctgaaagtttttcctctattctgatgtcacaatctccaaagttatcagaagcctgcatccaggagcacctgttaagagtttttgattataaagccaccttctaaagagaaccaaaacaagacaattgtctgtggatgacaaaaagttctaggacagccatagtcaaaaacacaattgaaaaggaaatttgttgggggatggggaggtggctcaaacctgtaatcccagcactgtgggaagccaaggcaggtgggtcacctgaggtcaggagttcgagaccagcctggacaacatggtgaaaccccatctctactaaaaatacaaaaattggccaggtgcagtggctcacatctgtaatcccagcactttgggaggccaaggcaggaggatgacaggagttcaagatcagcctgttcagcatggtgaaaccccgtctctactaaaaaaatacaaaaaataggctgggtgcagtggctcacacctctaatcccagcattttgggaggctgaggctggtggatcacctgaggttgggagttcaagaccagcctgaccaacatggagaaaccccctctccactaaaaatacaaaattagctgggtgtggtggcacatgcctgtaatcccagctactcaggaggctgaggcaggaaaattgcttgaacccaagaagtggaggttgcagtgagccgagatcgcgccaccacactccagcctaggtgacagagcaagactccatctcaaaaaaaagaaaaaaacaacaaaaaaaggccggtcgtggtgtctcacacctgtaatcccagcactttgggaggccaaggcaggcagatcatgaggtcaagagtttgagccatcctgcaaacatggtgaaaccccatctgtactaaaaatacaaaaattagctgggcctggctgggcgtggtggctcacacctgtaatcctagcactttgggaggccgaggcgggtggatcgcgaggtcaggagatcaagaccatcctggctaacacagtgaaaccccatctctactaaaaataaaaaaaaattagctgggtgtggtggcgggcacctgtagtcccagctactcgagaggctgaggcaggagaatgacgtgaacccaggaggcccagctggcagtgagccgagatcgcgccacggcactctagcctgggcgacagagcgagactccatctaaaaaaaaaaaaattagctgggcctggtgtcgtggacctgtagtcttagctccttgggaggctgaggcaggagaatcacttgaacctgagaggcggaggttgaagcgagccaagatcatgccattgagctccagcctggcgacagagcaagactccatctcaaaaagaaaaaagaaaaaggaaatttgttacctctgtggcacacaataattttaacataacaattatgattattactgataatatacactaagttatatcagaattataggagcttcccataattttggaacacatgccaataacatatttgtacaaatacagcccaaagaaaaaccaataccatttcatatttgacaatgcttcctgtataaattttgtaccaaataagtcaaattatgttattttttggactttagggaacctaatgtcttaaaggatttatttaggtcagaaaaagacataattcatcatttgattttgagaagtttgtcaaatatcaaaggttttaaacacttgatatcacaaaatagaaacacaggtcattgtaaaataagtcattcatttaaccaaagtgataactcatggatttcaaaaaaagccaagaatctttattctttaagagaggagacttaattttccaaacaataagcgctaataaaaacagcgtgaagccaattaaatttgtttttcaaaatcttataaacattctattaaattttaatcttgaccataagatatagcttccataatccttttataacctttattaaggagttggttaatgcttcaagaaaaccttgttaatctgggccgggcgcggtggctcacgcctgtaatcctagcactttggaaggcggaggcgggcggatcacaaggtcaggaaatcaagaccatgctggctaacacagtgaaaccccgtctttactgaaaatacaaaaaaattatccgggcgtggtggcgggcgcttgtaatcccagctactccggagcctgaggcaggagaatggtgtgaacctaggaggcggagcttgcagtgagctgagatagcgccactgcactccagcctgggagacagagtgagactccgtctcaaaaaaaaaaaaaaaaagaaaactttgttaatctgacacaggggcccatatgctggtcttgcattagtatgccttttgacattaaggatttatagagaaactgaacttattttatctttcaaaattaacccttacaatcttacaatcacttctccctgggccttgaggagttgaaatagctttaatttctggccctgtgtctcaaaaatgcagtttattttgatgggcatcttctacagggcctgaagctgaggcctttttttttttttgagacagagtttccctcttgctgcccaggctggattgcaatgacgagatctctgctcactgcaacctccgcctcctgggttcaagtgattatcctgcctcagtctcctgagtagctgggattacaggcatgtcacaccacgcccggctaattttgtattttcagtagagacgaggtttctccatgttggtcaggcttgtctcaaactcccgacctcaggtgatctgcctgccttggcctcccaaagtgctgagattacaggcgtgagccaccgttcccggggaagataaggctttaattgctgtcagtgcttaagattcagcaggacttggtgtcctttttagatacaggagtcaaagccctttaattcaatgtcacaaggacttttttttttaagatggagtttcgctcctgtaacccaggctggagtgcagtggcacaatcttggctcactgcaacctctgcctcctgggttcaagcgattatcctgcttcagcctcccgagtagctgggattacaggcgcccaccaccacgcccggctaatttttgtatttttagtagagacggggtttcaccaggttggccaggctagtctcgaactcttgacctcaggtgatccaccttcctcagcctcccaaagtgctgggattgcaggcatgagccaccatgcctggcctgtcacaaggactttaaaagtacatacaggaagacacatggatgtaataaccttaatattaaaaattttttattttatctaagttttttttcctaagcaaaccaaaacataataataatatttggtctttctggtttgtcctgaacatcccatcttcttctttttttttttttttgagatggaatactgctgtgtcacccaggctggagtgcagtggtacgatctcagctcactgcaacctccaccacccaggttcaagcgattcttctgcctcagcctccaagtagctggaattacaggcatgtgccaccacgcacggctaatttttgtatttttagtagagatggcgtttcaccattttggacaggctggtctcaaactcctgacctcaggtgatcaacctgcctcggcctcccaaagtgctgggattacaggtgtgagccaccgcgcccagctcatcccgccttctcaaacaaccagtcattttattttaggactaaaatttaccatatttttcctcatataaaattatttctctttaagctttcttgccacacacacaggaaaatacctttttataactttctttacatgtatttttattttctggttcttcttaccttgtttaatgttttttcatttttgttttgtttttttgaggcagagtctcacactgtcgcccaggctggagtgcagtggcgtgatctctgctcactgcaagctctgcctcctgggttcacgccattctcctgcctcagcctccctagtagctgggactacaggcacccgccaccacgcccggctaattttttgtatttttagtagagacggggtttcaccttgttagccaggatggtctcgatctcctggcctcgtgatccgcccgcctcggcctcccaaagtgctgggattacaggcatgagccagcaagcctggctcatttttgtattttttgtacagacggggtttcaccatgttaggcaggctgttctcgaactcctgacctcctcatccgcctgacctcctcatccgcctgacctcctcatccgcctgccttggactcctaaagtgctgggattacaggcatgagtcacctggcccggcatgtctgattcgtttttgaatcaccagtacaaagctagatgttgttctttttgtgaaaagttgccccttcaggccccaaagaggaccttatcaggcatttggcgtatgaagttccaaggccacttgcttctggagggaatgagtagtggcctgcagttgaaatgagatagagtggtggggagcactcatcagacctgtgggatgttgtggacggaaataatttttaagtgcctgcacctctaccatcctgtgaatcctcaaggacaaggacctgattggagtcacttctgtgttcccgacagaacccagaactctccccaggacatagcattcctcaataaaagattgctgaaatgaaccgaagcccagtagctcaaggttactggccagctgccagtggcgaaaagcttggcgaatctccccagcagctcagtcccagggtagcactgttggagctggagaaacagccctggaaagacacagctgaatgtcctaagtccaacatccagaagcttccagaagctccactcacagcatgaataatttaggacaggactggtggtcagatttgggaagggcattgaggttggagaggagaacttcttaggtgccctctcagccgggcagctgggccttctggcttcccaggtgactgcacagcctccgtgagccctcccagctggccctggctgctgagaccctgcccacaggacctggccctgtggaggttcaatttcacacccaggccccccacactcttcctgtgcatgcagcttggcttcccttggtattcatcttcccctctcaccagggaaggactgtggcacttacttctcttcttctccttcttcttctttcttcctcttcctcttcttattcttatttgtagacagggtcttgctgtgttgcccaaggtggtctcaagctcctggaggcctcaagtgatcatctcacctcagcctcacaaagcagtggaattacaggcatcagccactgtgacttgacactcatttatttatttatttattttctaagacagagtttcgctcgtcgcccaggctggagtgcaatggcgcaatctcagctcactgcaacctctgcagtgagttcaagcgattctcctgcctcagcctcccgagtagctgggattacaggcacccaccaccccccagctaatttttgtattttcagtagagatggggtttcaccatgttggccaggctggtctcaaactcctgatctcaggtgatctgcccgcctcggcctcccaaactgctgggattacaggcgtgagtcactgcgcccagcgacactcatttctaagttgaaaatgattcttcagcttcatgccttagtgccttctagcccccctcacaggatcttttttatttttattttttaaattttattttttatttttttgaggcagagtttagctcttgttgcccaggctggagtgcaattgcgcgatctcggctcgccgcaacctccacctccccagttcaagcgattctcctgcctcagcctcccgggtagctgggattacaggcatgcgccaccatgcctggctaattttgtatttttagtagacatggggtttctccatgttggtcaggctggtcttgaactcctgacctcaggtgatcagcccaccttggcttcccaaagtgctgggattacaggcatgagccaccgtgcccggcatatttttaaaatttatttgtttgtttccttttttgagatggagtctcgctttgttgcccaggctggagtgcagtggcacggtctcagctcactgcaacctccgcctcccaggttcaagcgattctcctgcgtcagcctcctgagtagctgggattacaggcacatgccaccacacccagctaatttttttttttttttgagatggagtcttgctctgtcgcccaggctggagtgcagtggcatgatctcagctcactgcaacctccgcctcccgggttcaagcgattctcctgcctcagcctactgagtagctgggattacaggtgtgtgccaccacacccagctaatttttgtatttttagtagagacggggtttcaccatgttgaccaggctggtctcgaacccctgaactcaggtgatctgcccatctctgccacccaaagtgctgggattacaggtctgagacaccgcacccggcctactctctgcctctatgagctcaatatttttaggttccacatgtaagtgagattaggcagtatttatctttctgtgcctggcttatttcacttaacagtgtcctccaggttaatccatgttgtcacaaatgacaggatttctttctttctttctttctttctttcttttttttttgagacagtgtcttgctcttgcccaggctggagtacagtggcttgatcacaactcactgtagcactgtagcttcaaaatctcctgggctcaagtgatcctcttgtcttagcctccagagtagttaggactacaggtgggagccactatgcccagctaatttatttttattttttatttttttgtacagttggggtctccccatgttgcccaggctggtctcaaactcctggcctcaagcaattctcccaccttggcctcccaaagtgttgggattacaggtgtgagccaccacacccagccaggattttcttctttttaaaggctgaatagtattctattgtcccatagggactttagaatcaggctgtagaattctctggtgctgaggcctctgcaggcttctgtggagaagatatcccacagagggacctgcacttaggcaggttacaactgtggtctcctgatctttctccggaagccccagaggtacctgtggtctaaggaaaagaacccttggctgagtcataggacctagattctttgccttcgcttcagacagtagagagcagtggttaaagcatgagatttggagacaggcttggatgagaaccccagctccaacactagtttgctttgtggacttcagcaagtcatttaacctcttgaagtctctgtttccttatttgtaaaatgagaataataaaggtaactactgtacagggtttttgggaagacttaatgagataatatacacaaaacgcccaggacattgcctagcacttagtaggagttttgtaaatggtggttgcttgcccttaatatgaaacagtgcctgcaaacacagaaccttagcaagtttcagtgagtggaaaacatcccttcccatctctatcgtccctgctccagttgacagcctcattacctttggctgggactagtacagtagcctcctagcgtaaccatccacacccatctggtacctgtccttgggttacttttgctgttactttgacacctttcttggctgcttataataattaaatttaggtcaaatttcttttccttccttccttccttccttccttccttccttccttcctctctttcttgatggaattttcactcttgttgcccaggctggagtgcaatggcacaatctcggctcatcacaacatccgcctcccgggttcaagtgattctcctgcctcagcctcctgactagctgggattacaggcatgcgccacaacgcctggctaatttttttgtatttttagtagagacagggtttctccatgttggtcaggctgatctcgaactcccgacctcaggtgatccgccagcctcggccttccaaagtgctgggattacaggcgtgagccaccacgcccagccaatttaagccaaattccttagctcaagggcctttgtaaattgctccaacacatttttttttctcccaatcgttatggtgaaaaagaacagaatgtgaatctgaagacaggggtccagtcctgggaccatccgctttctccctacatgactttgagtatatcatctcagttctacatctatacattagagatcattatgctgcctcatgggatgcatccatctttcctgctgaactgtgagcccatggtcaacaggcagggatgaaatgcatcttgctcacactgtatcttcagtaccctgcatggggcctgacaccgggtagatgttcagcacgtatttgttgaatgaatgaatgaatgaatgaatgaatgaacagggttgtgaggactggatttggcatgatccaggtaaagcgccttgcacagtcaccaggagacacgcccatatggtagctgttgctacgcaggttactaagtggaatgactttagacacattacttctctggaaacattcacgtatctgtcctaggagaggatagaagggactcctgcctcgttttgtgcatcattaggtcgtgtgtgcagaaggagctcaataaatgcttcctcggtggataaagcatctcccaaggctccacttggggctagcgctgtctaaagagactgagttccctcattggggcctggttcttttctgctctttaccctgcggggaagatgaggccttctttgggtccctccttcattcagttccagaagcccagcagataggtgggcctataggacccgcaggatctgcaggacgctctccgagagctggagcaagagcgccaagggcggggacggaatgtgggtttaagggaaacgggcgaaagaggggtgggcatggaaaagggtggtttgagaaccaggggccaggcagaaggacagcccggcagcctgagagagcgactggggggcggtgaggagctccaagggggcgggtcggctgagctggcccagggtcagctagcggccgcggggccggggcggagaggtcgcagagcagagggcggagaatttgggggtgtggttggcctgcgggggacaccggaggccactagggaggagttgcagaggggagggaccgagggagggcctgggggcggccagaggacgcggggtcagtgaggagggggcgccgggcgacggactggcaggggcgcgcggggcgggcggaagcgcaggggtggggtgcagggcggggggagcgcgcatcggggggcggggggcggcgtcggcgctgggagtgggggagcgggttgggcggctgcgccggctctgcttcagccgctgccgctagggcgcggggggcggggggctcggcgccgcgagctcggccattgtgggagccgctcccctcggctccgccacgctcccctcgactgcgctccagcctggggcgcgcccggccgccgccgccttcgctgccgccacgggcccgtcttcttcctccttcggctcccagggtaaggcgcggggcgcggggttggatgcaggcgccctgcccgctgcgcgggatgcgcagcggcggctggggagcggtgattgatcgcccccggcggccgcgccccagctcccggcagccggcggccgcggacaggcggcttgacaggcgcgggcacagcggcagtgcgggcctcgcacctgcagcccccgcggccggcgccggggagcggcgaagactcggagctggggtttcccctctgcgggcgctgccagggtggaagggaggcgtgttccccgggaaaatgggacacggagccctgcaggggccgggcgccggaccttagggagcgggcagggctgggcggggaaggtcgcttcccggccccagggctcggccgctggccccagcgccctgtcggtcctcggtcgcgcgtccccgctctttgtcagccacgcttggcgggcgcgcggacccacccgcgccgccccaggctcgcgggcgcgcccctcctccgccgtggggaaacgggtgggggccaggtggggagggtcgcagggggtgagagcacctggtgaggtgcctgtcgccgaccggcctgcggcctagggggtgggctggggcttgggcgggcactcctgtgagtctcggggattgatggaatgacccaggtggccgagggtcgggcgggagcccggaggtcaaacaagtgtttgtgaggggccggagattgacggggtagagcccagaagggacgctttctccgggaggaggccttgggagagccggcctttgtagtgaaatgggcgtcttggagaggaatgggcggttctgggatgaagcaggtggtctgagaggaggggcatttttcctgcccccgagctgagggtagggatggaagcttggcctgagtttcccagaggttgaggctgtcacttcagcacctgccctggcactagaaggtaatgggaggaggggtaagtgctaagataaagggcaaagctgtgccaggacctgggtcctttcctttctgcttcctgtggctagatcccttctagatgttctttgggaagagggctcacacgctgctggcacaaggggtgccctggcagggaggctctgctgcccgaggctgcctacaggcctcctacaccatttttatgtttcttttgttatctctttgaattctgtgcctatggtggggatctgccaatgacccaggaggctgtctctgccccttgctgcctctaggctcagattcgagaatcctggaggagctgtggccacagacccattgtgtccagccatgtaggctacctcgctgtctctacacagccctgtgagccggtattccctataagggaaaatcaacagctcttgccctccctggaccctacagccaaagtcaaagctgggtacccaggttggctttgcggcctgtaggaaggctggtagctgaaggcacataccatttgtgctctgatggagcccttggggtggtgggccatgtgtagcagatggttctgggaagtgagcttgacctggctgagggaaggctgcccagtctggctggtggagcccagctggccttagagcaattgtattttcactcaactctctccactagctaaactgaccagaaaaaatgggctttctgtcccatccccccaccccatcctcatgctgtgccacgtattcccacaacagcccttggaataagcaaatgaacatttaatctaatgcctgctgggtgccatgcatggcatcaaccagtactacatttgcatttgacaatgttcttaaaacatcctctgaggtggaatattacccccattttgcagatgaagaaactgagtctcagagaggtgaagtgacttgcccaagatcacagcaattgtaagtggccaaaccagaatatatacccaggtctgtcttacttcaagtctggtacattttctaatatatctgctggagacagagtgggagaaaccagagaatttctgcctattctagcccatggtttgcttcttgcctgctattgagatcatgctttagctttttgaaaatacttcccatctgttgccccactttatccttatagcattcctgggaggtaagtagaacaggaatataataatagatactgtgtattgagtgcttactgggcagtgggcactattaagcactttacatgcaggattaagcactttacatgcagaacccaaccctatagggctaggttctaactgtcgtcatccccattttgtagatgagggtgctgaggctcagagagattaaatggcttctccagggtcacacagctagtaaatggagtagaatttaaacccacagcattctgtcatgggatctgtgagcttaatcctccaaatgtgccaaatagcaataataatagttcctaggaataggagctaacttttacatgatgctttaagatttatagacatcatcacatataatatcccccaaacctcatgaattagttttgggatgtattgtcttcatcccattttccaggtgaagaaactgaggtacagataggcaggactagaacctctgcttcccaagtcccagtgttccttatgggaccccttttgtctgctcacattcacccctttctccttcag

SEQ ID NO: 11 - DNA, 93 bp, Homo sapiens
  source 1..93: mol_type = genomic DNA
gtcggcggaggcaaaaggagtcctcttaaagagaacgcggctgcgaccattgcgcgtgcgcgcaggccggcgccccctggaggtgcgaagggc

SEQ ID NO: 12 - DNA, 376 bp, Homo sapiens
  source 1..376: mol_type = genomic DNA
tttcagactgctatttttttcatggttgggggagcagcgggagacggggagggtgctaggaggagagggcgggaaaagagggctctctcctcttccaatcctctgatcttcctgctgttgggaggtgcagattcattgtgccccagcccacgtccccccacgacccccgcccccccacctccccccacgaccccccccacctccgagcgggttctgtgcccaggtggcgctggagaaaggaacagcggaccccgcccaagtcattactaatgtagcaatcgtctgcataaacccaggccacggttcctattggctgagtcctctgagggtgacgtcatcgggacgtactaagactagggttgggccgagagtcgga

SEQ ID NO: 13 - DNA, 791 bp, Homo sapiens
  source 1..791: mol_type = genomic DNA
aactggccggcggcgggtcacgtggcacaggcagttttccgggcctgagggtcccgcctcatccggcccccgccctacccggagggctcggcccctcctcagccaggaaaaggctggagagtgcgcccgagcgggacgcgtgctcctcctaagccggaaggagcagcaggcatgtctggtctggcccaggcaaccaggcggcagcggtccaggttacccttccaagggctggggccctcgagggctccgttttttctcagaggttcctgagttaggatgtggggtttggcataggaggctggtgggcgacggcagcagctgcaggagctgcccgtgcacaaacagccctcctctcctgtgccatgttaccggcagtgcaggggacagccagagggatctaggcttccggtctccctcctccctgtctccaaagcggcaccgccctcgtgcgggttttctaaccgggcagagtcctggaagctctgccccagcctccttaagtggagcccagtagctcgccgaggccaaggaaacgctgccggagtcctccctccctggcctcctcaggctgcattcaccctacccccaaatctgaaccagaatgcggtacagggtaatcatttccatgttgttgggagggaccagattagctgctgggtgggtgggaggtggggcacaagctagtcctttgcactcaacaaacctctccagggtgagctaggccaggaagtccttgcatcctcctacccccagccctggagagagcaacacaaacaacaggatacaggaag

SEQ ID NO: 14 - DNA, 696 bp, Homo sapiens
  source 1..696: mol_type = genomic DNA
gccgctagagccgctgccgccgcttttctagaaccttcccccccactaacgcgtcttccgctacgtcaggccgtcgcgtaaacgccctatccgccgccaatggcgggaaggctctacgccccaccttacgccaaatgcgtactcctcccacccttgcggccagagacagtacccgacgttacttccgtaaatgcgctcaatgaattgcggaaggctagagtcctgctagttactacctcttggaatagggtcccgccccctgccttggcgcaaggcaggtgagaaacggtcgcgcagtttgaaattaacgccgacgggaggggcttaatccgcagcctggagatccagccccctcaacccgggaggtggtccctgcagttacgccaatgataacccccgccagaaaaatcttagtagccttccctttttgttttccgtgccccaactcggcggattgactcggccccttccggaaacacccgaatcaacttctagtcaaattattgttcacgccgcaatgacccacccctggcccgcgtctgtggaactgacccctggtgtacaggagagttcgctgctgaaagtggtcccaaaggggtactagtttttaagctcccaactccccctcccccagcgtctggaggattccacaccctcgcaccgcaggggcgaggaagtgggcggagtccggttttg

SEQ ID NO: 15 - DNA, 362 bp, Homo sapiens
  source 1..362: mol_type = genomic DNA
gtgtccctctcttaaaggaatattcttctctcagcctattatccagctttgccaaggatcttctgcactaaagagatggactaacgtaatgactagggttagtatctctttcacaaggcctgcctctcttaatccaaaattggaaaacttgggagtgtctagtattttccatagatactattcaactaattactcagtacaactctgtgcttgttactacggtcaccattttaaaggagaaaaactacttcaaaaaatggatttataagccgaaatttcctcaaggtcacacattgcattatttccaaagcaatgttcatcctgaggttggttgtttccttggtcgcccaggttggagtgca

SEQ ID NO: 16 - DNA, 2722 bp, Homo sapiens
  source 1..2722: mol_type = genomic DNA
tgtgctttgtatgctgctcttgtttgatggaggtggggagaatgtcagttgaagtggtattggaactgttcctccacagtgccatgctctaccttcccaaaacgaggaatgtcttgacttgcagaagacacaaatcttagaatctttgatgatatagtaatctgctggagttgggtggtgtaattttctaagtagaaacagtcctcactgctaagatttgtttggggctaggacacaatgtctactgcaaaaacagtactgcaagtgtttatcttgaattcaagaagccaggaagaatggtacactagttttcagggccagactaaggtcagatgggtaattagtttatcttactacatgtcaggaattgctaaactagagatactacaagtgaacagaactgaacacaggtcaaatgtgtaatgatgtatatctaccattaaaatagagtagttttagtgccctaaaaatcctctgtgttttgcctgttcatctctctctccttgcttacgcatttgtccaaaggcaaagaatgtacaatgccaatgccaagagtgaaccctagtgtaaactaaactatggatttggggtgataatggtatatcaacgtaggttctattgttaacaaatgtaccactctggcagggtggagtgatgtttataaagggggaggctatatgtgtgggaaatgtctatacctttctcaattttgctgtgaacctaaaactgttctaaaaaattaagtctattaaaaaaagaaaaaaacagccaagtgcggtggcttatacctgtaattccagcactttgggaggccgacgcaggcagatcacctgaggtcaggagatcaagaccagcctggccaacatggtgaaaccccatctctactaaaaatataaaaagccaggcttagtggcgggcgcctgtaatcccagctacttgggaggctgaggcaggagaatcgcttgaacccaggaggcggaggttgcagtgagccaagatcgcgccactgcactccagcctgggtgacaggagtgagattccattccgtatcaaaaacaagcaaacaaaatttccctcaaagagcttataaactagttggcaatcatcagttgaaatcttggtggaactgatacaaaataattctgctaggattttttttgtttcaaaaaaattgtcatatatatttgggtggtgacaatactacaactacttagaaggaacctaattttaaaaacgtttttcagggattgaaagtgaaatgctatcctggaatataataaagggaaaccacaaaaaaagagggattgtaacaatcacatacaaaatgttacagtattttaaaactgtaagatgaagtctctatagggaaagggttctttttcatcccccattaactcaaaagtttcctgtagccaaatcttaggggatctggttgttttccagtgcttctcacctgagcatacaatcaaactgggaagttctttatcttaacaccaaatgtcatactttccctagatttaggtaattttctccctatgtccgttggttcgctcttcacccctagagaatcactgcataggtcactgcagcctcgacttcctgggctcgagattcccccacctcagcctcccaaagcgctgggattacaggcatgagccaccgtgcccagcccgctgacgtattttctgaacaaatgatgcattgtagccaggaatctctagtgatattttgcttcgggcatcgtgggggaagaccataatcagctattttattcttgaaatttaggaaaaataattgctctatcttaaacagtgagaaaatattttttgaagtatggcagctttcccgaatccccactttatatcttcctatgttctgaaatcttaggaccttcgatggttccggaatcgaatcaccctgcagcaatttattttagaaaataaggcaaaccggctgggcgcggtggttcatgcttgtaatcccagcactttgagaggccgaggcgggcggatcgcttgagctcaggagttcgagaccagcctgggcaacacagcgaaacctcgtctctactaaaaatacaaaaattagccaagtggcgcgcgcctgttgtcccagctgctcgggaagctgaggcgggagaatcgcttgtattcaggaggcggaaattgcggtgagccgagatcgcgtcactgcactccagcctgggtgacacagtgagatcctgtctcaaaaaagaaaaaaagaaagaaagaaagaaaagaaagaaagaaagaaagaaagaaagaaagaaagaaagaaagaaagaaagaaagaaagaaaagaaggcaaaccagtaaaacaagaatttctgtacagtccgcgcctatccatagcaaaatggaggacttctccagggaactgcaactcccaagaagcaatgcgagggaaggtggcggtcactattttttccccgtctgcacctctaggtccaatattggaaataggtgctgacgtttaaataacacagcgtcctcatactaaatctgggggggaactggtaactcgaaaaccaaatactcggtcttccgaaagaactaactcaacctacccttctacaagagggtccgaaaaccactgttacgcccattgggtagccccgcccttggggggggcaaagggcgtgaaagcggaagtgacgacacccggcgctccattaaatagccgtagacggaacttcgccttt

SEQ ID NO: 17 - DNA, 800 bp, Homo sapiens
  source 1..800: mol_type = genomic DNA
ttctcatttagcaagagtggaaaactgttgtggatcccaggagaagtctggagctaggtagtaggggctagataatagagggctttgactaataggagtttgggccttatcctgtggaaatctgaagggttttgggaaaatgaattcagggctaggtgcaagacagaagggcaaataccagactaagacacacagccactaagagcctctttttttttttttgtccagtcagaggtaatgaggcctatatatttattaggaaggatgattaaccatggctgtagcttcacctaagaggaacgtggctcttgggccgggtgcggtggctcacgcctgtaatcccagcactttgggaggtagaggtgggcggatcacctgaggtcaggagttcaagaccagcctgaccaacatggagaaacctcttctctactaaaaatacaaaattagccgggcgtggtggtgcatgcctgtaatccctgttactcgggaggctgaggcaggagaatcgcttgaacctgggaggtggaggttgcggtgagctgagatcacaccattgcactccagcctggacaacaagagcaaaactctatctcaaaaaaaaagagaggaatgtggctcttgaagaatctttctagtctcatcttccctgtccccttagacacaccttcaaagatcctttttcttttggtttggcttgtctttccccctcactttacctaatccagcccaattccatagccactgctggttcctttaatggaggagggaagtggggtgacatagattctcttaaacctacc

SEQ ID NO: 18 - DNA, 800 bp, Homo sapiens
  source 1..800: mol_type = genomic DNA
tatttaaaggcattaattagcgtttgcatgcactactttaaaattttcctctcaattgcttgagtccaggagttcgaggttacggtgaactatgatttcaccattgcagtccagctgaggcaacagagagagaccctcttaaagaaaaaaaaaactttttcctctcagactctgtccctagcattgactcaccccatcattttttttttttttcttgagatggagtttcaagaaaaaactccaggctggaggcagtggtatggtctcggctcactgcaacctccgcctcccgggttcaagcgattctccctgcctcagcctcccaaatagctgggattacaggcacccgccaccacacccagctaattttttgtatttttagcagagacggggcttctccatattagccaagctggtctggaactcctgacttcaagtgatccacccaccttggcctcccaaagtactgggattacaggcacgagccaccgcgcccggccttcacctattatatctattcttgtcttttaaatagtgagtgacttcacagctcagtctctttgttctgaacttccattctgagtcgcaacccaggtacaccttaggaagggcgggatcccgttatcctgtccctgaacatcaactactgagacagctgatgaagctggctcggtgtgtgacggaggccccctcagagtcagcagagccgagcgtacagcgcacatcaggcagagcagaccagaggcagggaagacacactggggcagggccagtgggcacatctggaccacctgcgaggg

SEQ ID NO: 19 - DNA, 800 bp, Homo sapiens
  source 1..800: mol_type = genomic DNA
ttattcttcacagaggacagctcccaactctttacctctcttcctaatgcaacactcactctcactctcagcagccttataatatctgtaaatgtagaaactatcagttatgaacttctgtaactcccttttcatctttggcacctcaaaactttaatgtatgtaaaagcatgggctttgaaattggtaaactggagctctatcaatctctggctgtgtcaacttgagcatgttgcccaatttctatgagcctcagttttcttatctaaaaaatgaaactaataatgcctactccaaagaattgttggatggattaagtaagacaacctaatataattgctgtgcatagtaaatcctaactgttggttgccttatacctctaatcttctttctgctctcttttctttagaagatggcatgcatcttttccaaggtaaattttacctctgttgaactctatcccatactctcttgccatggccctaattttgttcagttacttaactccttcccttccctctgacatccttggtcccttcctctccacagttttattctctcgtcttacacaaacatgcccaagtctcatcatccataaaaacctctgtcattaccacttcagctctcttcttctgttcatacccagctgtgttgggtgccatgaatcattcactactattatttttaaaattcctatcactacttctcccctctgcaatctggcttctcctgccaacatgctactgaaattaatgaattaattcatcaaatatttacaagcatttatcatgatactatgt

SEQ ID NO: 20 - DNA, 800 bp, Homo sapiens
  source 1..800: mol_type = genomic DNA
cacaggcactgtgctcagtaactcaagtcattataatactgtgtgcttgacaatatgttgtacttactggccaaaaatcaccaatgtctaattgtcaagctaaatgtcttctttcaatttcaatctatctgacatgctggaaacttccctttccaaggcttctttgctatcgctcccttggctcttctgttcctttgatcttgctgtatttactttgtagtatcctcctaattatgggggtgattccctaggcatccatcttcagctttcttctctctcactcttttaactttactctgtgaatttcctgtaattatacactacatttggtgtgtgtgtgtgtgtgtctgtgtgcacgtgcatttttctaggaaggagttcatagcttttatcagctctgcaaggaggtctatgaccctcccaataacaaccctgaccagttccacctcacgtgtagacacattcttccactgtgatgacttccattatcctccacatgctgctgattcctggatctccaaccctaaacctctttcccatgctccagcgaccatcttcccaactctgcctaggtccctcctggagggtccttctagaataatagttctcatcaggcaggtggcagaacctctgtctcccactccttgcatgcccctctgtgaggcttgggtgggttgggggtgatgggaggggtgcgtgtataatcagagtccctggggtgcagaagctctgttctgaccttgcctcttgctatccatggaattactgctctatcctggccattttcccattctctttct

SEQ ID NO: 21 - DNA, 800 bp, Homo sapiens
  source 1..800: mol_type = genomic DNA
gaagccgggtgtggtggtgcatgcctgtggtcccagccacttgggaaactgaggtgggaggattgctcaagcccaggaggtcaaggctgctgtgagctatgattgtgacactgcactccagcctgggcaacaaagggagaccccttaactaactaaatgaatgaatgaatgaatgaatgaaggcaacctgagctgcattccttggcctgccaacctgcccagccccatccctcagccctccctgagtctgagggccctgcaggtcccacacagggccaggctccatcttgtttctgcaaatttgcaccttccgttccattccctgccacactgctcacgggtcaccatggatgctggataactgccaccgtctccttggagaagccctccgtcatctcagcccctgatgtcaggtattcaacctgcaaatcctccctgtaggtgctgggctctccagagccctgggctgtgtgcaggggactcaggggagaacgggccccacccagctccttcctcacagagctttcagaggctgggggcctccctgatcccctcccagatcccaaggcccctgctgcccccaccctgcagactgggactccgtgaggctgggctctgacttggatattgtggttccagcacacagcaggcaccgtggctgtagtaggcgtgcatgggaagtcaggaggagcagacctgtcatctcccctgctgaggacacagcccggtcagcgtgtcttggcggcctggggccagtgactgagcctcttggcccagttccttgcccaccttgaagatct

SEQ ID NO: 22 - DNA, 800 bp, Homo sapiens
  source 1..800: mol_type = genomic DNA
gtagggagggataaagagatcaggtccaaggccaggtgcggtggttcacgcctgtaatcccagcactttgggaggccgagccaggcagatcgcgaggtcaggagttcgagacgagcctggccatcatggtgaaaccccgtctctactaaaaatacaaaaattggccaggcatggtggcaggcgcctgtaatcccagctgaggcaggagaatcgcttgaacccgggaggtggaggttgcagtgagccgagagcgagccactgcactcctgcctgggtaacaaagcaagactccatctcaaaaaaaaaaaaaaaaaaaaaaatcaagtctggcgcagccccaggaagcaatcttgggctgggcacaccgcatttctgtgacatgaaggcagcgacacctcgtcgctgtcacagggttgctggggttgggggctagaggagaggaggagcctctgttgagggctccaagaagggacagagaggtggtgctcatgggtcctggagacaccttttgggtggttcgtgccctccatcctgggccactttggggaggtgaaggagggaagcattaagggacaagaccccccgttcccaatttctctccggagccagggtttctctgattcgaagaaacaggtgtcacaacccaggaagtccactgatggcatctgccctggggcatcagcatttagggctgatcactgaggtctgcacctcccaaggctgctgtgcccattcctgggcgccccaaaggggaagaaaaactcctgaatgtgcaccgggacaggacccatcccatgcgg

SEQ ID NO: 23 - DNA, 800 bp, Homo sapiens
  source 1..800: mol_type = genomic DNA
agaaaattgccacctattgtattcaccatatgccaggcattgggctgggcacttgagtttatttttctaattcaagaacaaggccaggcacggtggctcatgcctgtaatcccagcactttgggaggctaaggcgggcagatcacaaggtcaggagtttgagaccagcctggccaacatggagaaatcccatctctactaaaaatacaaaaattagctgggtttcgtggtgtgcacctgtagtcccagctactcagggggctgaggcaggagaatctcttgaacccaggaggcggaggttgcagtgagccgagatcgcgccactgcactccagcctgagtgacagagtgagactccgtctaaaaaaaaagaacaaaagccgggcatggtggctcaagcctgtaatcccagcactttgggaggccaaggtgggcagatcatgaggtcagtagttccagaccagcctgaccaacatggtgaaaacctgtttctactaaaaatacaaaaattagctgggcgtggtggcaggcacttgcaatcccagctactcaggaggctgcagcaggagaatcacttgaacccgggaggcagaggttttagtgagctgagatcccgccactgcactccaacctgggcaacagagcaagactctgtctcaaaaaaaaaaaaaaagaaaaaagaaaaaaagaaaagaaaaaagaactaaaagagagtggagcacatttggcatgtgactatagtcccagctacttggtaggctgaggtgagaggatcacttgagccctggagtttgagcccagc

SEQ ID NO: 24 - DNA, 800 bp, Homo sapiens
  source 1..800: mol_type = genomic DNA
ctaggcagtgctgcaagaaccagtctctaaacaaacaaagaaaaaaaaacaggaaaaaaaaagagttatcctagcctagagaaatgttaacagtctatccccttaagatcataaaagtgaaataactggacagagtactgaaaaaagaagaaatctgttgaacaagtctaagtgatttgtagagtctctccaaaggacagtaaagatttcagaaccatggggaggaggctttcttgcagggtctgatgtatacctctagttctatttttagatatattccattgtgctctcttcatttgctcaccattagcatgggaattattccccttgttttatttatatttgtagtaacaagtctagcacagtaactggcatagaagtacttaataaatgttacaatgtgttgaagacttttgagatgtagaattaaattatttgaactacgtttcaggaagttgactctggaagctgtgtgatgaatgaatttagtgactaggagattaggcaaagagagcaattgggaggccacggcaatgatccagacaagagaagtctcagaactgtaatatgacattagcagcagggtagaaagaaggaaatacaaggtatatttcttcaggctttaggattaattggatttaggatgaaaggaaagagtaaattaaatccagtttcttggtgacatgagtgatgtcattaacctgtgacataggaaacactggaagaaaagcaggtttgaggtttaaaataatgttaaacaggccgggagcggtggctcacgcctgtaatcccaacatttt

SEQ ID NO: 25 - DNA, 20 bp, synthetic construct
  misc_feature 1..20: note = Oligonucleotides
  source 1..20: mol_type = other DNA
ttatcatgatactatgtccc

SEQ ID NO: 26 - DNA, 20 bp, synthetic construct
  misc_feature 1..20: note = Oligonucleotides
  source 1..20: mol_type = other DNA
agttactgagcacagtgcct

SEQ ID NO: 27 - DNA, 20 bp, synthetic construct
  misc_feature 1..20: note = Oligonucleotides
  source 1..20: mol_type = other DNA
gagttactgagcacagtgcc

SEQ ID NO: 28 - DNA, 20 bp, synthetic construct
  misc_feature 1..20: note = Oligonucleotides
  source 1..20: mol_type = other DNA
ttatcatgatactatgtccc

SEQ ID NO: 29 - DNA, 20 bp, synthetic construct
  misc_feature 1..20: note = Oligonucleotides
  source 1..20: mol_type = other DNA
agttactgagcacagtgcct

SEQ ID NO: 30 - DNA, 20 bp, synthetic construct
  misc_feature 1..20: note = Oligonucleotides
  source 1..20: mol_type = other DNA
gagttactgagcacagtgcc

SEQ ID NO: 31 - DNA, 21 bp, synthetic construct
  misc_feature 1..21: note = Oligonucleotides
  source 1..21: mol_type = other DNA
caagcatttatcatgatacta

SEQ ID NO: 32 - DNA, 21 bp, synthetic construct
  misc_feature 1..21: note = Oligonucleotides
  source 1..21: mol_type = other DNA
tcatgatactatgtcccaggc

SEQ ID NO: 33 - DNA, 20 bp, synthetic construct
  misc_feature 1..20: note = Oligonucleotides
  source 1..20: mol_type = other DNA
gcccaccttgaagatctgta

SEQ ID NO: 34 - DNA, 20 bp, synthetic construct
  misc_feature 1..20: note = Oligonucleotides
  source 1..20: mol_type = other DNA
tgcccaccttgaagatctgt

SEQ ID NO: 35 - DNA, 20 bp, synthetic construct
  misc_feature 1..20: note = Oligonucleotides
  source 1..20: mol_type = other DNA
caccttgaagatctgtaggg

SEQ ID NO: 36 - DNA, 20 bp, synthetic construct
  misc_feature 1..20: note = Oligonucleotides
  source 1..20: mol_type = other DNA
tgaaccaccgcacctggcct

SEQ ID NO: 37 - DNA, 21 bp, synthetic construct
  misc_feature 1..21: note = Oligonucleotides
  source 1..21: mol_type = other DNA
tccctccctacagatcttcaa

SEQ ID NO: 38 - DNA, 20 bp, synthetic construct
  misc_feature 1..20: note = Oligonucleotides
  source 1..20: mol_type = other DNA
gcccaccttgaagatctgta

SEQ ID NO: 39 - DNA, 20 bp, synthetic construct
  misc_feature 1..20: note = Oligonucleotides
  source 1..20: mol_type = other DNA
tgcccaccttgaagatctgt

SEQ ID NO: 40 - DNA, 20 bp, synthetic construct
  misc_feature 1..20: note = Oligonucleotides
  source 1..20: mol_type = other DNA
gggccaagaggctcagtcac

SEQ ID NO: 41 - DNA, 20 bp, synthetic construct
  misc_feature 1..20: note = Oligonucleotides
  source 1..20: mol_type = other DNA
ggataaagagatcaggtcca

SEQ ID NO: 42 - DNA, 20 bp, synthetic construct
  misc_feature 1..20: note = Oligonucleotides
  source 1..20: mol_type = other DNA
caccttgaagatctgtaggg

SEQ ID NO: 43 - DNA, 20 bp, synthetic construct
  misc_feature 1..20: note = Oligonucleotides
  source 1..20: mol_type = other DNA
tccctacagatcttcaaggt

SEQ ID NO: 44 - DNA, 20 bp, synthetic construct
  misc_feature 1..20: note = Oligonucleotides
  source 1..20: mol_type = other DNA
ttcaaggtgggcaaggaact

SEQ ID NO: 45 - DNA, 20 bp, synthetic construct
  misc_feature 1..20: note = Oligonucleotides
  source 1..20: mol_type = other DNA
tccctccctacagatcttca

SEQ ID NO: 46 - DNA, 20 bp, synthetic construct
  misc_feature 1..20: note = Oligonucleotides
  source 1..20: mol_type = other DNA
ctccctacagatcttcaagg

SEQ ID NO: 47 - DNA, 20 bp, synthetic construct
  misc_feature 1..20: note = Oligonucleotides
  source 1..20: mol_type = other DNA
accttgaagatctgtaggga

SEQ ID NO: 48 - DNA, 20 bp, synthetic construct
  misc_feature 1..20: note = Oligonucleotides
  source 1..20: mol_type = other DNA
cttcaaggtgggcaaggaac

SEQ ID NO: 49 - DNA, 20 bp, synthetic construct
  misc_feature 1..20: note = Oligonucleotides
  source 1..20: mol_type = other DNA
acagatcttcaaggtgggca

SEQ ID NO: 50 - DNA, 20 bp, synthetic construct
  misc_feature 1..20: note = Oligonucleotides
  source 1..20: mol_type = other DNA
aagagatcaggtccaaggcc

SEQ ID NO: 51 - DNA, 20 bp, synthetic construct
  misc_feature 1..20: note = Oligonucleotides
  source 1..20: mol_type = other DNA
gggcaaggaactgggccaag

SEQ ID NO: 52 - DNA, 20 bp, synthetic construct
  misc_feature 1..20: note = Oligonucleotides
  source 1..20: mol_type = other DNA
tagggagggataaagagatc

SEQ ID NO: 53 - DNA, 20 bp, synthetic construct
  misc_feature 1..20: note = Oligonucleotides
  source 1..20: mol_type = other DNA
atcaggtccaaggccaggtg

SEQ ID NO: 54 - DNA, 20 bp, synthetic construct
  misc_feature 1..20: note = Oligonucleotides
  source 1..20: mol_type = other DNA
aggtccaaggccaggtgcgg

SEQ ID NO: 55 - DNA, 20 bp, synthetic construct
  misc_feature 1..20: note = Oligonucleotides
  source 1..20: mol_type = other DNA
attaatgcctttaaataggt

SEQ ID NO: 56 - DNA, 20 bp, synthetic construct
  misc_feature 1..20: note = Oligonucleotides
  source 1..20: mol_type = other DNA
tcttaaacctacctatttaa

SEQ ID NO: 57 - DNA, 20 bp, synthetic construct
  misc_feature 1..20: note = Oligonucleotides
  source 1..20: mol_type = other DNA
gctaattaatgcctttaaat

SEQ ID NO: 58 - DNA, 20 bp, synthetic construct
  misc_feature 1..20: note = Oligonucleotides
  source 1..20: mol_type = other DNA
attaatgcctttaaataggt

SEQ ID NO: 59 - DNA, 20 bp, synthetic construct
  misc_feature 1..20: note = Oligonucleotides
  source 1..20: mol_type = other DNA
tcttaaacctacctatttaa

SEQ ID NO: 60 - DNA, 20 bp, synthetic construct
  misc_feature 1..20: note = Oligonucleotides
  source 1..20: mol_type = other DNA
ccacttccctcctccattaa

SEQ ID NO: 61 - DNA, 20 bp, synthetic construct
  misc_feature 1..20: note = Oligonucleotides
  source 1..20: mol_type = other DNA
gctaattaatgcctttaaat

SEQ ID NO: 62 - DNA, 20 bp, synthetic construct
  misc_feature 1..20: note = Oligonucleotides
  source 1..20: mol_type = other DNA
ctttaatggaggagggaagt

SEQ ID NO: 63 - DNA, 20 bp, synthetic construct
  misc_feature 1..20: note = Oligonucleotides
  source 1..20: mol_type = other DNA
cctttaatggaggagggaag

SEQ ID NO: 64 - DNA, 20 bp, synthetic construct
  misc_feature 1..20: note = Oligonucleotides
  source 1..20: mol_type = other DNA
tttaatggaggagggaagtg

SEQ ID NO: 65 - DNA, 21 bp, synthetic construct
  misc_feature 1..21: note = Oligonucleotides
  source 1..21: mol_type = other DNA
atggaggagggaagtggggtg

SEQ ID NO: 66 - DNA, 21 bp, synthetic construct
  misc_feature 1..21: note = Oligonucleotides
  source 1..21: mol_type = other DNA
agagaatctatgtcaccccac

SEQ ID NO: 67 - DNA, 21 bp, synthetic construct
  misc_feature 1..21: note = Oligonucleotides
  source 1..21: mol_type = other DNA
aaggcattaattagcgtttgc

SEQ ID NO: 68 - DNA, 21 bp, synthetic construct
  misc_feature 1..21: note = Oligonucleotides
  source 1..21: mol_type = other DNA
aataggtaggtttaagagaat

SEQ ID NO: 69 - DNA, 21 bp, synthetic construct
  misc_feature 1..21: note = Oligonucleotides
  source 1..21: mol_type = other DNA
catgcactactttaaaatttt

SEQ ID NO: 70 - DNA, 21 bp, synthetic construct
  misc_feature 1..21: note = Oligonucleotides
  source 1..21: mol_type = other DNA
aagtagtgcatgcaaacgcta

SEQ ID NO: 71 - DNA, 20 bp, synthetic construct
  misc_feature 1..20: note = Oligonucleotides
  source 1..20: mol_type = other DNA
cttgcagcactgcctaggct

SEQ ID NO: 72 - DNA, 20 bp, synthetic construct
  misc_feature 1..20: note = Oligonucleotides
  source 1..20: mol_type = other DNA
tggttcttgcagcactgcct

SEQ ID NO: 73 - DNA, 20 bp, synthetic construct
  misc_feature 1..20: note = Oligonucleotides
  source 1..20: mol_type = other DNA
tcttgcagcactgcctaggc

SEQ ID NO: 74 - DNA, 20 bp, synthetic construct
  misc_feature 1..20: note = Oligonucleotides
  source 1..20: mol_type = other DNA
cttgcagcactgcctaggct

SEQ ID NO: 75 - DNA, 20 bp, synthetic construct
  misc_feature 1..20: note = Oligonucleotides
  source 1..20: mol_type = other DNA
tggttcttgcagcactgcct

SEQ ID NO: 76 - DNA, 20 bp, synthetic construct
  misc_feature 1..20: note = Oligonucleotides
  source 1..20: mol_type = other DNA
tcttgcagcactgcctaggc

SEQ ID NO: 77 - DNA, 20 bp, synthetic construct
  misc_feature 1..20: note = Oligonucleotides
  source 1..20: mol_type = other DNA
taggctgggctcaaactcca

SEQ ID NO: 78 - DNA, 19 bp, synthetic construct
  misc_feature 1..19: note = Oligonucleotides
  source 1..19: mol_type = other DNA
ctaggctgggctcaaactc

SEQ ID NO: 79 - DNA, 20 bp, synthetic construct
  misc_feature 1..20: note = Oligonucleotides
  source 1..20: mol_type = other DNA
tgagaggatcacttgagccc

SEQ ID NO: 80 - DNA, 20 bp, synthetic construct
  misc_feature 1..20: note = Oligonucleotides
  source 1..20: mol_type = other DNA
ctggagtttgagcccagcct

SEQ ID NO: 81 - DNA, 20 bp, synthetic construct
  misc_feature 1..20: note = Oligonucleotides
  source 1..20: mol_type = other DNA
ttctttgtttgtttagagac

SEQ ID NO: 82 - DNA, 21 bp, synthetic construct
  misc_feature 1..21: note = Oligonucleotides
  source 1..21: mol_type = other DNA
agcccagcctaggcagtgctg

SEQ ID NO: 83 - DNA, 21 bp, synthetic construct
  misc_feature 1..21: note = Oligonucleotides
  source 1..21: mol_type = other DNA
gagactggttcttgcagcact

SEQ ID NO: 84 - DNA, 21 bp, synthetic construct
  misc_feature 1..21: note = Oligonucleotides
  source 1..21: mol_type = other DNA
tttagagactggttcttgcag

SEQ ID NO: 85 - DNA, 21 bp, synthetic construct
  misc_feature 1..21: note = Oligonucleotides
  source 1..21: mol_type = other DNA
tttgtttagagactggttctt

SEQ ID NO: 86 - DNA, 21 bp, synthetic construct
  misc_feature 1..21: note = Oligonucleotides
  source 1..21: mol_type = other DNA
tttgtttgtttagagactggt

SEQ ID NO: 87 - DNA, 20 bp, synthetic construct
  misc_feature 1..20: note = Synthetic
  source 1..20: mol_type = other DNA
aagggccaagggagttagtg

SEQ ID NO: 88 - DNA, 20 bp, synthetic construct
  misc_feature 1..20: note = Synthetic
  source 1..20: mol_type = other DNA
agctccctcactaactccct

SEQ ID NO: 89 - DNA, 20 bp, synthetic construct
  misc_feature 1..20: note = Synthetic
  source 1..20: mol_type = other DNA
agggccaagggagttagtga

SEQ ID NO: 90 - DNA, 20 bp, synthetic construct
  misc_feature 1..20: note = Synthetic
  source 1..20: mol_type = other DNA
atgaaaaataagcccggtag

SEQ ID NO: 91 - DNA, 20 bp, synthetic construct
  misc_feature 1..20: note = Synthetic
  source 1..20: mol_type = other DNA
caggctgagtcaccactacc

SEQ ID NO: 92 - DNA, 20 bp, synthetic construct
  misc_feature 1..20: note = Synthetic
  source 1..20: mol_type = other DNA
acaggctgagtcaccactac

SEQ ID NO: 93 - DNA, 800 bp, synthetic construct
  misc_feature 1..800: note = Synthetic
  source 1..800: mol_type = other DNA
aataatattaatactttattgtgattatctatgtagttcagtacagtacttagttcttattataaattagaaatgttagctgctattagtataactcttccatccttcacacttataacacccagaacacagtgtagttgtttcaagccttcagacttttttttttaagatggaatttcgcttttcttgcccaggcaggagtgcaatggcacgacttctgctcactgcaccctccacctccagcgttcaagcaattctcctgcctcagcctcccaagtagctgggattacaggcgcccaccaccacgcccggctaattttttgtatttttagtagagacggggtttcagtatgtcagccaggctggtctcaaactcctgacctcagttgatctacctgcctcagcctcccaaagtgctgggattacaggagtgagccactgagcccggccgccttcagacttttgtttgaacctggaaaactctttccctggagaaaacatgtctttcagcattactttctctccggctgtttcttacctttactcattcccaggatttacaattcccctcctttgtgtccaaacaatagcttgtatagccaaaaaaatcttttaagcaattttagttttacatttcttcctctgcctgctagagtgactttctggagagtaggatgtgggtgacatctgtctctgtgtccccaaagtcacaacaaagcatctgatataaaataggagctcagtcagtgagcattcaatcaatagtatcatgtttctgtctctgtccccagctccctcac

SEQ ID NO: 94 - DNA, 800 bp, synthetic construct
  misc_feature 1..800: note = Synthetic
  source 1..800: mol_type = other DNA
taactcccttggcccttatagtgggataatgaatgaggaagtgctgtctcatgctcattgagcatctcaatttttgttatttatcatactactttgcatagacgtgatttatttgctttttccttctttgcctgaggctgaatagttagttggcattattcctttttttaatttttatgtaacttttattcttttttatcaggcagccacccgagccagcagccagcgtaggctcagagagactcccctttctttcttgcaggagcaagggcagcatgaaagttcagagcttaataatggatggttattggctatgggcaggctaaaccaatacacagtacatacagtcatacttcagcccaaagaaaattgccacctattgtattcaccatatgccaggcattgggctgggcacttgagtttatttttctaattcaagaacaaggccaggcacggtggctcatgcctgtaatcccagcactttgggaggctaaggcgggcagatcacaaggtcaggagtttgagaccagcctggccaacatggagaaatcccatctctactaaaaatacaaaaattagctgggtttcgtggtgtgcacctgtagtcccagctactcagggggctgaggcaggagaatctcttgaacccaggaggcggaggttgcagtgagccgagatcgcgccactgcactccagcctgagtgacagagtgagactccgtctaaaaaaaaagaacaaaagccgggcatggtggctcaagcctgtaatcccagcactttgggaggccaaggtgggcagat

SEQ ID NO: 95 - DNA, 800 bp, synthetic construct
  misc_feature 1..800: note = Synthetic
  source 1..800: mol_type = other DNA
tgttcacgtgtttgtctgctgaccctctccccacaattgtcttgtgaccctgacacatccccctctccgagaaacacccacaaatgatcaataaatactaagggaactcagaggctggcgggatcctccatatgctgaacgctggtcccctgggtccccttatttctttctctatactttgtctctgtgtctttttcttttccaagtctctcgttccacctaacaagaaacacccacaggtgtggaggggcaacccaccccttcaccatctccacaaaaaataaaaaattagcccagtgtggtggtgtgtgcctgtggtcctaactagtccagaggctgaggtgggaggattgcttgagtcccagagctcgaggctgcagtgagccgagatggcaccactgtactccagcctgggtgacagagtgagcagagtgagaccctgtctcaaaaaataataaaataaaataatgggaacccaaatcacaagacagcactcatttttcttttatttattttattttttttgagacagagtcttgctctgctgcccagactggagtgcagcagcatggtttcggctcactgcaacctccacctcctgggttcaagtgattcttgtgcctcagcctcccaagtagctggaattacaggtatgtgccaccatgcctgactgatttttgtatttttaatagagacagggttacgccatgttgtccagtctggtttcgaactcctggtctcaagggattcacccatctcggcctctcaaagtactgggattacaggctgagtcaccacta

SEQ ID NO: 96 - DNA, 800 bp, synthetic construct
  misc_feature 1..800: note = Synthetic
  source 1..800: mol_type = other DNA
ccgggcttatttttcatacagatgatttagaatatgtttatgttaaatttttttagattatcactaaaagaaaacagttgaattcttcaatttacaaggattcaatttttaaaattatatttgaatgtacagaggcctacaaataccaatttcagaaacactgttatcaaggatatctatttgttaaaattgaatgcaactgaaagtaacagattaactgcaaataacaagcttaaccaaataggggattaatttttcccatttaacaaggagacagggccgggcacagtggctcacgcctgtaatcccagcactttgggaggtcgaggcgggtggatcacttgaggttgggagctcaagaccagcctgaccaacatggagaaaccccgtgtctacgaaaaatacaaaattagccggacgtgatggtacatgcctgtaatctcaactactcgggaggctgaggcaggagaatggcttgaacctgggaggtggaggttgcagtgagccaagatcgcgccatttgcattccagcctgggcaacaagacgagctccgtctcaaaaaaaaaaaaaaaaaaaaaaaagcaagtagacagggccaggtggagtggctcatggttgtaatcccagcattttgggaggccgagatgagaggatcatttgagctcaggagttggagaccagcctgggcgacataggggtacccatctctacaaaaaattaaacaaacaaacaaaaaatagccaaacatgatgacgcatgcctgtagtcccagctacccaggaggctgagatgagagaat

SEQ ID NO: 97 - DNA, 82 bp, synthetic construct
  misc_feature 1..82: note = Synthetic
  source 1..82: mol_type = other DNA
cctgtgtgtctggtgtgatctattggccccacgccccagattcaaaccaccactaaccatgcaggacacgggaaaaaaacag

SEQ ID NO: 98 - protein, 1307 aa, synthetic construct
  REGION 1..1307: note = Synthetic
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 99 - protein, 1331 aa, synthetic construct
  REGION 1..1331: note = Synthetic
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQRPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAARLGEKMLNKKLKDQKTPIPDTLYQELYDYVNHRLSHDLSDEARALLPNVITKEVSHEIIKDRRFTSDKFLFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNGRSSDDEATADSQHAAPPKKKRKV

SEQ ID NO: 100 - protein, 1103 aa, synthetic construct
  REGION 1..1103: note = Synthetic
MTIRSMKLKLKIYSGRSAPQLRQGLWRLHRLLNEGTAYYMDWLVHMRQEALPGKSKEEIRAELERRVRQQQEKNGVQNDQVPMDEVLSALRQLYELLVPSAVNNSGDAQTLSRKFLSPLVDPNSEGGKGTSNAGAKPGWRKKQEAGDPSWEKDYERWLKRKQADPTAEILGKLETAGLKPLFPLYTNEVKDIRWMPLTSKQYVRNWDRDMFQQAIEHLLSWETWNRKVNEERAKLKETVRRFEEQHLANGKDWLSPLQAYEANREQALRDMAISPSDRFRITRRQIKGWSELYERWNKLAPTASVEAYMQEVRHVQKKLGGTFGDADLYRFLAKPENVHIWRDHQERLHYYAAYNDLHKRLMSAKEQAAFTLPDPVAHPLWVRFDARDGNLFTYILQADSSKQRSRRYVNFSRFLWPVEDGYFEETENVKVELALSKQFYRQVIVHDNPTGKQKITFQDYSSKEILEGHLGGAKLQLDRNFLRKSGRDFETGDFGPAFLNVVLDLKPKQEVKNGRLQSPLGQALLVKSRPNDIPKVYGYKPDALAAWLEQASGEESLGSESLRQGFRVMSIDLGVRSAAAISVFSVKGEKTREGDKVCYPVGETGLFAVHDRSFLLRLPGESSEKRVNVERDKRKTERMQIRYHIRTLARVLRLANKATPMDRIKAVQDVLNDIESTRFMNDHDHHVYNHALETLRTYAPDHQGIWEEQVIAAHRQLEHHVGVIVGEWRKNWGKDRRGTVGLSMDNIEELDEMRRLLISWSRRARYPREAKPFQVNESNPVHLLRHLQNLKEDRLKQLANLIVMTALGYVYDSKEKKWKAAYPACQLILFEDLQRYRFHLDRSARENSQLMKWAHRSIPKYVWMQGEPYGLQIGDVWAGFTSRYHAKTGAPGIRCKALTEKDFQQGRLLESLVAEGMFTLQEVGTLKPGDIVPAEGGELFVTLADDSGDRIVITHADINAAQNVQKRFWLANSERFRVACRSVQIASQECFIPSSESVAKKMGKGVFVRDFSFHKDMEVYHWNNQVKLTAKNVPTDHSDDLQDLQDYQAILEEARESSSSYKTLFRDPSGFFFPDDVWVPQNIYWREVKKTITALLRKRIMST

SEQ ID NO: 101 - protein, 1091 aa, synthetic construct
  REGION 1..1091: note = Synthetic
MPIRSFKLKLVTHNGDSTYMDKLRRGLWKTHVIINRGIAYYMNTLALMRQEPYGSKSREEVRLDLLSTLREQQRRNNWSEQTGTDDELLSLSRRVYELLVPSAIGEKGDAQMLSRKFLSPLVDPNSEGGRGTAKSGRKPRWKKMMEEGHPDWEKEKEKDAAKKAEDPTASILADLEAVGLLPLFPLFSDEQKEIRWLPKKKRQFVRTWDRDMFQQALERMLSWESWNRRVAEEYLKLQAQRDEVYAKYLEDAGSWLNDLQTFEKQREEELAEVSFEPNSEYLITRRQIRGWKEVYEKWSKTSENASQEQLWRMVADVQTAMAGAFGDPKVYQFLSQPKHHHIWREHPNRLFYYSKYNEVREKLNRAKKQAAFTLPDPVEHPLWTRFDARGGNIHDYEISKVGKQYHVTFSSLILPEAQSWVEIENVTVGIGNSLQLKRQIRLDGYADKKQKVKYYDYSSRFELTGVLGGAKIQFDRKHLKKAAHRLAEGETGPIFLNVVVDVEPFLEVKNGRLRTPLGQVLQVNTRDWPKVVDYKAKELSVLMENTQIGNENGVSTIEAGMRIMSIDLGQRTAAAVSIFEVISKKPDEKETKLFYPIADTDLYAVHRRSLLLRLPGEEISSKKMIEKRKERARIRSLVRYQIRLLSEVLRLHTQGTAEQRRFKLDELLVSIQKKLELDQSEWISELEKLFDYIDESAEKWKEALVVAHRTLEPIVVEAVRNWKKSLSKENKDRRRIAGISIWSIEELEETRKLLIAWSKHSREPGIPKRLEKEETFAPEHLQHIQNVKDDRLKQMANLFVMTALGYKYDEGNKRWVEAYPACQVILFEDLSRYRFALDRPRRENNRLMKWAHRSIPRLTYMQAELFGIQVGDVYSAYTSRFHAKTGAPGIRCHALTEADLQSNSYVVNQLIKDKFIQDNQTEILKAGQIVPWQGGELFVTFADRSGASLAVIHADINAAQNLQKRFWQHNSEVFRVPCKVVKGGLVPVYEKMRKLFGKGLFVNIDDPESKEVYRWEHSTKMKSKTTPVDLESEDIDHEELSDEWEDMQEGYKTLLRDPSGFFWSSDSWIPQKDFWIRVKSRIGKSLREQIR

SEQ ID NO: 102 - protein, 1091 aa, synthetic construct
  REGION 1..1091: note = Synthetic
MPIRSFKLKLVTHNGDSTYMDKLRRGLWKTHVIINRGIAYYMNTLALMRQEPYGSKSREEVRLDLLSTLREQQRRNNWSEQTGTDDELLSLSRRVYELLVPSAIGEKGDAQMLSRKFLSPLVDPNSEGGRGTAKSGRKPRWKKMMEEGHPDWEKEKEKDAAKKAEDPTASILADLEAVGLLPLFPLFSDEQKEIRWLPKKKRQFVRTWDRDMFQQALERMLSWESWNRRVAEEYQKLQAQRDEVYAKYLEDAGSWLNDLQTFEKQREEELAEVSFEPNSEYLITRRQIRGWKEVYEKWSKTSENASQEQLWRMVADVQTAMAGAFGDPKVYQFLSQPKHHHIWREHPNRLFYYSKYNEVREKLNRAKKQAAFTLPDPVEHPLWTRFDARGGNIHDYEISKVGKQYHVTFSSLILPEAQSWVEIENVTVGIGNSLQLKRQIRLDGYADKKQKVKYYDYSSRFELTGVLGGAKIQFDRKHLKKAAHRLAEGETGPIFLNVVVDVEPFLEVKNGRLRTPLGQVLQVNTRDWPKVVDYKAKELSVLMENTQIGNENGVSTIEAGMRIMSIDLGQRTAAAVSIFEVISKKPDEKETKLFYPIADTDLYAVHRRSLLLRLPGEEISSKKMIEKRKERARIRSLVRYQIRLLSEVLRLHTQGTAEQRRFKLDELLVSIQRKLELDQSEWISELEKLFDYIDESAEKWKEALVVAHRTLEPIVVEAVRNWKKSLSKENKDRRRIAGISIWSIEELEETRKLLIAWSKHSREPGIPKRLEKEETFAPEHLQHIQNVKDDRLKQMANLFVMTALGYKYDEGNKRWVEAYPACQVILFEDLSRYRFALDRPRRENNRLMKWAHRSIPRLTYMQAELFGIQVGDVYSAYTSRFHAKTGAPGIRCHALTEADLQSNSYVVNQLIKDKFIQDNQTEILKAGQIVPWQGGELFVTFADRSGASLAVIHADINAAQNLQKRFWQHNSEVFRVPCKVVKGGLVPVYEKMRKLFGKGLFVNIDDPESKEVYRWEHSTKMKSKTTPVDLESEDIEHEELSDEWEDMQEGYKTLLRDPSGFFWSSDSWIPQKDFWIRVKSRIGKSLREQIR